(19) United States
(12) Patent Application Publication
(10) Pub. No.: US 2025/0276069 A1
(43) Pub. Date: Sep. 4, 2025

(54) HETEROBIFUNCTIONAL COMPOSITIONS FOR TARGETED PROTEIN DEGRADATION AND METHODS FOR THEIR USE

(71) Applicant: THE SCRIPPS RESEARCH INSTITUTE, La Jolla, CA (US)

(72) Inventors: Christopher Parker, Palm Beach Gardens, FL (US); Louis Patrick Conway, Palm Beach Gardens, FL (US); Ines Forrest, Jupiter, FL (US); Christian Chaheine, San Diego, CA (US)

(21) Appl. No.: 18/548,635

(22) PCT Filed: Mar. 4, 2022
(86) PCT No.: PCT/US22/18944

§ 371 Date: Sep. 1, 2023

Related U.S. Application Data
(60) Provisional application No. 63/156,593, filed on Mar. 4, 2021.

(51) Int. Cl. A61K 47/55; A61K 47/54

(52) U.S. Cl. CPC A61K 47/55; A61K 47/545

(57) ABSTRACT

Compositions and methods for control and/or modification of endogenous protein degradation are described. The compositions are directed to heterobifunctional molecules having protein and enzyme system binding moieties linked together by an organic linker group. The compositions are selective for binding to certain endogenous proteins and function to recruit endogenous decomposition systems such as the polyubiquitin system for peptide cleavage and reassimilation.

### CROSS-REFERENCE TO RELATED APPLICATIONS

The subject patent application claims the benefit of priority to U.S. Provisional Patent Application Nos. 63/156,593 (filed Mar. 4, 2021; now pending). The full disclosure of the priority application is incorporated herein by reference in its entirety and for all purposes.

### BACKGROUND

A powerful approach was recently developed that integrates fragment-based ligand discovery with chemical proteomics, called fragment-based ligand mapping in cells, to globally survey ligandable proteins and their ligandable sites (FbLMiC, FIG. 1A) (11). In FbLMiC, small-molecule probes, called fully functionalized fragment (FFF) probes are designed to contain: 1) a structurally minimized “constant” region bearing a photoactivatable diazirine group and alkyne handle, which together enable UV light-induced covalent modification and detection, enrichment, and identification of compound-bound protein targets; and 2) a “variable” recognition region consisting of structurally diverse small-molecule fragments (MW<300 Da) to promote interactions with a subset of the proteome. See FIGS. 1B and 1C.

Notable strengths of FbLMiC are: 1) fragments can be optimized into higher affinity ligands through FbLMiC-guided medicinal chemistry, resulting in lead chemical probes that can selectively modulate protein function (as has been shown with first-in-class chemical probes for PTGR2, SLC25A20 and PGRMC2, (11, 12)); 2) FFFs can map ligand binding site on endogenous protein targets, revealing fragments that interact at a variety of protein sites (e.g. active sites, cofactor binding sites, allosteric sites); 3) FFF probes engage proteins reversibly, overcoming the limitations of other chemical proteomic profiling techniques, such as activity-based protein profiling (ABPP), which require covalent reactions with amino acid side chains. Recently this platform has been expanded and has demonstrated that enantiomerically matched FFF's can be used to expedite the discovery of selective fragment-protein interactions (13). See enantioprobes; FIG. 1C.

To streamline the identification of high affinity and selectivity chemical probes against proteins with established FFF leads, a strategy has recently been developed and is termed competitive FbLMiC (FIGS. 2A and 2B). Here, the potency and selectivity of candidate small-molecule ligands is assessed across 100s-1000s of proteins. This method integrates targeting and tandem mass tagging (TMT) methods (14) to enable the optimization of chemical probes for protein targets directly within their endogenous biological environments. In competitive FbLMiC, up to nine different competitor analogs (and a DMSO control) are evaluated per experiment for blockade of FFF interactions with endogenous targets. Competitor libraries consist of members that share parent fragment core structures, either purchased or synthesized in-house. By performing targeted MS experiments, MS runtimes can be dramatically shortened, enabling the screening of ˜150 compounds/day. Analogs that show the strongest blockade are optimized for potency and proteomic selectivity by an iterative cycle of MS-based FbLMiC-guided medicinal chemistry to furnish lead ‘binders.’ The original pilot FFF library consisted of only 13 members for proof of principle studies (11). For this work, a specialized library of ˜150 “scout” FFF probes was synthesized and was based upon fragment (<250 Da) cores commonly found in bioactive compounds (e.g. drugs, natural products, human metabolites) (15) and possessed structures that are synthetically accessible for derivatization. Initial profiling of this library has demonstrated outstanding coverage with an unprecedented ligandability map of 5000+ proteins, including those that fall out of traditional “druggable” classes (e.g. adaptor proteins, transcription factors). However, the protein binding activity accomplished with members of the FFF library does not enable refined selection of the proteins of interest (POIs), modification of the POIs and/or their selective, programmed degradation.

A rudimentary solution involves reliance upon rationally designed heterobifunctional small molecules. These heterobifunctional molecules facilitate the study of an increasingly wide range of biological phenomena and have proven enabling in drugging challenging therapeutic targets and processes. Such molecules typically consist of two ligands or binders that are connected via a covalent linker, yielding a chimeric compound that can mediate the formation of ternary complexes between two unique proteins (6, 7). The ability to “recruit” enzymes to proteins of interest (POIs) endows these molecules with great potential as a generalizable strategy to influence PTMs.

Recently, it has been shown that one class of PTM enzymes, E3 ubiquitin ligases, can be recruited to target POIs via heterobifunctional molecules (8-10). These molecules, often referred to as proteolysis targeting chimeras (PROTACs), induce molecular proximity between an E3 ligase and a POI, leading to ubiquitination and targeted protein degradation (TPD). Despite the tremendous therapeutic potential of TPD, two major challenges overshadow the generalization of this approach, i) the small number of E3 ligase recruiters that have been identified, despite the excess of 600 predicted E3 ligases; and ii) the undetermined fraction of the proteome accessible to TPD, and correspondingly, the availability of small molecule ligands capable of binding to target POIs that can be coopted into such modalities.

Nearly all PROTACs utilize established small molecule ligands (e.g. inhibitors), limiting the scope of proteins that can be targeted and requiring synthetic ‘retrofitting’ that may be disruptive to binding to the POI. Further, due to this dearth of available ligands, the fraction of the proteome that can be targeted with PROTAC-type strategies is unknown. Therefore, a goal of the present invention is the development of PROTAC's that are based upon surveys of the entire proteome for proteins that may be tractable to targeted protein degradation in parallel to the identification of bifunctional degrader leads instead of choosing singular targets with a priori established ligands. Therefore, a goal of the present invention is the development of heterobifunctional compositions that functions to bind endogenous proteins and to modify them and/or to enable programmed selective degradation.

### SUMMARY

An aspect of the present invention is directed to heterobifunctional fragment based degrader molecules, FragTACS, having protein binding target moieties that are selected from unbiased whole proteome affinity interactions. Another aspect of the present invention is directed to methods for in vitro and/or in vivo endogenous protein degradation through the agency of heterobifunctional FragTACs.

Embodiments of the heterobifunctional FragTACS incorporate small molecule fragments that are preferably endogenous protein binding target moieties, small molecule recruiter moieties for preferably endogenous degradation enzymes and an organic group linking these two moieties together. The FragTACS may be depicted by a generic Formula I:

PBF-L-RBF Formula I

The acronym PBF is a protein binding fragment of a small organic molecule moiety that enables selection of the protein of interest from a milieu of proteins, preferably endogenous proteins. The acronym RBF is a recruiter binding fragment of moiety of a small organic molecule moiety that is capable of recruiting in a cytoplasm context an enzyme that degrades, fragments and/or divided endogenous proteins into fragments for re-assimilation. The acronym L is an organic linker group having combinable, reactive functions at its termini that enable covalent attachment to the PBF and RBF. Exemplary PBF fragments include but are not limited to:

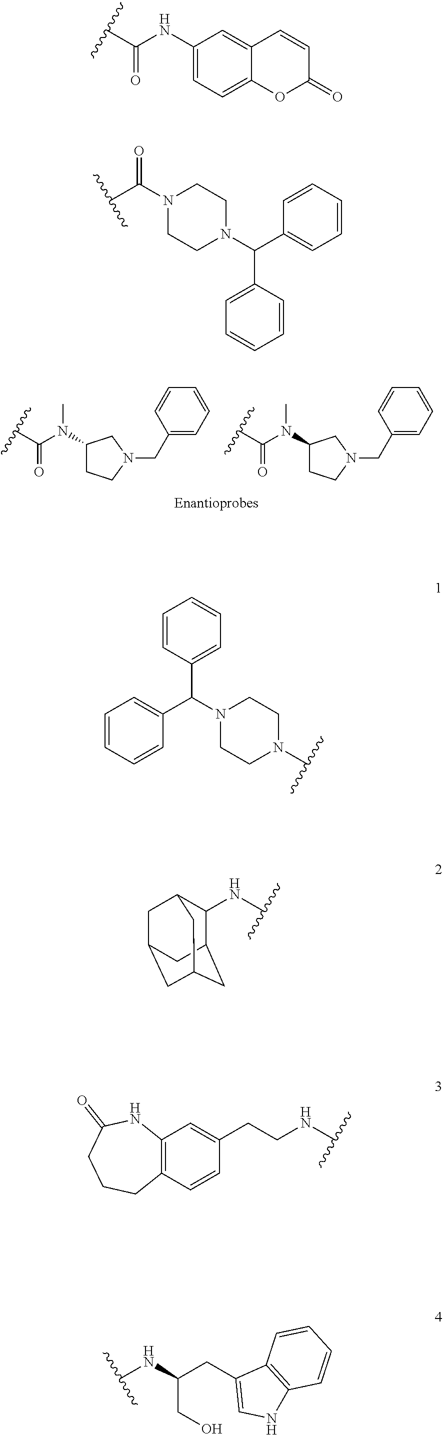

Exemplary RBF fragments include but are not limited to thalidomide derivatives for the CRBN ligase and the N-(4-thiazol-1-yl phenethyl) 4-hydroxyprolinamide for the VHL ligase

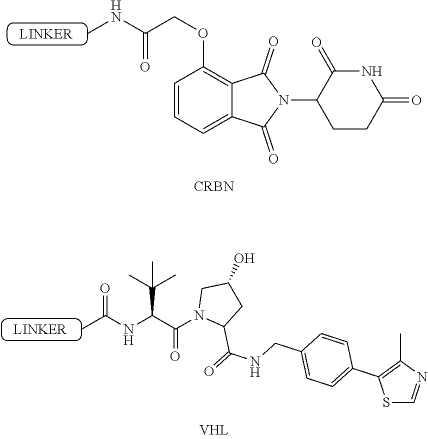

These examples of RBF fragments enable recruitment of E3 ubiquitin ligase activity. Exemplary linker groups include short and oligomeric polyol (PEG) and alkylenyl, moieties having amine and/or carboxyl termini for binding to the PBF and RBF fragments.

### BRIEF DESCRIPTION OF THE DRAWINGS

FIGS. 1A, 1B and 1C depict Fragment-based Ligand-ability Mapping in Cells. FIG. 1A depicts Fully functionalized fragment (FFF) probes are composed of a drug-like fragment as well as a retrieval tag, enabling the covalent capture of fragment-bound protein targets directly in cells upon UV irradiation. Fragment targets, as well as the site of fragment interaction, can be identified and quantified by mass spectrometry- and gel-based methods. FIG. 1B depicts the general structure of FFF showing the constant affinity tag region (red), which consists of a photoreactive group (diazirine) and a latent affinity (alkyne) group, as well as the variable region (blue), which contains fragment recognition elements for binding to proteins in cells. FIG. 1C depicts example structures of fragment scout- and enantioprobes.

FIGS. 2A and 2B depict targeted competitive FbLMiC workflow for chemical probe development for prioritized proteins. FIG. 2A depicts optimization of scout probe to lead binder through iterative competitive FbLMiC. FIG. 2B depicts targeted MS-FbLMiC accelerates chemical probe development by enabling rapid, multiplexed analyses of a defined set of prioritized targets in native biological systems. Shown is a targeted MS-FbLMiC workflow that we have developed for assessing small molecule interactions in prioritized targets, where a cell preparation is treated with up to nine compounds (and a DMSO control) and a corresponding FFF scout probe, click conjugation to a desthiobiotin-azide tag, streptavidin enrichment, elution, and labeling of enriched peptides with tandem-mass tags (TMT). Samples are then combined and analyzed in a single LC-MS/MS/MS experiment, where peptides of interest in prioritized proteins are selected for sequential MS1, MS2, and MS3 analysis. MS3 signals are used to quantify peptides, where reductions in signal (e.g., >80%) mark compound-sensitive sites.

FIGS. 3A, 3B and 3C depict fragment-based discovery of degradable proteins. FIG. 3A depicts chemical structures of a preliminary set of fragment-based PROTACs (FragTACs), which consist of a small molecule fragment chemically linked to established E3 recruiting ligands. FIG. 3B shows representative data for a FragTAC (1, 100 μM, 6 hrs) incubated with HCC1806 cells. A relative abundance of ˜7000 proteins was determined using quantitative multiplexed proteomics. Vertical dashed lines mark 3-fold changes relative to DMSO and horizontal dashed lines mark p value=0.05. Red box (inset) shows targets with >3-fold decreased abundance. FIG. 3C depicts western blots confirming dose-dependent downregulation of 3 example targets (TDP-43, FAM136A, and CCAR2).

FIGS. 4A, 4B, 4C and 4D depict expanding the druggable landscape of for targeted protein degradation. FIG. 4A provides the strategy to expand the number of targets and E3 proteins available for TPD approaches. FIG. 4B shows a synthesizable library of ˜300 FragTACs and examine their ability to modulate protein levels via unbiased proteome-wide proteomic analyses in primary human immune cells. FIG. 4C shows a representative subset of E3 ligases for which FbLDisC has identified hit ligands. Note that hit ligands have been discovered for members of most E3 subfamilies. FIG. 4D shows a model system for testing whether druggable sites on E3 ligases support TPD. Ligands for a given E3 are coupled to a compound AP11867 that binds to the FKBP12-F36V protein and assayed for inducing degradation of recombinant FKBP12-F36V in cells that endogenously express the E3 ligase.

FIG. 5 depicts in schematic style how a FragTAC binds to a target protein and recruits a fragmentation ligase, which in this example is the ubiquitin ligase complex. The ubiquitin ligase ubiquitinizes the protein to add ubiquitin peptide chains to the protein. This ubiquitination marks the protein for proteolysis through proteasome enzymatic degradation to yield amino acid fragments for re-assimilation.

FIG. 6 shows results of Western blot studies of proteasome/neddylation inhibition of several target proteins in HCC1806 cells.

FIG. 7 shows results of Western blot studies of dose-dependent response of several target proteins.

FIG. 8 shows results of Western blot studies of time course of the responses of several target proteins.

FIG. 9 shows results of cell viability assays of representative examples.

### DETAILED DESCRIPTION

In general, the invention provides heterobifunctional FragTACs that contain a protein binding fragment (PBF) and a recruiter binding fragment (RBF) that are connected via a linker moiety (L). As exemplified herein, the PBF moiety can be any small molecule that targets one or more endogenous proteins, the RBF moiety can be any compound that recruits one or more endogenous degradation enzymes, and the L moiety can be any organic group that optimally links the two moieties with no or minimum impact on their biological functions.

In various embodiments, the PBF moiety can be selected from any suitable commercially available fragments or synthetically accessible fragments, as described herein. Exemplary commercially available and synthetically derivatizable PBF fragments include but are not limited to:

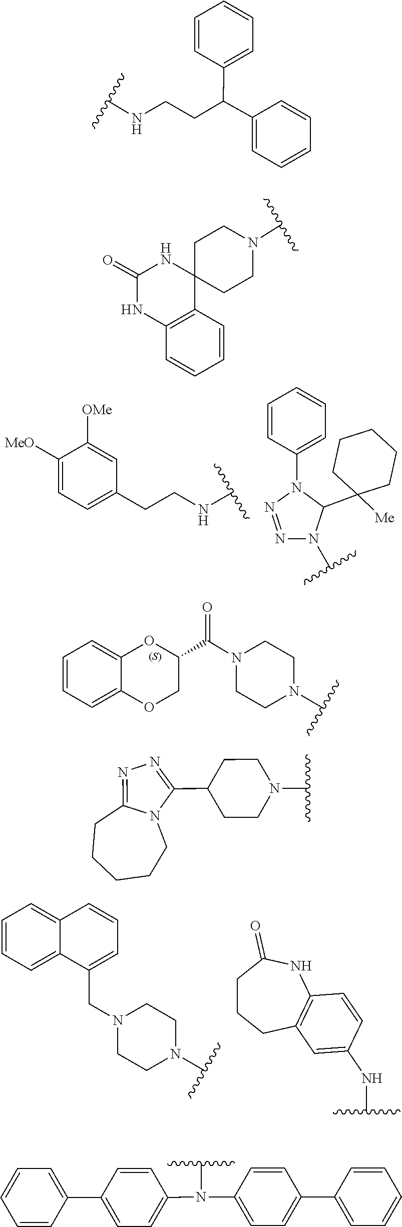

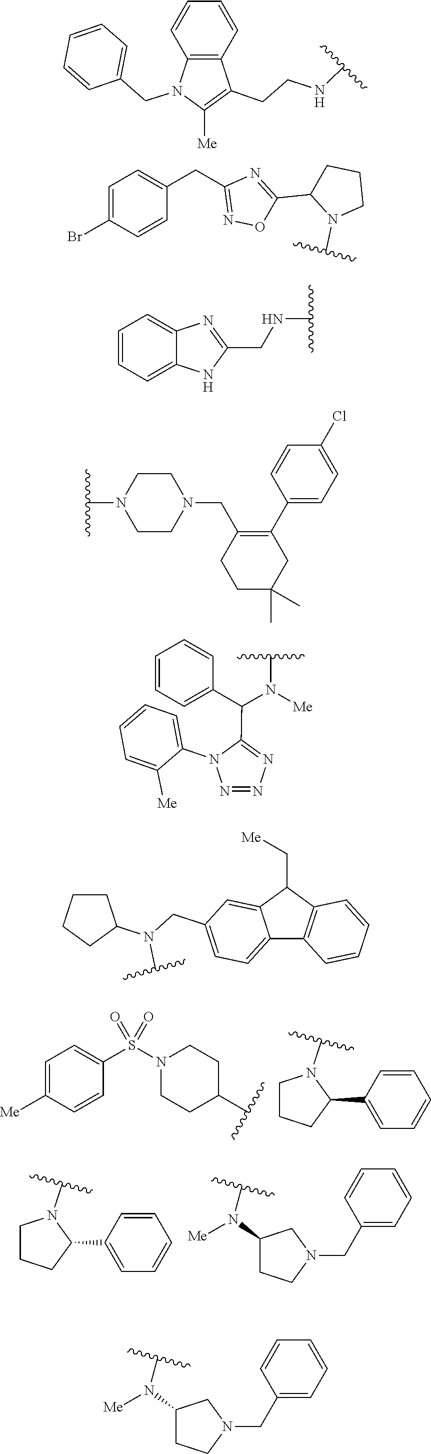

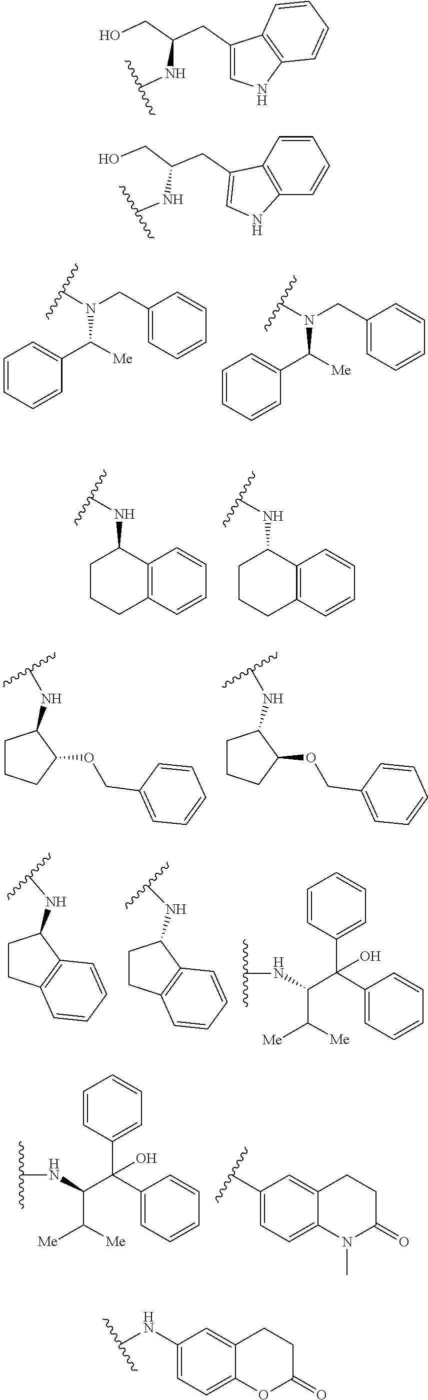

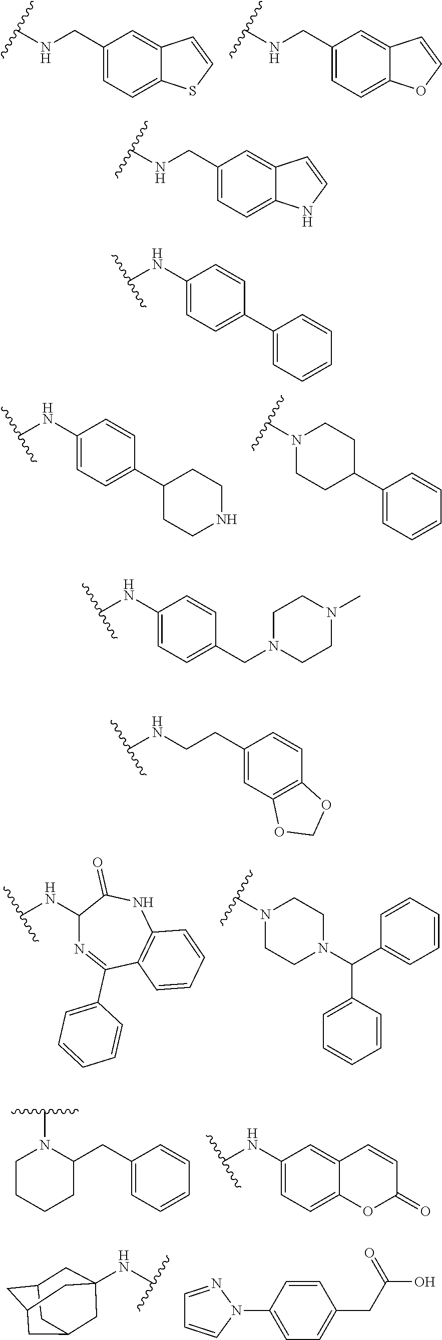

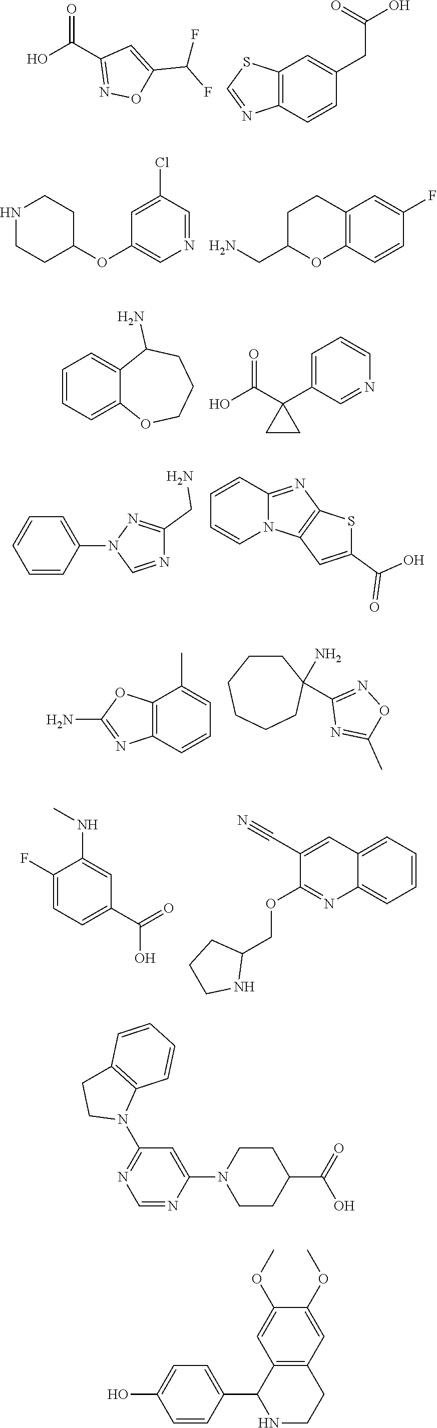

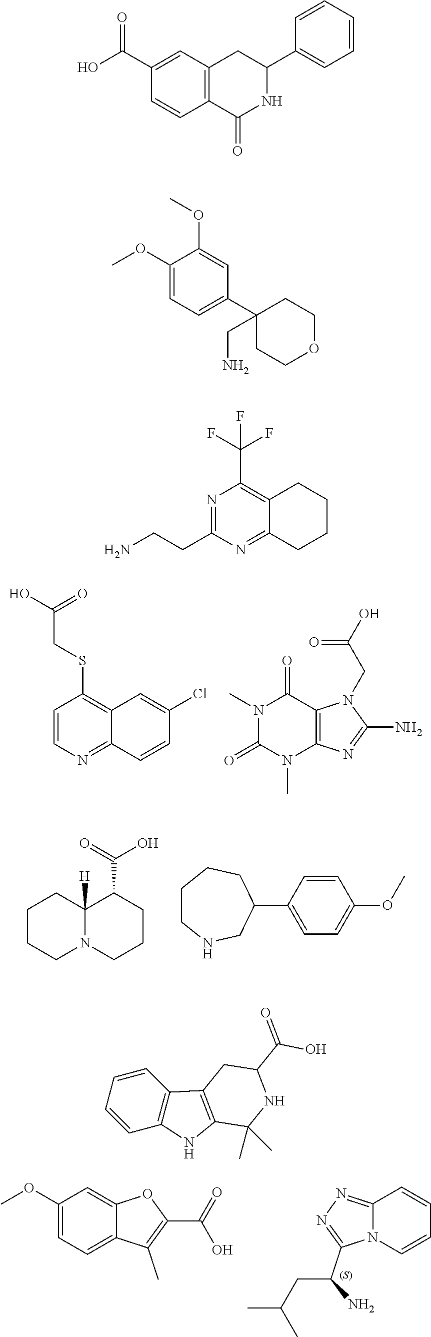

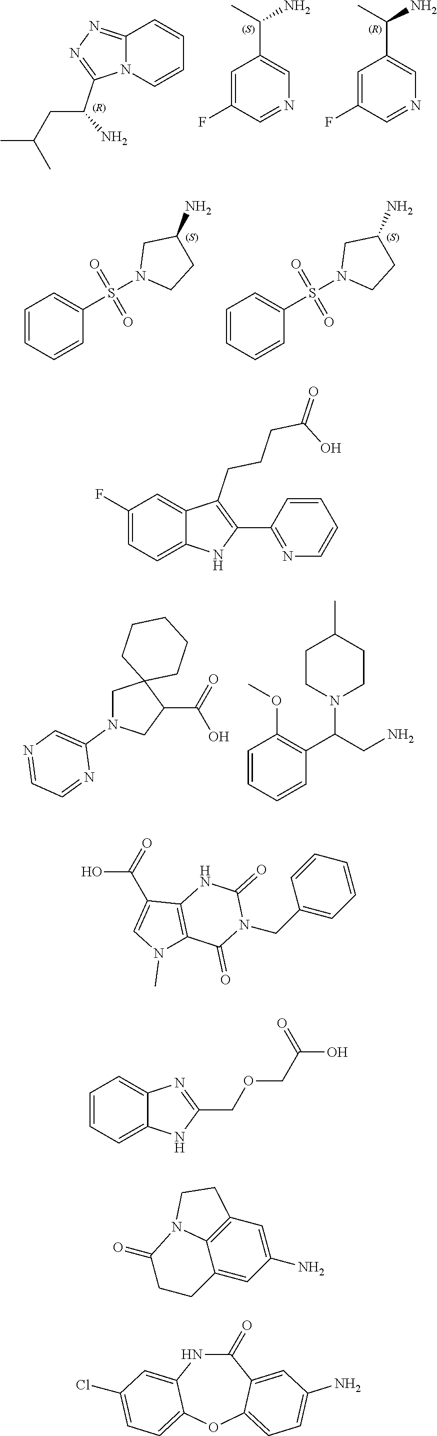

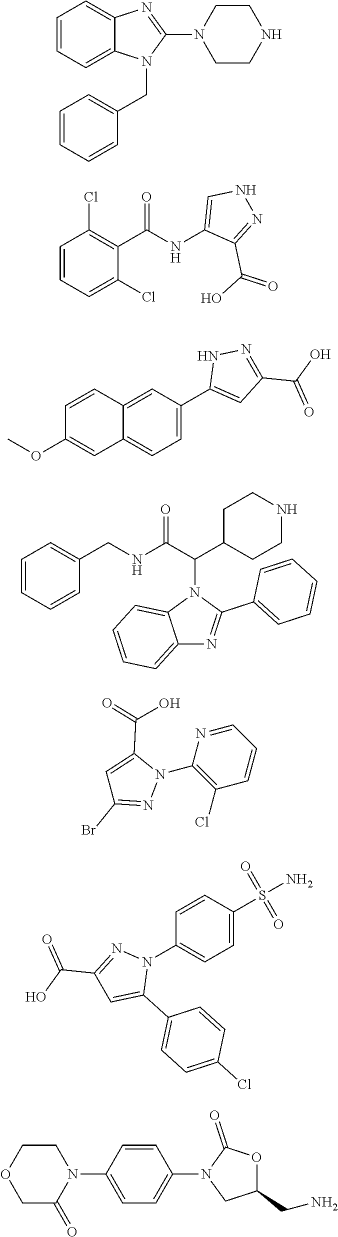

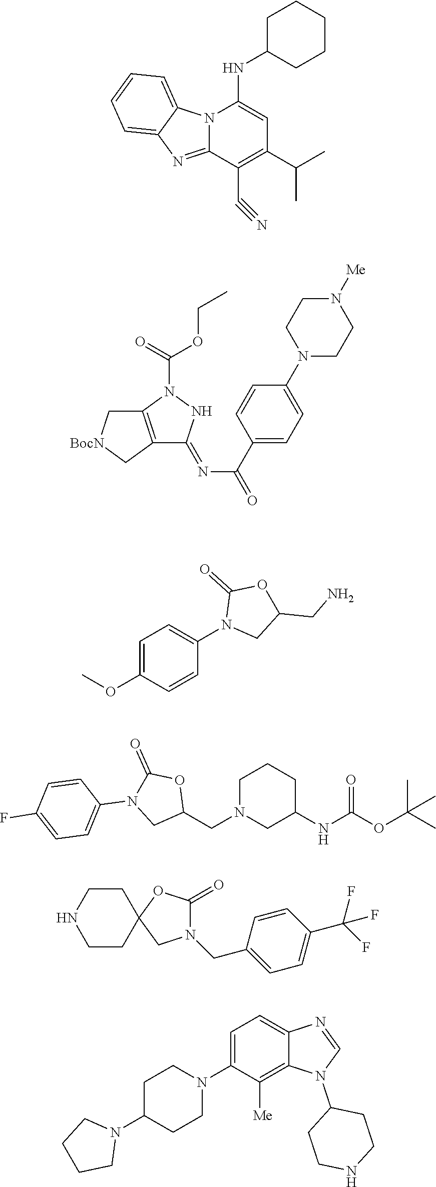

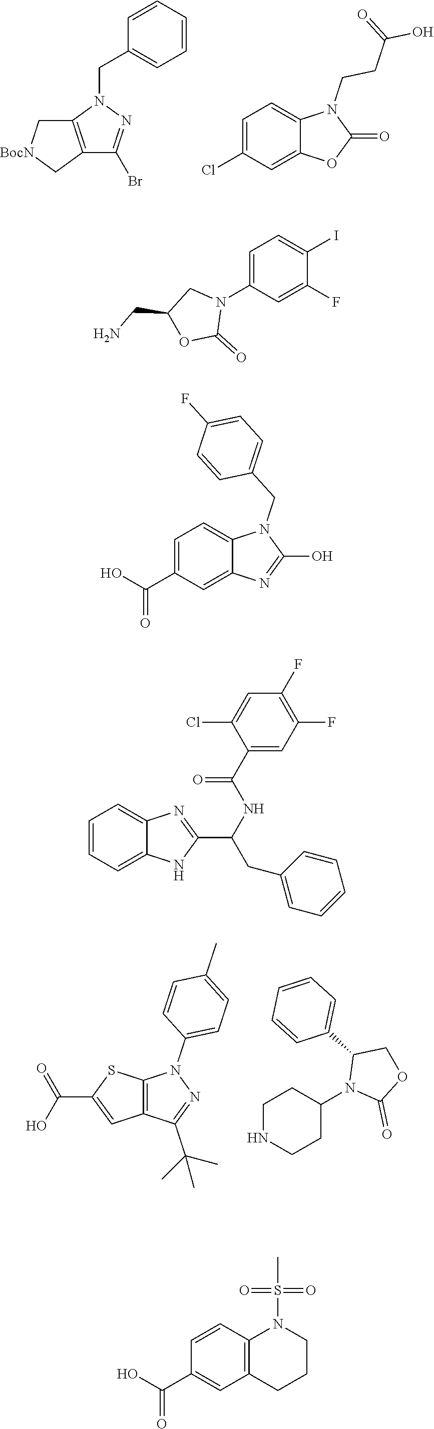

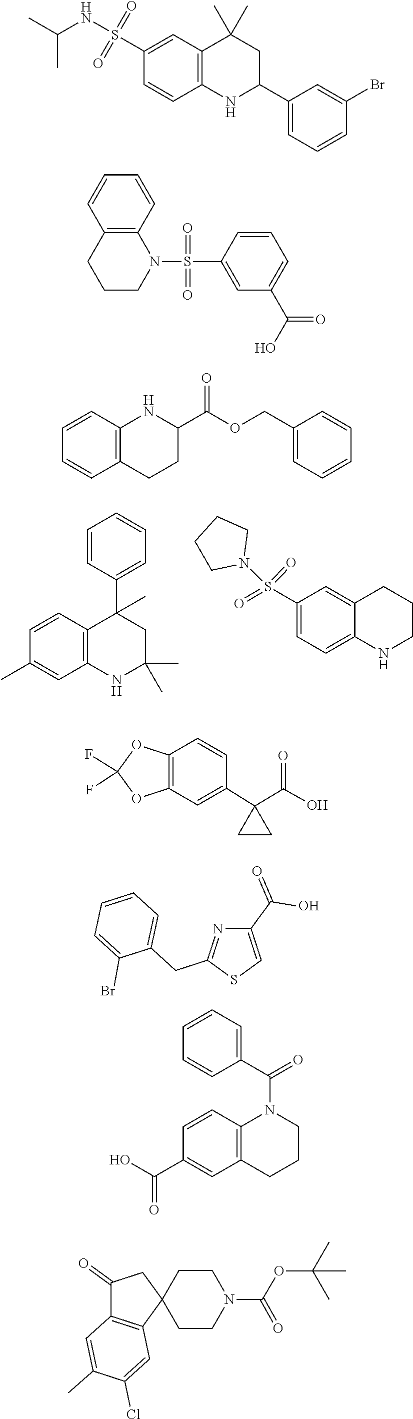

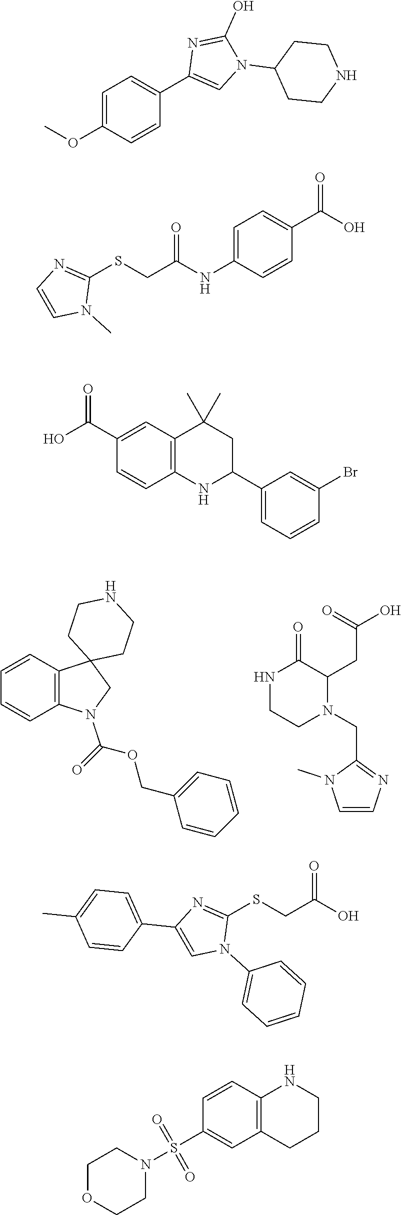

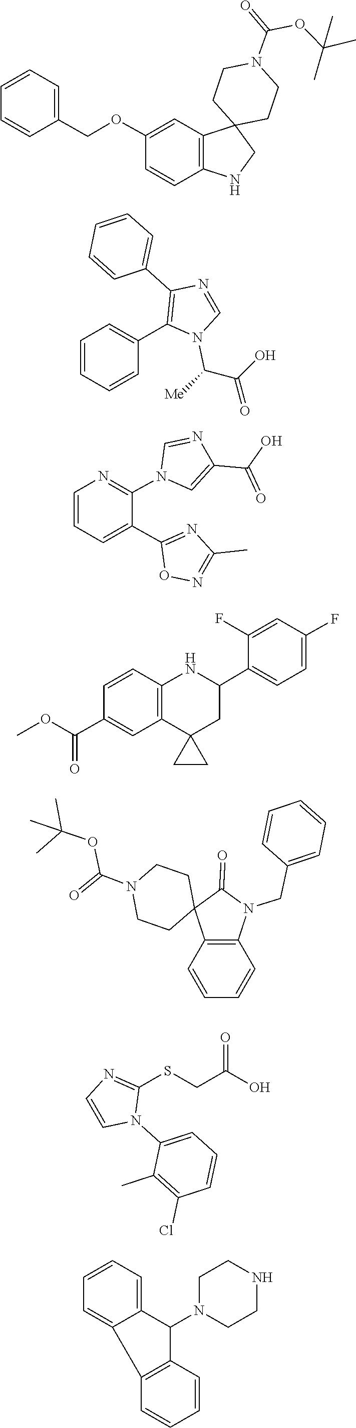

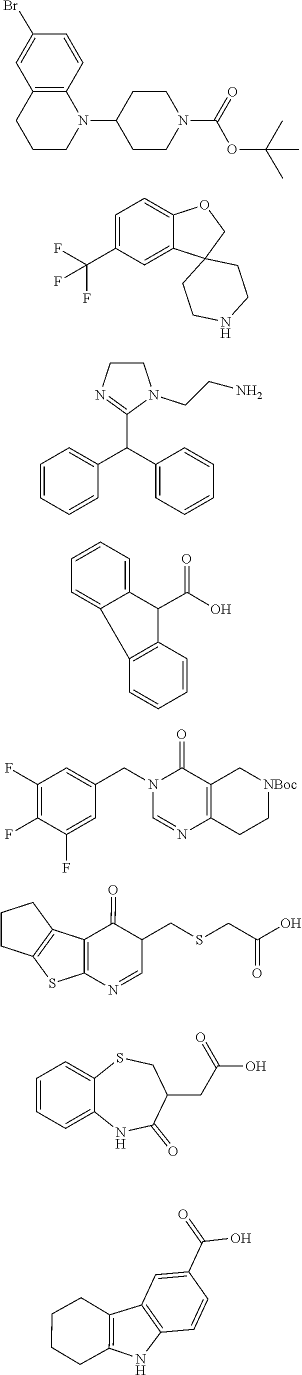

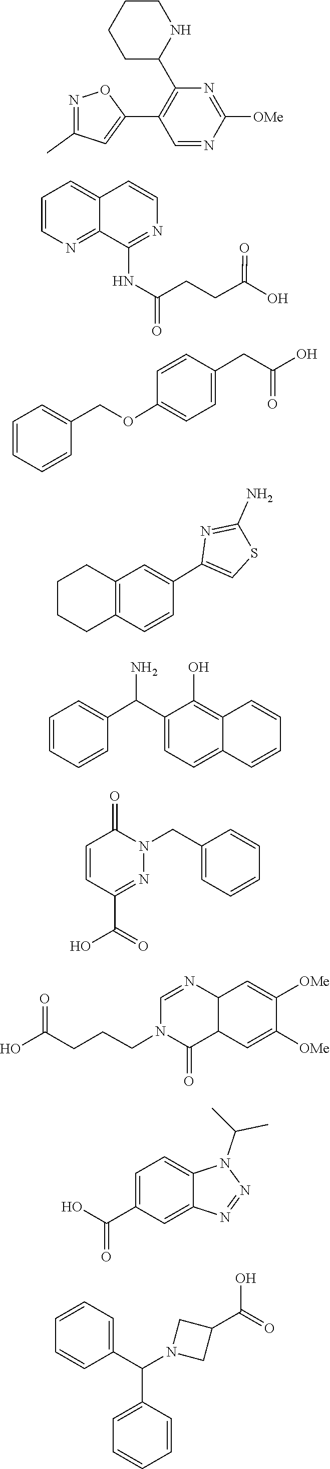

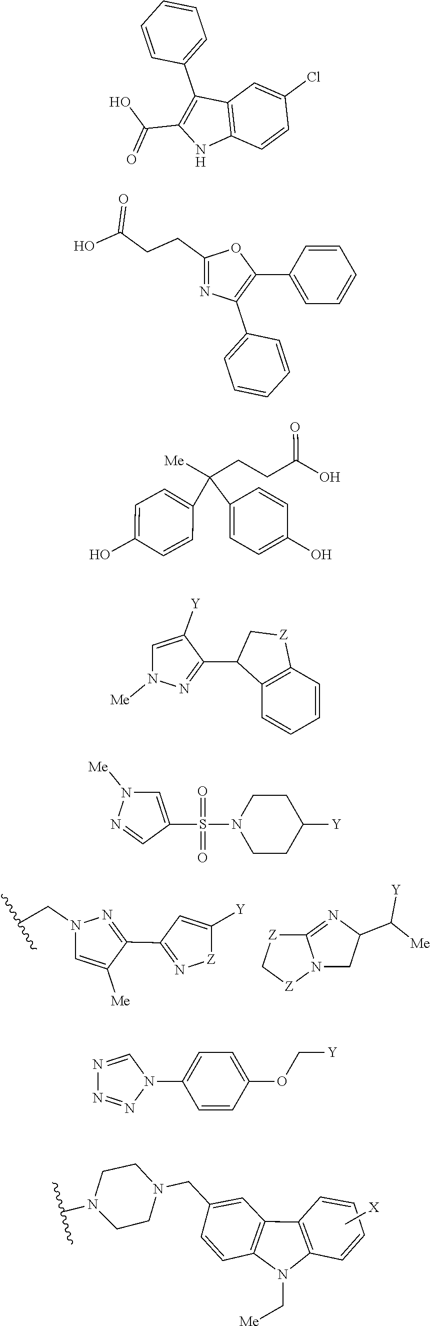

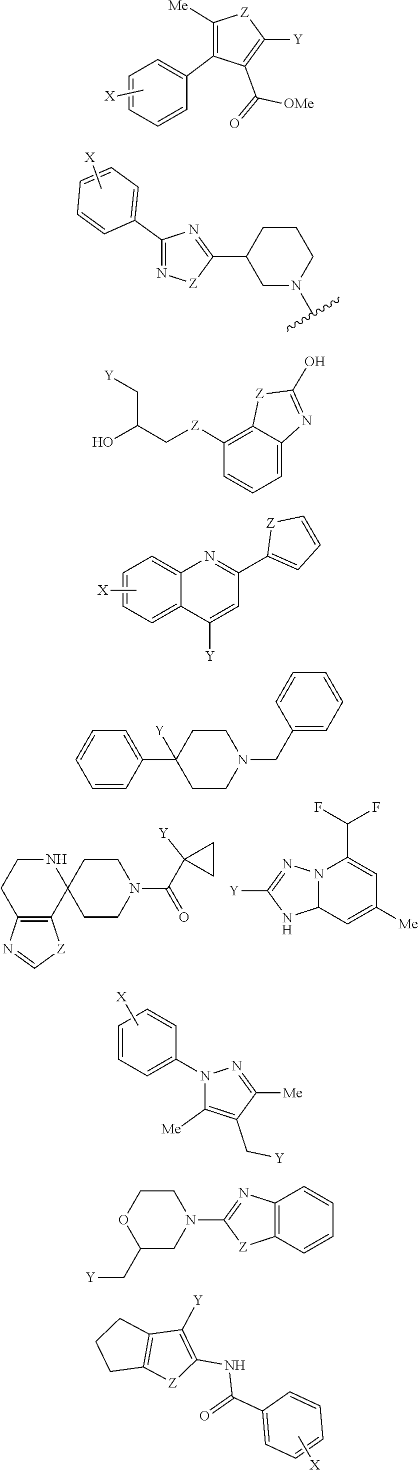

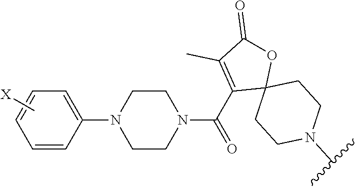

In these compound structures, X is F, Cl, Br or I; Y is COOH or NH₂; Z is O, NH or S.

In some embodiments, the PBF moiety is a synthetically accessible fragment. Exemplary synthetically accessible PBF fragments include but are not limited to:

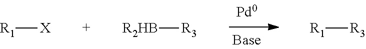

Suzuki-Miyaura Coupling
Organoboranes
General Examples

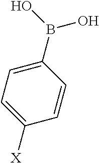

X = F, Cl, Br, I

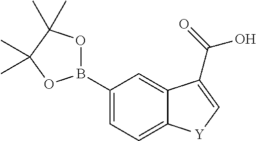

Y = NH, S, O

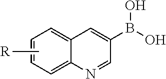

R = F, Cl, Br, I, alkyl, acyl

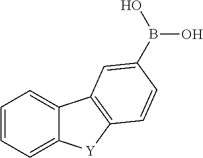

Y = NH, S, O
Specific Examples

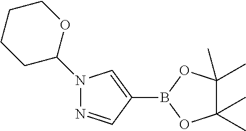 [SMILES: CC1(C)OB(c2cnn(C3CCCCO3)c2)OC1(C)C]

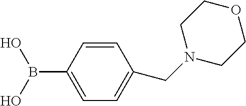 [SMILES: OB(O)c1ccc(CN2CCOCC2)cc1]

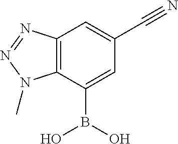

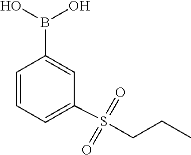

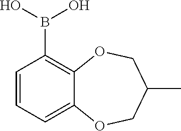

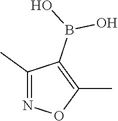

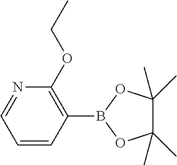 [SMILES: CCOc1ncccc1B1OC(C)(C)C(C)(C)O1]

Organohalides
Aliphatic

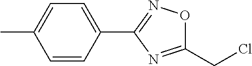

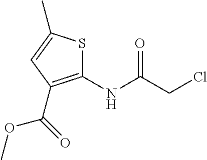

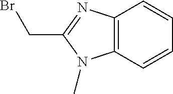

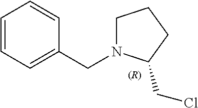

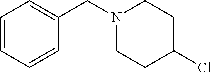 [SMILES: ClC1CCN(Cc2ccccc2)CC1]

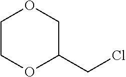

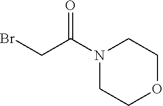

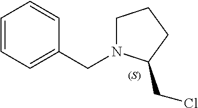

Aromatic

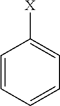

X = Cl, Br, I

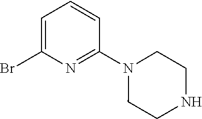 [SMILES: Brc1cccc(N2CCNCC2)n1]

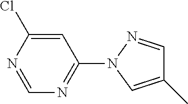

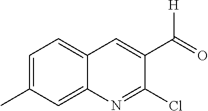

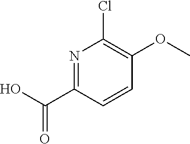

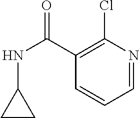 [SMILES: O=C(NC1CC1)c1cccnc1Cl]

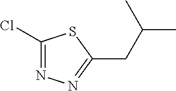

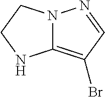

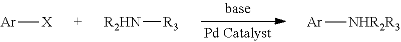

Buchwald-Hartwig Amination
Aryl Halides
Bromides

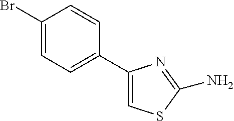 [SMILES: Nc1nc(-c2ccc(Br)cc2)cs1]

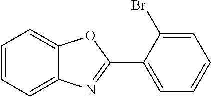 [SMILES: Brc1ccccc1-c1nc2ccccc2o1]

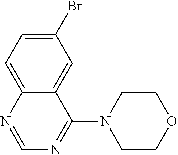 [SMILES: Brc1ccc2ncnc(N3CCOCC3)c2c1]

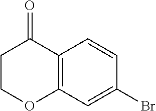 [SMILES: O=C1CCOc2cc(Br)ccc21]

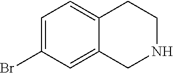 [SMILES: Brc1ccc2c(c1)CNCC2]

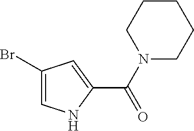 [SMILES: O=C(c1cc(Br)c[nH]1)N1CCCCC1]

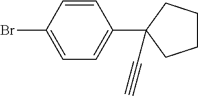

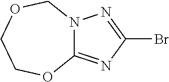 [SMILES: Brc1nc2n(n1)COCCO2]

Iodides

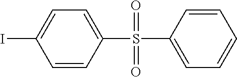 [SMILES: O=S(=O)(c1ccccc1)c1ccc(I)cc1]

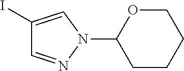 [SMILES: Ic1cnn(C2CCCCO2)c1]

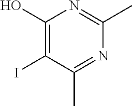

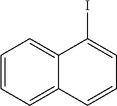 [SMILES: Ic1cccc2ccccc12]

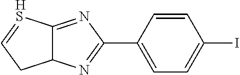

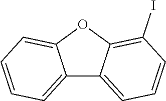 [SMILES: Ic1cccc2c1oc1ccccc12]

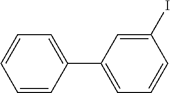 [SMILES: Ic1cccc(-c2ccccc2)c1]

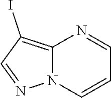

Secondary Amines

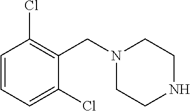 [SMILES: Clc1cccc(Cl)c1CN1CCNCC1]

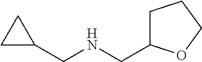 [SMILES: C1COC(CNCC2CC2)C1]

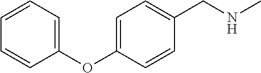 [SMILES: CNCc1ccc(Oc2ccccc2)cc1]

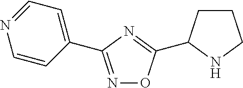 [SMILES: c1cc(-c2noc(C3CCCN3)n2)ccn1]

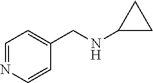 [SMILES: c1cc(CNC2CC2)ccn1]

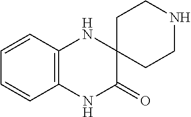 [SMILES: O=C1Nc2ccccc2NC12CCNCC2]

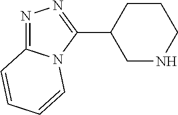 [SMILES: c1ccn2c(C3CCCNC3)nnc2c1]

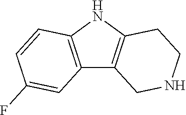 [SMILES: Fc1ccc2[nH]c3c(c2c1)CNCC3]

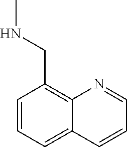

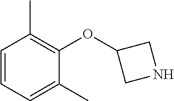

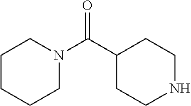 [SMILES: O=C(C1CCNCC1)N1CCCCC1]

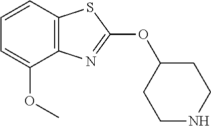 [SMILES: COc1cccc2sc(OC3CCNCC3)nc12]

Pictet Spengler Reaction

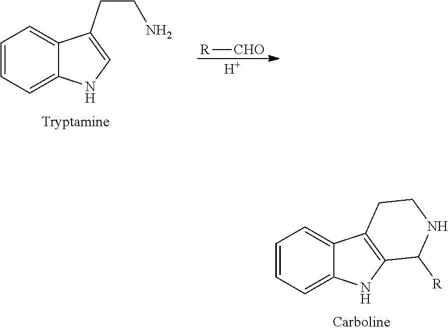

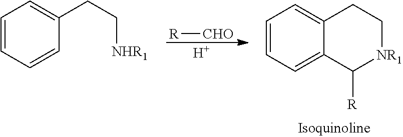

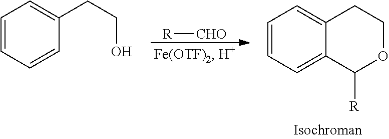

Aldehydes

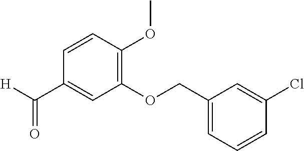 [SMILES: COc1ccc(C=O)cc1OCc1cccc(Cl)c1]

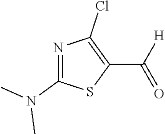

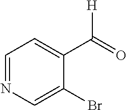

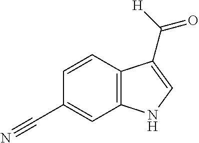 [SMILES: N#Cc1ccc2c(C=O)c[nH]c2c1]

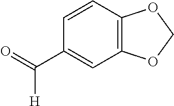 [SMILES: O=Cc1ccc2c(c1)OCO2]

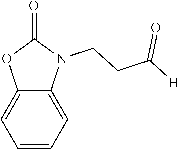 [SMILES: O=CCCn1c(=O)oc2ccccc21]

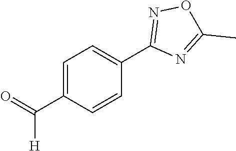

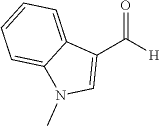

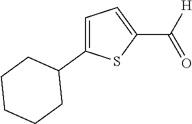 [SMILES: O=Cc1ccc(C2CCCCC2)s1]

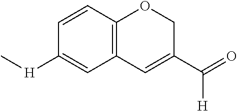

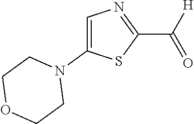 [SMILES: O=Cc1ncc(N2CCOCC2)s1]

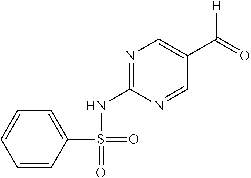 [SMILES: O=Cc1cnc(NS(=O)(=O)c2ccccc2)nc1]

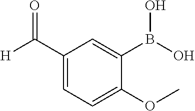

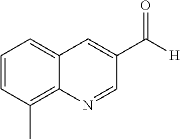

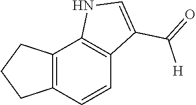 [SMILES: O=Cc1c[nH]c2c3c(ccc12)CCC3]

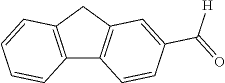 [SMILES: O=Cc1ccc2c(c1)Cc1ccccc1-2]

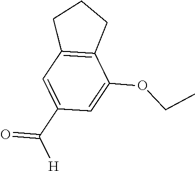

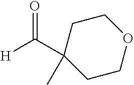

In some embodiments, the PBF moiety is a synthetically accessible benzhydrylpiperazine derived fragment with a structure shown in Formula XL VII.

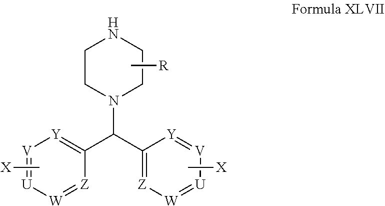

In the structure, R is alkyl group, aryl group, COOEt or H; U is CH or N; V is CH or N; W is CH or N; X is H, F, Cl, Br, I, alkyl group or aryl group; Y is CH or N; Z is CH or N. Exemplary synthetically accessible benzhydrylpiperazine derivative fragments include but are not limited to:

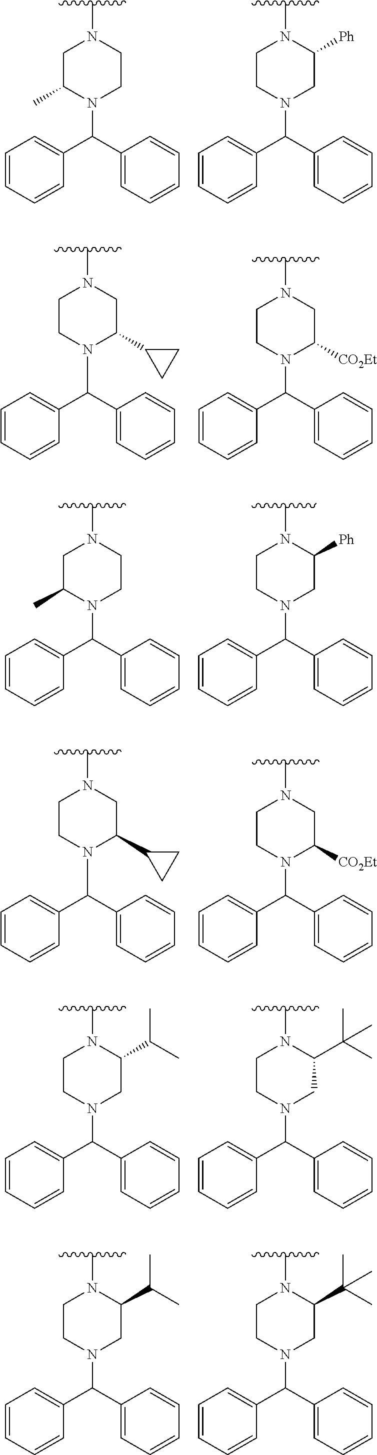

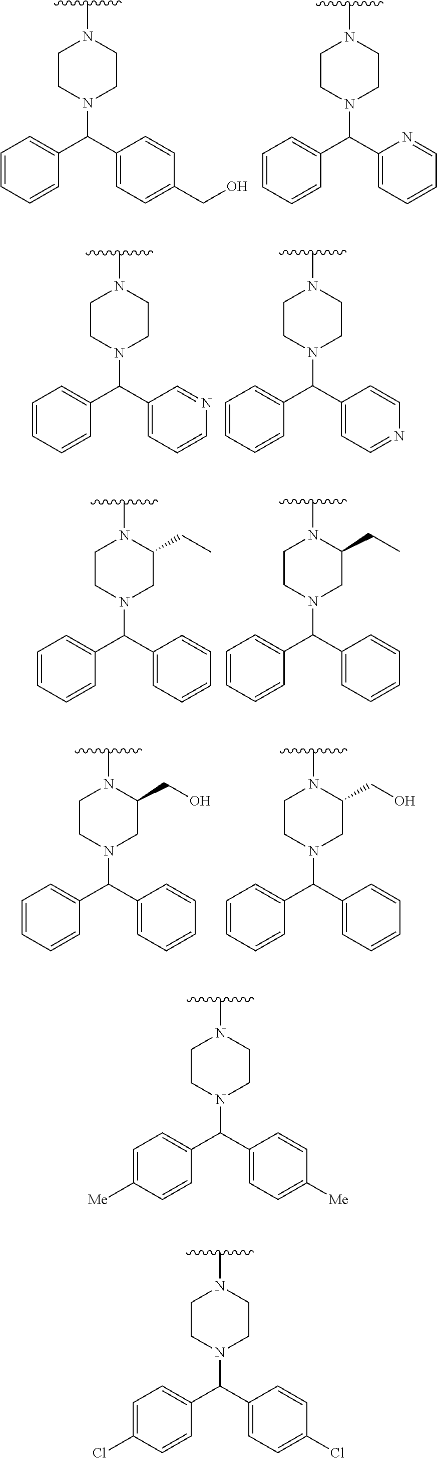

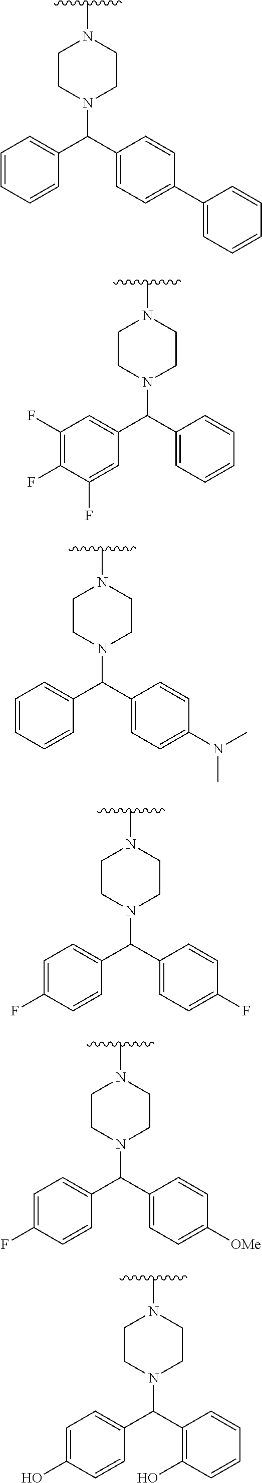

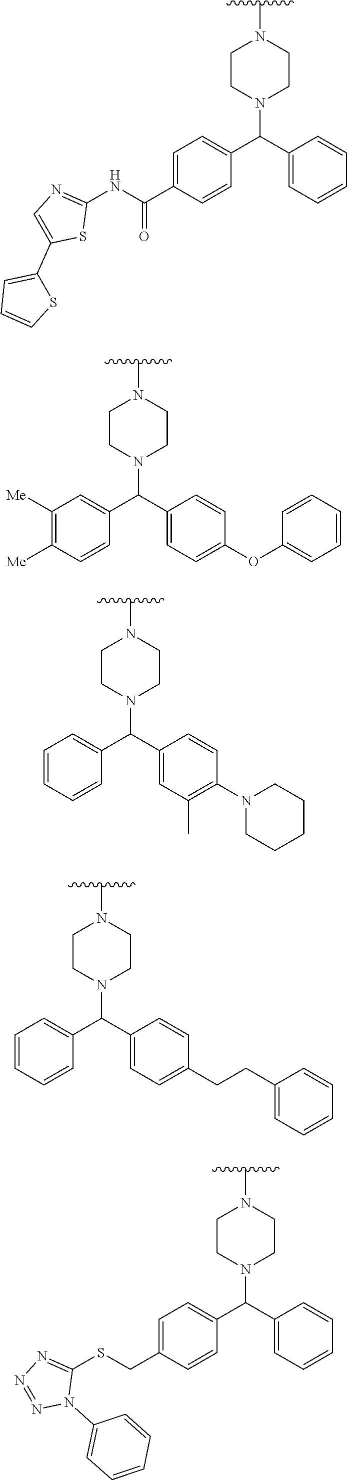 [SMILES: *N1CCN(C(c2ccccc2)c2ccc(C(=O)Nc3ncc(-c4cccs4)s3)cc2)CC1 ; *N1CCN(C(c2ccc(Oc3ccccc3)cc2)c2ccc(C)c(C)c2)CC1 ; *N1CCN(C(c2ccccc2)c2ccc(N3CCCCC3)c(C)c2)CC1 ; *N1CCN(C(c2ccccc2)c2ccc(CCc3ccccc3)cc2)CC1 ; *N1CCN(C(c2ccccc2)c2ccc(CSc3nnnn3-c3ccccc3)cc2)CC1]

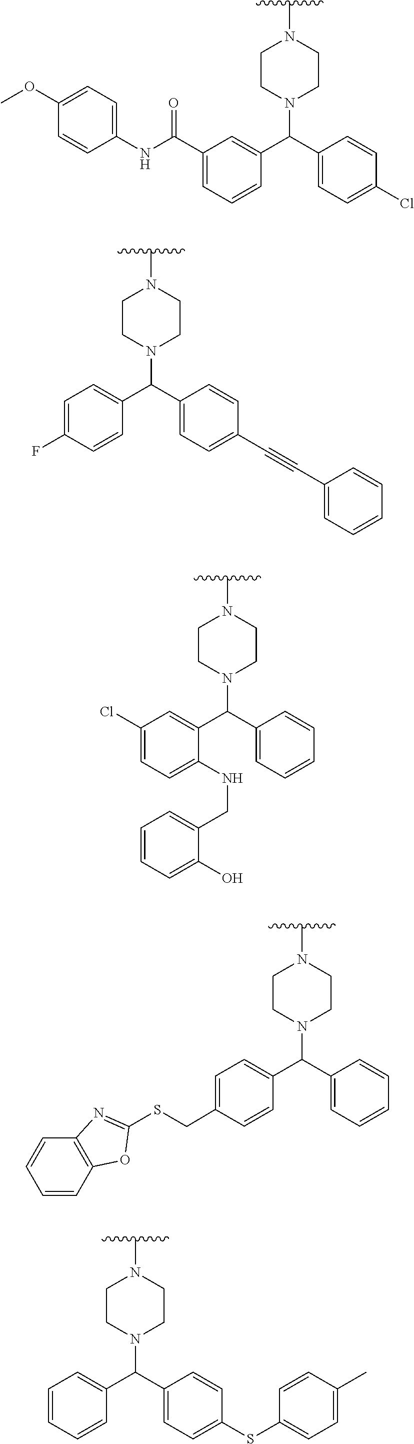 [SMILES: *N1CCN(C(c2ccc(Cl)cc2)c2cccc(C(=O)Nc3ccc(OC)cc3)c2)CC1 ; *N1CCN(C(c2ccc(F)cc2)c2ccc(C#Cc3ccccc3)cc2)CC1 ; *N1CCN(C(c2ccccc2)c2cc(Cl)ccc2NCc2ccccc2O)CC1 ; *N1CCN(C(c2ccccc2)c2ccc(CSc3nc4ccccc4o3)cc2)CC1 ; *N1CCN(C(c2ccccc2)c2ccc(Sc3ccc(C)cc3)cc2)CC1]

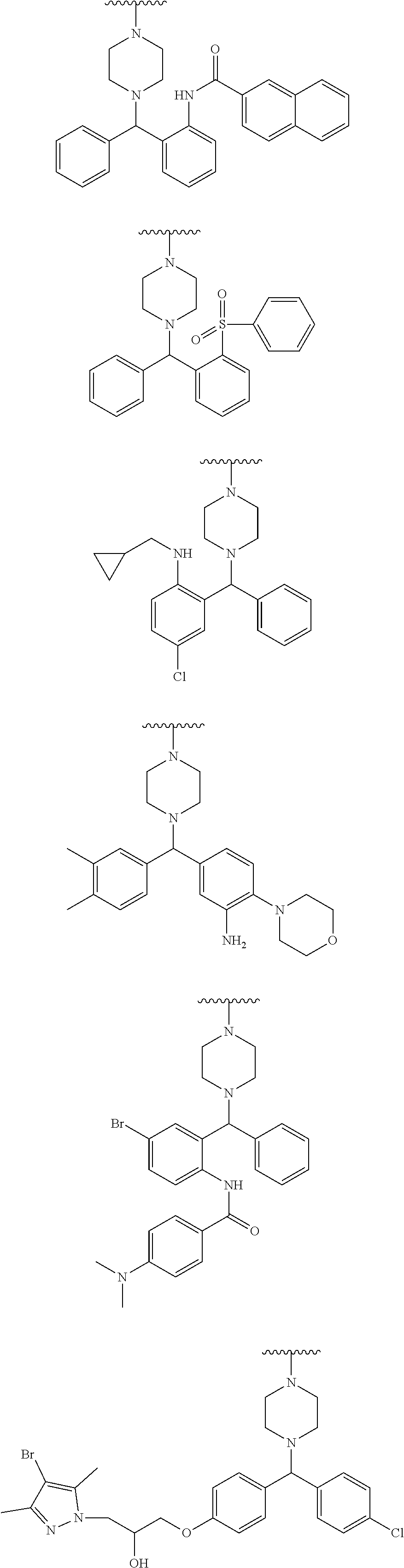 [SMILES: *N1CCN(C(c2ccccc2)c2ccccc2NC(=O)c2ccc3ccccc3c2)CC1 ; *N1CCN(C(c2ccccc2)c2ccccc2S(=O)(=O)c2ccccc2)CC1 ; *N1CCN(C(c2ccccc2)c2cc(Cl)ccc2NCC2CC2)CC1 ; *N1CCN(C(c2ccc(C)c(C)c2)c2ccc(N3CCOCC3)c(N)c2)CC1 ; *N1CCN(C(c2ccccc2)c2cc(Br)ccc2NC(=O)c2ccc(N(C)C)cc2)CC1 ; *N1CCN(C(c2ccc(Cl)cc2)c2ccc(OCC(O)Cn3nc(C)c(Br)c3C)cc2)CC1]

In some other embodiments, the employed PBF moiety is a synthetically accessible natural product-derived fragment. Exemplary synthetically accessible natural product-derived PBF fragments include but are not limited to:

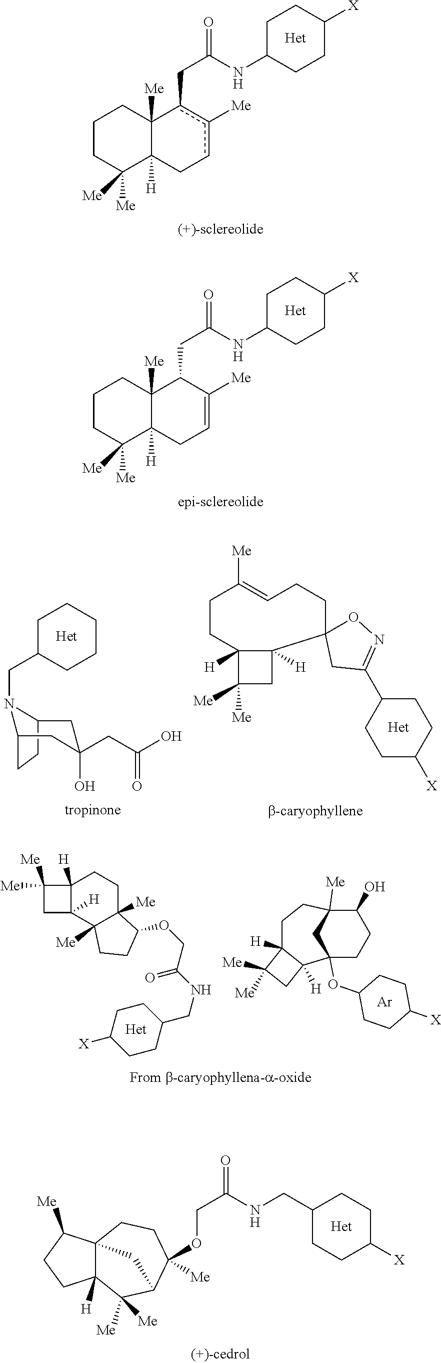

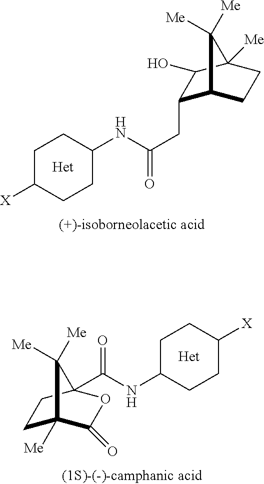

Like the PBF moiety, the RBF moiety in the FragTACs of the invention can also employ a number of suitable compounds. These include, e.g., CRBN ligands, VHL ligands, IAP ligands, MDM2 ligands, RNF ligands, DCAF ligands, KEAP1 ligands and FEMIB ligands. In some embodiments, the RBF moiety can be a CRBN ligand with a structure shown in any one of Formulae II-V below:

### CRBN Ligands (IMiDs):

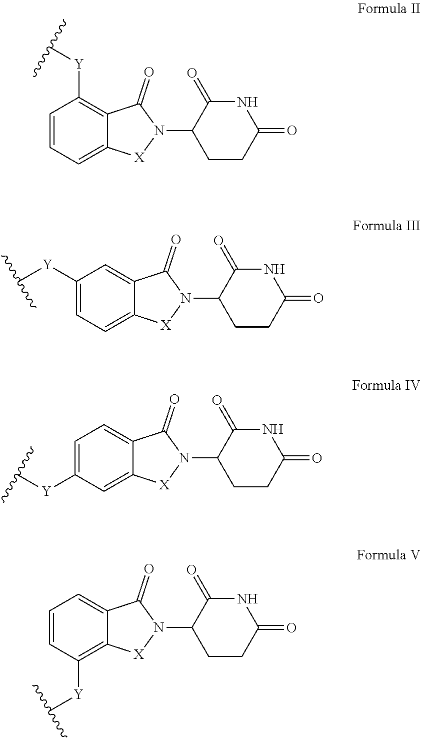

In these structures, X is CH₂ or CO; Y is NH, O, alkyne or CH₂. Exemplary CRBN-derived RBFs include but are not limited to:

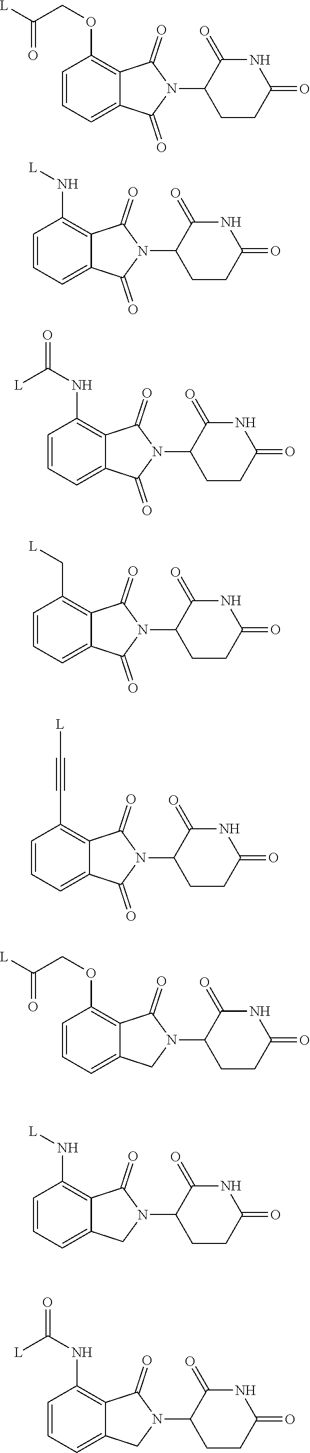

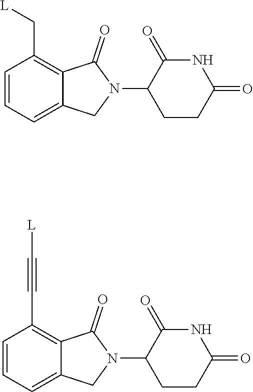

In some embodiments, the RBF moiety can be a VHL ligand with a structure shown in any one of Formulae VI-IX.

### VHL Ligands (Von-Hippel Lindau):

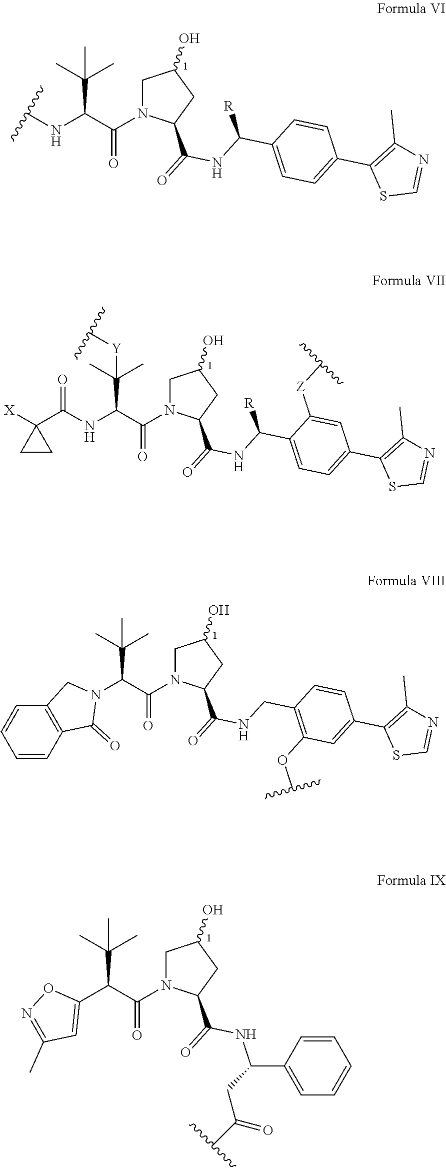

In these structures, stereochemistry of C1 is I or(S); R is H, CH₂ or CH₃; X is F or CN; Y is S or H; Z is O or H. Specific examples of VHL-derived RBFs include but are not limited to:

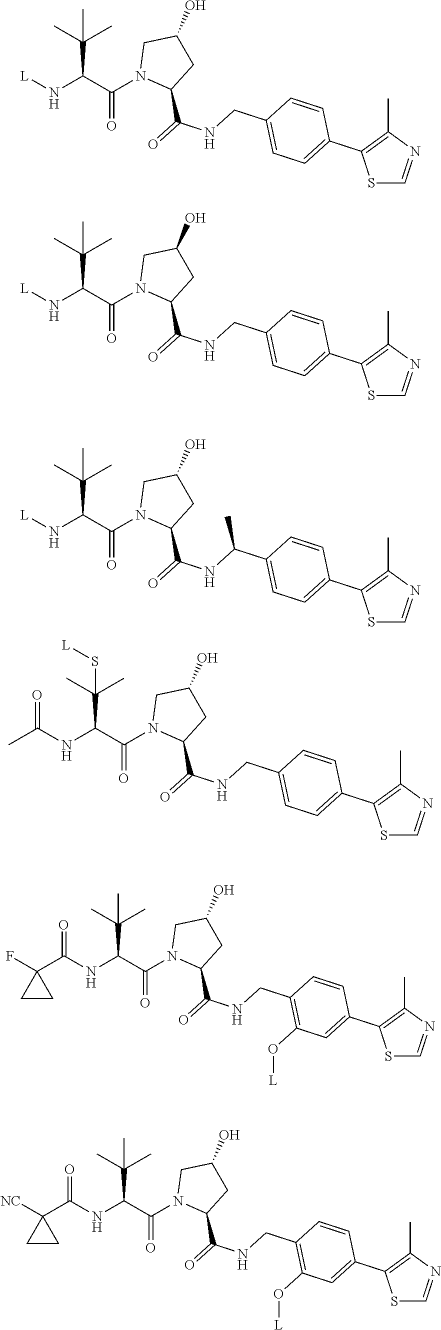

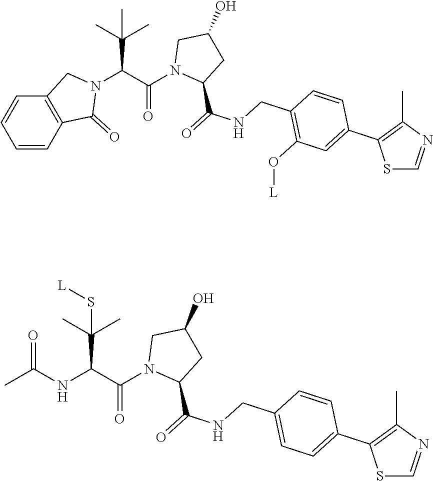

In some embodiments, the RBF moiety can be an IAP ligand with a structure shown in any one of Formulae X-XIII.

### IAP Ligands (Inhibitor of Apoptosis Proteins):

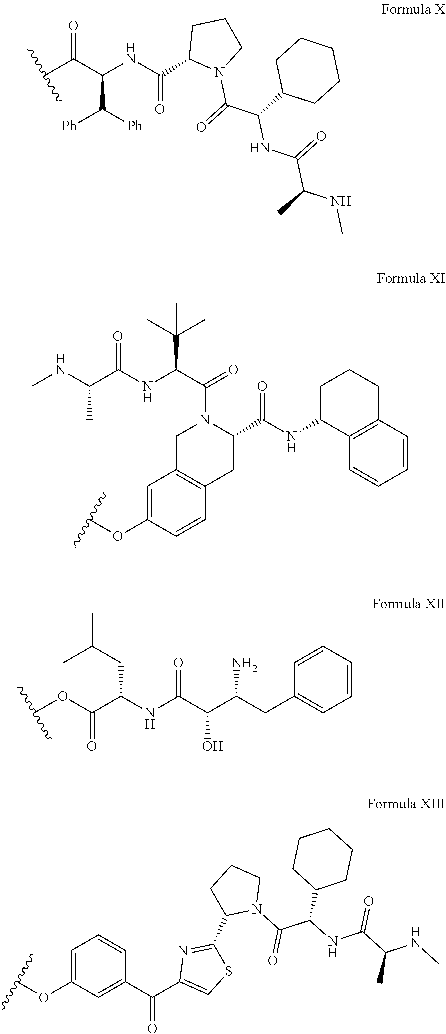

In some other embodiments, the RBF moiety can be a MDM2 ligand with a structure shown in any one of Formulae XIV-XVI.

### MDM2 Ligands:

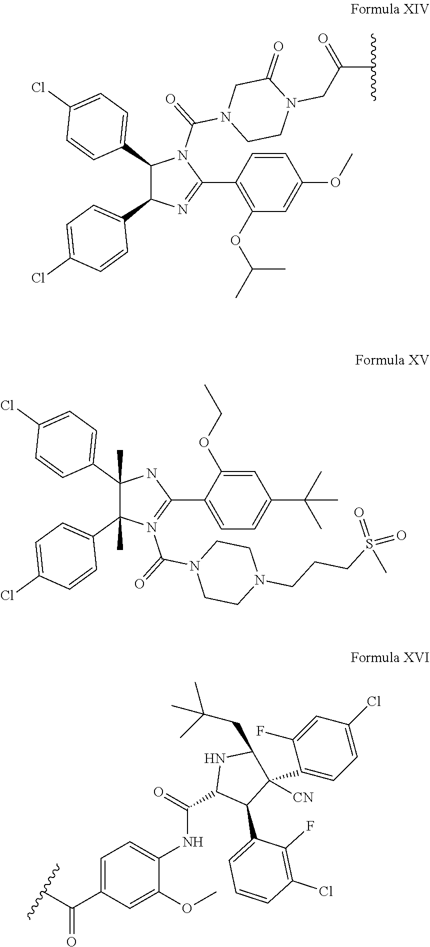

In still some other embodiments, the RBF moiety can be a RNF ligand with a structure shown in any one of Formulae XVII-XIX.

### RNF Ligands (Ring Finger Proteins):

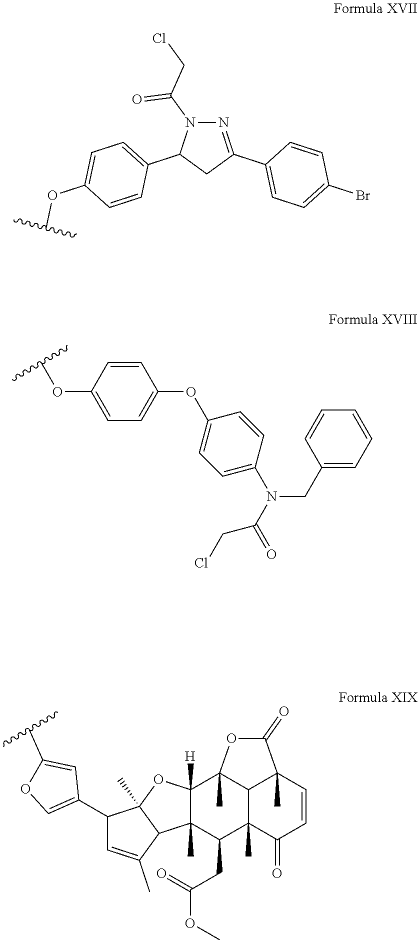

In some embodiments, the RBF moiety can be a DCAF ligand with a structure shown in any one of Formulae XX-XXI.

### DCAF Ligands:

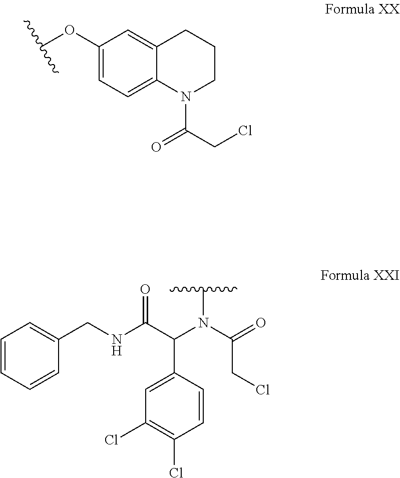 [SMILES: *Oc1ccc2c(c1)CCCN2C(=O)CCl ; *N(C(=O)CCl)C(C(=O)NCc1ccccc1)c1ccc(Cl)c(Cl)c1]

In some other embodiments, the RBF moiety can be a KEAP1 ligand or a FEM1B ligand with a structure shown in Formula XXII or XXIII, respectively.

### KEAP1 Ligand:

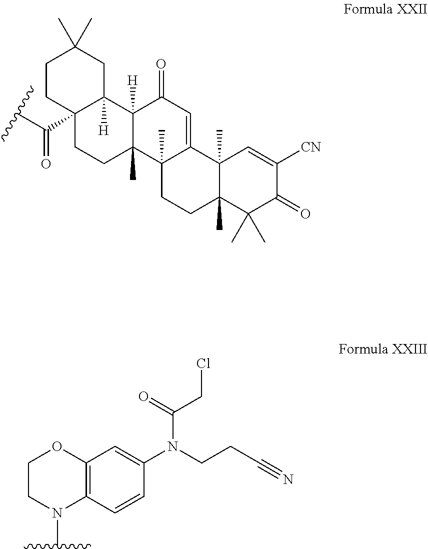

The linker (L) moiety in the FragTACs of the invention can employ any suitable compound or moiety that is capable of conjugating the PBF and RBF moieties without significant impact on their interactions with their cognate protein partners. In some embodiments, the L moiety can be a polyethylene glycol (PEG) linker with a structure shown in any one of Formulae XXIV-XXVI. In some other embodiments, the L moiety can be an aliphatic linker with a structure shown in any one of Formulae XXVII-XXX. In still some other embodiments, the L moiety can be a hybrid linker with a structure shown in any one of Formulae XXXI-XXXIX. In still some other embodiments, the L moiety can be an aryl-based linker with a structure shown in any one of Formulae XL and XLI. In some other embodiments, the L moiety can be a heterocycle-based linker with a structure shown in any one of Formulae XLII and XLIII. In some other embodiments, the L moiety can be a click chemistry-generated linker with a structure shown in any one of Formulae XLIV-XL VI. Polyethylene glycol (PEG) linkers:

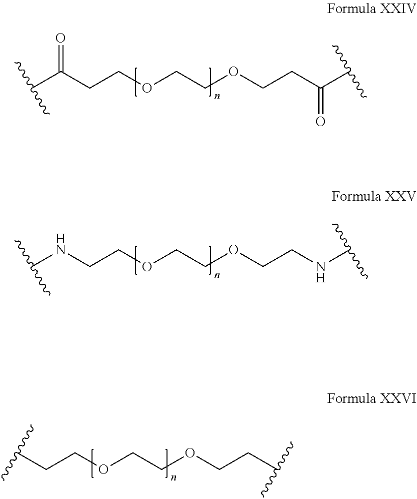

In these structures, n is an integer between 0 and 10.

### Aliphatic Linkers:

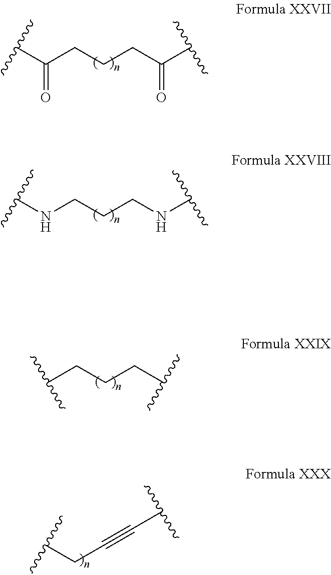

In these structures, n is an integer between 0 and 10.

### Hybrid Linkers:

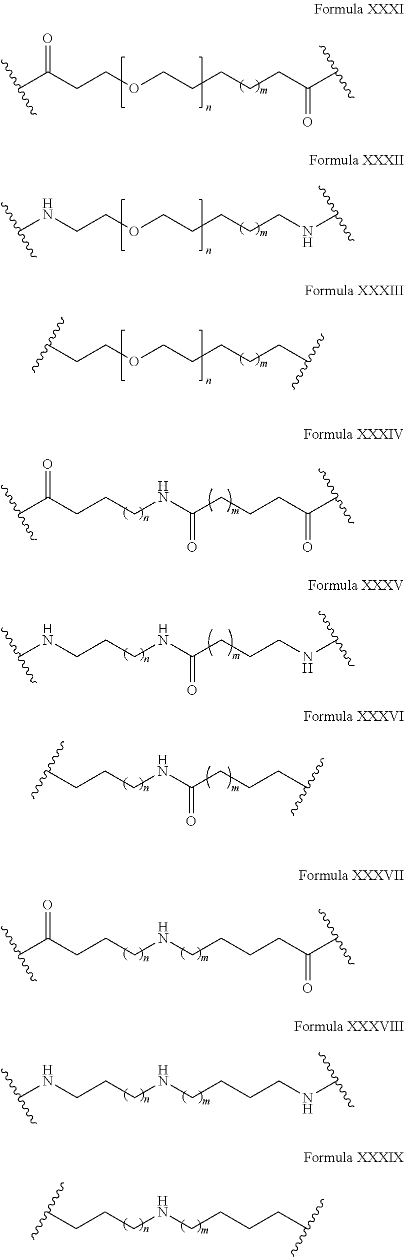

In these structures, n and m are each independently integers between 0 and 10.

### Aryl-Based Linkers:

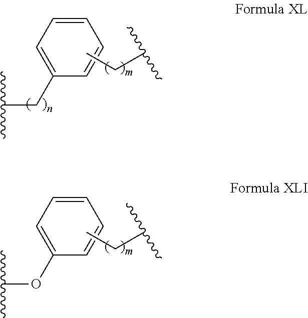

In these structures, n and m are each independently integers between 0 and 10.

### Heterocycle-Based Linkers:

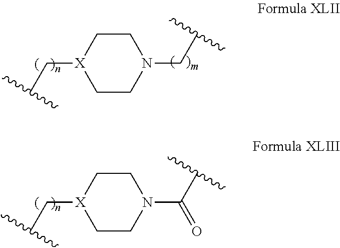

In these structures, n and m are each independently integers between 0 and 10; X is CH or N.

### Click Chemistry-Generated Linkers:

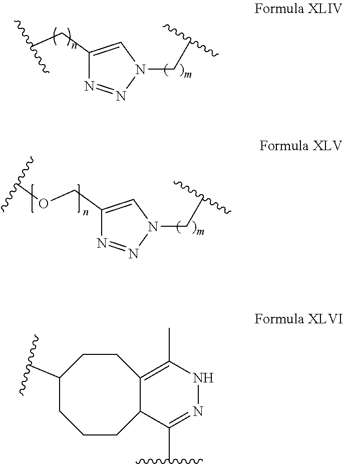

In these structures, n and m are each independently integers between 0 and 10. Formulae XLIV and XLV are generated through the cycloaddition of an azide with an alkyne. Formula XLVI is generated through the cycloaddition of tetrazine and trans-cyclooctene.

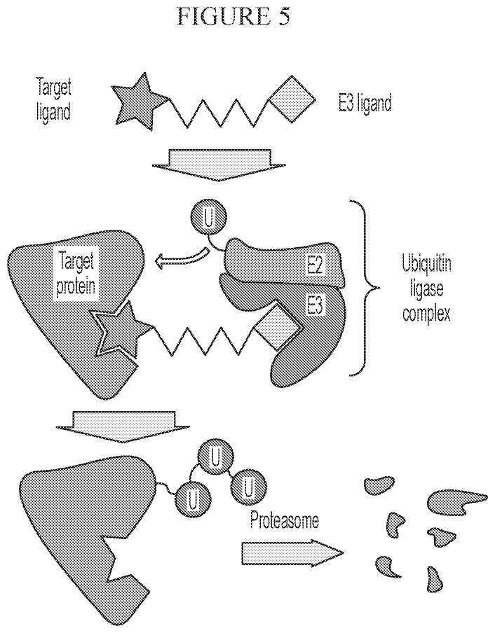

One aspect of the present invention is directed to heterobifunctional compositions that use small molecule fragments appended to established E3 ligands. As illustrated in FIG. 5, these heterobifunctional compositions identify degradable targets and synthetically progress-able ligands via unbiased whole proteome MS-based proteomics. This aspect of the invention has established that low target affinity interactions can lead to potent protein degradation.

Embodiments of this aspect of the heterobifunctional compositions are directed to a small library which was designed and synthesized to incorporate of bifunctional, fragment-based degrader molecules (‘FragTACs’). Generic Formula I, depicted above in the Summary, incorporates the protein binding terminus (PBT), the linker (L) and the recruiter binding terminus (RBT) as an embodiments of FragTACs. Exemplary embodiments of the PBT moiety include heterocyclic multicyclic organic molecular fragments listed below.

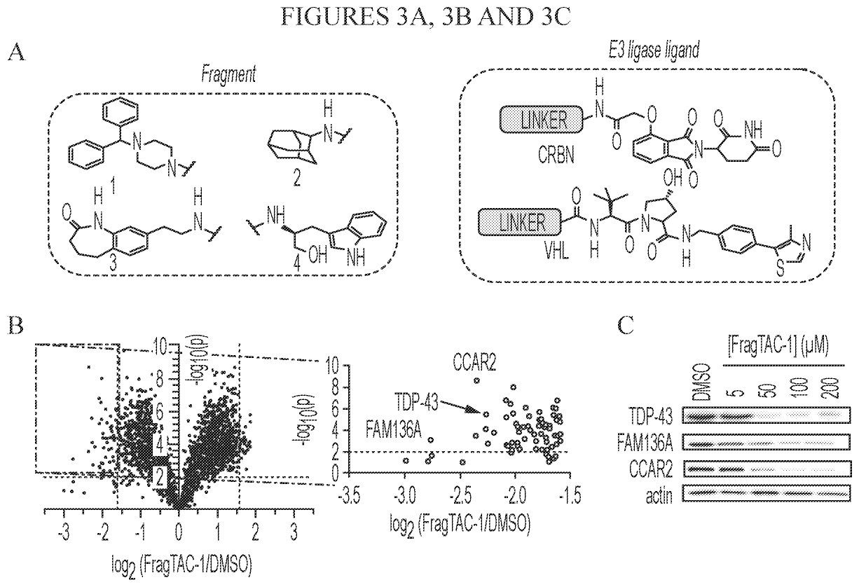

Exemplary embodiments of the L moiety include the linear organic chain fragments listed below. Exemplary embodiments of the RBT moieties include the organic molecular fragments listed below. These RBT moieties bind with the ligases as shown in the list including VHL, Cereblon, RNF-4, MDMR, RNF114, SNIPER and KEAP. Exemplary Cereblon and VHL ligand based FragTACs are listed below. The doses and percentages given in the list indicate the percent fragmentation at the given doses when the example is administered to a corresponding cell culture. See also FIG. 3A, eight members.

Focused embodiments of the heterobifunctional compositional FragTACs incorporate established von-Hippel Lindau (VHL) or Cereblon (CRBN) E3 ligands (18-20) which are chemically linked to any one of four fragment scaffolds. The fragment scaffolds were previously demonstrated to exhibit broad proteomic interactions (11). The ability of the FragTAC library to induce proteomic changes in breast cancer cells was profiled and identified a combined ˜120 downregulated proteins that span a broad range of classes (e.g. enzymes, transcription factors, chaperone proteins) as well as several members of protein complexes. FragTAC-1, in particular, substantially downregulated 43 proteins, including transcriptional repressor TDP-43, regulator protein CCAR2 and functionally uncharacterized protein FAM136A, which was confirmed to occur in a dose-dependent fashion (FIGS. 3B and 3C). Protein loss was blocked with the proteosome inhibitor MG132, suggesting that FragTAC-1 promotes proteasomal degradation (not shown). Notably, there are no known small molecule ligand for these proteins.

Examples of heterocyclic multicyclic organic molecular PBT fragments:

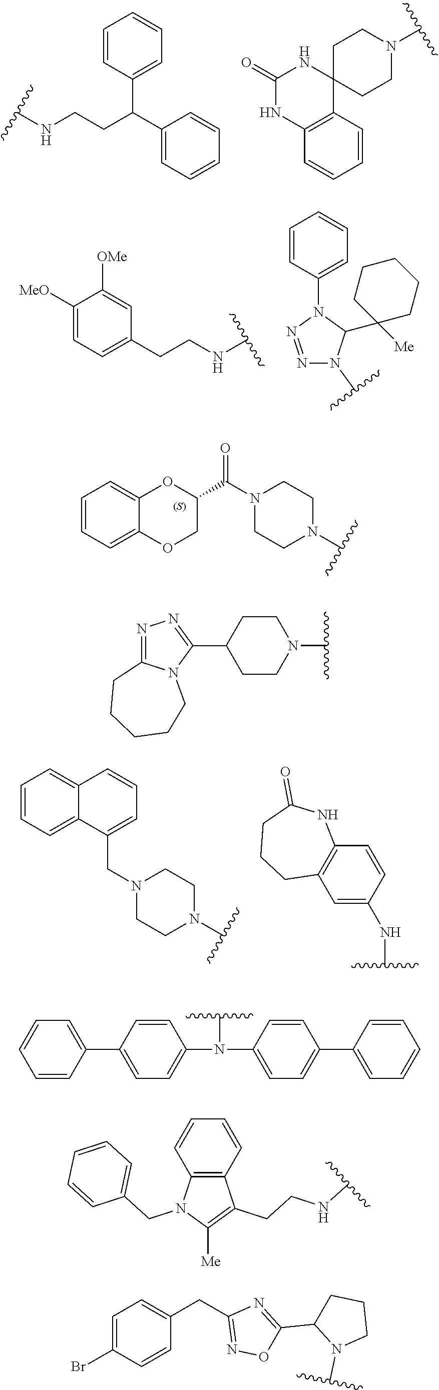

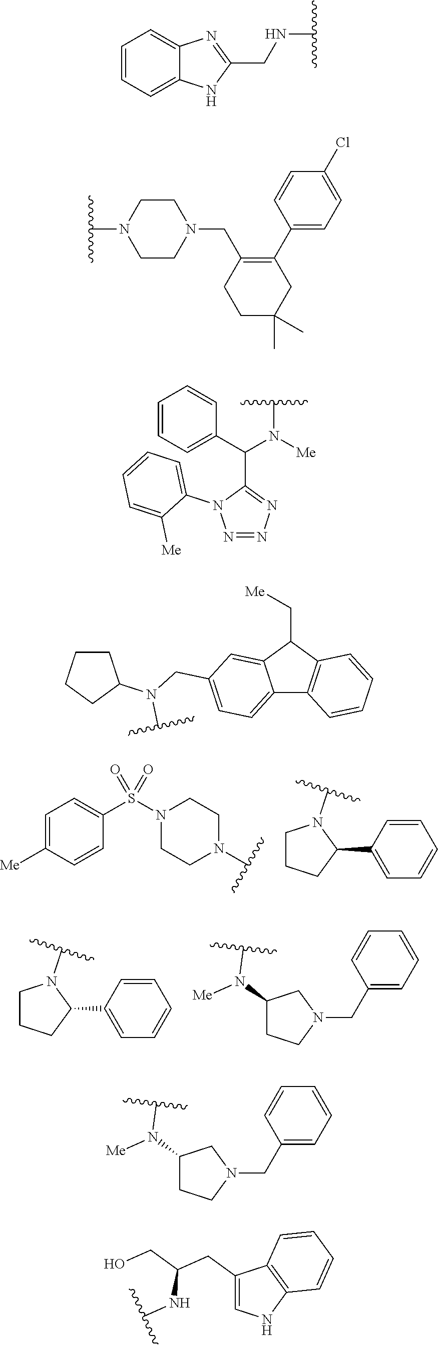

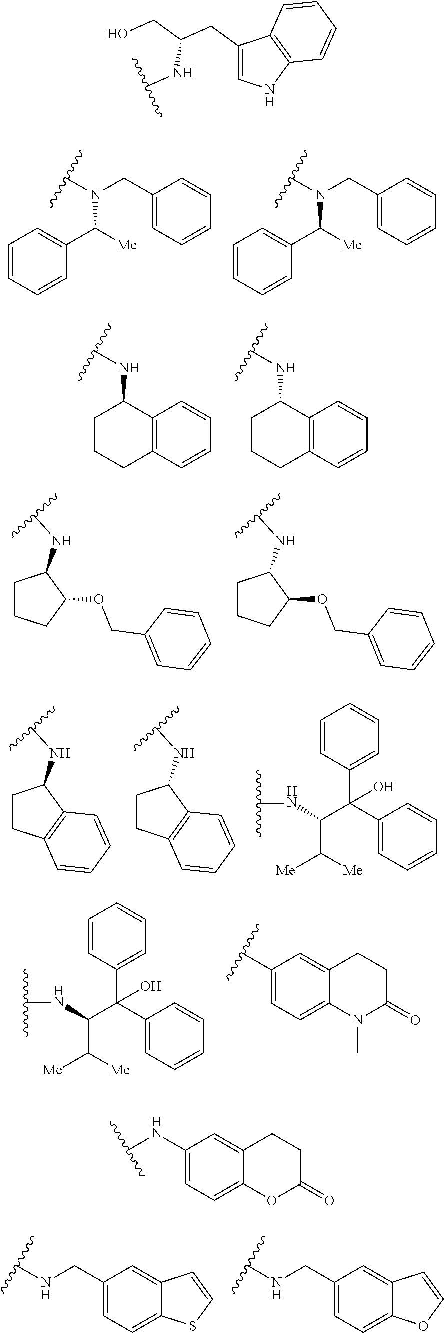

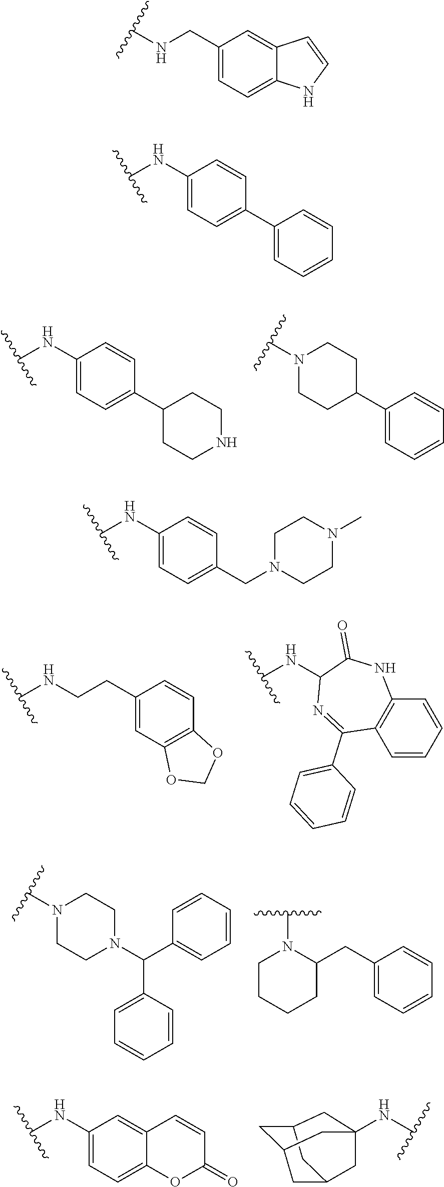

Examples of linear organic chain L moieties:

### Peg Linkers

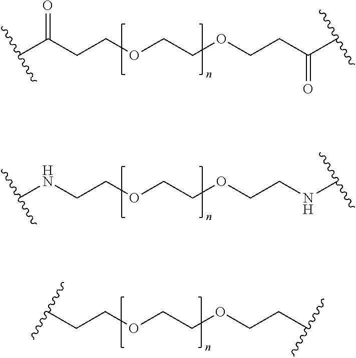

### Aliphatic Linkers

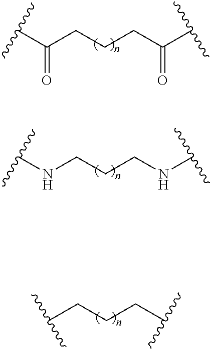

Examples of organic molecular RBT moieties:

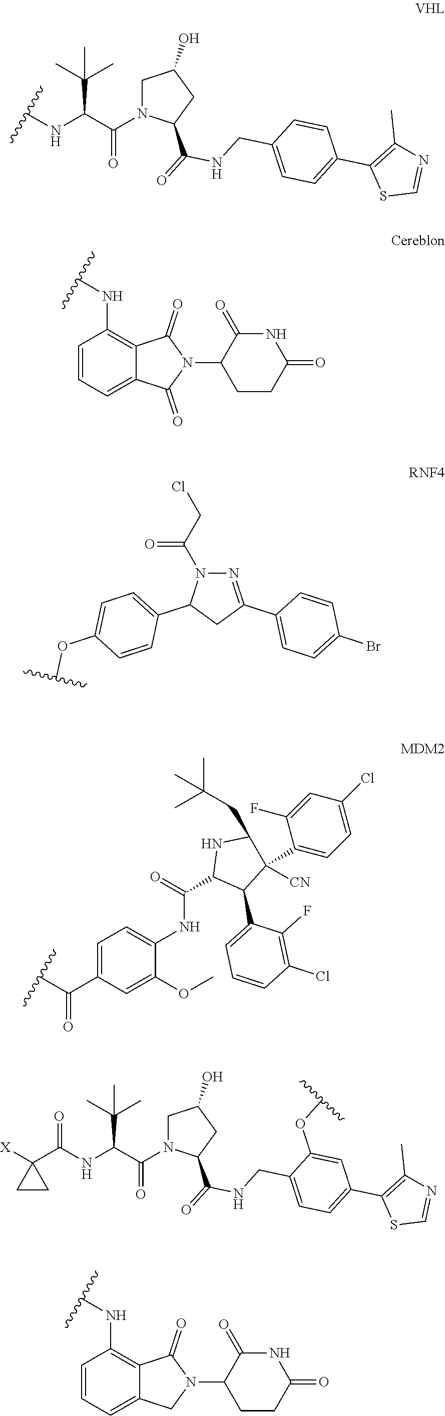

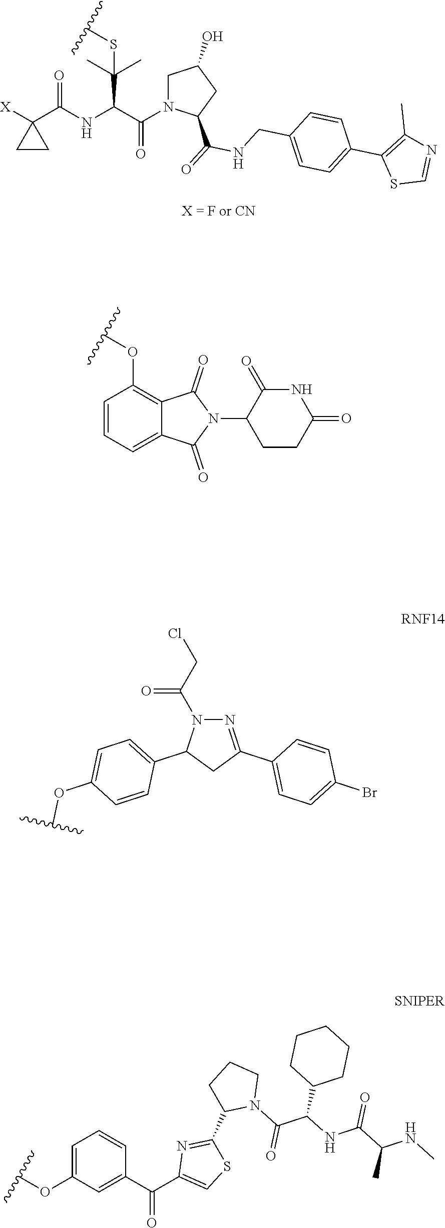

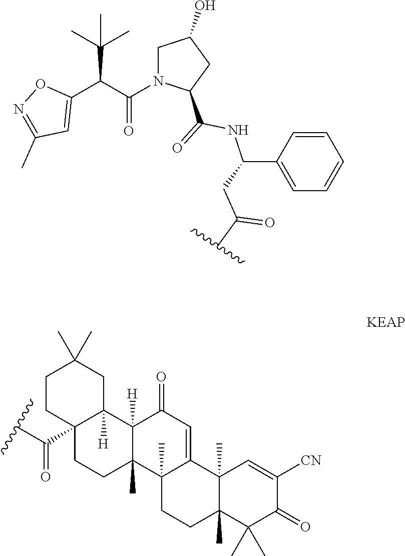

### Cereblon Ligand-Based FragTAC Probes:

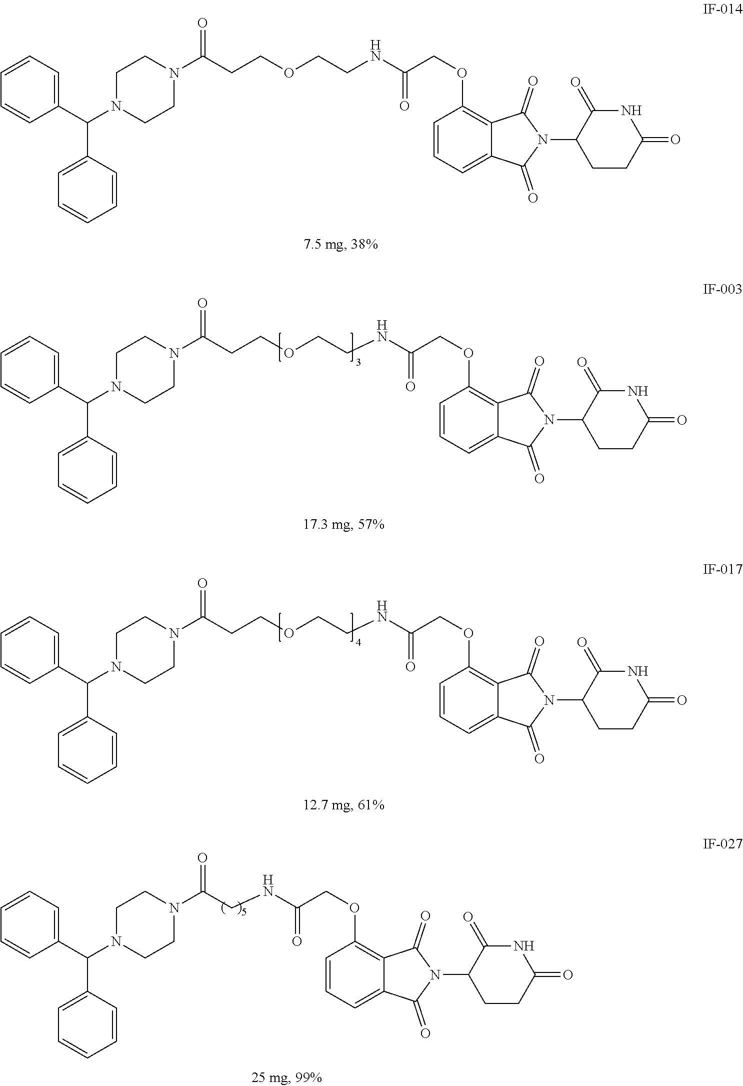

### VHL Ligand-Based FragTAC Probes:

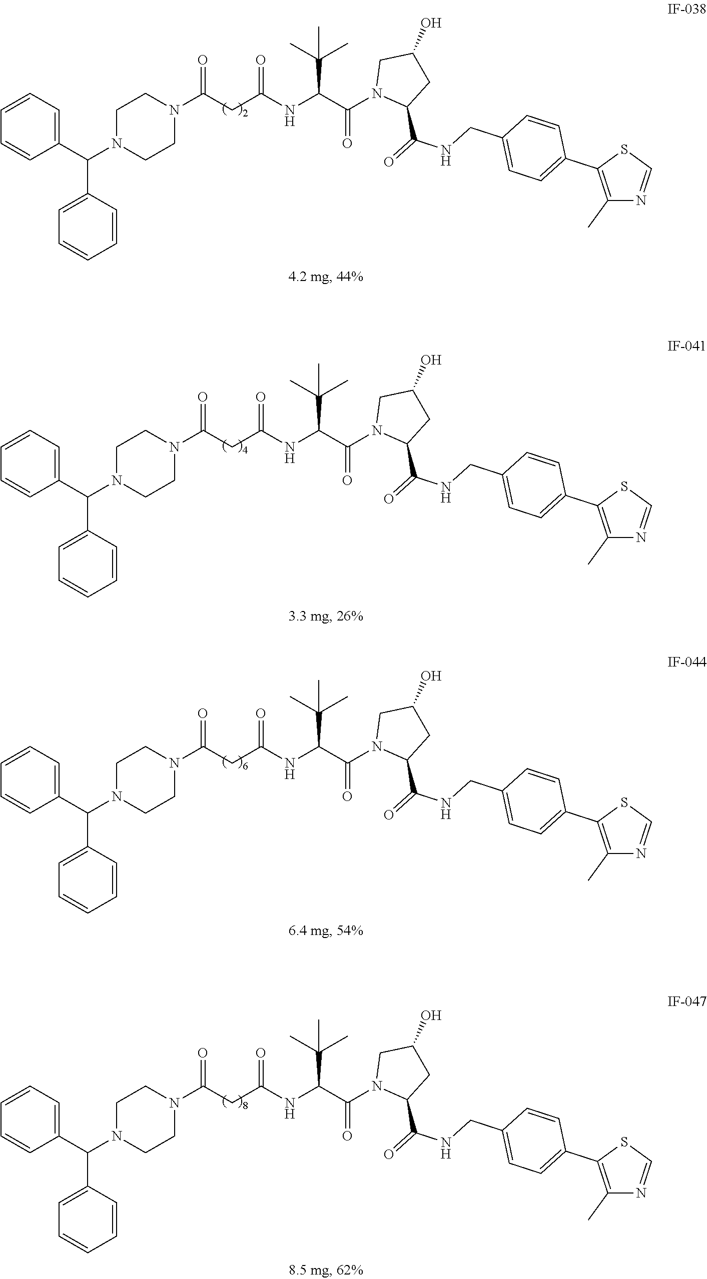

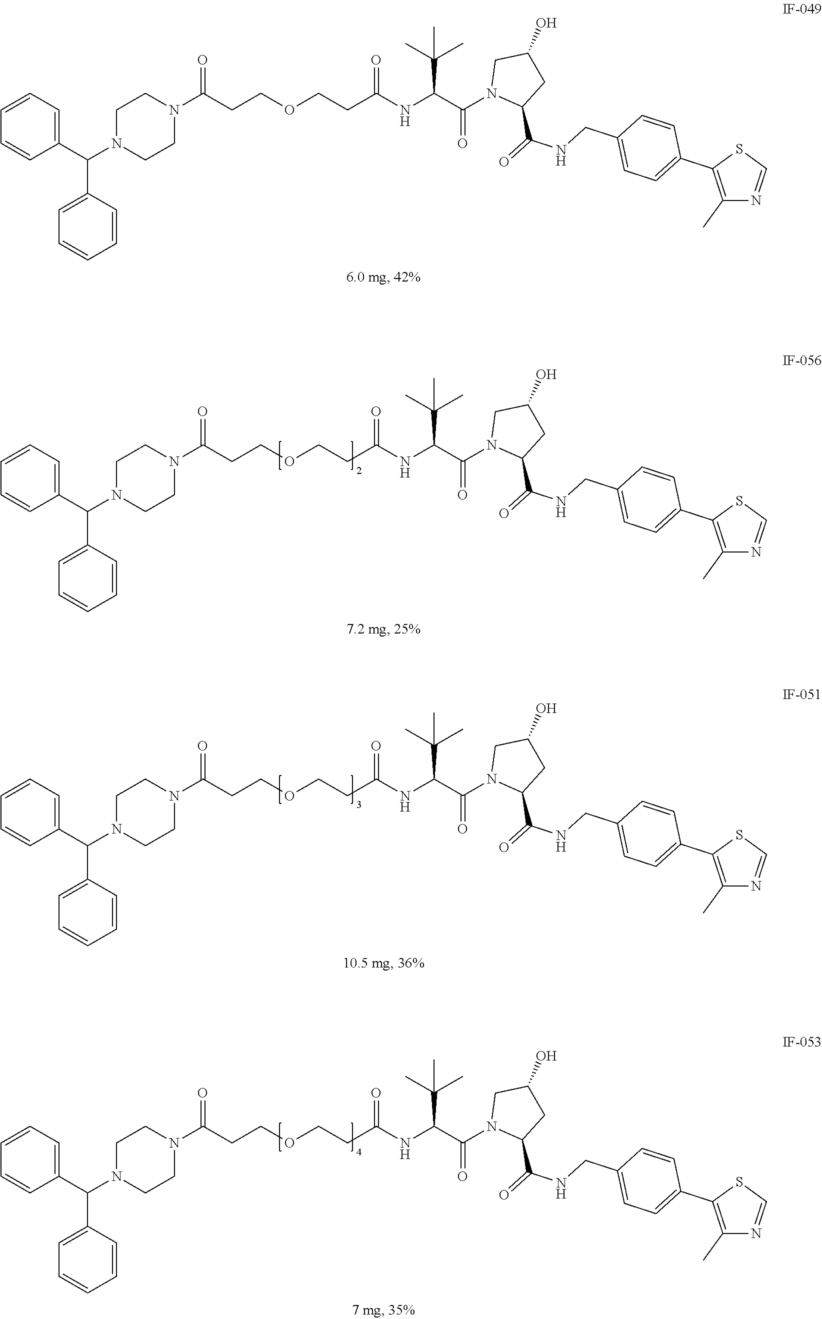

### De Novo Discovery of Degradable Protein Targets Using Fragment-Based PROTACs (FragTACs).

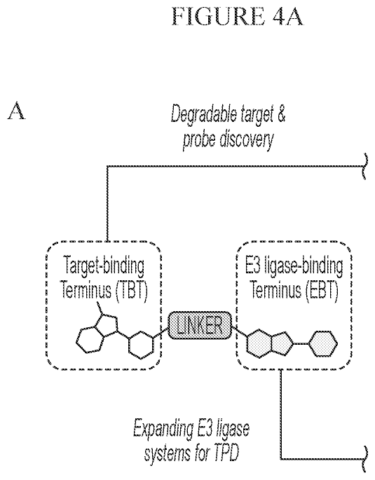

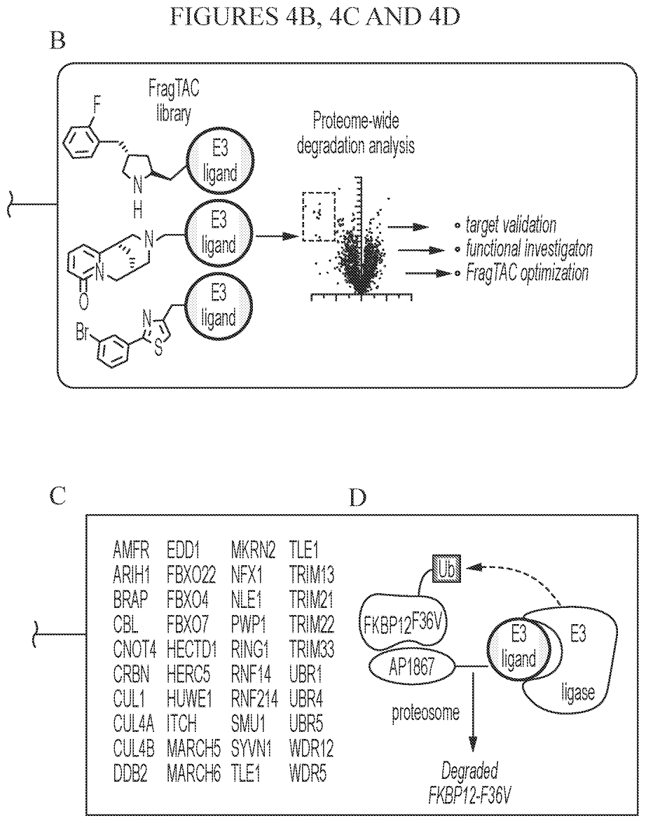

With the success in identifying degradable proteins using a small library of FragTACs, the strategy enveloping embodiments of the invention is expanded by synthesizing a larger FragTAC library (˜300) and applying them to identify degradable targets in therapeutically relevant human immune model systems using multiplex proteomic workflows (FIGS. 4A-4D). The goal of this strategy enables 1) identification of characteristic chemical features of PROTACs that lead to successful TPD; 2) a gain of an understanding of the impact of ligand affinity and promiscuity in selectivity and efficiency; 3) comparatively assessment of routinely employed E3 ligands for their ability to induce TPD; 4) broad annotation of human immune targets that may be tractable to TPD; 5) establishment of a template to transition and optimize promiscuous fragment-based degraders to selective PROTACs for targets with compelling therapeutic or biological value.

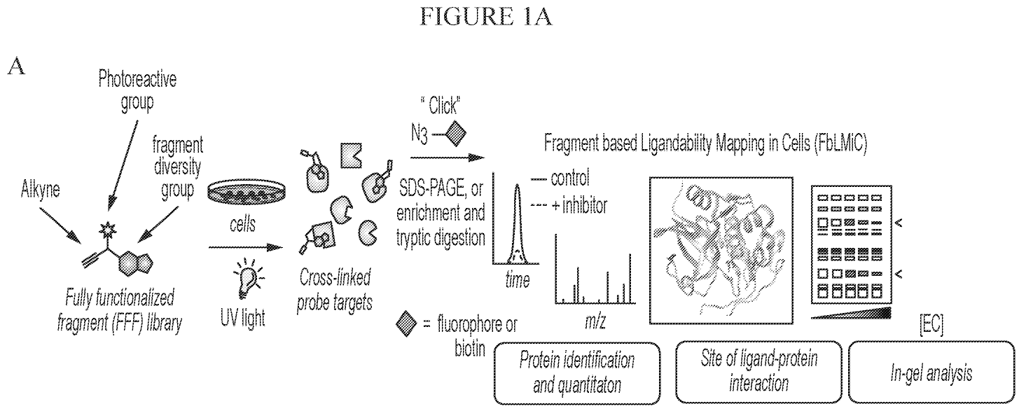

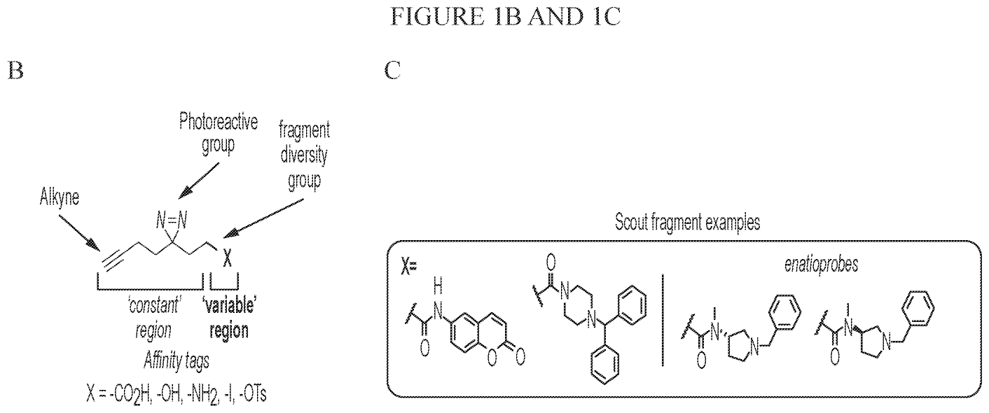

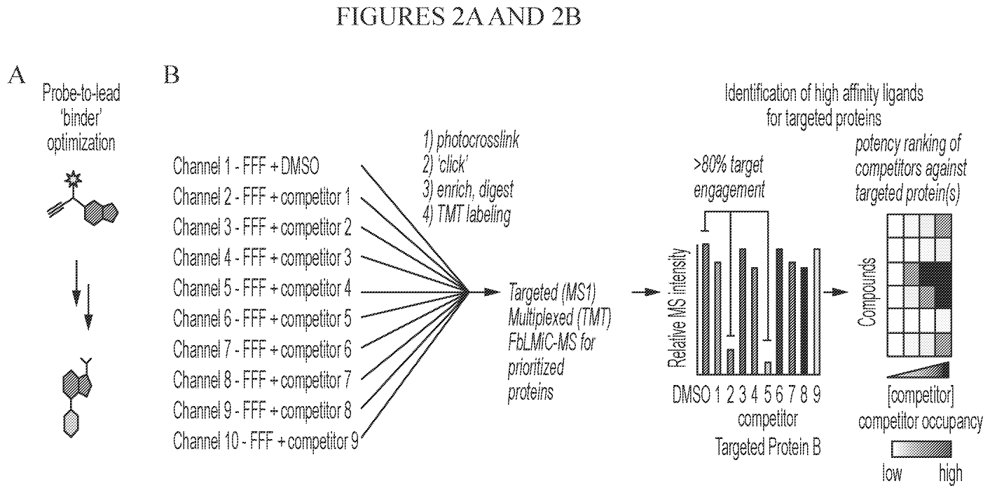

Towards this end, a library may be prepared using ˜30 small molecule fragments chemically linked to either CRBN or VHL E3 ligands (FIG. 4B). Fragments may be chosen based on their chemical diversity and non-overlapping proteomic interactions determined via the FbLDiSC workflows (FIGS. 1A-1C) to maximize proteomic coverage. In initial studies, 4-5 unique linkers (e.g. PEG, aliphatic) may be utilized, enabling the generation of mini-libraries around each fragment that explore variables such as E3s and linker composition. This library may be screened in pooled primary peripheral blood mononuclear cells (PBMCs) from de-identified donors for their ability to induce TPD via unbiased quantitative TMT proteomics as established in our preliminary studies (FIG. 3B). Human PBMCs may be chosen as an initial model system, as they are composed of a diverse cell population (T cells, B cells, dendritic cells, etc.) that contain uniquely expressed, immune-relevant targets, thus increasing the probability to uncover degradable, therapeutically translatable targets that serve critical roles in inflammation, infection, and cancer progression, for example. The variables discussed above may be encoded in the library to assess their independent contributions as well as establish a baseline of proteins susceptible to this TPD system. The FbLDiSC workflows may be used to assess and optimize potency and selectivity of lead FragTACs, as needed, for prioritized targets. See FIGS. 2A-2B and FIGS. 3A-3C.

Chemoproteomic-Enabled Discovery of E3 Ligase-Binding Compounds that Support TPD in Human Cells.

Multiple E3 ligase components, including CRBN (31), VHL (32), RNF114 (33), and DCAF16 (34) have been shown to engage in tripartite complexes where bridging small molecules can direct specific protein substrates to ubiquitination and degradation. These findings, combined with the large number of E3 ligases (>600 (35)) in humans, have stimulated interest in the broader draggability potential of TPD (36). The recent proteome-wide ligandability maps using FbLDisC have identified hit ligands (or ‘binders’) for >40 E3 ligases (11, 13) (FIG. 4C). The capacity of these E3-binders to support TPD may be tested using a recently described dTAG model system (37) (FIG. 4D). E3 binders that induce degradation may be optimized into lead chemical probes using FbLDisC-guided medicinal chemistry as described in FIGS. 2A-2B. The chemical probes may be investigated for their ability to degrade targets with established ligands. Enzyme ligase E3 systems may be prioritized with restricted tissue expression, (e.g. in cancer or immune cells), as they may offer safer paths for drugs compared to broadly expressed E3 systems. To this end several fragment-based ligands for several E3 ligases have been validated. See Table 1.

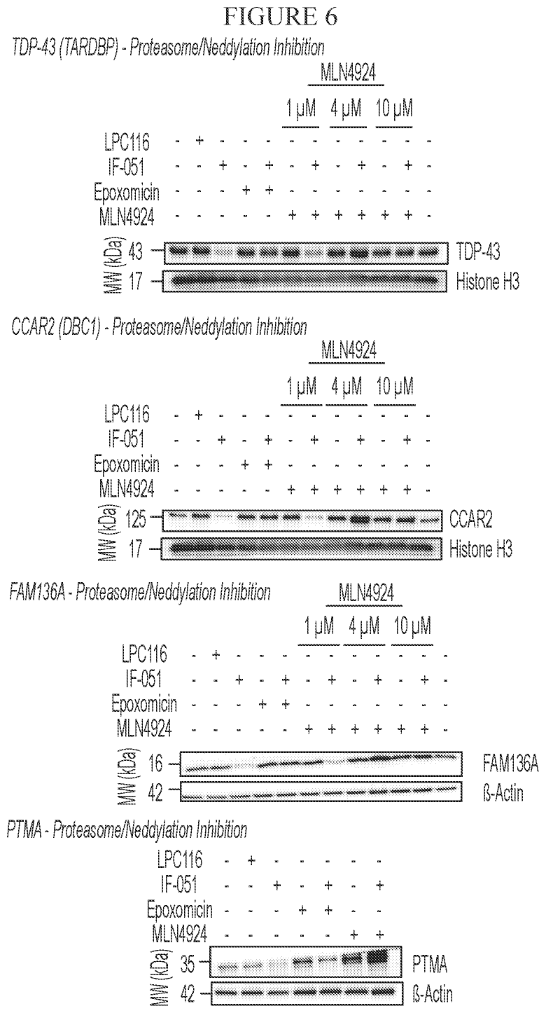

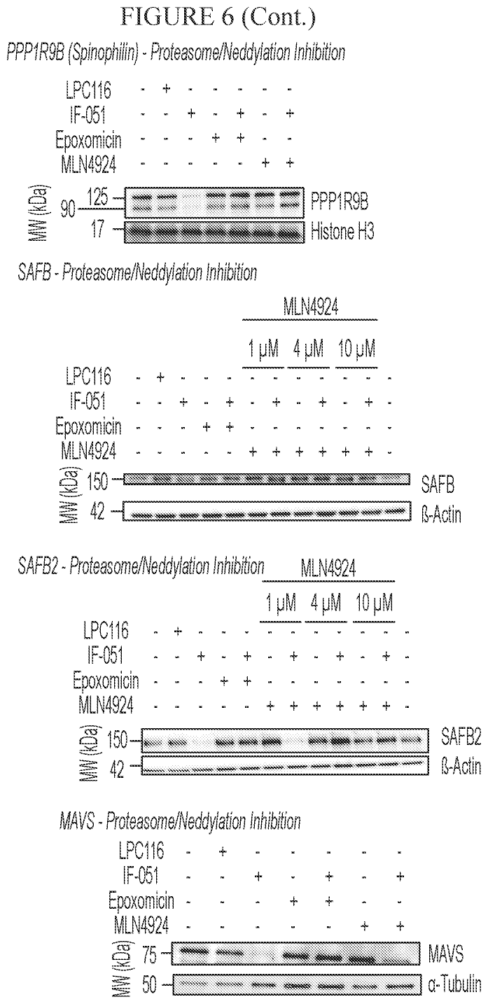

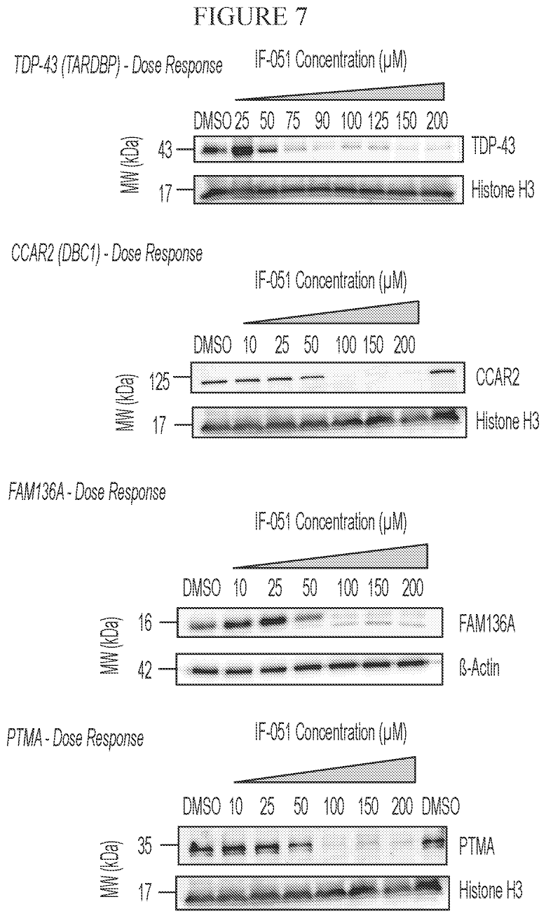

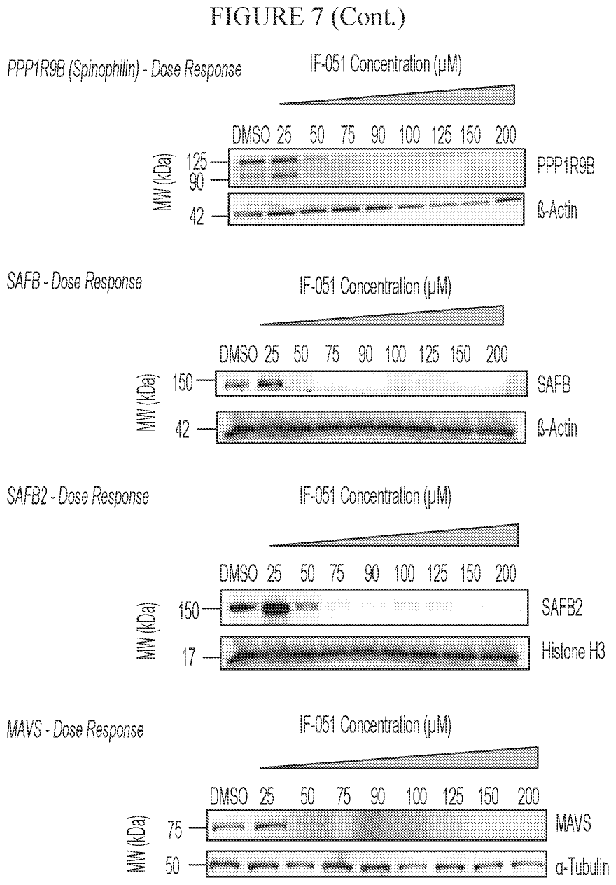

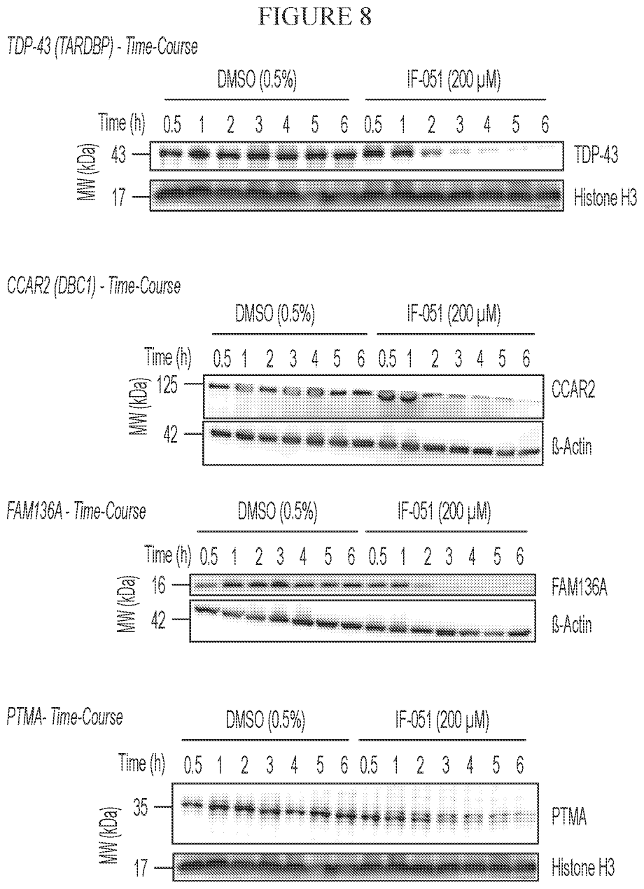

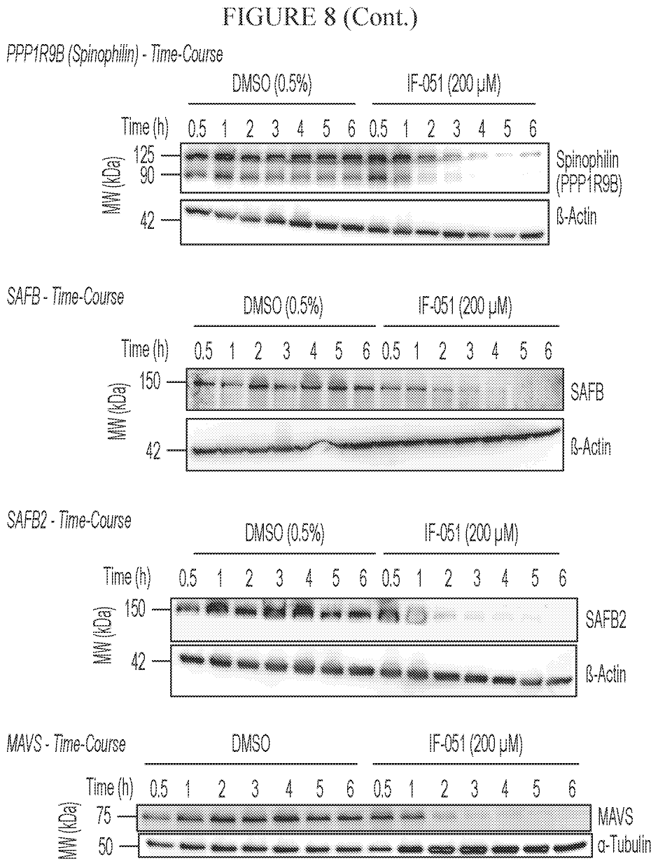

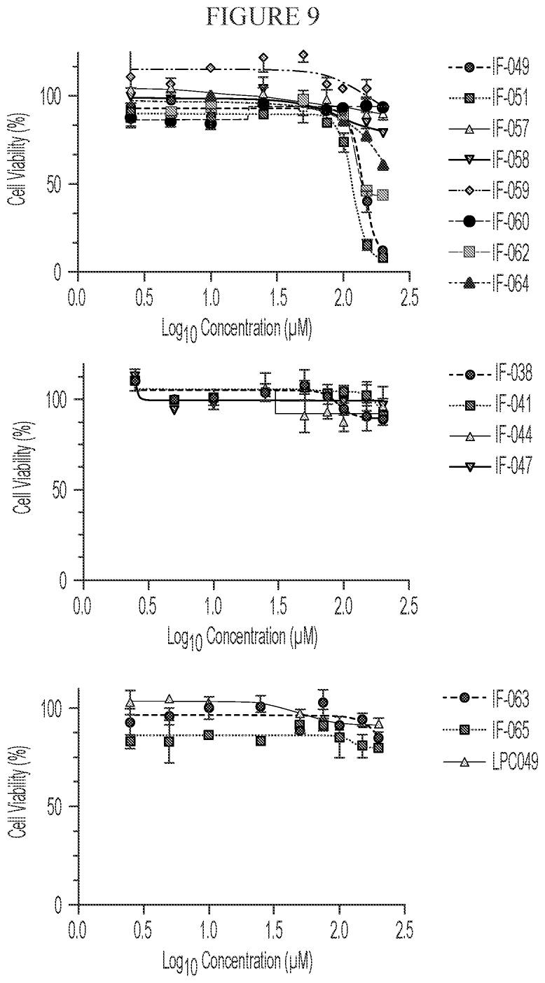

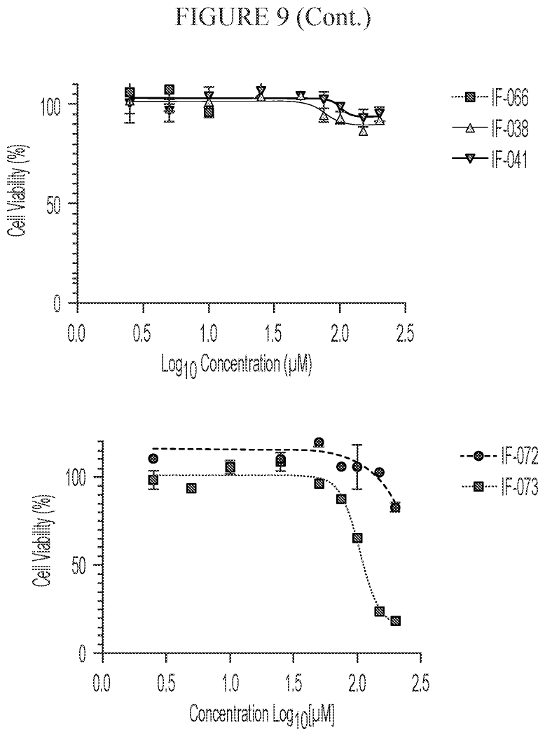

Additional studies were performed to validate the activities of the FragTAC probes in HCC1806 cells via Western blot analyses. Results from these studies are shown in FIGS. 6-9.

TABLE 1
Examples E3s with newly validated ligands

| Gene | Type | Localization | Tissue |
|---|---|---|---|
| CUL1 | Cullin | | Lung fibroblasts |
| CUL4A | Cullin | | |
| CUL4B | Cullin | Nucleus | |
| DDB2 | DDB1- | Nucleus; Accumulates at sites of | DNAUbiquitously expressed; corneal |
| | Adaptor | damage following UV irradiation. | endothelium |
| EDD1 | HECT | Nucleus | Testis, brain, pituitary and |
| | | | kidney. |
| FBXO3 | FBOX | Nucleus; Colocalizes with PML | |
| FBXO7 | FBOX | Cytoplasm, Nucleus, Mitochondrion, | |
| | | Cytoplasm, Cytosol | |
| FBXO9 | FBOX | Cytoplasm | |
| HUWE1 | HECT | Cytoplasm, Nucleus | Heart, brain and placenta |
| PWP1 | DDB1- | Nucleus; Nucleolus; Chromosome | Placenta, skeletal muscle, kidney |
| | Adaptor | | and pancreas |
| SYVN1 | VBM | ER membrane | Ubiquitously expressed |
| TRIM21 | RING | Cytoplasm; Cytoplasmic vesicle; | Heart and fetal lung. |
| | | Autophagosome; Nucleus; P-body | |
| UBR1 | RING | Cytoplasm, Cytosol | Broadly, skeletal muscle, kidney |
| | | | and pancreas |
| UBR4 | UIM | Membrane, Cytoplasm, Cytoplasm, | |
| | | Cytoskeleton, Nucleus | |
| UHRF1 | RING | Nucleus; Localizes to replication foci | Thymus, bone marrow, testis, |
| | | | lung and heart. Overexpressed in |
| | | | breast cancer. |

### Results of Proteomics Studies

Examples of downregulated proteins targeted by FragTACs are listed in Table 2. The results show targets with 2 unique peptides or more and depleted 2-fold or more relative to DMSO control (P value <0.05). Compound structures can be found in the examples of FragTAC Probes described herein (Table 3).

TABLE 2
Types of proteins targeted by FragTAC probes

| | Accession | |
|---|---|---|
| Proteins | Number | Probe Code |
| A2M | P01023 | IF-051 |
| AATF | Q9NY61 | LPC020 |
| ABCC5 | O15440 | LPC020 |
| ABHD10 | Q9NUJ1 | LPC020 |
| ABLIM1 | O14639 | IF-051, CMC-6-123 |
| ACAA2 | P42765 | LPC020 |
| ACADM | P11310 | LPC020 |
| ACADSB | P45954 | LPC020 |
| ACAT1 | P24752 | LPC020 |
| ACO2 | Q99798 | LPC020 |
| ACOT1 | Q86TX2 | LPC020 |
| ADAM15 | Q13444 | CMC-6-109, CMC-6-111, IF-072 |
| ADIRF | Q15847 | IF-051, IF-085 |
| AGR2 | O95994 | LPC020 |
| AHCTF1 | Q8WYP5 | LPC020 |
| AHNAK | Q09666 | IF-051 |
| AHNAK2 | Q8IVF2 | IF-051 |
| AHSG | P02765 | LPC020, IF-085 |
| AIDA | Q96BJ3 | IF-051 |
| AK2 | P54819 | LPC022, LPC020, IF-051, CMC-6-123, IF-049 |
| AK3 | Q9UIJ7 | LPC020 |
| AK4 | P27144 | LPC020 |
| AKAP8 | O43823 | LPC020 |
| ALAS1 | P13196 | CMC-6-123 |
| ALCAM | Q13740 | IF-051, LPC020, IF-049 |
| ALDH1B1 | P30837 | LPC020 |
| ALYREF | Q86V81 | IF-051, LPC022, LPC020 |
| ANAPC5 | Q9UJX4 | LPC020, LPC011 |
| ANLN | Q9NQW6 | CMC-6-109, CMC-6-111, IF-072, IF-080 |
| ANPEP | P15144 | IF-072 |
| API5 | Q9BZZ5 | IF-051 |
| APOB | P04114 | LPC020 |
| ARID1B | Q8NFD5 | IF-051 |
| ARMC10 | Q8N2F6-2 | IF-051, IF-049 |
| ARPC5L | Q9BPX5 | IF-051 |
| ATAD2 | Q6PL18 | LPC011 |
| ATP1A2 | P50993 | LPC020 |
| ATP2B1 | P20020 | IF-051 |
| ATP2B4 | P23634 | IF-051 |
| ATP5F1A | P25705 | LPC020 |
| ATP5F1B | P06576 | LPC020 |
| ATP5F1C | P36542 | LPC022, LPC020 |
| ATP5F1D | P30049 | LPC022, LPC020 |
| ATP5PD | O75947 | LPC020 |
| ATP5PO | P48047 | LPC020 |
| ATPAF1 | Q5TC12 | LPC020 |
| B4GALT1 | P15291 | LPC020 |
| BACH1 | O14867 | IF-051, IF-049 |
| BAZ1B | Q9UIG0 | CMC-6-109, CMC-6-111, IF-072 |
| BBX | Q8WY36 | IF-051 |
| BCAM | P50895 | IF-051, CMC-6-109 |
| BCKDK | O14874 | IF-051 |
| BCL2L12 | Q9HB09 | LPC020 |
| BCLAF1 | Q9NYF8 | IF-051, LPC020 |
| BICRA | Q9NZM4 | IF-051 |
| BOD1L1 | Q8NFC6 | IF-051, IF-049 |
| BOLA3 | Q53S33 | IF-051 |
| BPTF | Q12830 | LPC020 |
| BRD2 | P25440 | IF-051, IF-065 |
| BRD8 | Q9H0E9 | IF-051 |
| BRD9 | Q9H8M2 | IF-051 |
| BTF3L4 | Q96K17 | IF-051 |
| BUD13 | Q9BRD0 | IF-051, LPC020, IF-049 |
| BUD31 | P41223 | IF-051 |
| C2orf49 | Q9BVC5 | IF-051 |
| C9orf78 | Q9NZ63 | IF-051 |
| CACTIN | Q8WUQ7 | IF-051 |
| CAMLG | P49069 | IF-051 |
| CARD19 | Q96LW7-2 | IF-051, CMC-6-123 |
| CASK | O14936 | IF-051 |
| CBLL1 | Q75N03 | IF-051 |
| CBR4 | Q8N4T8 | LPC020 |
| CBX1 | P83916 | IF-051, CMC-6-109, IF-072 |
| CBX3 | Q13185 | IF-051, CMC-6-109, IF-072 |
| CBX5 | P45973 | IF-051 |
| CCAR2 | Q8N163 | LPC020 |
| CCDC12 | Q8WUD4 | IF-051, LPC022, LPC020, CMC-6-123 |
| CCDC124 | Q96CT7 | IF-051 |
| CCDC137 | Q6PK04 | IF-051 |
| CCDC58 | Q4VC31 | IF-051, LPC022, LPC020, CMC-6-123 |
| CCDC90B | Q9GZT6 | LPC011 |
| CCNE2 | O96020 | LPC011 |
| CCNL1 | Q9UK58 | LPC011 |
| CCNT1 | O60563 | IF-051 |
| CD109 | Q6YHK3 | IF-051, LPC020 |
| CD151 | P48509 | IF-072 |
| CD3EAP | O15446 | IF-051 |
| CD46 | P15529 | IF-051 |
| CD58 | P19256 | IF-051, CMC-6-123 |
| CD59 | P13987 | IF-051 |
| CD74 | P04233-2 | CMC-6-109, CMC-6-111, IF-072 |
| CD9 | P21926 | IF-049 |
| CDC40 | O60508 | IF-051, LPC022, LPC020, IF-049 |
| CDC5L | Q99459 | IF-051, LPC022, LPC020 |
| CDH1 | P12830 | IF-051, LPC022, LPC020, IF-049 |
| CDH13 | P55290 | CMC-6-109, CMC-6-111, IF-072 |
| CDH2 | P19022 | IF-051 |
| CDH3 | P22223 | CMC-6-109, CMC-6-111, IF-072 |
| CDK12 | Q9NYV4 | IF-051, IF-049 |
| CDK2AP1 | O14519 | IF-051 |
| CDK2AP2 | O75956 | IF-051, IF-049 |
| CDK5RAP1 | Q96SZ6 | CMC-6-109, CMC-6-111, IF-072, LPC020 |
| CDK5RAP1 | Q96SZ6-3 | LPC022 |
| CENPC | Q03188 | IF-051 |
| CFL2 | Q9Y281 | IF-051 |
| CHAF1B | Q13112 | IF-051 |
| CHAMP1 | Q96JM3 | CMC-6-109, CMC-6-111, IF-072 |
| CHCHD1 | Q96BP2 | IF-051 |
| CHCHD2 | Q9Y6H1 | IF-051, LPC020, IF-072, CMC-6-123 |
| CHCHD2P9 | Q5T1J5 | IF-051 |
| CHCHD4 | Q8N4Q1 | IF-051 |
| CHD6 | Q8TD26 | IF-051 |
| CHD7 | Q9P2D1 | LPC020 |
| CHTOP | Q9Y3Y2 | IF-051 |
| CIRBP | Q14011 | IF-051 |
| CKAP2 | Q8WWK9 | LPC020 |
| CKMT1A | P12532 | IF-051, LPC022, LPC020 |
| CLCC1 | Q96S66 | LPC022, LPC020 |
| CLIC4 | Q9Y696 | IF-051 |
| CLPB | Q9H078 | IF-051, LPC020, CMC-6-123 |
| CLPB | Q9H078-2 | IF-051 |
| CNPY2 | Q9Y2B0 | LPC020 |
| CNST | Q6PJW8 | IF-051, IF-049 |
| COA7 | Q96BR5 | LPC022, LPC020, IF-072, CMC-6-123, IF-085 |
| COL17A1 | Q9UMD9 | IF-051 |
| COQ9 | O75208 | LPC020 |
| COX20 | Q5RI15 | LPC020 |
| COX6B1 | P14854 | LPC020 |
| COX7A2 | P14406 | LPC020 |
| COX7A2L | O14548 | LPC022, LPC020 |
| COX7C | P15954 | CMC-6-123, LPC020 |
| CPOX | P36551 | LPC022, LPC020, IF-072, IF-051, IF-049 |
| CPSF7 | Q8N684 | IF-051 |
| CRYZ | Q08257 | LPC020 |
| CSRP1 | P21291 | IF-051 |
| CSTF2 | P33240 | IF-051, LPC022, LPC020 |
| CSTF2T | Q9H0L4 | LPC022, LPC020 |
| CSTF3 | Q12996 | IF-051, LPC022, LPC020, IF-049 |
| CT45A10 | P0DMU9 | IF-051, IF-049 |
| CTNNA1 | P35221 | IF-051 |
| CTNNB1 | P35222 | IF-051 |
| CTNND1 | O60716-3 | LPC020 |
| CTSB | P07858 | IF-051, LPC020, IF-072 |
| CTSD | P07339 | CMC-6-123, LPC020, IF-072 |
| CTSH | P09668 | IF-072, IF-051, CMC-6-123, CMC-6-111, CMC-6-109, |
| | | IF-085 |
| CTSS | P25774 | IF-072 |
| CTSV | O60911 | IF-051 |
| CTSZ | Q9UBR2 | IF-051, IF-080, IF-085 |
| CTTN | Q14247 | IF-051 |
| CUSTOS | Q96C57 | IF-051 |
| CUX1 | P39880 | IF-051, IF-049 |
| CWC15 | Q9P013 | IF-051, LPC020 |
| CYCS | P99999 | IF-051, LPC022, LPC020, CMC-6-123 |
| CYP1B1 | Q16678 | IF-073 |
| DAP3 | P51398 | LPC020 |
| DAZAP1 | Q96EP5 | IF-051, LPC020 |
| DBN1 | Q16643 | IF-051 |
| DDX21 | Q9NR30 | IF-051 |
| DDX52 | Q9Y2R4 | LPC011 |
| DFFA | O00273 | IF-051 |
| DHTKD1 | Q96HY7 | LPC020 |
| DHX9 | Q08211 | IF-051 |
| DIABLO | Q9NR28 | LPC022, LPC020, IF-051, CMC-6-123, IF-049 |
| DIDO1 | Q9BTC0 | IF-051, LPC022, LPC020 |
| DNAJA3 | Q96EY1 | LPC020 |
| DNAJB14 | Q8TBM8 | LPC020 |
| DNAJC8 | O75937 | IF-051 |
| DNASE2 | O00115 | LPC020, IF-072 |
| DOLPP1 | Q86YN1 | LPC011 |
| DPP7 | Q9UHL4 | CMC-6-123, IF-072 |
| DSG2 | Q14126 | IF-051, IF-049 |
| ECHS1 | P30084 | LPC020 |
| ECI2 | O75521 | LPC020 |
| ECSIT | Q9BQ95 | LPC020 |
| EDF1 | O60869 | IF-051 |
| EFNB1 | P98172 | IF-051, IF-049 |
| EFNB2 | P52799 | IF-051, CMC-6-123 |
| EFR3A | Q14156 | LPC020 |
| EIF1 | P41567 | IF-051 |
| ELAVL1 | Q15717 | LPC022, LPC020 |
| ELOA | Q14241 | IF-051 |
| EMG1 | Q92979 | IF-051 |
| EPN1 | Q9Y613 | IF-051, LPC022, LPC020 |
| EPN2 | O95208 | IF-051 |
| EPPK1 | P58107 | LPC020 |
| ERAL1 | O75616 | LPC020 |
| ERBIN | Q96RT1 | LPC020 |
| ERH | P84090 | IF-051, LPC020 |
| ERP29 | P30040 | IF-051, LPC020 |
| ETFA | P13804 | LPC020 |
| ETFDH | Q16134 | LPC020 |
| EZR | P15311 | IF-051 |
| FAHD1 | Q6P587 | IF-051, LPC020, IF-049 |
| FAM136A | Q96C01 | IF-051, LPC022, LPC020, IF-049 |
| FAM169A | Q9Y6X4 | IF-051, IF-049 |
| FAM177A1 | Q8N128 | IF-073 |
| FAM49B | Q9NUQ9 | IF-051 |
| FAM83B | Q5T0W9 | LPC020 |
| FAM83H | Q6ZRV2 | LPC020 |
| FASTKD3 | Q14CZ7 | LPC020 |
| FCF1 | Q9Y324 | IF-049 |
| FDX1 | P10109 | LPC020 |
| FDXR | P22570 | LPC020 |
| FECH | P22830 | LPC020 |
| FIP1L1 | Q6UN15 | IF-051, IF-049 |
| FKBP1A | P62942 | IF-051 |
| FNBP4 | Q8N3X1 | IF-051 |
| FSTL1 | Q12841 | IF-051 |
| FTL | P02792 | CMC-6-123, IF-085 |
| FUBP3 | Q96I24 | LPC020 |
| FUCA2 | Q9BTY2 | LPC020 |
| FUS | P35637 | IF-051, LPC022, LPC020 |
| FYTTD1 | Q96QD9 | IF-051, LPC020, IF-049, CMC-6-123 |
| FYTTD1 | Q96QD9-2 | LPC022, LPC020 |
| GADD45GIP1 | Q8TAE8 | LPC020 |
| GATAD2B | Q8WXI9 | IF-051 |
| GATB | O75879 | LPC022, LPC020 |
| GCSH | P23434 | CMC-6-123, LPC020 |
| GFER | P55789 | IF-051, LPC020, IF-072, CMC-6-123 |
| GFM2 | Q969S9 | LPC020 |
| GGH | Q92820 | IF-072 |
| GHITM | Q9H3K2 | LPC022, LPC020, IF-072, IF-051, CMC-6-123, IF-060, |
| | | IF-073, |
| | | IF-062, IF-064, IF-085 |
| GIPC2 | Q8TF65 | LPC011 |
| GLA | P06280 | IF-051 |
| GLB1L2 | Q8IW92 | LPC020 |
| GLO1 | Q04760 | IF-051 |
| GLRX5 | Q86SX6 | LPC020 |
| GLS | O94925-3 | LPC022, LPC020 |
| GLUD1 | P00367 | LPC020 |
| GM2A | P17900 | IF-051, CMC-6-123, IF-085 |
| GMFB | P60983 | IF-051 |
| GNG12 | Q9UBI6 | LPC020 |
| GPATCH4 | Q5T3I0 | IF-051 |
| GPC1 | P35052 | IF-051, CMC-6-111, IF-072, LPC020 |
| GPKOW | Q92917 | CMC-6-109, IF-051, IF-072 |
| GPT2 | Q8TD30 | LPC022, LPC020 |
| GRPEL2 | Q8TAA5 | LPC022, LPC020 |
| GRSF1 | Q12849 | LPC020 |
| GSTK1 | Q9Y2Q3 | LPC020 |
| GTF2B | Q00403 | IF-051, IF-049 |
| GTF2I | P78347 | CMC-6-109, IF-072 |
| H1FX | Q92522 | CMC-6-109, CMC-6-111, IF-072 |
| H3F3A | P84243 | LPC011 |
| HADHB | P55084 | LPC020 |
| HARS2 | P49590 | LPC020 |
| HAX1 | O00165 | CMC-6-109, IF-072 |
| HCCS | P53701 | LPC020 |
| HCLS1 | P14317 | IF-051 |
| HEBP1 | Q9NRV9 | IF-051, LPC020 |
| HEXB | P07686 | CMC-6-123 |
| HIBCH | Q6NVY1 | LPC020 |
| HINT1 | P49773 | IF-051 |
| HINT2 | Q9BX68 | IF-051, LPC020 |
| HIST1H1C | P16403 | CMC-6-123 |
| HIST1H1T | P22492 | IF-049 |
| HIST2H2AB | Q8IUE6 | IF-051 |
| HIST2H2BF | Q5QNW6 | IF-051 |
| HIST2H3A | Q71DI3 | LPC011 |
| HMGA1 | P17096-2 | IF-051 |
| HMGB2 | P26583 | IF-051 |
| HMGCL | P35914 | LPC020 |
| HMGN1 | P05114 | IF-051 |
| HMGN2 | P05204 | IF-051 |
| HMGN4 | O00479 | IF-051 |
| HMGN5 | P82970 | IF-051 |
| HNRNPA0 | Q13151 | IF-051, LPC022, LPC020 |
| HNRNPA1 | P09651 | IF-051, LPC020 |
| HNRNPA1 | P09651-2 | LPC020 |
| HNRNPA2B1 | P22626 | LPC022, LPC020 |
| HNRNPA3 | P51991 | LPC022, LPC020 |
| HNRNPAB | Q99729-2 | IF-051 |
| HNRNPAB | Q99729-3 | IF-051, LPC020 |
| HNRNPCL1 | O60812 | LPC020 |
| HNRNPD | Q14103 | IF-051 |
| HNRNPDL | O14979 | IF-051, LPC022, LPC020 |
| HNRNPH1 | P31943 | LPC022, LPC020 |
| HNRNPH2 | P55795 | LPC022, LPC020 |
| HNRNPH3 | P31942 | LPC020 |
| HNRNPK | P61978-2 | IF-051, LPC020, LPC011 |
| HNRNPL | P14866 | IF-051, LPC022, LPC020 |
| HNRNPM | P52272 | LPC020 |
| HNRNPR | O43390 | IF-051, LPC020 |
| HNRNPR | O43390-2 | LPC020 |
| HNRNPUL2 | Q1KMD3 | LPC020 |
| HPCAL1 | P37235 | IF-051 |
| HPDL | Q96IR7 | LPC020 |
| HSD17B10 | Q99714 | LPC020 |
| HSDL2 | Q6YN16 | LPC020 |
| HSPD1 | P10809 | LPC020 |
| HSPE1 | P61604 | CMC-6-123 |
| HTRA2 | O43464 | IF-051, LPC022, LPC020, IF-049 |
| IBA57 | Q5T440 | LPC020 |
| ICE1 | Q9Y2F5 | CMC-6-111, IF-072 |
| IDH3G | P51553 | IF-051, LPC020, IF-049 |
| IFI30 | P13284 | IF-072, IF-051, CMC-6-111, CMC-6-109, IF-049 |
| IGFBP7 | Q16270 | IF-051 |
| IK | Q13123 | IF-051, LPC020, IF-049 |
| IL6ST | P40189 | IF-051, LPC020, CMC-6-123 |
| ILF2 | Q12905 | IF-051 |
| ILF3 | Q12906-7 | IF-051 |
| IMMT | Q16891 | LPC020 |
| INCENP | Q9NQS7 | IF-080 |
| INF2 | Q27J81 | IF-051, LPC020, IF-049, CMC-6-123 |
| ISCA1 | Q9BUE6 | LPC020 |
| ISCU | Q9H1K1 | IF-051, CMC-6-123 |
| ISG20L2 | Q9H9L3 | IF-051, CMC-6-123 |
| ISOC2 | Q96AB3 | LPC020 |
| ITGA3 | P26006 | CMC-6-109, CMC-6-111, IF-072 |
| ITIH4 | Q14624 | IF-051 |
| ITM2B | Q9Y287 | LPC020 |
| JUP | P14923 | IF-051 |
| KCTD5 | Q9NXV2 | LPC011 |
| KDELC2 | Q7Z4H8 | LPC020 |
| KDM2A | Q9Y2K7 | LPC020, LPC011 |
| KHSRP | Q92945 | IF-051 |
| KIAA0391 | O15091 | LPC020 |
| KIAA1217 | Q5T5P2 | IF-051 |
| KIF20A | O95235 | IF-080 |
| KIF22 | Q14807 | CMC-6-109, CMC-6-111, IF-072, IF-080 |
| KIF23 | Q02241 | CMC-6-109, CMC-6-111, IF-072 |
| KIFC1 | Q9BW19 | CMC-6-109, CMC-6-111, IF-072 |
| KLF5 | Q13887 | LPC020 |
| KRT1 | P04264 | CMC-6-111 |
| KRT10 | P13645 | CMC-6-111, LPC034 |
| KRT13 | P13646 | LPC020 |
| KRT14 | P02533 | LPC020 |
| KRT17 | Q04695 | LPC020 |
| KRT18 | P05783 | LPC020 |
| KRT19 | P08727 | IF-051, LPC020, IF-049 |
| KRT2 | P35908 | CMC-6-111, LPC034 |
| KRT5 | P13647 | LPC020 |
| KRT6A | P02538 | LPC020 |
| KRT7 | P08729 | LPC020 |
| KRT79 | Q5XKE5 | LPC020 |
| KRT8 | P05787 | LPC020 |
| KRT9 | P35527 | CMC-6-111 |
| KTN1 | Q86UP2 | IF-051 |
| L1RE1 | Q9UN81 | LPC011 |
| L2HGDH | Q9H9P8 | LPC020 |
| LAMA3 | Q16787-1 | IF-051 |
| LAMTOR2 | Q9Y2Q5 | LPC020 |
| LARP1 | Q6PKG0 | IF-051 |
| LASP1 | Q14847 | IF-051 |
| LCOR | Q96JN0-3 | IF-051, IF-049 |
| LGALS1 | P09382 | IF-051 |
| LGMN | Q99538 | LPC022, LPC020, IF-072, IF-051, CMC-6-123, IF-049, |
| | | IF-073, |
| | | IF-085 |
| LMNA | P02545 | IF-051 |
| LMNB1 | P20700 | IF-051 |
| LMNB2 | Q03252 | IF-051 |
| LMO7 | Q8WWI1 | IF-051, IF-072, CMC-6-123 |
| LMTK2 | Q8IWU2 | IF-051 |
| LNPEP | Q9UIQ6 | IF-051 |
| LPXN | O60711 | CMC-6-109, CMC-6-111, IF-072 |
| LRCH3 | Q96II8 | LPC011 |
| LRIF1 | Q5T3J3 | LPC022, LPC020 |
| LSM14A | Q8ND56 | IF-051 |
| LSM2 | Q9Y333 | IF-051 |
| LSM6 | P62312 | IF-051 |
| LSM7 | Q9UK45 | IF-051 |
| LSM8 | O95777 | IF-051 |
| LVRN | Q6Q4G3 | CMC-6-109, CMC-6-111, IF-072 |
| LYPD3 | O95274 | IF-051, IF-049 |
| LYRM7 | Q5U5X0 | IF-049 |
| MAGOH | P61326 | IF-051 |
| MALSU1 | Q96EH3 | LPC020 |
| MAN1A2 | O60476 | IF-051 |
| MAN2B1 | O00754 | IF-072 |
| MANF | P55145 | IF-051 |
| MAP7 | Q14244 | LPC011 |
| MARCH5 | Q9NX47 | LPC020 |
| MATR3 | P43243 | IF-051, LPC022, LPC020, IF-049 |
| MAVS | Q7Z434 | IF-051, IF-049 |
| MBP | P02686 | IF-051 |
| MCAT | Q8IVS2 | LPC020 |
| MCM3AP | O60318 | LPC020 |
| MDC1 | Q14676 | IF-051, LPC020 |
| ME2 | P23368 | LPC020 |
| MED1 | Q15648 | LPC020 |
| MED11 | Q9P086 | LPC011 |
| MED21 | Q13503 | IF-051 |
| MED4 | Q9NPJ6 | LPC020 |
| MEN1 | O00255 | CMC-6-109, CMC-6-111, IF-072 |
| MESD | Q14696 | IF-051 |
| MFSD6 | Q6ZSS7 | IF-049 |
| MGA | Q8IWI9 | LPC020 |
| MGME1 | Q9BQP7 | IF-049 |
| MIC13 | Q5XKP0 | LPC022, LPC020 |
| MICA | Q29983 | IF-051 |
| MICU1 | Q9BPX6 | LPC020 |
| MIF | P14174 | IF-051 |
| MKI67 | P46013 | IF-072, IF-051, CMC-6-111, CMC-6-109, IF-049 |
| MMP1 | P03956 | IF-051 |
| MMTAG2 | Q9BU76 | LPC020 |
| MNAT1 | P51948 | LPC020 |
| MOB1B | Q7L9L4 | IF-051 |
| MPRIP | Q6WCQ1 | IF-051 |
| MRPL1 | Q9BYD6 | LPC020 |
| MRPL10 | Q7Z7H8 | LPC020 |
| MRPL12 | P52815 | IF-051, CMC-6-123 |
| MRPL13 | Q9BYD1 | LPC022, LPC020 |
| MRPL14 | Q6P1L8 | LPC022, LPC020 |
| MRPL15 | Q9P015 | LPC020 |
| MRPL16 | Q9NX20 | LPC022, LPC020 |
| MRPL17 | Q9NRX2 | LPC022, LPC020 |
| MRPL20 | Q9BYC9 | LPC020 |
| MRPL21 | Q7Z2W9 | LPC020 |
| MRPL23 | Q16540 | LPC022, LPC020 |
| MRPL24 | Q96A35 | LPC020 |
| MRPL3 | P09001 | LPC020 |
| MRPL33 | O75394 | CMC-6-123, LPC020 |
| MRPL34 | Q9BQ48 | CMC-6-123 |
| MRPL37 | Q9BZE1 | LPC022, LPC020 |
| MRPL38 | Q96DV4 | LPC020 |
| MRPL39 | Q9NYK5 | LPC020 |
| MRPL4 | Q9BYD3 | LPC020 |
| MRPL40 | Q9NQ50 | LPC020 |
| MRPL41 | Q8IXM3 | LPC022, LPC020, IF-049, LPC011 |
| MRPL42 | Q9Y6G3 | LPC020 |
| MRPL45 | Q9BRJ2 | LPC022, LPC020 |
| MRPL46 | Q9H2W6 | LPC020 |
| MRPL47 | Q9HD33 | LPC020 |
| MRPL49 | Q13405 | LPC020 |
| MRPL50 | Q8N5N7 | LPC020 |
| MRPL52 | Q86TS9 | LPC020 |
| MRPL54 | Q6P161 | IF-049 |
| MRPL9 | Q9BYD2 | LPC020 |
| MRPS10 | P82664 | LPC020 |
| MRPS11 | P82912 | LPC020 |
| MRPS12 | O15235 | LPC022 |
| MRPS15 | P82914 | LPC020 |
| MRPS16 | Q9Y3D3 | LPC020 |
| MRPS17 | Q9Y2R5 | LPC020 |
| MRPS18B | Q9Y676 | LPC020 |
| MRPS18C | Q9Y3D5 | LPC022, LPC020 |
| MRPS2 | Q9Y399 | LPC020 |
| MRPS22 | P82650 | LPC020 |
| MRPS23 | Q9Y3D9 | LPC022, LPC020 |
| MRPS25 | P82663 | LPC020 |
| MRPS26 | Q9BYN8 | LPC020 |
| MRPS28 | Q9Y2Q9 | LPC020 |
| MRPS30 | Q9NP92 | LPC022, LPC020 |
| MRPS31 | Q92665 | LPC020 |
| MRPS34 | P82930 | LPC020 |
| MRPS35 | P82673 | LPC020 |
| MRPS6 | P82932 | LPC020 |
| MRPS7 | Q9Y2R9 | LPC020 |
| MRPS9 | P82933 | LPC020 |
| MSLN | Q13421 | IF-051, LPC020, CMC-6-123 |
| MSLN | Q13421-2 | IF-051, IF-049 |
| MSN | P26038 | IF-051 |
| MT-CO2 | P00403 | LPC020 |
| MT-ND1 | P03886 | LPC020 |
| MT1X | P80297 | IF-051 |
| MTDH | Q86UE4 | IF-051 |
| MTERF3 | Q96E29 | LPC020 |
| MTFR1 | Q15390 | IF-051, CMC-6-123, IF-085 |
| MTG1 | Q9BT17 | LPC022, LPC020 |
| MTHFD1L | Q6UB35 | LPC020 |
| MTHFD2 | P13995 | LPC020 |
| MTREX | P42285 | IF-051 |
| MXRA7 | P84157 | IF-051, IF-049 |
| MYDGF | Q969H8 | IF-051 |
| MYO19 | Q96H55 | IF-072 |
| NAGA | P17050 | IF-072 |
| NCBP3 | Q53F19 | IF-051 |
| NCL | P19338 | IF-051 |
| NCOA4 | Q13772 | LPC020 |
| NCOA5 | Q9HCD5 | IF-051, IF-049 |
| NDUFA10 | O95299 | LPC020 |
| NDUFA13 | Q9P0J0 | LPC020 |
| NDUFA2 | O43678 | LPC020 |
| NDUFA4 | O00483 | LPC022, LPC020, IF-049 |
| NDUFA5 | Q16718 | LPC020 |
| NDUFA7 | O95182 | IF-049 |
| NDUFA8 | P51970 | LPC020 |
| NDUFA9 | Q16795 | LPC020 |
| NDUFAB1 | O14561 | IF-051, IF-049 |
| NDUFAF2 | Q8N183 | LPC020 |
| NDUFAF4 | Q9P032 | LPC020 |
| NDUFB10 | O96000 | LPC020 |
| NDUFB4 | O95168 | LPC020 |
| NDUFS1 | P28331 | LPC020 |
| NDUFS2 | O75306 | LPC020 |
| NDUFS3 | O75489 | LPC020 |
| NDUFS6 | O75380 | LPC020 |
| NDUFS8 | O00217 | LPC020 |
| NDUFV1 | P49821 | LPC020 |
| NDUFV2 | P19404 | LPC020 |
| NECTIN1 | Q15223 | IF-051, LPC020, IF-049, CMC-6-123 |
| NECTIN2 | Q92692 | IF-051, LPC020 |
| NENF | Q9UMX5 | IF-051 |
| NFS1 | Q9Y697 | LPC020 |
| NFU1 | Q9UMS0 | IF-051, IF-049 |
| NGRN | Q9NPE2 | LPC020 |
| NOC4L | Q9BVI4 | LPC020 |
| NOL12 | Q9UGY1 | IF-051, IF-049 |
| NOL7 | Q9UMY1 | IF-051 |
| NOLC1 | Q14978-2 | IF-051 |
| NOLC1 | Q14978-3 | LPC011 |
| NONO | Q15233 | IF-051, LPC020 |
| NOP53 | Q9NZM5 | LPC020 |
| NPC2 | P61916 | IF-072, IF-051, CMC-6-123, IF-073, IF-085 |
| NPM3 | O75607 | LPC020 |
| NRAS | P01111 | LPC020 |
| NSD2 | O96028 | IF-051 |
| NSUN5 | Q96P11 | LPC020 |
| NT5E | P21589 | IF-051 |
| NUCB1 | Q02818 | CMC-6-123, IF-085 |
| NUMA1 | Q14980 | IF-051, LPC020, IF-049 |
| NUP37 | Q8NFH4 | LPC020 |
| NUP50 | Q9UKX7 | IF-051 |
| NUP54 | Q7Z3B4 | LPC020 |
| NUP62 | P37198 | LPC020 |
| NUSAP1 | Q9BXS6 | IF-051 |
| NVL | O15381 | IF-051, CMC-6-109, CMC-6-111, IF-072 |
| NXF1 | Q9UBU9 | IF-051 |
| NXT1 | Q9UKK6 | LPC020 |
| OAS1 | P00973 | IF-051 |
| OAT | P04181 | LPC020 |
| OCLN | Q16625 | IF-051 |
| OGDH | Q02218 | LPC020 |
| OVOL1 | O14753 | IF-051 |
| OXA1L | Q15070 | LPC020 |
| P4HB | P07237 | IF-051 |
| PABPN1 | Q86U42 | IF-051, LPC020, IF-049 |
| PAM16 | Q9Y3D7 | LPC020 |
| PANX1 | Q96RD7 | LPC020 |
| PARL | Q9H300 | IF-051, LPC020 |
| PATZ1 | Q9HBE1 | CMC-6-111, IF-072 |
| PAWR | Q96IZ0 | IF-051 |
| PBRM1 | Q86U86 | LPC020 |
| PCF11 | O94913 | IF-051 |
| PCLAF | Q15004 | IF-073 |
| PDAP1 | Q13442 | IF-051 |
| PDE6C | P51160 | IF-051 |
| PDHB | P11177 | LPC020 |
| PDIA3 | P30101 | IF-051 |
| PDIA6 | Q15084 | LPC020 |
| PEBP1 | P30086 | IF-051 |
| PHB | P35232 | LPC020 |
| PHF14 | O94880 | IF-072, LPC011 |
| PHF3 | Q92576 | IF-051, LPC022, LPC020 |
| PHIP | Q8WWQ0 | IF-072 |
| PICALM | Q13492 | IF-051 |
| PKP1 | Q13835 | CMC-6-111, IF-072 |
| PKP2 | Q99959-2 | IF-051 |
| PLEC | Q15149-3 | LPC020 |
| PLEC | Q15149-4 | IF-051 |
| PLEKHA6 | Q9Y2H5 | IF-072 |
| PLRG1 | O43660 | IF-051 |
| PLSCR1 | O15162 | LPC020 |
| PLSCR3 | Q9NRY6 | LPC020 |
| POFUT1 | Q9H488 | IF-051, LPC020 |
| POFUT2 | Q9Y2G5-1 | LPC020 |
| POGZ | Q7Z3K3 | IF-072 |
| POLDIP2 | Q9Y2S7 | LPC020 |
| POLR2D | O15514 | LPC020 |
| POLR2F | P61218 | IF-051, CMC-6-123 |
| POLR2G | P62487 | IF-051 |
| POLRMT | O00411 | LPC020 |
| POM121C | A8CG34 | IF-051, LPC020 |
| POSTN | Q15063 | IF-051 |
| PPIB | P23284 | IF-051 |
| PPIF | P30405 | IF-051, IF-049 |
| PPP1R9B | Q96SB3 | IF-051, IF-049 |
| PPT1 | P50897 | IF-051, IF-049, IF-072 |
| PQBP1 | O60828 | IF-051, LPC020, IF-049 |
| PRC1 | O43663 | IF-080 |
| PRKN | O60260-5 | LPC020 |
| PRKRIP1 | Q9H875 | IF-051 |
| PRNP | P04156 | IF-051, LPC022, LPC020 |
| PRPF19 | Q9UMS4 | IF-051 |
| PRSS37 | A4D1T9 | LPC020 |
| PSAP | P07602 | IF-051, LPC020, IF-049 |
| PSEN1 | P49768 | IF-051 |
| PSIP1 | O75475 | IF-051 |
| PSPC1 | Q8WXF1 | IF-051 |
| PTBP1 | P26599 | IF-051, LPC020 |
| PTBP3 | O95758-1 | LPC020 |
| PTCD1 | O75127 | LPC020 |
| PTK7 | Q13308 | IF-051, IF-049 |
| PTMA | P06454 | IF-051, IF-085 |
| PTPMT1 | Q8WUK0 | LPC020 |
| PTRH1 | Q86Y79 | LPC020, LPC011 |
| PYM1 | Q9BRP8 | IF-051, CMC-6-123 |
| QRSL1 | Q9HOR6 | LPC022 |
| RACGAP1 | Q9H0H5 | IF-072, IF-080, IF-051, CMC-6-111, CMC-6-109, IF-049 |
| RAD21 | O60216 | IF-051 |
| RALY | Q9UKM9 | LPC020 |
| RAVER1 | Q8IY67 | LPC020 |
| RBBP6 | Q7Z6E9 | IF-051 |
| RBBP7 | Q16576 | IF-051 |
| RBM14 | Q96PK6 | LPC022, LPC020 |
| RBM15 | Q96T37 | LPC020 |
| RBM19 | Q9Y4C8 | IF-051, IF-049 |
| RBM22 | Q9NW64 | IF-051 |
| RBM27 | Q9P2N5 | LPC020 |
| RBM3 | P98179 | CMC-6-109, IF-051 |
| RBM4 | Q9BWF3 | LPC020 |
| RBM6 | P78332 | IF-051 |
| RBM8A | Q9Y5S9 | IF-051 |
| RBMX | P38159 | IF-051, LPC020, IF-049 |
| RBMXL1 | Q96E39 | IF-051 |
| RDX | P35241 | IF-051 |
| REEP5 | Q00765 | IF-051, LPC022, LPC020, IF-049 |
| REXO4 | Q9GZR2 | IF-051, LPC020, IF-049 |
| RGS10 | O43665 | IF-051, IF-049 |
| RIF1 | Q5UIP0 | IF-051, LPC020, IF-049 |
| RMDN1 | Q96DB5 | IF-051, LPC020, CMC-6-123 |
| RNASET2 | O00584 | CMC-6-123 |
| RNF121 | Q9H920 | IF-051 |
| RNMT | O43148 | IF-051 |
| RP9 | Q8TA86 | IF-051 |
| RPF1 | Q9H9Y2 | LPC020, LPC011 |
| RPLP1 | P05386 | IF-073 |
| RPS21 | P63220 | IF-051 |
| RPUSD3 | Q6P087 | LPC020 |
| RREB1 | Q92766 | IF-051 |
| RRN3 | Q9NYV6 | LPC011 |
| RRP36 | Q96EU6 | IF-051 |
| RSBN1 | Q5VWQ0 | IF-051 |
| RSF1 | Q96T23 | IF-051 |
| RSRC1 | Q96IZ7 | IF-051 |
| S100A10 | P60903 | IF-051 |
| S100A14 | Q9HCY8 | IF-051, LPC020, IF-049 |
| SAFB | Q15424 | IF-051, LPC020, IF-049 |
| SAFB2 | Q14151 | IF-051, LPC022, LPC020, IF-049 |
| SAMD1 | Q6SPF0 | IF-051, IF-049 |
| SAMM50 | Q9Y512 | LPC020 |
| SAP130 | Q9H0E3 | IF-051 |
| SARNP | P82979 | IF-051 |
| SCAF11 | Q99590 | IF-051 |
| SCAF8 | Q9UPN6 | IF-051 |
| SCFD1 | Q8WVM8 | IF-051 |
| SCO1 | O75880 | IF-051 |
| SDC4 | P31431 | IF-051 |
| SDF2L1 | Q9HCN8 | IF-051 |
| SDHA | P31040 | LPC020 |
| SDHAF2 | Q9NX18 | LPC020 |
| SDHB | P21912 | LPC020 |
| SELENOI | Q9C0D9 | LPC011 |
| SERPINH1 | P50454 | IF-051 |
| SETX | Q7Z333 | IF-073 |
| SF1 | Q15637-6 | LPC020 |
| SF3A1 | Q15459 | IF-051 |
| SF3A3 | Q12874 | IF-051 |
| SF3B2 | Q13435 | IF-051 |
| SF3B4 | Q15427 | LPC020 |
| SFPQ | P23246 | IF-051 |
| SFSWAP | Q12872 | IF-051 |
| SFXN1 | Q9H9B4 | LPC020 |
| SFXN3 | Q9BWM7 | LPC020 |
| SHMT2 | P34897 | LPC020 |
| SLC16A1 | P53985 | IF-051 |
| SLC1A4 | P43007 | IF-051 |
| SLC1A5 | Q15758 | IF-051 |
| SLC25A10 | Q9UBX3 | LPC020 |
| SLC25A6 | P12236 | LPC020 |
| SLC30A1 | Q9Y6M5 | LPC020, IF-072 |
| SLC3A2 | P08195 | IF-051 |
| SLC43A3 | Q8NBI5 | IF-051 |
| SLC48A1 | Q6P1K1 | LPC020 |
| SLC6A6 | P31641 | LPC020 |
| SLC9A1 | P19634 | IF-051 |
| SLC9A3R1 | O14745 | IF-051 |
| SLC9A3R2 | Q15599 | IF-051 |
| SLIRP | Q9GZT3 | LPC020 |
| SLMAP | Q14BN4 | IF-051, IF-049 |
| SLU7 | O95391 | LPC022, LPC020 |
| SMARCB1 | Q12824 | IF-051, LPC020 |
| SMARCD1 | Q96GM5 | IF-051 |
| SMARCD2 | Q92925-2 | LPC020 |
| SMIM12 | Q96EX1 | IF-051 |
| SMU1 | Q2TAY7 | IF-051 |
| SNIP1 | Q8TAD8 | LPC020 |
| SNRNP70 | P08621 | IF-051 |
| SNRPA1 | P09661 | IF-051 |
| SNRPC | P09234 | IF-051 |
| SNRPD1 | P62314 | LPC011 |
| SNW1 | Q13573 | IF-051 |
| SON | P18583-5 | LPC020 |
| SON | P18583-6 | LPC020 |
| SORL1 | Q92673 | IF-049 |
| SORT1 | Q99523 | CMC-6-109, IF-072 |
| SOX15 | O60248 | IF-051 |
| SPECC1 | Q5M775 | IF-051 |
| SPIN1 | Q9Y657 | IF-051 |
| SPINT1 | O43278 | IF-051, IF-049 |
| SPRY4 | Q9C004 | IF-051 |
| SPRYD4 | Q8WW59 | IF-049 |
| SRCAP | Q6ZRS2 | IF-051 |
| SREK1 | Q8WXA9-2 | IF-051 |
| SRP9 | P49458 | IF-051 |
| SRRT | Q9BXP5 | IF-051 |
| SRSF9 | Q13242 | LPC020 |
| SSBP1 | Q04837 | LPC020 |
| ST14 | Q9Y5Y6 | IF-051, LPC020, IF-049, IF-072 |
| STK24 | Q9Y6E0 | IF-051 |
| STOML2 | Q9UJZ1 | LPC020 |
| STX12 | Q86Y82 | IF-051 |
| STX18 | Q9P2W9 | IF-051 |
| STX5 | Q13190 | IF-051 |
| STX7 | O15400 | IF-051 |
| SUGP1 | Q8IWZ8 | IF-051 |
| SUMF2 | Q8NBJ7 | LPC020 |
| SYAP1 | Q96A49 | IF-051 |
| SYF2 | O95926 | IF-051 |
| SYMPK | Q92797 | IF-049 |
| SYNE2 | Q8WXH0 | IF-051 |
| TACC1 | O75410 | IF-051, IF-049 |
| TACO1 | Q9BSH4 | LPC020 |
| TAF11 | Q15544 | IF-049 |
| TAF15 | Q92804 | IF-051, LPC020 |
| TAGLN2 | P37802 | IF-051 |
| TARDBP | Q13148 | LPC022, LPC020 |
| TBCA | O75347 | IF-051 |
| TBL1XR1 | Q9BZK7 | IF-051 |
| TBP | P20226 | LPC020 |
| TCF20 | Q9UGU0 | IF-051 |
| TCOF1 | Q13428-3 | IF-051 |
| TCOF1 | Q13428-7 | LPC020 |
| TEX264 | Q9Y619 | IF-051 |
| TGFBI | Q15582 | IF-051, CMC-6-123 |
| THOC7 | Q619Y2 | LPC020 |
| THRAP3 | Q9Y2W1 | LPC020 |
| TIMM13 | Q9Y5L4 | IF-051, LPC022, LPC020 |
| TIMM17A | Q99595 | LPC020 |
| TIMM23B | Q5SRD1 | LPC022, LPC020 |
| TIMM29 | Q9BSF4 | IF-051, IF-049 |
| TIMM50 | Q3ZCQ8 | LPC020 |
| TK1 | P04183 | CMC-6-109, CMC-6-111, IF-072 |
| TMBIM6 | P55061 | LPC049, LPC011, IF-065, IF-063 |
| TMEM106B | Q9NUM4 | IF-051, CMC-6-123 |
| TMEM126A | Q9H061 | LPC020 |
| TMEM214 | Q6NUQ4 | IF-072 |
| TMEM237 | Q96Q45 | IF-049 |
| TMEM30A | Q9NV96 | IF-051 |
| TMEM40 | Q8WWA1 | LPC020 |
| TMPO | P42166 | LPC020 |
| TMSB4X | P62328 | IF-051 |
| TNFRSF12A | Q9NP84 | LPC020 |
| TOMM6 | Q96B49 | LPC020 |
| TOMM7 | Q9P0U1 | LPC020 |
| TOP2A | P11388 | CMC-6-109, CMC-6-111, IF-072, LPC011 |
| TOR1AIP2 | Q8NFQ8 | IF-051 |
| TOR4A | Q9NXH8 | LPC049 |
| TP53BP1 | Q12888 | IF-051, LPC020 |
| TPP1 | O14773 | LPC020, IF-072 |
| TPST1 | O60507 | LPC020 |
| TPX2 | Q9ULW0 | CMC-6-109, CMC-6-111, IF-072, IF-080 |
| TRIM28 | Q13263 | CMC-6-109, CMC-6-111, IF-072 |
| TRIM33 | Q9UPN9 | IF-072 |
| TRMT10C | Q7L0Y3 | LPC020 |
| TSFM | P43897 | LPC020 |
| TSNAX | Q99598 | LPC011 |
| TST | Q16762 | LPC020 |
| TTC19 | Q6DKK2 | IF-051, IF-049, CMC-6-123 |
| TUFM | P49411 | LPC020 |
| TXN2 | Q99757 | IF-051, LPC020 |
| TXNDC12 | O95881 | IF-051 |
| TXNDC5 | Q8NBS9 | IF-051 |
| TXNIP | Q9H3M7 | IF-073, IF-080, IF-085 |
| TXNL4A | P83876 | IF-051 |
| TYW1 | Q9NV66 | LPC011 |
| U2AF2 | P26368 | IF-051 |
| U2SURP | O15042 | LPC020 |
| U2SURP | O15042-2 | IF-051 |
| UBE2G2 | P60604 | IF-051 |
| UBE2J2 | Q8N2K1 | IF-051 |
| UHRF1 | Q96T88 | CMC-6-109, CMC-6-111, IF-072, IF-080 |
| URB1 | O60287 | LPC020 |
| USP36 | Q9P275 | IF-051, IF-049 |
| UTP15 | Q8TED0 | LPC011 |
| UTP18 | Q9Y5J1 | LPC011 |
| VAMP7 | P51809 | IF-051, IF-049 |
| VDAC1 | P21796 | LPC020 |
| VDAC2 | P45880 | LPC020 |
| VIM | P08670 | IF-051, CMC-6-123 |
| VIRMA | Q69YN4 | LPC020 |
| VSIR | Q9H7M9 | CMC-6-109, CMC-6-111, IF-072 |
| VTI1B | Q9UEU0 | IF-051 |
| WDFY1 | Q8IWB7 | IF-072 |
| WDR33 | Q9C0J8 | LPC020 |
| WDR76 | Q9H967 | CMC-6-109, CMC-6-111, IF-072 |
| WTAP | Q15007 | IF-051, IF-049 |
| XPNPEP3 | Q9NQH7 | LPC020 |
| XRN2 | Q9H0D6 | IF-051 |
| YAP1 | P46937 | IF-051 |
| YARS2 | Q9Y2Z4 | LPC020 |
| YIPF3 | Q9GZM5 | IF-051, LPC020, IF-049 |
| YLPM1 | P49750 | IF-051, IF-049 |
| ZBTB1 | Q9Y2K1 | IF-051, CMC-6-123, IF-085 |
| ZBTB11 | O95625 | IF-080 |
| ZC3H13 | Q5T200 | IF-051, LPC020 |
| ZC3H14 | Q6PJT7 | IF-051, IF-072 |
| ZC3H18 | Q86VM9 | IF-051 |
| ZC3HC1 | Q86WB0 | LPC020 |
| ZCCHC8 | Q6NZY4 | IF-051 |
| ZFR | Q96KR1 | LPC022, LPC020 |
| ZMYM4 | Q5VZL5 | LPC022, LPC020 |
| ZNF148 | Q9UQR1 | IF-051 |
| ZNF185 | O15231 | IF-051, IF-049 |
| ZNF217 | O75362 | IF-051, CMC-6-123, IF-085 |
| ZNF512B | Q96KM6 | IF-051 |
| ZNF638 | Q14966 | IF-051, LPC020 |
| ZNF800 | Q2TB10 | IF-072 |
| ZNF830 | Q96NB3 | IF-051, LPC020 |

### Biological Materials and Methods

Materials: Rabbit anti-SAFB1 (PA5-2135P, 1:3,000 dilution) and rabbit anti-FAM136A (PA5-56345, 1:1,000 dilution) were ordered from Life Technologies. Rabbit anti-SAFB2 (11642-1-AP, 1:3,000 dilution), rabbit anti-TARDBP (10782-2-AP, 1:1,000 dilution) were ordered from Proteintech. Rabbit anti-PTMA (PA5-71580, 1:1,000 dilution) was ordered from Invitrogen. Rabbit anti-LGMN (93627S, 1:1,000), rabbit anti-PPP1R9B (14136S, 1:1,000 dilution), rabbit anti-DBC1 (5693S, 1:1,000 dilution), rabbit anti-MAVS (3993S, 1:1,000 dilution), rabbit anti-Beta-actin (4967S, 1:1,000 dilution), rabbit anti-histone H3 (9715S, 1:1,000 dilution), mouse anti-HA (2367S, 1:2,000 dilution), rabbit anti-HA (3724S, 1:2,000 dilution), rabbit anti-FLAG (14793, 1:1,000 dilution), mouse anti-FLAG (8146S, 1:1,000 dilution), anti-rabbit HRP (7074P2, 1:10,000 dilution) were ordered from Cell Signaling Technology. Epoxomicin (A2606, proteasome inhibitor) was ordered from APEx Bio. MG132 (HY-13259, proteasome inhibitor) and MLN4924 (HY-70062, neddylation inhibitor) were ordered from MedChem Express.

Mammalian cell culture: MDA-MB-231 cells (ATCC) were maintained in DMEM supplemented with 10% (v/v) fetal bovine serum (FBS), 1% (v/v) penicillin/streptomycin, and 2 mM glutamine. HCC1806 cells (ATCC) were maintained in RPMI supplemented with 10% (v/v) fetal bovine serum (FBS), 1% (v/v) penicillin/streptomycin, and 2 mM glutamine. All cell lines were grown at 37° C. in a humidified 5% CO₂ atmosphere.

Treatment of Live Cells with FragTAC Probes: Separate 6 cm (for gel-based analyses) or 10 cm (for MS-based analyses) dishes of MDA-MB-231 cells or HCC1806 cells were grown to 80-95% confluency with DMEM media and RPMI media respectively. The growth medium was aspirated, and the cells were washed with Dulbecco's phosphate buffered saline (DPBS). The cells were incubated with serum-free media containing fragment probes (200 μM) or control probes (200 μM) for 6 hours at 37° C. under a 5% CO₂ atmosphere. The cultures were scraped, washed with cold DPBS, and collected into 15 mL centrifuge tubes, then transferred to 1.5 mL Eppendorf tubes. The cell suspensions were centrifuged (1,400 g, 3 min) and the pellets were stored at −80° C. until the next stage of processing.

Sample preparation for TMT MS Quantitative Proteomics: For treatment of cell lysates, cells were collected, cell pellets were resuspended in 100 μL DPBS containing 1× Halt protease inhibitor cocktail, and lysed by sonication (15 ms on, 40 ms off, 15% amplitude, 1 s total). Protein concentrations were normalized (2 mg/mL in 100 μL with cold DPBS) using the Lowry Protein Assay (Pierce). Urea (48 mg) was weighed for each sample in 2 mL LoBind Eppendorf tubes and cell lysates (100 μL=200 μg) were added (final urea concentration=8 M). Pellets were resuspended in a freshly prepared 1:1 solution (50 μL) TCEP (200 mM in DPBS) and K₂CO₃ (600 mM in DPBS) and incubated (30 min, 37° C.) while shaking. After reaction, a solution of freshly prepared iodoacetamide (70 μL, 400 mM in DPBS) was added and incubated for 30 min at room temperature while protected from light. After reaction, 1.8 mL of cold 3:1:2 MeOH/CHCl₃/H₂O solution was added to each tube, and the samples were centrifuged (10,000×g, 10 min, 4° C.), forming a disc. The supernatant was carefully removed, MeOH (600 μL) was added, samples were centrifuged as previously described and the supernatant was removed. Cell pellets were resuspended in TEAB (160 μL, 100 mM, pH 8.5) and sonicated as described previously. Endoproteinase LysC (20 μL, ½ vial, 10 μg dissolved in 220 μL of 100 mM TEAB pH 8.5) was added to each and incubated at 37° C. for 2 h with shaking. Sequencing-grade modified porcine trypsin (20 μL, 1 vial, 20 μg dissolved in 220 μL of 100 mM TEAB pH 8.5), a solution of Protease Max (2 μL, 1% (w/v) in 100 mM TEAB pH 8.5) and a solution of CaCl₂) (2 μL, 100 mM) were added to the samples and incubated at 37° C. overnight (14 h) with shaking. The digest was separated by centrifugation (12,000×g, 10 min, 4° C.). Peptide concentration was determined using Pierce Quantitative Fluorometric Peptide Assay Kit (Thermo Fisher Scientific, 23290) according to manufacturer's instructions. MS-grade acetonitrile (12.5 μL) was added to each and samples were labeled with respective TMT 10 plex isotope (8 μL, 20 μg/μL) for 1 h with occasional vortexing at RT. To quench, hydroxylamine (3 μL, 5% v/v) was added to each sample, vortexed, and incubated for 15 min at RT. Formic acid (5 μL) was added to each tube to acidify and the samples were dried under vacuum centrifugation. The samples were combined by redissolving the contents of one tube in a solution of trifluoroacetic acid (TFA, 400 μL, 0.1% in water) and transferred into each sample until all samples were redissolved. The stepwise process was repeated with formic acid (Buffer A, 200 μL, 0.1% in water) for a final volume of 600 μL. The samples were fractionated using a fractionation kit (Pierce high pH Reversed-Phase Fractionation Kit, Thermo Fisher Scientific 84868) according to manufacturer's instructions. The peptide fractions were eluted from the spin column with consecutive solutions of 0.1% triethylamine combined with MeCN (5-75% MeCN). The fractions were combined pairwise (fraction 1 and fraction 10, fraction 2 and fraction 11, etc.), dried via vacuum centrifugation, and stored at −80° C. until ready for mass spectrometer injection.

LC-MS analysis of TMT samples: TMT labeled samples were redissolved in MS buffer A (20 μL, 0.1% formic acid in water). 3 μL of each sample was loaded onto an Acclaim PepMap 100 precolumn (75 μm×2 mm) and eluted on an Acclaim PepMap RSLC analytical column (75 μm×15 cm) using the UltiMate 3000 RSLCnano system (Thermo Fisher Scientific). Buffer A was prepared as described above and buffer B (0.1% formic acid in MeCN) were used in a 220 min gradient (flow rate 0.3 mL min, 35° C.) of 2% buffer B for 10 min, followed by an incremental increase to 30% buffer B over 192 min, 60% buffer B for 5 min, 60-95% buffer B for 1 min, hold at 95% buffer B for 5 min, followed by descent to 2% buffer B for 1 min followed by re-equilibration at 2% for 6 min. The elutions were analyzed with a Thermo Fisher Scientific Orbitrap Fusion Lumos mass spectrometer with a cycle time of 3 s and nano-LC electrospray ionization source applied voltage of 2.0 kV. MS1 spectra were recorded at a resolution of 120,000 with an automatic gain control (AGC) value of 1×10⁶ ions, maximum injection time of 50 ms (dynamic exclusion enabled, repeat count 1, duration 20 s). The scan range was specified from 375 to 1,500 m/z. Peptide fragmentation MS2 spectra was recorded via collision-induced diffusion (CID) and quadrupole ion trap analysis (AGC 1.8×10⁴, 30% collision energy, maximum inject time 120 ms, isolation window 1.6). MS3 spectra was generated by high-energy collision-induced dissociation (HCD) with collision energy of 65%. Precursor selection included up to 10 MS² ions for the MS³ spectrum.

TMT proteomics data analysis: Proteomic analysis was performed with the processing software Proteome Discoverer 2.4 (Thermo Fisher Scientific). Peptide sequences were identified by matching proteome databases with experimental fragmentation patterns via the SEQUEST HT algorithm. Fragment tolerances were set to 0.6 Da, and precursor mass tolerances set to 10 ppm with one missed cleavage site allowed. Spectra were searched against the Homo Sapiens proteome database (42,358 sequences) using a false discovery rate of 1% (Percolator). MS³ peptide quantitation was performed with a mass tolerance of 20 ppm. Identified proteins were required to have at least two unique peptides. TMT ratios obtained by Proteome Discoverer were transformed with log₂(x), and p-values were calculated via Student's two-tailed t-tests with two biological replicates.

Cell viability assays: Cells were seeded in white-opaque 96-well plates in full growth media at a density of 2,000 cells/well (50 μL) and were allowed to grow for 14 h at 37° C. in a humidified 5% CO₂ atmosphere. The cells were then treated with compounds in triplicate and incubated at 37° C. in a humidified 5% CO₂ atmosphere for 6 hours. Cell viability was determined using the luciferase-based Cell Titer-Glo Luminescent Cell Viability Assay (Promega) following manufacturer's guidelines. Data represents the average and standard deviation of triplicates in measured luminescence.

Immunoblots: After cells were harvested, cell pellets were resuspended in 100 μL DPBS containing 1× Halt protease inhibitor cocktail, and lysed by sonication (15 ms on, 40 ms off, 15% amplitude, 1 s total). Protein concentrations were normalized (2 mg/mL in 100 μL with cold DPBS) as previously described. 4X SDS gel loading buffer (33 μL) was added to the solution of protein lysate and the resulting mixture was heated at 95° C. for 15 min. Proteins (15 μg total protein loaded per gel lane) were resolved by SDS-PAGE (10% acrylamide) made in-house, and transferred to PVDF membrane (0.2 μM, 1620177, Bio-Rad). The membrane was blocked with 5% BSA in Tris-buffered saline with Tween (TBST) buffer (0.1% Tween 20, 20 mM Tris-HCl 7.6, 150 mM NaCl) at room temperature for 1 h. The antibody was diluted with fresh 5% BSA in TBST buffer (dilutions were performed following manufacturer's guidelines) and incubated with membrane overnight (14 h) at 4° C. Membrane was washed three times with TBST buffer, left 5 minutes between each wash on a rocker and then incubated with secondary antibody in 5% dry milk in TBST at room temperature for 2 h on a rocker. Membrane was washed three times with TBST buffer and visualized by in-gel fluorescence on a Bio-Rad ChemiDoc MP Imaging System. The images were processed using Image Lab (version 5.2.1) software.

Quantification and Statistical Analysis: All data fitting and statistical analysis were performed using GraphPad Prism version 9.00 for Windows and Mac. Statistical significance was defined as p<0.05 and determined by 2-tailed Student's/tests.

### Compound Synthesis and Characterization

General Synthetic Information: All commercial reagents acquired from Sigma-Aldrich, Fisher Scientific, Combi-Blocks, MedChemExpress, AstaTech, Matrix Scientific and BroadPharm were used without further purification. Distilled water was used for all water necessities in synthetic procedures (e.g., reagent, solvent, work-up). All reactions were run with anhydrous solvents. TLC analyses were completed with EMD Millipore silica gel coated (250 μM) F254 glass plates and visualized with UV light (254 nm) or by employing diverse stains (e.g., iodine, CAM, PMA, Ninhydrin, DNP, KMnO₄), followed by gentle heating. Flash column chromatography was performed with silica gel 60. Automated purifications were performed on Biotage Isolera One purification system using manually packed columns of 5G, 10G or 25G of silica. Preparative Thin Layer Chromatography (pTLC) was carried out using EMD Millipore silica gel coated (250 μM) F254 glass plates or glass backed plates 1000-2000 μm thickness (Analtech). ¹H- and ¹³C-NMR spectra were recorded on a Bruker AVANCE NEO 400 MHz instrument with SampleXpress, Bruker AVANCE NEO 500 MHz NMR instrument equipped with 5 mm BBFO SP probe, and SampleCase-24 sample changer, and Bruker AVANCE III HD 600, instrument equipped with a 5 mm CPDCH CryoProbe. Data was collected at ambient temperature unless otherwise stated using standard pulse methods as supplied by Bruker software. Coupling constants are quoted to the nearest 0.1 Hz and multiplicities are given by the following abbreviations and combinations: m (multiplet), s (singlet), d (doublet), dd (doublet of doublets), ddd (doublet of doublet of doublets), t (triplet), td (triplet of doublets), tt (triplet of triplets), q (quartet), br (broad). All NMR data was processed in MestReNova v12.0.2. Chemical shifts for proton and carbon resonances are reported in parts per million (ppm) on the 8 scale relative to the residual protons of the deuterated solvent of relevance. Mass spectrometry data were collected on a Thermo Scientific ISQ single-quadrupole instrument (ESI; low resolution), Agilent 6125 and 6135 single-quadrupole instruments (ESI; low resolution), and Agilent 6230 single-quadrupole TOF (ESI-TOF; high resolution).

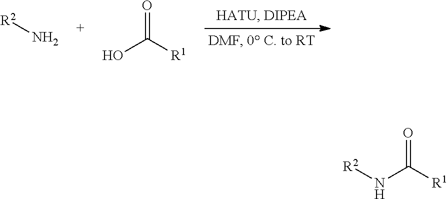

General Procedure 1: Synthesis of amides using HATU as coupling reagent To a solution of amine (1.1 eq.), carboxylic acid (1.0 eq.) and HATU (1.5 eq.) in dry DMF at 0° C. was added DIPEA (3.0 eq.). The reaction mixture was stirred for 2-18 h and then quenched with DI H₂O and diluted with Ethyl acetate (EtOAc). The aqueous layer was extracted with EtOAc (3 times), and the combined organic layers were washed with H₂O (2 times), a saturated aqueous solution of NH₄Cl (2 times) and a saturated aqueous solution of NaCl (2 times) before being dried over anhydrous Na₂SO₄ and filtered. Volatiles were removed by rotary evaporation and the crude product was purified by automated column chromatography, pTLC, or organic solvent washes (i.e., the organic solvent was added into the vial and mixed with the solid decanting).

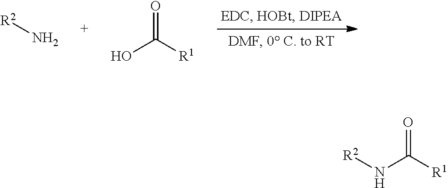

General Procedure 2: Synthesis of amides using EDC as coupling reagent. To a solution of amine (1.1 eq.), carboxylic acid (1.0 eq.), EDC (1.5 eq.) and HOBt (1.5 eq.) in dry DCM at 0° C. was added DIPEA (3.0 eq.). The reaction mixture was stirred overnight, then quenched with DI H₂O. The aqueous layer was extracted with CH₂Cl₂ (3 times), and the combined organic layers were washed with H₂O (2 times), a saturated aqueous solution of NH₄Cl (2 times) and a saturated aqueous solution of NaCl (2 times) before being dried over anhydrous Na₂SO₄ and filtered. Volatiles were removed by rotary evaporation and the crude product was purified by automated column chromatography, pTLC, or organic solvent washes (i.e., the organic solvent was added into the vial and mixed with the solid decanting).

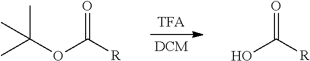

General Procedure 3: Tert-butyl ester deprotection of carboxylic acids using TFA. To a solution of t-Butyl protected acid (1.0 eq.) in dry CH₂Cl₂ was added a solution of TFA (20-50%.). The reaction mixture was stirred at RT for 0.5-12 h. The crude product was concentrated through a nitrogen flow, diluted in CH₂Cl₂ and evaporated again. No purification was needed.

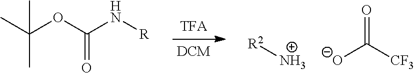

General Procedure 4: Boc deprotection of amines using TFA. To a solution of Boc protected amine (1.0 eq.) in dry CH₂Cl₂ was added a solution of TFA (20-50%). The reaction mixture was stirred at RT for 0.5-12 h. The crude product was concentrated through a nitrogen flow, diluted in CH₂Cl₂ and evaporated again. No purification was needed.

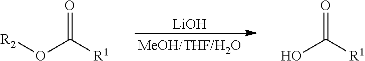

General Procedure 5: Ester hydrolysis. The ester (1.0 eq.) was dissolved in a mixture of THF/MeOH/H₂O (3:1:2) and LiOH, H₂O (7.0 eq.) was added. The reaction mixture was stirred at RT for 3-12 h. The solvents were evaporated, and the residue was acidified with IN aqueous until pH=1, before being extracted with EtOAc, dried over anhydrous Na₂SO₄ and filtered. The organic solvent was evaporated, and no purification was needed.

### Starting Materials:

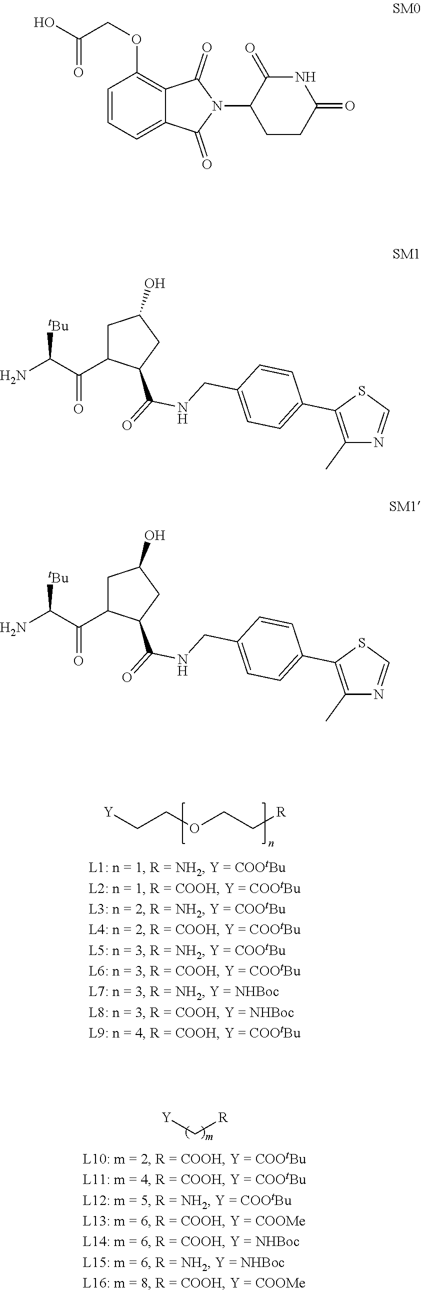

### Characterization Data:

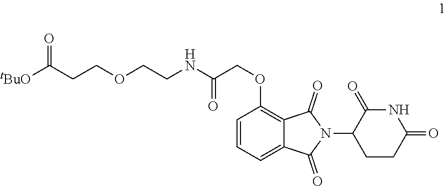 [SMILES: CC(C)(C)OC(=O)CCOCCNC(=O)COc1cccc2c1C(=O)N(C1CCC(=O)NC1=O)C2=O]

### Tert-butyl 3-(2-(2-((2-(2,6-dioxopiperidin-3-yl)-1,3-dioxoisoindolin-4yl)oxy) acetamido) ethoxy) propanoate (1)

General Procedure 1. Reaction scale: 10.5 mg (31.60 μmol, 1.0 eq.) of SM0 and 6.4 μL (33.55 μmol, 1.1 eq.) of L1. Purified by pTLC (10% MeOH/CH₂Cl₂) to afford 1 as a white foam (14.7 mg, 97%). R_{f}=0.63 (10% MeOH/CH₂Cl₂, UV-active); LC-MS (ESI+) calc'd for C₂₄H₂₉N₃O₉ 503.2, found [M+H]⁺ 504.0. 1H NMR (400 MHZ, CDCl₃) δ 8.69 (s, 1H), 7.77-7.69 (t, 1H), 7.65 (d, J=5.9 Hz, 1H), 7.54 (d, J=7.3 Hz, 1H), 7.18 (d, J=8.4 Hz, 1H), 5.04-4.96 (m, 1H), 4.64 (s, 2H), 3.71 (s, 2H), 3.67-3.54 (m, 4H), 3.50 (ddt, J=14.6, 7.8, 3.1 Hz, 1H), 2.89 (t, J=11.3 Hz, 2H), 2.56 (dd, J=7.9, 6.1 Hz, 2H), 2.21-2.09 (m, 2H), 1.42 (d, J=1.7 Hz, 9H). ¹³C NMR (101 MHZ, CDCl₃) δ 171.05, 168.21, 166.82, 166.68, 154.43, 136.96, 133.69, 119.30, 118.11, 117.29, 80.99, 77.36, 77.04, 76.94, 76.73, 69.11, 67.84, 66.68, 49.24, 38.98, 38.63, 36.09, 31.34, 29.72, 28.08, 22.78.

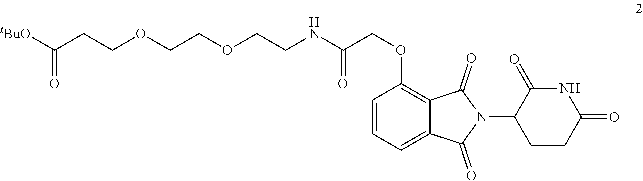 [SMILES: CC(C)(C)OC(=O)CCOCCOCCNC(=O)COc1cccc2c1C(=O)N(C1CCC(=O)NC1=O)C2=O]

### Tert-butyl 3-(2-(2-(2-((2-(2,6-dioxopiperidin-3-yl)-1,3-dioxoisoindolin-4-yl)oxy) acetamido) ethoxy) ethoxy) propanoate (2)

General Procedure 1. Reaction scale: 20 mg (60.0 μmol, 1.0 eq.) of SM0 and 15 μL (65 μmol. 1.1 eq.) of L3. Purified by pTLC (10% MeOH/CH₂Cl₂) to afford 2 as a yellow foam (19.9 mg, 59%). R/=0.57 (10% MeOH/CH₂Cl₂, UV-active); LC-MS (ESI+) calc'd for C₂₆H₃₃N₃O₁₀ 547.2, found [M+H]⁺ 548.4. ¹H NMR (400 MHZ, CDCl₃) δ 8.70 (s, 1H), 7.73 (dd, J=8.4, 7.4 Hz, 1H), 7.59 (br. t, 1H), 7.55-7.48 (m, 1H), 7.18 (d, J=8.5 Hz, 1H), 5.04-4.92 (m, 1H), 4.65 (s, 2H) 3.73-3.52 (m, 10H), 2.89-2.70 (m, 4H), 1.42 (s, 11H). ¹³C NMR (101 MHz, CDCl₃) δ 171.72, 171.45, 168.54, 167.24, 167.13, 166.17, 154.88, 137.40, 134.14, 119.69, 118.47, 117.71, 81.24, 77.79, 77.68, 77.48, 77.16, 70.67, 70.59, 69.93, 68.30, 67.31, 49.71, 39.47, 39.06, 36.62, 31.85, 30.15, 28.53, 23.12.

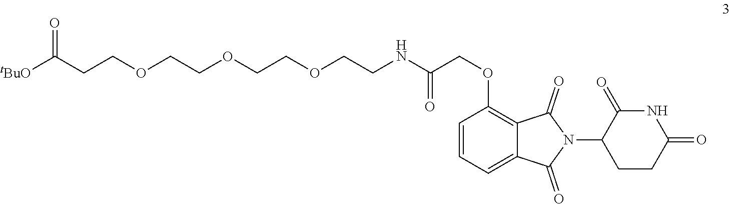 [SMILES: CC(C)(C)OC(=O)CCOCCOCCOCCNC(=O)COc1cccc2c1C(=O)N(C1CCC(=O)NC1=O)C2=O]

### Tert-butyl 1-((2-(2,6-dioxopiperidin-3-yl)-1,3-dioxoisoindolin-4-yl)oxy)-2-oxo-6,9,12-trioxa-3-azapentadecan-15-oate (3)

General Procedure 1. Reaction scale: 20 mg (60.2 μmol, 1.0 eq.) of SM0 and 15 μL (66.2 μmol. 1.1 eq.) of L5. Purified by pTLC (10% MeOH/CH₂Cl₂) to afford 3 as a yellow foam (21.5 mg, 61%). R_{f}=0.76 (8% MeOH/CH₂Cl₂, UV-active); LC-MS (ESI−) calc'd for C₂₈H₃₇N₃O₁₁ 591.2, found [M−H]⁻ 590.0. 1H NMR (400 MHZ, CDCl₃) δ 8.92 (s, 1H), 7.72 (dd, J=8.4, 7.3 Hz, 1H), 7.63 (br. t, 1H), 7.53 (d, J=7.3 Hz, 1H), 7.18 (d, J=8.3 Hz, 1H), 4.99-4.88 (m, 1H), 4.64 (s, 2H), 3.70-3.57 (m, 14H), 2.92-2.80 (m, 2H), 2.48 (t, J=6.6 Hz, 2H), 1.42 (s, 11H). ¹³C NMR (101 MHZ, CDCl₃) δ 171.10, 170.97, 168.15, 166.88, 166.67, 165.80, 154.49, 136.94, 133.70, 119.39, 118.09, 117.28, 80.62, 77.37, 77.25, 77.05, 76.93, 76.73, 70.39, 70.35, 70.32, 70.24, 69.43, 67.96, 66.87, 49.32, 39.13, 38.62, 36.15, 31.42, 29.69, 28.09, 22.71.

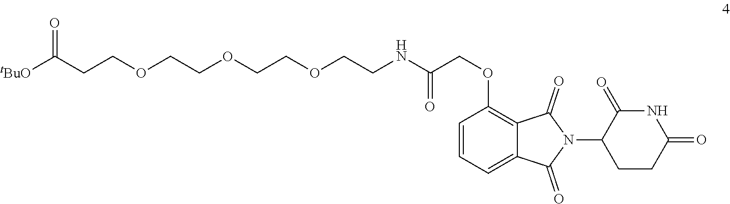 [SMILES: CC(C)(C)OC(=O)CCOCCOCCOCCNC(=O)COc1cccc2c1C(=O)N(C1CCC(=O)NC1=O)C2=O]

### Tert-butyl (1-((2-(2,6-dioxopiperidin-3-yl)-1,3-dioxoisoindolin-4-yl)oxy)-2-oxo-6,9,12-trioxa-3-azatetradecan-14-yl) carbamate (4)

Reaction scale: 100 mg (301.0 μmol, 1.0 eq.) of SM0 and 96.8 mg (331.1 μmol. 1.1 eq.) of L7. Purified by Biotage Isolera (2-14% MeOH/CH₂Cl₂ gradient, 16 CV) to afford 4 as a yellow foam (113 mg, 62%). R_{f}=0.63 (8% MeOH/CH₂Cl₂, UV-active); LC-MS (ESI+) calc'd for C₂₈H₃₈N₃O₁₁ 606.3, found [M+H]⁺ 607.2. ¹H NMR (500 MHZ, CDCl₃) δ 9.23 (s, 1H), 7.69 (t, J=7.9 Hz, 1H), 7.58 (t, J=5.6 Hz, 1H), 7.49 (d, J=7.3 Hz, 1H), 7.15 (s, 1H), 4.96-4.87 (m, 1H), 4.61 (s, 2H), 3.62-3.48 (m, 17H), 2.87-2.76 (m, 2H), 1.38 (s, 11H).

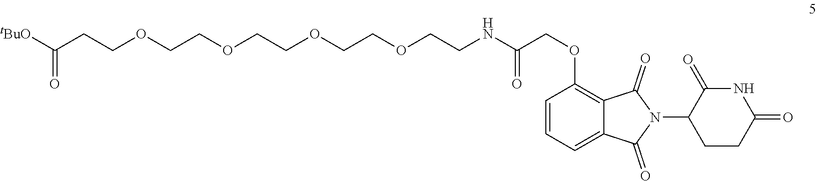 [SMILES: CC(C)(C)OC(=O)CCOCCOCCOCCOCCNC(=O)COc1cccc2c1C(=O)N(C1CCC(=O)NC1=O)C2=O]

### Tert-butyl 1-((2-(2,6-dioxopiperidin-3-yl)-1,3-dioxoisoindolin-4-yl)oxy)-2-oxo-6,9,12,15-tetraoxa-3-azaoctadecan-18-oate (5)

General Procedure 1. Reaction scale: 10 mg (32.5 μmol, 1.0 eq.) of SM0 and 10.18 μL (35.8 μmol. 1.1 eq.) of L9. Purified by pTLC (10% MeOH/CH₂Cl₂) to afford 5 as a white foam (15.7 mg, 76%). R/=0.53 (10% MeOH/CH₂Cl₂, UV-active); LC-MS (ESI+) calc'd for C₃₀H₄₁N₃O₁₂ 635.3, found [M+H]⁺ 636.0. ¹H NMR (400 MHZ, CDCl₃) δ 8.99 (s, 1H), 7.72 (dd, J=8.4, 7.4 Hz, 1H), 7.63 (t, J=5.2 Hz, 1H), 7.53 (d, J=7.3 Hz, 1H), 7.18 (d, J=8.4 Hz, 1H), 5.03-4.86 (m, 1H), 4.64 (s, 2H), 3.69-3.59 (m, 18H), 2.88-2.78 (m, 2H), 2.47 (t, J=6.5 Hz, 3H), 1.42 (s, 11H). ¹³C NMR (101 MHz, CDCl₃) δ 171.34, 168.34, 167.10, 166.70, 154.44, 136.97, 133.65, 119.42, 117.25, 80.61, 77.42, 77.10, 76.79, 70.55, 70.38, 70.28, 69.50, 67.87, 66.79, 60.40, 49.31, 39.08, 38.63, 36.19, 31.46, 29.70, 28.08, 22.68, 21.05, 14.19.

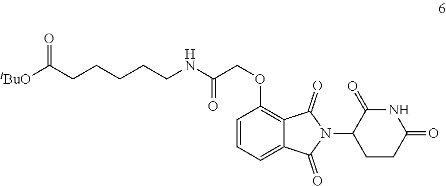 [SMILES: CC(C)(C)OC(=O)CCCCCNC(=O)COc1cccc2c1C(=O)N(C1CCC(=O)NC1=O)C2=O]

### Tert-butyl 6-(2-((2-(2,6-dioxopiperidin-3-yl)-1,3-dioxoisoindolin-4-yl)oxy) acetamido) hexanoate (6)

General Procedure 1. Reaction scale: 15 mg (45.1 μmol, 1.0 eq.) of SM0 and 10.0 μL (49.7 μmol. 1.1 eq.) of L13. Purified by pTLC (8% MeOH/CH₂Cl₂) to afford 6 as an off-white solid (19.6 mg, 87%). R_{f}=0.79 (8% MeOH/CH₂Cl₂, UV-active); LC-MS (ESI+) calc'd for C₂₅H₃₁N₃O₈ 501.2, found [M+H]⁺ 502.0. ¹H NMR (400 MHZ, CDCl₃) δ 9.66 (s, 1H), 7.75-7.71 (m, 1H), 7.61 (s, 1H), 7.55 (d, J=7.3 Hz, 1H), 7.39 (d, J=1.4 Hz, 2H), 7.28 (d, J=7.3 Hz, 2H), 7.20 (d, J=1.4 Hz, 1H), 4.98-4.91 (m, 1H), 4.72-4.55 (m, 3H), 3.63 (q, J=3.8 Hz, 3H), 3.45 (t, J=5.1 Hz, 2H), 2.35 (t, J=4.9 Hz, 4H), 1.25 (s, 9H). ¹³C NMR (101 MHz, CDCl₃) δ 171.77, 171.13, 168.15, 166.66, 166.41, 166.08, 154.62, 141.96, 136.86, 133.40, 128.43, 127.68, 126.98, 120.06, 118.32, 117.43, 77.16, 77.05, 76.84, 76.62, 76.52, 75.77, 68.48, 51.92, 51.44, 49.17, 45.70, 41.64, 39.03, 32.85, 31.22, 29.53, 28.69, 26.59, 25.16, 22.67.

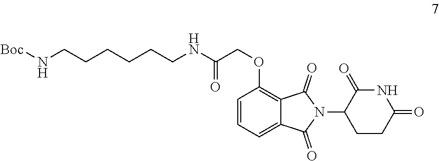 [SMILES: CC(C)(C)OC(=O)NCCCCCCNC(=O)COc1cccc2c1C(=O)N(C1CCC(=O)NC1=O)C2=O]

### Tert-butyl (6-(2-((2-(2,6-dioxopiperidin-3-yl)-1,3-dioxoisoindolin-4-yl)oxy) acetamido) hexyl) carbamate (7)

¹H NMR (400 MHZ, CDCl₃) δ 7.74 (m, 1H), 7.55 (d, J=7.3 Hz, 1H), 7.46 (m, 1H), 7.19 (d, J=8.3 Hz, 1H), 4.99 (m, 1H), 4.71-4.60 (m, 3H), 3.46-3.28 (m, 2H), 3.17-3.04 (m, 2H), 2.93-2.76 (m, 3H), 2.14 (m, 1H), 1.64-1.56 (m, 2H), 1.48-1.33 (m, 16H).

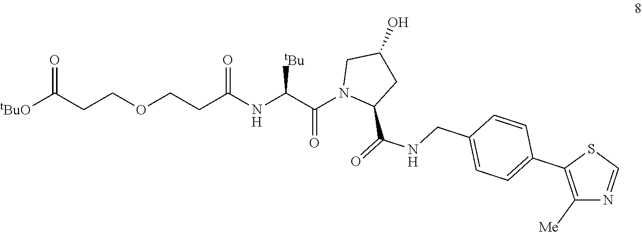 [SMILES: Cc1ncsc1-c1ccc(CNC(=O)[C@@H]2C[C@@H](O)CN2C(=O)[C@@H](NC(=O)CCOCCC(=O)OC(C)(C)C)C(C)(C)C)cc1]

### Tert-butyl 3-(3-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-3-oxopropoxy) propanoate (8)

General Procedure 1. Reaction scale: 20 mg (46.5 μmol, 1.0 eq.) of SM1 and 11.2 mg (51.1 μmol. 1.1 eq.) of L2. Purified by pTLC (8% MeOH/CH₂Cl₂) to afford 8 as a clear oil (22.6 mg, 77%). R_{f}=0.59 (8% MeOH/CH₂Cl₂, UV-active); LC-MS (ESI+) calc'd for C₃₂H₄₆N₄O₇S 630.3, found [M+H]⁺ 631.1. ¹H NMR (600 MHZ, CDCl₃) δ 8.67 (s, 1H), 7.43 (t, J=6.0 Hz, 1H), 7.36-7.30 (m, 4H), 6.95 (d, J=8.3 Hz, 1H), 4.72 (t, J=7.9 Hz, 1H), 4.55 (dd, J=15.0, 6.7 Hz, 1H), 4.50 (dt, J=4.3, 2.1 Hz, 1H), 4.44 (d, J=8.2 Hz, 1H), 4.32 (dd, J=15.0, 5.3 Hz, 1H), 4.09-4.07 (m, 1H), 3.71-3.64 (m, 4H), 3.59 (dd, J=11.3, 3.7 Hz, 1H), 2.54-2.44 (m, 7H), 2.13-2.07 (m, 1H), 1.42 (s, 9H), 0.92 (s, 9H). ¹³C NMR (151 MHz, CDCl₃) δ 172.08, 171.82, 171.19, 170.79, 170.63, 150.34, 148.45, 138.16, 131.63, 130.92, 129.52, 128.12, 128.09, 80.85, 77.26, 77.05, 76.84, 70.10, 66.82, 66.79, 66.66, 66.29, 60.42, 58.39, 57.77, 56.62, 55.99, 43.22, 36.69, 36.08, 35.85, 34.79, 31.93, 30.17, 29.71, 29.67, 29.37, 29.33, 28.93, 28.10, 28.08, 26.64, 26.41, 22.70, 21.27, 21.07, 17.45, 16.04, 14.14.

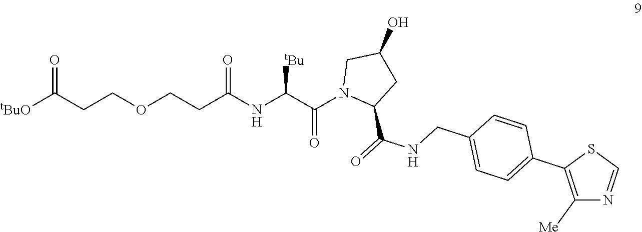 [SMILES: Cc1ncsc1-c1ccc(CNC(=O)[C@@H]2C[C@H](O)CN2C(=O)[C@@H](NC(=O)CCOCCC(=O)OC(C)(C)C)C(C)(C)C)cc1]

### Tert-butyl 3-(3-(((S)-1-((2S,4S)-4-hydroxy-2-((4-(4-methylthiazol-5-yl)benzyl) carbamoyl) pyrrolidin-1-yl)-3,3-dimethyl-1-oxobutan-2-yl)amino)-3-oxopropoxy) propanoate (9)

General Procedure 1. Reaction scale: 20 mg (46.5 μmol, 1.0 eq.) of SM1′ and 11.3 mg (51.1 μmol. 1.1 eq.) of L2. Purified by pTLC (8% MeOH/CH₂Cl₂) to afford 9 as a white foam (0.6 mg, 77%). R/=0.57 (8% MeOH/CH₂Cl₂, UV-active); LC-MS (ESI+) calc'd for C₃₂H₄₆N₄O₇S 630.3, found [M+H]⁺ 631.3. ¹H NMR (600 MHZ, CDCl₃) δ 8.67 (s, 1H), 7.43 (t, J=6.0 Hz, 1H), 7.36-7.30 (m, 4H), 6.95 (d, J=8.3 Hz, 1H), 4.72 (t, J=7.9 Hz, 1H), 4.55 (dd, J=15.0, 6.7 Hz, 1H), 4.50 (dt, J=4.3, 2.1 Hz, 1H), 4.44 (d, J=8.2 Hz, 1H), 4.32 (dd, J=15.0, 5.3 Hz, 1H), 4.09-4.07 (m, 1H), 3.71-3.64 (m, 4H), 3.59 (dd, J=11.3, 3.7 Hz, 1H), 2.54-2.44 (m, 7H), 2.13-2.07 (m, 1H), 1.42 (s, 9H), 0.92 (s, 9H). ¹³C NMR (151 MHz, CDCl₃) δ 172.08, 171.82, 171.19, 170.79, 170.63, 150.34, 148.45, 138.16, 131.63, 130.92, 129.52, 128.12, 128.09, 80.85, 77.26, 77.05, 76.84, 70.10, 66.82, 66.79, 66.66, 66.29, 60.42, 58.39, 57.77, 56.62, 55.99, 43.22, 36.69, 36.08, 35.85, 34.79, 31.93, 30.17, 29.71, 29.67, 29.37, 29.33, 28.93, 28.10, 28.08, 26.64, 26.41, 22.70, 21.27, 21.07, 17.45, 16.04, 14.14.

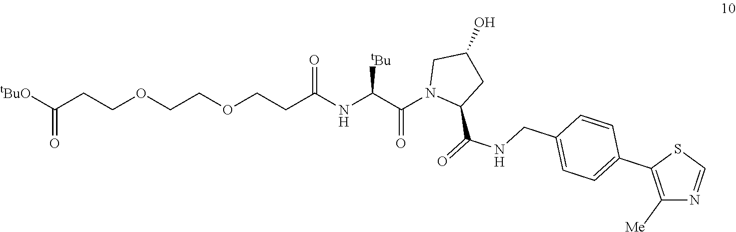 [SMILES: Cc1ncsc1-c1ccc(CNC(=O)[C@@H]2C[C@@H](O)CN2C(=O)[C@@H](NC(=O)CCOCCOCCC(=O)OC(C)(C)C)C(C)(C)C)cc1]

### Tert-butyl 3-(2-(3-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-3-oxopropoxy) ethoxy) propanoate (10)

General Procedure 1. Reaction scale: 20 mg (46.5 μmol, 1.0 eq.) of SM1 and 13.5 mg (51.1 μmol. 1.1 eq.) of L4. Purified by pTLC (10% MeOH/CH₂Cl₂) to afford 10 as a yellow oil (14.9 mg, 51%). R/=0.54 (10% MeOH/CH₂Cl₂, UV-active); LC-MS (ESI+) calc'd for C₃₄H₅₀N₄O₈S 674.3, found [M+H]⁺ 675.2. ¹H NMR (400 MHZ, CDCl₃) δ 8.67 (s, 1H), 7.48 (t, J=6.0 Hz, 1H), 7.38-7.30 (m, 4H), 7.05 (d, J=8.2 Hz, 1H), 4.73 (t, J=8.0 Hz, 1H), 4.56 (dd, J=15.0, 6.6 Hz, 1H), 4.50 (dt, J=4.4, 2.1 Hz, 1H), 4.44 (d, J=8.2 Hz, 1H), 4.32 (dd, J=15.0, 5.3 Hz, 1H), 4.09 (dt, J=11.5, 1.8 Hz, 1H), 3.72-3.65 (m, 4H), 3.61-3.56 (m, 5H), 2.52-2.45 (m, 8H), 2.13-2.06 (m, 1H), 1.42 (s, 9H), 0.93 (s, 9H).

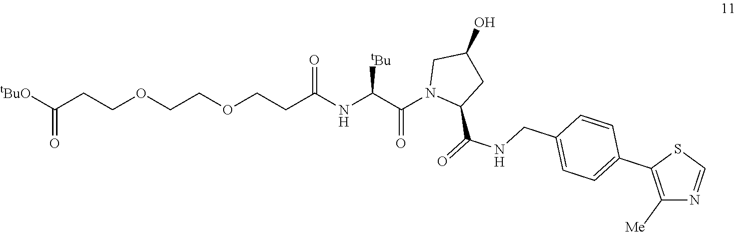 [SMILES: Cc1ncsc1-c1ccc(CNC(=O)[C@@H]2C[C@H](O)CN2C(=O)[C@@H](NC(=O)CCOCCOCCC(=O)OC(C)(C)C)C(C)(C)C)cc1]

### Tert-butyl 3-(2-(3-(((S)-1-((2S,4S)-4-hydroxy-2-((4-(4-methylthiazol-5-yl)benzyl) carbamoyl) pyrrolidin-1-yl)-3,3-dimethyl-1-oxobutan-2-yl)amino)-3-oxopropoxy) ethoxy) propanoate (11)

General Procedure 1. Reaction scale: 20 mg (46.5 μmol, 1.0 eq.) of SM1′ and 13.4 mg (51.1 μmol. 1.1 eq.) of L4. Purified by pTLC (8% MeOH/CH₂Cl₂) to afford 11 as a clear oil (19.3 mg, 62%). R$=0.55 (10% MeOH/CH₂Cl₂, UV-active); LC-MS (ESI+) calc'd for C₃₄H₅₀N₄O₈S 674.3, found [M+H]⁺ 675.1. ¹H NMR (400 MHZ, CDCl₃) δ 8.67 (s, 1H), 7.49 (t, J=6.0 Hz, 1H), 7.39-7.29 (m, 4H), 7.07 (d, J=8.1 Hz, 1H), 4.72 (t, J=8.0 Hz, 1H), 4.60-4.41 (m, 3H), 4.32 (dd, J=15.0, 5.3 Hz, 1H), 4.14-4.05 (m, 1H), 3.68 (q, J=6.1 Hz, 4H), 3.60 (s, 5H), 2.55-2.42 (m, 8H), 2.11 (dd, J=13.4, 8.1 Hz, 1H), 1.42 (s, 9H), 0.93 (s, 9H). ¹³C NMR (101 MHZ, CDCl₃) δ 172.23, 171.79, 170.98, 170.88, 150.32, 148.44, 138.23, 131.64, 130.88, 129.49, 128.11, 80.70, 77.37, 77.25, 77.05, 76.73, 70.43, 70.17, 70.10, 67.14, 66.88, 58.40, 57.82, 56.63, 43.19, 36.66, 36.15, 35.92, 34.76, 29.71, 28.10, 26.41, 16.05.

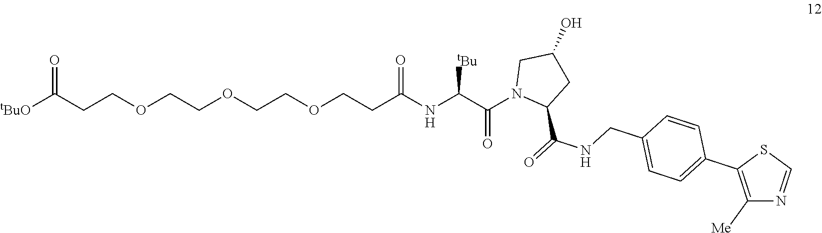 [SMILES: Cc1ncsc1-c1ccc(CNC(=O)[C@@H]2C[C@@H](O)CN2C(=O)[C@@H](NC(=O)CCOCCOCCOCCC(=O)OC(C)(C)C)C(C)(C)C)cc1]

### Tert-butyl(S)-15-((2S,4R)-4-hydroxy-2-((4-(4-methylthiazol-5-yl)benzyl) carbamoyl) pyrrolidine-1-carbonyl)-16,16-dimethyl-13-oxo-4,7,10-trioxa-14-azaheptadecanoate (12)

General Procedure 1. Reaction scale: 70 mg (162.6 μmol, 1.0 eq.) of SM1 and 54.8 mg (178.8 μmol. 1.1 eq.) of L6. Purified by Biotage Isolera (2-15% MeOH/CH₂Cl₂ gradient, 16 CV) to afford 12 as a white foam (81 mg, 70%). R/=0.56 (10% MeOH/CH₂Cl₂, UV-active); LC-MS (ESI−) calc'd for C₃₆H₅₄N₄O₉S 718.4, found [M+HCOO]⁻ 763.2. ¹H NMR (400 MHZ, CDCl₃) δ 8.60 (s, 1H), 7.48 (t, J=6.0 Hz, 1H), 7.26 (s, 4H), 7.02 (d, J=8.5 Hz, 1H), 4.60 (t, J=8.0 Hz, 1H), 4.45 (dd, J=15.4, 7.2 Hz, 3H), 4.25 (dd, J=15.1, 5.4 Hz, 1H), 3.94 (d, J=11.2 Hz, 1H), 3.60 (td, J=6.1, 3.8 Hz, 4H), 3.57-3.49 (m, 9H), 2.44-2.34 (m, 7H), 2.31 (td, J=8.3, 4.1 Hz, 1H), 2.04 (ddt, J=13.0, 8.1, 1.9 Hz, 1H), 1.35 (s, 9H), 0.87 (s, 9H). ¹³C NMR (101 MHz, CDCl₃) δ 172.00, 171.50, 171.20, 170.94, 150.35, 148.36, 138.30, 131.65, 130.76, 129.41, 128.21, 128.01, 80.60, 77.43, 77.31, 77.11, 76.79, 70.43, 70.39, 70.36, 70.26, 69.98, 67.10, 66.84, 58.69, 57.64, 56.73, 50.39, 45.73, 43.09, 36.67, 36.33, 36.20, 35.11, 29.66, 28.08, 26.38, 16.00, 8.54.

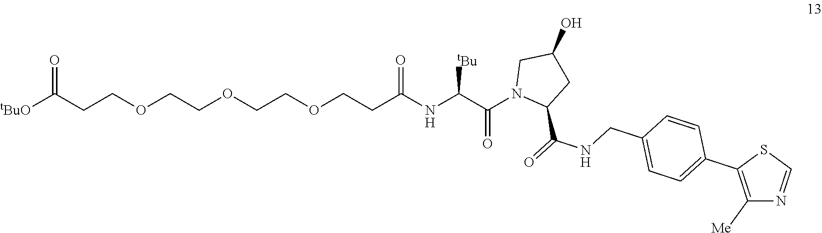 [SMILES: Cc1ncsc1-c1ccc(CNC(=O)[C@@H]2C[C@H](O)CN2C(=O)[C@@H](NC(=O)CCOCCOCCOCCC(=O)OC(C)(C)C)C(C)(C)C)cc1]

### Tert-butyl(S)-15-((2S,4S)-4-hydroxy-2-((4-(4-methylthiazol-5-yl)benzyl) carbamoyl) pyrrolidine-1-carbonyl)-16,16-dimethyl-13-oxo-4,7,10-trioxa-14-azaheptadecanoate (13)

General Procedure 1. Reaction scale: 15 mg (34.8 μmol, 1.0 eq.) of SM1′ and 11.7 mg (38.3 μmol. 1.1 eq.) of L6. Purified by Biotage Isolera (2-15% MeOH/CH₂Cl₂ gradient, 16 CV) to afford 13 as a white foam (81 mg, 70%). R/=0.62 (10% MeOH/CH₂Cl₂, UV-active); LC-MS (ESI+) calc'd for C₃₆H₅₄N₄O₉S 718.4, found [M+H]⁺ 719.2. ¹H NMR (400 MHz, CDCl₃) δ 8.69 (s, 1H), 7.67-7.59 (m, 1H), 7.42-7.32 (m, 4H), 6.95 (d, J=8.7 Hz, 1H), 5.59 (s, 1H), 4.73 (d, J=9.0 Hz, 1H), 4.65 (dd, J=15.0, 7.0 Hz, 1H), 4.49 (d, J=8.7 Hz, 2H), 4.31 (dd, J=15.0, 5.1 Hz, 1H), 3.96 (dd, J=11.0, 4.2 Hz, 1H), 3.80 (dd, J=11.1, 1.5 Hz, 1H), 3.74-3.69 (m, 3H), 3.66 (d, J=12.6 Hz, 5H), 3.60 (q, J=1.7 Hz, 4H), 2.55-2.45 (m, 7H), 2.35 (d, J=14.2 Hz, 1H), 2.19 (ddd, J=14.1, 8.9, 4.8 Hz, 1H), 1.44 (s, 9H), 0.93 (s, 9H). ¹³C NMR (101 MHz, CDCl₃) δ 172.72, 172.18, 171.57, 170.95, 150.40, 137.45, 131.18, 129.61, 128.17, 80.62, 77.35, 77.23, 77.03, 76.72, 71.15, 70.51, 70.47, 70.43, 70.32, 67.20, 66.88, 59.87, 58.62, 57.12, 43.48, 36.80, 36.23, 35.10, 34.89, 29.70, 28.10, 26.32, 16.03.

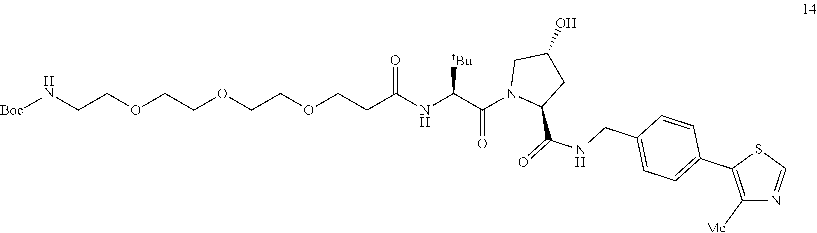 [SMILES: Cc1ncsc1-c1ccc(CNC(=O)[C@@H]2C[C@@H](O)CN2C(=O)[C@@H](NC(=O)CCOCCOCCOCCNC(=O)OC(C)(C)C)C(C)(C)C)cc1]

### Tert-butyl ((S)-14-((2S,4R)-4-hydroxy-2-((4-(4-methylthiazol-5-yl)benzyl) carbamoyl) pyrrolidine-1-carbonyl)-15,15-dimethyl-12-oxo-3,6,9-trioxa-13-azahexadecyl) carbamate (14)

General Procedure 1. Reaction scale: 30 mg (69.9 μmol, 1.0 eq.) of SM1 and 24.6 mg (76.6 μmol. 1.1 eq.) of L8. Purified by Biotage Isolera (2-15% MeOH/CH₂Cl₂ gradient, 16 CV) to afford 14 as a yellow oil (40 mg, 78%). R$=0.52 (10% MeOH/CH₂Cl₂, UV-active); LC-MS (ESI−) calc'd for C₃₆H₅₅N₅O₉S 733.4, found [M+HCOO]⁻ 778.2. ¹H NMR (400 MHZ, CDCl₃) δ 8.67 (s, 1H), 7.43 (br. s, 1H), 7.36-7.29 (m, 4H), 7.02 (d, J=8.4 Hz, 1H), 5.11 (d, J=7.5 Hz, 1H), 4.70 (t, J=8.0 Hz, 1H), 4.57-4.45 (m, 3H), 4.32 (dd, J=15.0, 5.3 Hz, 1H), 4.07 (br. s, 1H), 3.70 (t, J=5.7 Hz, 2H), 3.60 (d, J=15.4 Hz, 10H), 3.50 (t, J=5.2 Hz, 2H), 2.52-2.41 (m, 7H), 2.15-2.07 (m, 1H), 1.42 (s, 9H), 0.93 (s, 9H). ¹³C NMR (101 MHz, CDCl₃) δ 172.10, 171.73, 170.93, 156.08, 150.35, 148.42, 138.23, 131.65, 130.86, 129.47, 128.09, 79.26, 77.37, 77.26, 77.06, 76.74, 70.46, 70.23, 70.12, 70.06, 67.15, 60.40, 58.51, 57.71, 56.69, 43.18, 40.34, 36.64, 36.04, 34.92, 29.70, 28.44, 26.41, 21.06, 16.03, 14.20.

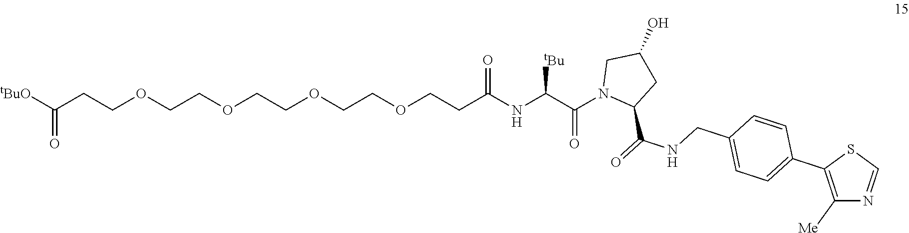 [SMILES: Cc1ncsc1-c1ccc(CNC(=O)[C@@H]2C[C@@H](O)CN2C(=O)[C@@H](NC(=O)CCOCCOCCOCCOCCC(=O)OC(C)(C)C)C(C)(C)C)cc1]

### Tert-butyl(S)-18-((2S,4R)-4-hydroxy-2-((4-(4-methylthiazol-5-yl)benzyl) carbamoyl) pyrrolidine-1-carbonyl)-19,19-dimethyl-16-oxo-4,7,10,13-tetraoxa-17-azaicosanoate (15)

General Procedure 1. Reaction scale: 20 mg (46.5 μmol, 1.0 eq.) of SM1 and 12.7 mg (51.1 μmol. 1.1 eq.) of L9. Purified by pTLC (10% MeOH/CH₂Cl₂) to afford 15 as a yellow foam (16.3 mg, 51%). R_{f}=0.54 (10% MeOH/CH₂Cl₂, UV-active); LC-MS (ESI+) calc'd for C₃₈H₅₈N₄O₁₀S 762.4, found [M+H]⁺ 763.3.

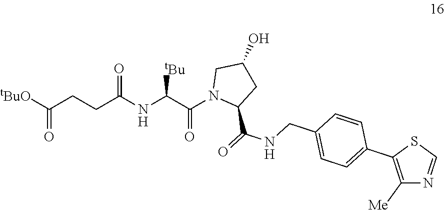 [SMILES: Cc1ncsc1-c1ccc(CNC(=O)[C@@H]2C[C@@H](O)CN2C(=O)[C@@H](NC(=O)CCC(=O)OC(C)(C)C)C(C)(C)C)cc1]

### Tert-butyl 4-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-4-oxobutanoate (16)

General Procedure 1. Reaction scale: 10 mg (23.2 μmol, 1.0 eq.) of SM1 and 4.5 mg (25.6 μmol. 1.1 eq.) of L10. Purified by pTLC (10% MeOH/CH₂Cl₂) to afford 16 as a white solid (7.5 mg, 53%). R_{f}=0.54 (10% MeOH/CH₂Cl₂, UV-active); LC-MS (ESI+) calc'd for C₃₀H₄₂N₄O₆S 586.3, found [M+H]⁺ 587.1. ¹H NMR (400 MHZ, CDCl₃) δ 8.67 (s, 1H), 7.44 (t, J=6.0 Hz, 1H), 7.37-7.27 (m, 4H), 6.56 (d, J=8.6 Hz, 1H), 4.73 (t, J=8.0 Hz, 1H), 4.47 (d, J=8.6 Hz, 3H), 4.35 (d, J=5.3 Hz, 1H), 4.05 (d, J=11.6 Hz, 1H), 3.58 (dd, J=11.4, 3.5 Hz, 1H), 2.60-2.42 (m, 10H), 1.42 (s, 9H), 0.93 (s, 9H).

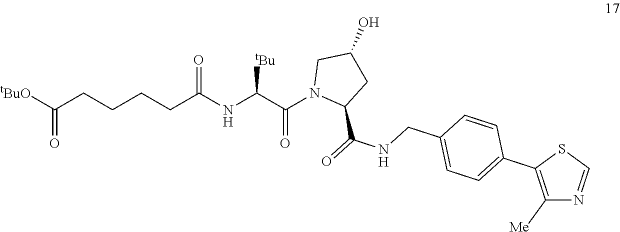 [SMILES: Cc1ncsc1-c1ccc(CNC(=O)[C@@H]2C[C@@H](O)CN2C(=O)[C@@H](NC(=O)CCCCC(=O)OC(C)(C)C)C(C)(C)C)cc1]

### Tert-butyl 6-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-6-oxohexanoate (17)

General Procedure 1. Reaction scale: 20 mg (46.5 μmol, 1.0 eq.) of SM1 and 10.3 mg (51.1 μmol. 1.1 eq.) of L11. Purified by pTLC (8% MeOH/CH₂Cl₂) to afford 17 as a white solid (24.6 mg, 86%). R_{f}=0.55 (8% MeOH/CH₂Cl₂, UV-active); LC-MS (ESI+) calc'd for C₃₂H₄₆N₄O₆S 614.3, found [M+H]⁺ 615.1. ¹H NMR (400 MHZ, CDCl₃) δ 8.60 (s, 1H), 7.27 (t, J=4.6 Hz, 5H), 6.30 (d, J=8.7 Hz, 1H), 4.63 (t, J=8.0 Hz, 1H), 4.51-4.40 (m, 3H), 4.29-4.20 (m, 1H), 4.00 (d, J=11.4 Hz, 1H), 3.60-3.48 (m, 2H), 2.43 (s, 4H), 2.14-2.08 (m, 4H), 1.51 (m, 4H), 1.34 (s, 9H), 0.85 (s, 9H).

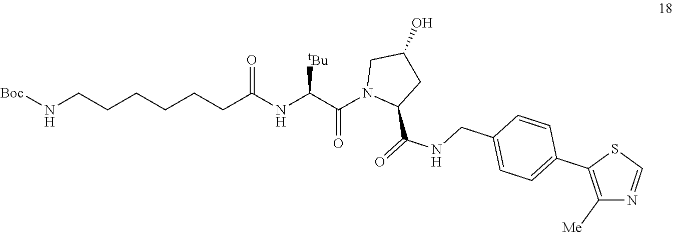 [SMILES: Cc1ncsc1-c1ccc(CNC(=O)[C@@H]2C[C@@H](O)CN2C(=O)[C@@H](NC(=O)CCCCCCNC(=O)OC(C)(C)C)C(C)(C)C)cc1]

### Tert-butyl (7-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-7-oxoheptyl) carbamate (18)

The protocols are the same as that described in Dolle et al., J. Med. Chem. 2021, 64, 15, 10682-10710.

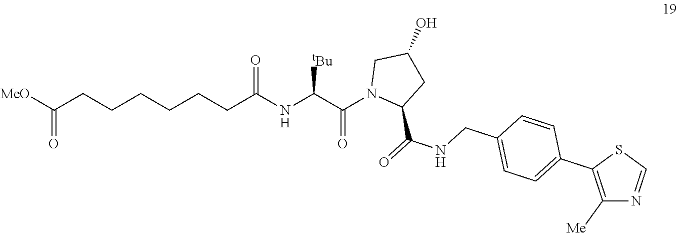 [SMILES: COC(=O)CCCCCCC(=O)N[C@H](C(=O)N1C[C@H](O)C[C@H]1C(=O)NCc1ccc(-c2scnc2C)cc1)C(C)(C)C]

### Methyl 8-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-8-oxooctanoate (19)

General Procedure 1. Reaction scale: 10.0 mg (23.2 μmol, 1.0 eq.) of SM1 and 4.6 μL (25.6 μmol. 1.1 eq.) of L13. Purified by pTLC (10% MeOH/CH₂Cl₂) to afford 19 as a white solid (10.0 mg, 72%). R_{f}=0.61 (10% MeOH/CH₂Cl₂, UV-active); LC-MS (ESI+) calc'd for C₃₁H₄N₄O₆S 600.3, found [M+H]⁺ 601.1. ¹H NMR (400 MHz, CDCl₃) δ 8.68 (s, 1H), 7.40-7.29 (m, 5H), 6.15 (d, J=8.7 Hz, 1H), 4.71 (t, J=7.9 Hz, 1H), 4.61-4.46 (m, 3H), 4.33 (dd, J=14.9, 5.2 Hz, 1H), 4.15-4.03 (m, 1H), 3.64 (s, 3H), 2.51 (s, 3H), 2.27 (d, J=7.5 Hz, 2H), 2.19-2.15 (m, 2H), 1.63-1.56 (m, 4H), 1.31-1.25 (m, 7H), 0.93 (s, 9H).

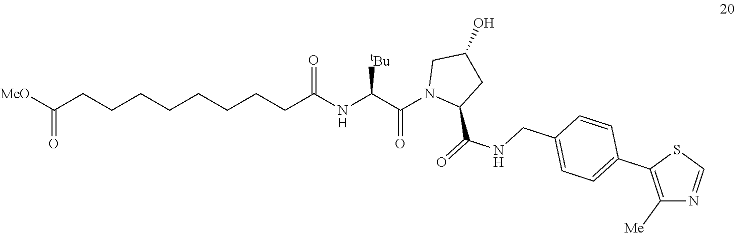 [SMILES: COC(=O)CCCCCCCCC(=O)N[C@H](C(=O)N1C[C@H](O)C[C@H]1C(=O)NCc1ccc(-c2scnc2C)cc1)C(C)(C)C]

### Methyl 10-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-10-oxodecanoate (20)

General Procedure 1. Reaction scale: 10.0 mg (23.2 μmol, 1.0 eq.) of SM1 and 5.53 mg (25.6 μmol. 1.1 eq.) of L16. Purified by pTLC (10% MeOH/CH₂Cl₂) to afford 20 as a white solid (10.3 mg, 71%). R_{f}=0.64 (10% MeOH/CH₂Cl₂, UV-active); LC-MS (ESI+) calc'd for C₃₃H₄₈N₄O₆S 628.3, found [M+H]⁺ 629.1. ¹H NMR (400 MHZ, CDCl₃) δ 8.68 (s, 1H), 7.40-7.28 (m, 5H), 6.14 (d, J=8.7 Hz, 1H), 4.71 (t, J=7.9 Hz, 1H), 4.60-4.45 (m, 3H), 4.33 (dd, J=15.0, 5.2 Hz, 1H), 4.15-4.06 (m, 1H), 3.65 (d, J=4.8 Hz, 4H), 2.51 (s, 3H), 2.30-2.23 (m, 2H), 2.20-2.12 (m, 3H), 1.59 (d, J=9.7 Hz, 4H), 1.29-1.26 (m, 9H), 0.92 (s, 9H).

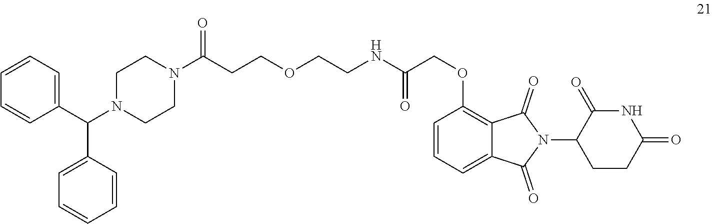 [SMILES: O=C(COc1cccc2c1C(=O)N(C1CCC(=O)NC1=O)C2=O)NCCOCCC(=O)N1CCN(C(c2ccccc2)c2ccccc2)CC1]

### N-(2-(3-(4-benzhydrylpiperazin-1-yl)-3-oxopropoxy)ethyl)-2-((2-(2,6-dioxopiperidin-3-yl)-1,3-dioxoisoindolin-4-yl)oxy) acetamide (21)

General Procedure 3 then 2. Reaction scale: 13.0 mg (29.1 μmol) of 1 and 8.1 mg (32.0 μmol) of benzhydrylpiperazine. Purified by pTLC (10% MeOH/DCM) to afford 21 as a white foam (7.5 mg, 38%). R/=0.63 (10% MeOH/CH₂Cl₂, UV-active); LC-MS (ESI+) calc'd for C₃₇H₃₉N₅O₈ 681.3, found [M+H]⁺ 682.0. ¹H NMR (400 MHZ, CDCl₃) δ 9.10 (s, 1H), 7.72 (d, J=1.1 Hz, 1H), 7.54 (dd, J=7.4, 0.6 Hz, 1H), 7.38 (s, 3H), 7.26 (d, J=1.4 Hz, 4H), 7.20-7.16 (m, 4H), 4.99-4.92 (m, 2H), 4.63 (d, J=2.9 Hz, 2H), 3.78 (p, J=1.5 Hz, 2H), 3.62 (t, J=5.1 Hz, 6H), 3.46 (d, J=4.8 Hz, 3H), 2.85-2.66 (m, 7H), 1.60 (m 3H). ¹³C NMR (101 MHZ, CDCl₃) δ 171.14, 169.63, 168.26, 166.95, 166.63, 165.95, 154.60, 142.10, 136.96, 133.65, 128.63, 127.86, 127.18, 119.69, 118.25, 117.39, 77.34, 77.23, 77.02, 77.02, 76.71, 75.90, 68.90, 68.19, 67.36, 60.41, 51.97, 51.51, 49.24, 45.85, 41.78, 39.37, 33.38, 31.29, 29.71, 29.37, 22.91, 14.21, 14.12.

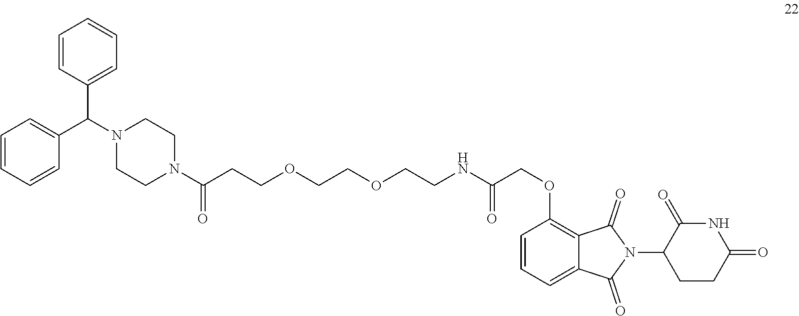 [SMILES: O=C(COc1cccc2c1C(=O)N(C1CCC(=O)NC1=O)C2=O)NCCOCCOCCC(=O)N1CCN(C(c2ccccc2)c2ccccc2)CC1]

### N-(2-(2-(3-(4-benzhydrylpiperazin-1-yl)-3-oxopropoxy) ethoxy)ethyl)-2-((2-(2,6-dioxopiperidin-3-yl)-1,3-dioxoisoindolin-4-yl)oxy) acetamide (22)

General Procedure 3 then 2. Reaction scale: 19.9 mg (36.3 μmol, 1 eq) of 2 and 10.1 mg (39.9 μmol, 1.1 eq) of benzhydrylpiperazine. Purified by pTLC (10% MeOH/DCM) to afford 22 as a white foam (7.5 mg, 38%). R_{f}=0.61 (10% MeOH/CH₂Cl₂, UV-active); LC-MS (ESI+) calc'd for C₃₉H₄₃N₅O₉ 725.3, found [M+H]⁺ 726.2. ¹H NMR (400 MHz, CDCl₃) δ 9.10 (s, 1H), 7.72 (dd, J=8.4, 7.4 Hz, 1H), 7.63 (t, J=4.9 Hz, 1H), 7.54 (dd, J=7.4, 0.6 Hz, 1H), 7.38 (m, 3H), 7.26 (m, 4H), 7.20-7.16 (m, 4H), 4.99-4.92 (m, 2H), 4.63 (s, 2H), 3.80 (td, J=6.9, 1.6 Hz, 3H), 3.78-3.62 (m, 13H), 3.46 (d, J=4.8 Hz, 3H), 2.85-2.66 (m, 5H). ¹³C NMR (101 MHZ, CDCl₃) δ 171.14, 169.63, 168.26, 166.95, 166.63, 165.95, 154.60, 142.10, 136.96, 133.65, 128.63, 127.86, 127.18, 119.69, 118.25, 117.39, 77.34, 77.23, 77.02, 77.02, 76.71, 75.90, 68.90, 68.19, 67.36, 60.41, 51.97, 51.51, 49.24, 45.85, 41.78, 39.37, 33.38, 31.29, 29.71, 29.37, 22.91, 14.21, 14.12.

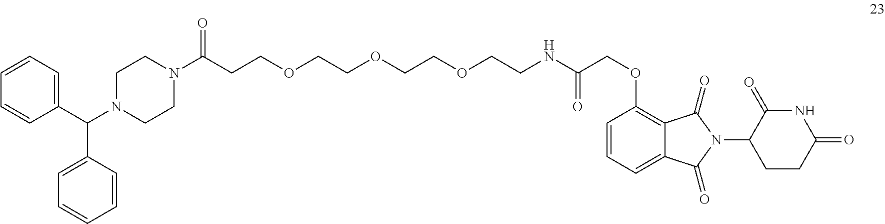 [SMILES: O=C(COc1cccc2c1C(=O)N(C1CCC(=O)NC1=O)C2=O)NCCOCCOCCOCCC(=O)N1CCN(C(c2ccccc2)c2ccccc2)CC1]

### N-(2-(2-(2-(3-(4-benzhydrylpiperazin-1-yl)-3-oxopropoxy) ethoxy) ethoxy)ethyl)-2-((2-(2,6-dioxopiperidin-3-yl)-1,3-dioxoisoindolin-4-yl)oxy) acetamide (23)

General Procedure 3 then 2. Reaction scale: 16.11 mg (30.08 μmol, 1.0 eq) of 3 and 8.35 mg (33.09 μmol, 1.1 eq) of benzhydrylpiperazine. Purified by pTLC (8% MeOH/DCM) to afford 23 as a white foam (17.3 mg, 75%). R_{f}=0.58 (10% MeOH/CH₂Cl₂, UV-active); LC-MS (ESI+) calc'd for C₄₁H₄₇N₅O₁₀ 769.3, found [M+H]⁺ 770.1. ¹H NMR (400 MHZ, CDCl₃) δ 9.08 (s, 1H), 7.73 (dd, J=8.4, 7.3 Hz, 1H), 7.62 (t, J=5.2 Hz, 1H), 7.54 (d, J=7.3 Hz, 1H), 7.40 (d, J=7.0 Hz, 4H), 7.30-7.24 (m, 4H), 7.23-7.14 (m, 3H), 4.98-4.89 (m, 1H), 4.64 (s, 2H), 4.22 (s, 1H), 3.78 (t, J=6.8 Hz, 2H), 3.67-3.51 (m, 14H), 3.49-3.42 (m, 2H), 2.91-2.66 (m, 4H), 2.60 (t, J=6.8 Hz, 2H), 2.36 (q, J=5.4 Hz, 4H). ¹³C NMR (101 MHz, CDCl₃) δ 169.3, 168.1, 166.8, 166.7, 165.8 (2C), 154.5, 142.2 (2C), 136.9, 128.6 (4C), 127.9 (4C), 127.2 (2C), 119.3, 117.3, 77.4, 77.0, 76.7, 75.9, 70.4, 70.3, 70.3, 69.5, 67.9, 67.2, 52.0, 51.5, 49.3, 41.8, 33.3, 31.5, 29.7, 29.4, 22.7.

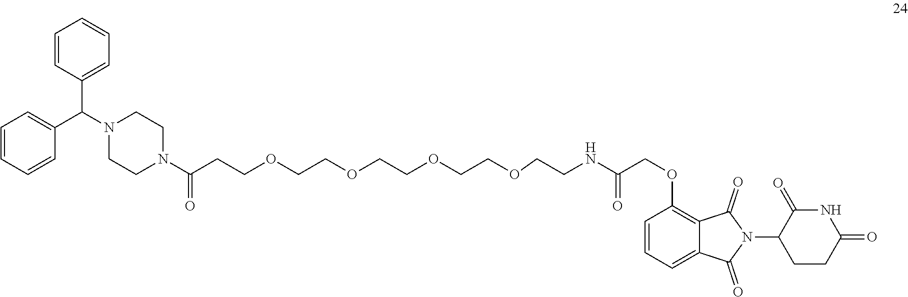 [SMILES: O=C(COc1cccc2c1C(=O)N(C1CCC(=O)NC1=O)C2=O)NCCOCCOCCOCCOCCC(=O)N1CCN(C(c2ccccc2)c2ccccc2)CC1]

### N-(15-(4-benzhydrylpiperazin-1-yl)-15-oxo-3,6,9,12-tetraoxapentadecyl)-2-((2-(2,6-dioxopiperidin-3-yl)-1,3-dioxoisoindolin-4-yl)oxy) acetamide (24)

General Procedure 3 then 2. Reaction scale: 15.0 mg (25.9 μmol, 1.0 eq) of 5 and 7.2 mg (28.5 μmol, 1.1 eq) of benzhydrylpiperazine. Purified by pTLC (10% MeOH/DCM) to afford 24 as a white foam (12.7 mg, 61%). R/=0.73 (10% MeOH/CH₂Cl₂, UV-active); LC-MS (ESI+) calc'd for C₄₃H₅₁N₅O₁₁ 813.4, found [M+H]⁺ 814.1. ¹H NMR (400 MHZ, CDCl₃) δ 9.08 (s, 1H), 7.73 (dd, J=8.4, 7.3 Hz, 1H), 7.62 (t, J=5.2 Hz, 1H), 7.54 (d, J=7.3 Hz, 1H), 7.40 (d, J=7.0 Hz, 4H), 7.30-7.24 (m, 4H), 7.23-7.14 (m, 3H), 4.98-4.89 (m, 1H), 4.64 (s, 2H), 4.22 (s, 1H), 3.78 (t, J=6.8 Hz, 2H), 3.67-3.51 (m, 18H), 3.49-3.42 (m, 2H), 2.91-2.66 (m, 4H), 2.60 (t, J=6.8 Hz, 2H), 2.36 (q, J=5.4 Hz, 4H). ¹³C NMR (101 MHz, CDCl₃) δ 171.11, 169.32, 168.16, 166.82, 154.46, 142.20, 136.93, 133.72, 128.61, 127.87, 127.15, 119.28, 117.27, 77.34, 77.23, 77.03, 76.71, 75.94, 70.59, 70.45, 70.35, 70.30, 69.47, 67.88, 67.26, 60.41, 52.01, 51.55, 49.34, 45.76, 41.73, 39.12, 33.41, 31.47, 29.71, 22.70, 14.21.

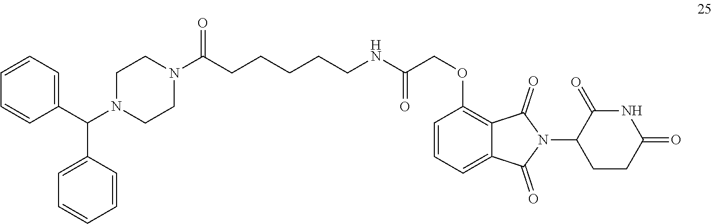 [SMILES: O=C(COc1cccc2c1C(=O)N(C1CCC(=O)NC1=O)C2=O)NCCCCCC(=O)N1CCN(C(c2ccccc2)c2ccccc2)CC1]

### N-(6-(4-benzhydrylpiperazin-1-yl)-6-oxohexyl)-2-((2-(2,6-dioxopiperidin-3-yl)-1,3-dioxoisoindolin-4-yl)oxy) acetamide (25)

General Procedure 3 then 2. Reaction scale: 17 mg (38.17 μmol, 1.0 eq) of 6 and 10.4 mg (41.21 μmol, 1.1 eq) of benzhydrylpiperazine. Purified by pTLC (8% MeOH/DCM) to afford 25 as a white foam (25 mg, 99%). R_{f}=0.67 (8% MeOH/CH₂Cl₂, UV-active); LC-MS (ESI+) calc'd for C₃₈H₄₁N₅O₇ 679.3, found [M+H]⁺ 680.0. ¹H NMR (400 MHZ, CDCl₃) δ 9.08 (br. s, 1H), 7.73 (dd, J=8.4, 7.4 Hz, 1H), 7.54 (dd, J=7.4, 0.6 Hz, 1H), 7.39 (d, J=7.3 Hz, 4H), 7.26 (s, 4H), 7.20-7.17 (m, 3H), 4.98-4.93 (m, 1H), 4.63 (q, J=3.1 Hz, 2H), 4.21 (s, 1H), 3.78 (ddd, J=9.1, 7.1, 2.4 Hz, 2H), 3.48-3.27 (m, 7H), 2.90-2.76 (m, 1H), 2.73-2.58 (m, 3H), 2.36 (s, 3H), 1.60-1.30 (m, 6H). ¹³C NMR (101 MHZ, CDCl₃) δ 171.96, 171.31, 168.33, 166.84, 166.60, 166.27, 154.81, 142.14, 137.04, 133.58, 128.62, 127.87, 127.17, 120.24, 118.51, 117.61, 77.35, 77.23, 77.03, 76.81, 76.71, 75.96, 68.67, 52.11, 51.63, 49.35, 45.89, 41.83, 39.21, 33.04, 31.41, 29.71, 28.87, 26.78, 25.35, 22.86.

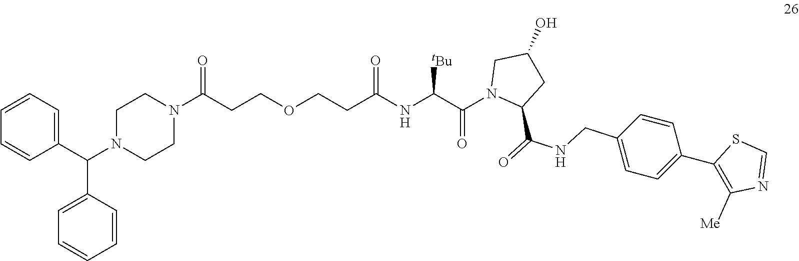 [SMILES: Cc1ncsc1-c1ccc(CNC(=O)[C@@H]2C[C@@H](O)CN2C(=O)[C@@H](NC(=O)CCOCCC(=O)N2CCN(C(c3ccccc3)c3ccccc3)CC2)C(C)(C)C)cc1]

### (2S,4R)-1-((S)-2-(3-(3-(4-benzhydrylpiperazin-1-yl)-3-oxopropoxy) propanamido)-3,3-dimethylbutanoyl)-4-hydroxy-N-(4-(4-methylthiazol-5-yl)benzyl) pyrrolidine-2-carboxamide (26)

General Procedure 3 then 2. Reaction scale: 10.2 mg (17.8 μmol, 1.0 eq) of 8 and 4.93 mg (19.52 μmol, 1.1 eq) of benzhydrylpiperazine. Purified by pTLC (10% MeOH/DCM) to afford 26 as a pale yellow solid (6 mg, 42%). R_{f}=0.46 (10% MeOH/CH₂Cl₂, UV-active); LC-MS (ESI+) calc'd for C₄₅H₅₆N₆O₆S 808.4, found [M+H]⁺ 809.3. ¹H NMR (400 MHz, CDCl₃) δ 8.68 (s, 1H), 7.47-7.30 (m, 11H), 7.20 (d, J=7.4 Hz, 2H), 6.95 (d, J=8.3 Hz, 1H), 4.73-4.65 (m, 1H), 4.59-4.52 (m, 1H), 4.45 (d, J=8.6 Hz, 1H), 4.29 (dd, J=15.1, 5.1 Hz, 1H), 3.79-3.49 (m, 10H), 2.52 (s, 7H), 2.03 (d, J=15.4 Hz, 1H), 1.61 (m, 9H), 0.90 (s, 9H).

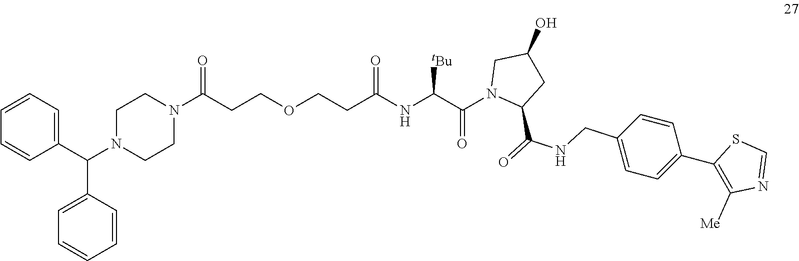 [SMILES: Cc1ncsc1-c1ccc(CNC(=O)[C@@H]2C[C@H](O)CN2C(=O)[C@@H](NC(=O)CCOCCC(=O)N2CCN(C(c3ccccc3)c3ccccc3)CC2)C(C)(C)C)cc1]

### (2S,4S)-1-((S)-2-(3-(3-(4-benzhydrylpiperazin-1-yl)-3-oxopropoxy) propanamido)-3,3-dimethylbutanoyl)-4-hydroxy-N-(4-(4-methylthiazol-5-yl)benzyl) pyrrolidine-2-carboxamide (27)

General Procedure 3 then 2. Reaction scale: 11.4 mg (19.9 μmol, 1 eq.) of 9 and 5.6 mg (22 μmol) of benzhydrylpiperazine. Purified by pTLC (10% MeOH/DCM) to afford 27 as a white solid (6 mg, 42%). R_{f}=0.46 (10% MeOH/CH₂Cl₂, UV-active); LC-MS (ESI+) calc'd for C₄₅H₅₆N₆O₆S 808.4, found [M+H]⁺ 809.2. ¹H NMR (400 MHZ, CDCl₃) δ 8.68 (s, 1H), 7.41-7.26 (m, 12H), 7.20 (d, J=7.4 Hz, 2H), 4.68-4.56 (m, 2H), 4.48 (d, J=8.9, 2H), 4.25 (dd, J=15.1, 5.1 Hz, 1H), 3.92 (dd, J=10.6, 4.1 Hz, 1H), 3.81-3.65 (m, 7H), 3.59 (s, 1H), 3.45 (d, J=5.3 Hz, 1H), 2.66-2.55 (m, 2H), 2.52 (s, 4H), 2.18 (dd, J=8.9, 5.4 Hz, 2H), 1.56 (s, 9H), 0.89 (s, 9H).

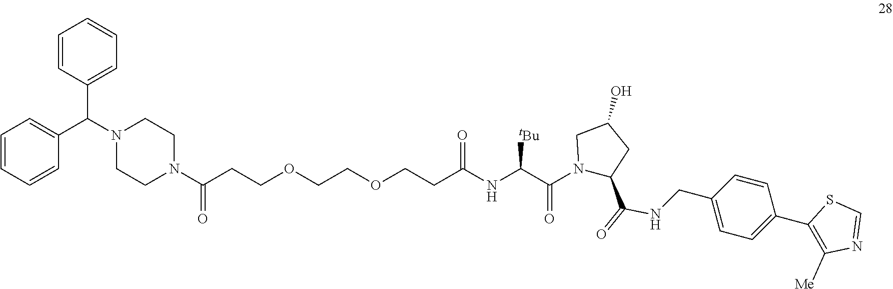 [SMILES: Cc1ncsc1-c1ccc(CNC(=O)[C@@H]2C[C@@H](O)CN2C(=O)[C@@H](NC(=O)CCOCCOCCC(=O)N2CCN(C(c3ccccc3)c3ccccc3)CC2)C(C)(C)C)cc1]

### (2S,4R)-1-((S)-2-(3-(2-(3-(4-benzhydrylpiperazin-1-yl)-3-oxopropoxy) ethoxy) propanamido)-3,3-dimethylbutanoyl)-4-hydroxy-N-(4-(4-methylthiazol-5-yl)benzyl) pyrrolidine-2-carboxamide (28)

General Procedure 3 then 2. Reaction scale: 7.5 mg (11.1 μmol, 1.0 eq) of 10 and 3.1 mg (12.2 μmol, 1.1 eq) of benzhydrylpiperazine. Purified by pTLC (10% MeOH/DCM) to afford 28 as a white foam (6.4 mg, 25%). R/=0.46 (8% MeOH/CH₂Cl₂, UV-active); LC-MS (ESI+) calc'd for C₄₇H₆₀N₆O₇S 852.4, found [M+H]⁺ 853.3. ¹H NMR (400 MHZ, CDCl₃) δ 8.67 (s, 1H), 7.52 (t, J=6.0 Hz, 1H), 7.41-7.35 (m, 5H), 7.34 (s, 3H), 7.28 (d, J=7.1 Hz, 3H), 7.21-7.16 (m, 2H), 7.03 (d, J=8.3 Hz, 1H), 4.71 (t, J=8.1 Hz, 1H), 4.57 (dd, J=15.0, 6.6 Hz, 1H), 4.50-4.42 (m, 2H), 4.32 (dd, J=15.0, 5.2 Hz, 1H), 4.20 (s, 1H), 4.12-4.06 (m, 1H), 3.80-3.65 (m, 5H), 3.59 (ddd, J=10.3, 6.3, 4.3 Hz, 7H), 3.49-3.41 (m, 3H), 2.56 (td, J=6.8, 1.3 Hz, 2H), 2.51 (s, 4H), 2.46 (q, J=4.6 Hz, 3H), 2.34 (dt, J=10.7, 5.2 Hz, 4H), 0.93 (s, 9H). ¹³C NMR (101 MHz, CDCl₃) δ 172.14, 171.70, 170.88, 169.32, 150.27, 148.45, 142.08, 138.26, 131.63, 130.86, 129.47, 128.63, 128.10, 127.83, 127.19, 77.34, 77.22, 77.02, 76.70, 75.92, 70.48, 70.38, 70.13, 67.38, 67.16, 58.34, 57.70, 56.67, 51.97, 51.50, 45.75, 43.18, 41.72, 36.77, 35.95, 34.85, 33.38, 26.41, 16.08.

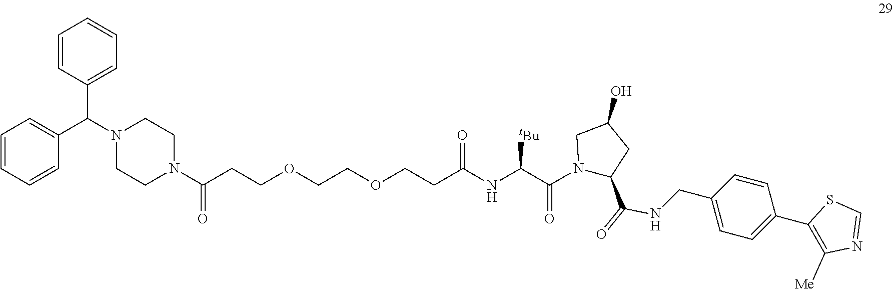 [SMILES: Cc1ncsc1-c1ccc(CNC(=O)[C@@H]2C[C@H](O)CN2C(=O)[C@@H](NC(=O)CCOCCOCCC(=O)N2CCN(C(c3ccccc3)c3ccccc3)CC2)C(C)(C)C)cc1]

### (2S,4S)-1-((S)-2-(3-(2-(3-(4-benzhydrylpiperazin-1-yl)-3-oxopropoxy) ethoxy) propanamido)-3,3-dimethylbutanoyl)-4-hydroxy-N-(4-(4-methylthiazol-5-yl)benzyl) pyrrolidine-2-carboxamide (29)

General Procedure 3 then 1. Reaction scale: 16.2 mg (26.2 μmol, 1.0 eq) of 11 and 7.3 mg (28.8 μmol, 1.1 eq) of benzhydrylpiperazine. Purified by pTLC (10% MeOH/DCM) to afford 29 as a white foam (19.6 mg, 88%). R/=0.51 (10% MeOH/CH₂Cl₂, UV-active); LC-MS (ESI+) calc'd for C₄₇H₆₀N₆O₇S 852.4, found [M+H]⁺ 853.3. ¹H NMR (600 MHZ, CDCl₃) δ 8.59 (s, 1H), 7.44 (t, J=6.0 Hz, 1H), 7.32-7.28 (m, 4H), 7.26 (d, J=1.2 Hz, 4H), 7.21-7.17 (m, 5H), 7.12-7.08 (m, 2H), 6.98 (d, J=8.4 Hz, 1H), 4.63 (t, J=8.1 Hz, 1H), 4.48 (dd, J=15.0, 6.7 Hz, 1H), 4.43-4.38 (m, 2H), 4.23 (dd, J=15.0, 5.3 Hz, 1H), 4.12 (s, 1H), 4.06-3.99 (m, 1H), 3.69-3.57 (m, 5H), 3.51 (dddd, J=15.6, 12.6, 8.7, 5.3 Hz, 7H), 3.36 (dd, J=6.2, 4.0 Hz, 2H), 2.48 (td, J=6.9, 1.8 Hz, 2H), 2.43 (s, 3H), 2.38 (t, J=5.6 Hz, 2H), 2.26 (dt, J=17.0, 5.2 Hz, 4H), 0.85 (s, 9H). ¹³C NMR (151 MHz, CDCl₃) δ 172.16, 171.69, 171.20, 170.96, 169.38, 150.32, 148.43, 142.09, 138.31, 131.67, 130.83, 130.08, 129.47, 129.41, 128.64, 128.09, 127.84, 127.21, 77.26, 77.04, 76.83, 75.93, 70.47, 70.38, 70.14, 67.37, 67.18, 60.42, 60.37, 58.45, 57.73, 56.71, 56.00, 51.98, 51.51, 45.76, 43.18, 41.74, 36.74, 36.06, 34.94, 33.38, 31.94, 29.72, 29.38, 26.42, 22.71, 21.08, 20.80, 16.07, 14.22, 14.14.

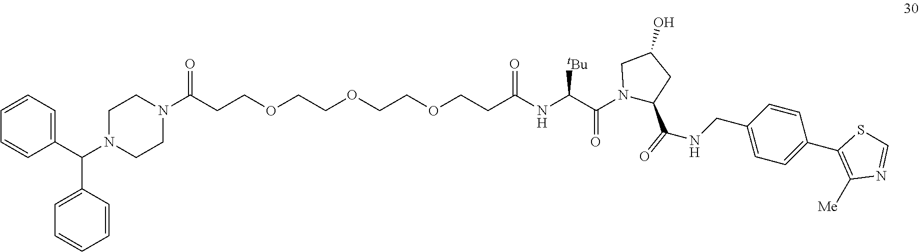 [SMILES: Cc1ncsc1-c1ccc(CNC(=O)[C@@H]2C[C@@H](O)CN2C(=O)[C@@H](NC(=O)CCOCCOCCOCCC(=O)N2CCN(C(c3ccccc3)c3ccccc3)CC2)C(C)(C)C)cc1]

### (2S,4R)-1-((S)-16-(4-benzhydrylpiperazin-1-yl)-2-(tert-butyl)-4,16-dioxo-7,10,13-trioxa-3-azahexadecanoyl)-4-hydroxy-N-(4-(4-methylthiazol-5-yl)benzyl) pyrrolidine-2-carboxamide (30)

General Procedure 3 then 1. Reaction scale: 21.9 mg (33.0 μmol, 1.0 eq) of 12 and 9.2 mg (36.35 μmol, 1.1 eq) of benzhydrylpiperazine. Purified by pTLC (10% MeOH/DCM) to afford 30 as a white foam (10.5 mg, 35%). R/=0.52 (10% MeOH/CH₂Cl₂, UV-active); LC-MS (ESI+) calc'd for C₄₉H₆₄N₆O₈S 896.5, found [M+H]⁺ 897.3. ¹H NMR (600 MHZ, CDCl₃) δ 8.67 (s, 1H), 7.47 (t, J=6.0 Hz, 1H), 7.40-7.38 (m, 4H), 7.36-7.33 (m, 4H), 7.29-7.25 (m, 5H), 7.21-7.16 (m, 2H), 7.02 (d, J=8.4 Hz, 1H), 4.72 (t, J=8.1 Hz, 1H), 4.56 (dd, J=15.0, 6.7 Hz, 1H), 4.50-4.46 (m, 2H), 4.32 (dd, J=15.0, 5.3 Hz, 1H), 4.21 (s, 1H), 4.10 (d, J=11.0 Hz, 1H), 3.74 (tt, J=6.1, 3.1 Hz, 3H), 3.69 (dddd, J=9.8, 8.5, 5.0, 2.7 Hz, 3H), 3.60 (dd, J=13.1, 2.0 Hz, 9H), 3.45 (t, J=5.1 Hz, 2H), 2.57 (td, J=6.8, 4.1 Hz, 2H), 2.51 (s, 3H), 2.47 (ddd, J=7.2, 4.7, 3.1 Hz, 2H), 2.35 (dt, J=16.0, 4.1 Hz, 4H), 0.93 (s, 9H). ¹³C NMR (151 MHz, CDCl₃) δ 172.19, 171.75, 170.91, 169.39, 150.32, 148.45, 142.12, 138.26, 131.65, 130.90, 130.88, 129.49, 128.64, 128.12, 127.85, 127.20, 77.26, 77.14, 77.04, 76.83, 75.94, 70.45, 70.37, 70.30, 70.19, 70.12, 67.17, 67.14, 58.39, 57.76, 56.70, 51.96, 51.52, 50.82, 45.73, 43.20, 41.74, 36.61, 35.98, 34.84, 33.71, 33.38, 30.37, 30.18, 29.72, 26.42, 16.08.

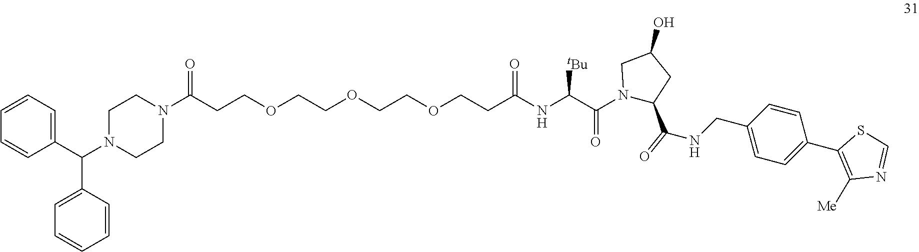 [SMILES: Cc1ncsc1-c1ccc(CNC(=O)[C@@H]2C[C@H](O)CN2C(=O)[C@@H](NC(=O)CCOCCOCCOCCC(=O)N2CCN(C(c3ccccc3)c3ccccc3)CC2)C(C)(C)C)cc1]

### (2S,4S)-1-((S)-16-(4-benzhydrylpiperazin-1-yl)-2-(tert-butyl)-4,16-dioxo-7,10,13-trioxa-3-azahexadecanoyl)-4-hydroxy-N-(4-(4-methylthiazol-5-yl)benzyl) pyrrolidine-2-carboxamide (31)

General Procedure 3 then 1. Reaction scale: 10 mg (15.1 μmol, 1.0 eq) of 13 and 4.2 mg (16.6 μmol, 1.1 eq) of benzhydrylpiperazine. Purified by pTLC (10% MeOH/DCM) to afford 30 as a white foam (10.5 mg, 35%). R_{f}=0.57 (10% MeOH/CH₂Cl₂, UV-active); LC-MS (ESI−) calc'd for C₄₉H₆₄N₆O₈S 896.5, found [M+HCOO]⁻ 941.1. ¹H NMR (400 MHZ, CDCl₃) δ 8.68 (s, 1H), 7.46 (d, J=6.2 Hz, 1H), 7.42-7.37 (m, 4H), 7.34 (d, J=1.7 Hz, 4H), 7.29-7.24 (m, 5H), 7.21-7.16 (m, 2H), 7.05 (d, J=8.3 Hz, 1H), 4.72 (t, J=8.0 Hz, 1H), 4.57 (dd, J=15.0, 6.6 Hz, 1H), 4.47 (d, J=8.8 Hz, 2H), 4.32 (dd, J=15.0, 5.2 Hz, 1H), 4.21 (s, 1H), 4.14-4.09 (m, 1H), 3.72 (dt, J=17.1, 4.5 Hz, 5H), 3.60 (d, J=8.8 Hz, 9H), 3.45 (t, J=5.0 Hz, 2H), 2.61-2.54 (m, 2H), 2.51 (s, 3H), 2.46 (d, J=5.1 Hz, 2H), 2.35 (dt, J=10.0, 5.0 Hz, 4H), 0.93 (s, 9H). ¹³C NMR (151 MHz, CDCl₃) δ 172.85, 172.00, 171.51, 169.28, 150.36, 148.50, 142.08, 129.73, 129.55, 128.63, 128.24, 128.11, 127.92, 127.83, 127.57, 127.19, 77.22, 77.01, 76.80, 75.94, 71.16, 70.51, 70.44, 70.32, 70.23, 67.25, 67.21, 59.82, 58.57, 57.01, 55.98, 51.97, 51.52, 45.72, 43.42, 41.70, 36.79, 35.22, 34.97, 33.69, 33.43, 31.92, 30.16, 30.03, 29.70, 29.65, 29.60, 29.47, 29.36, 29.32, 27.21, 26.70, 26.31, 25.53, 23.17, 22.69, 21.49, 16.06, 14.18, 14.12.

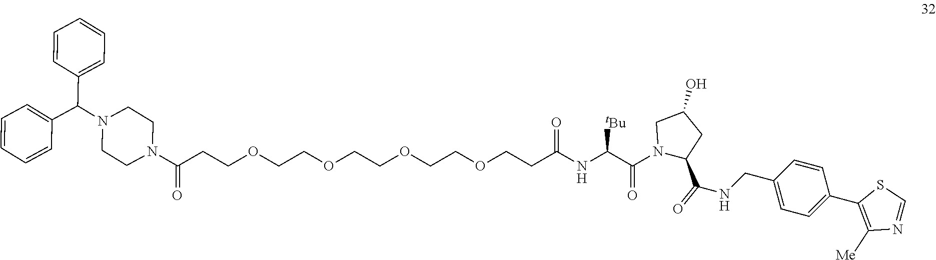 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(C(c3ccccc3)c3ccccc3)CC2)C(C)(C)C)cc1]

### (2S,4R)-1-((S)-19-(4-benzhydrylpiperazin-1-yl)-2-(tert-butyl)-4,19-dioxo-7,10,13,16-tetraoxa-3-azanonadecanoyl)-4-hydroxy-N-(4-(4-methylthiazol-5-yl)benzyl) pyrrolidine-2-carboxamide (32)

General Procedure 3 then 2. Reaction scale: 15.3 mg (21.65 μmol) of 15 and 6.01 mg (23.81 μmol) of benzhydrylpiperazine. Purified by pTLC (10% MeOH/DCM) to afford 32 as a white solid (7.0 mg, 35%). R/=0.47 (10% MeOH/CH₂Cl₂, UV-active); LC-MS (ESI+) calc'd for C₅₁H₆₈N₆O₉S 940.5, found [M+H]⁺ 941.4. ¹H NMR (400 MHZ, CDCl₃) δ 8.68 (s, 1H), 7.43-7.31 (m, 9H), 7.27 (d, J=5.3 Hz, 5H), 7.23-7.15 (m, 2H), 7.03 (d, J=8.2 Hz, 1H), 4.73 (t, J=8.0 Hz, 1H), 4.57 (dd, J=14.9, 6.6 Hz, 1H), 4.52-4.43 (m, 2H), 4.33 (dd, J=14.9, 5.2 Hz, 1H), 4.22 (s, 1H), 4.14 (d, J=7.1 Hz, 1H), 3.78-3.70 (m, 5H), 3.66-3.58 (m, 15H), 3.46 (dd, J=11.2, 6.2 Hz, 2H), 2.61-2.57 (m, 2H), 2.52 (d, J=1.7 Hz, 3H), 2.50-2.46 (m, 2H), 2.39-2.30 (m, 4H), 0.93 (s, 9H).

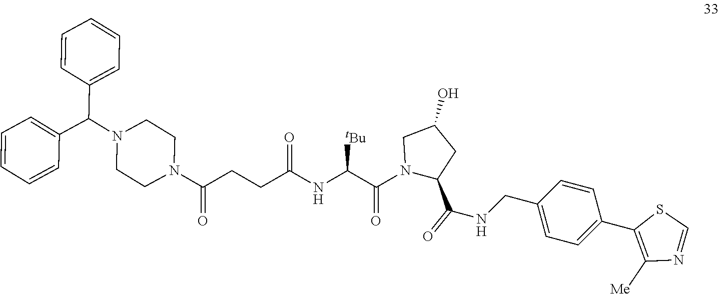 [SMILES: Cc1ncsc1-c1ccc(CNC(=O)[C@@H]2C[C@@H](O)CN2C(=O)[C@@H](NC(=O)CCC(=O)N2CCN(C(c3ccccc3)c3ccccc3)CC2)C(C)(C)C)cc1]

### (2S,4R)-1-((S)-2-(4-(4-benzhydrylpiperazin-1-yl)-4-oxobutanamido)-3,3-dimethylbutanoyl)-4-hydroxy-N-(4-(4-methylthiazol-5-yl)benzyl) pyrrolidine-2-carboxamide (33)

General Procedure 3 then 2. Reaction scale: 6.7 mg (12.63 μmol) of 16 and 3.5 mg (13.89 μmol) of benzhydrylpiperazine. Purified by pTLC (10% MeOH/DCM) to afford 33 as a pale yellow solid (4.2 mg, 44%). R_{f}=0.58 (10% MeOH/CH₂Cl₂, UV-active); LC-MS (ESI+) calc'd for C₄₃H₅₂N₆O₅S 764.4, found [M+H]⁺ 766.1. ¹H NMR (400 MHZ, CDCl₃) δ 8.67 (s, 1H), 7.42-7.32 (m, 12H), 7.27 (s, 3H), 7.20 (d, J=7.3 Hz, 2H), 4.75 (t, J=8.1 Hz, 2H), 4.57 (dd, J=14.9, 6.6 Hz, 1H), 4.49 (d, J=8.3 Hz, 2H), 4.33 (dd, J=14.9, 5.1 Hz, 1H), 4.22 (s, 1H), 4.12 (d, J=6.8 Hz, 1H), 3.65 (d, J=7.6 Hz, 2H), 3.56 (d, J=13.7 Hz, 4H), 3.49 (s, 1H), 3.41 (d, J=5.5 Hz, 3H), 2.51 (s, 3H), 2.35 (d, J=4.9 Hz, 4H), 0.92 (s, 9H).

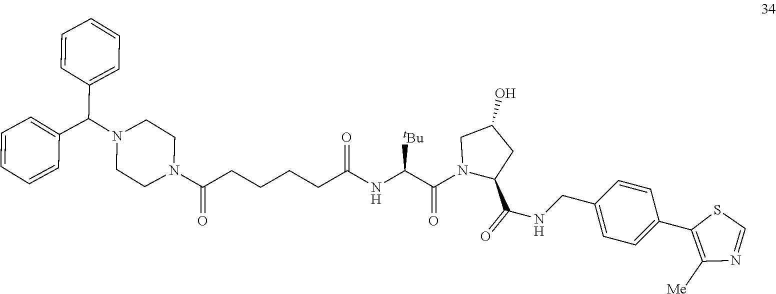 [SMILES: Cc1ncsc1-c1ccc(CNC(=O)[C@@H]2C[C@@H](O)CN2C(=O)[C@@H](NC(=O)CCCCC(=O)N2CCN(C(c3ccccc3)c3ccccc3)CC2)C(C)(C)C)cc1]

### (2S,4R)-1-((S)-2-(6-(4-benzhydrylpiperazin-1-yl)-6-oxohexanamido)-3,3-dimethylbutanoyl)-4-hydroxy-N-(4-(4-methylthiazol-5-yl)benzyl) pyrrolidine-2-carboxamide (34)

General Procedure 3 then 2. Reaction scale: 9 mg (16.11 μmol) of 17 and 4.47 mg (17.72 μmol) of benzhydrylpiperazine. Purified by pTLC (10% MeOH/DCM) to afford 21 as a pale yellow solid (3.3 mg, 26%). R_{f}=0.54 (10% MeOH/CH₂Cl₂, UV-active); LC-MS (ESI+) calc'd for C₄₅H₅₆N₆O₅S 792.4, found [M+H]⁺ 793.9. ¹H NMR (400 MHZ, CDCl₃) δ 8.86 (br. s, 1H), 7.73 (dd, J=8.4, 7.4 Hz, 1H), 7.54 (dd, J=7.4, 0.6 Hz, 1H), 7.50 (br. t, 2H), 7.39 (d, J=7.3 Hz, 5H), 7.30 (s, 1H), 7.25 (br. t, 2H), 7.20-7.17 (m, 3H), 4.72 (t, 2H), 4.58 (s, 1H) 4.50 (s, 1H), 4.60-4.30 (dd, J=7 Hz, 1H), 4.20 (s, 1H), 4.15-4.10 (d, J=7 Hz, 2H), 3.62 (q, J=5.4 Hz, 4H), 3.47 (q, J=6.7, 4.9 Hz, 4H), 2.60-2.35 (m, 13H), 2.20-2.10 (m, 1H), 2.05 (s, 1H), 0.93 (s, 9H).

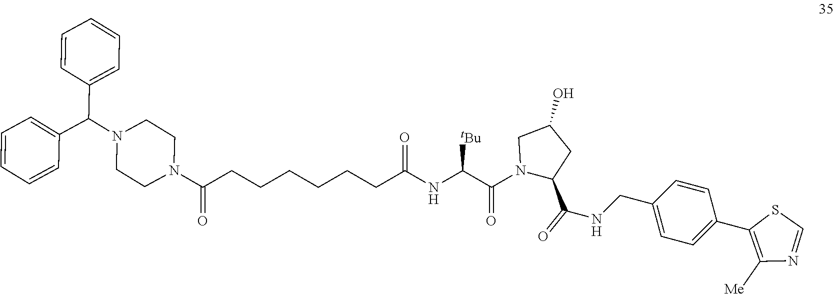 [SMILES: Cc1ncsc1-c1ccc(CNC(=O)[C@@H]2C[C@@H](O)CN2C(=O)[C@@H](NC(=O)CCCCCCC(=O)N2CCN(C(c3ccccc3)c3ccccc3)CC2)C(C)(C)C)cc1]

### (2S,4R)-1-((S)-2-(8-(4-benzhydrylpiperazin-1-yl)-8-oxooctanamido)-3,3-dimethylbutanoyl)-4-hydroxy-N-(4-(4-methylthiazol-5-yl)benzyl) pyrrolidine-2-carboxamide (35)

General Procedure 5 then 2. Reaction scale: 8.5 mg (14.49 μmol) of 19 and 4.02 mg (15.94 μmol) of benzhydrylpiperazine. Purified by pTLC (10% MeOH/DCM) to afford 23 as a pale yellow solid (6.4 mg, 54%). R_{f}=0.53 (10% MeOH/CH₂Cl₂, UV-active); LC-MS (ESI+) calc'd for C₄₇H₆₀N₆O₅S 820.4, found [M+H]⁺ 821.0. ¹H NMR (400 MHZ, CDCl₃) δ 8.86 (br. s, 1H), 7.73 (dd, J=8.4, 7.4 Hz, 1H), 7.54 (dd, J=7.4, 0.6 Hz, 1H), 7.50 (br. t, 2H), 7.39 (d, J=7.3 Hz, 5H), 7.30 (s, 1H), 7.25 (br. t, 2H), 7.20-7.17 (m, 3H), 4.72 (t, 2H), 4.58 (s, 1H) 4.50 (s, 1H), 4.60-4.30 (dd, J=7 Hz, 1H), 4.20 (s, 1H), 4.15-4.10 (d, J=7 Hz, 2H), 3.62 (q, J=5.4 Hz, 4H), 3.47 (q, J=6.7, 4.9 Hz, 4H), 2.60-2.35 (m, 17H), 2.20-2.10 (m, 1H), 2.05 (s, 1H), 0.93 (s, 9H).

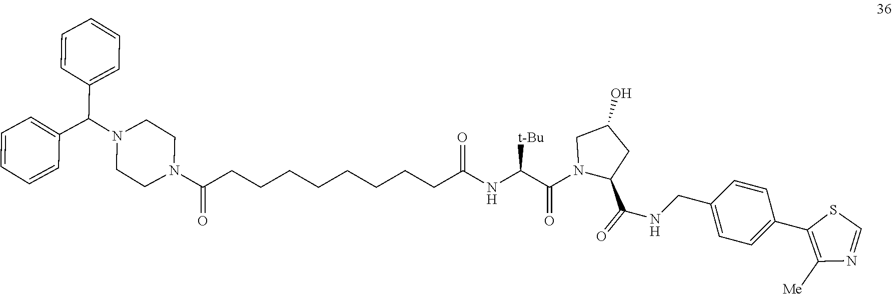 [SMILES: Cc1ncsc1-c1ccc(CNC(=O)[C@@H]2C[C@@H](O)CN2C(=O)[C@@H](NC(=O)CCCCCCCCC(=O)N2CCN(C(c3ccccc3)c3ccccc3)CC2)C(C)(C)C)cc1]

### (2S,4R)-1-((S)-2-(10-(4-benzhydrylpiperazin-1-yl)-10-oxodecanamido)-3,3-dimethylbutanoyl)-4-hydroxy-N-(4-(4-methylthiazol-5-yl)benzyl) pyrrolidine-2-carboxamide (36)

General Procedure 5 then 2. Reaction scale: 10 mg (16.27 μmol) of 20 and 4.52 mg (17.89 μmol) of benzhydrylpiperazine. Purified by pTLC (10% MeOH/DCM) to afford 36 as a pale yellow solid (8.5 mg, 62%). R/=0.62 (10% MeOH/CH₂Cl₂, UV-active); LC-MS (ESI+) calc'd for C₄₉H₆₄N₆O₅S 848.5, found [M+H]⁺ 849.3. ¹H NMR (400 MHZ, CDCl₃) δ 8.86 (br. s, 1H), 7.73 (dd, J=8.4, 7.4 Hz, 1H), 7.54 (dd, J=7.4, 0.6 Hz, 1H), 7.50 (br. t, 2H), 7.39 (d, J=7.3 Hz, 5H), 7.30 (s, 1H), 7.25 (br. t, 2H), 7.20-7.17 (m, 3H), 4.72 (t, 2H), 4.58 (s, 1H) 4.50 (s, 1H), 4.60-4.30 (dd, J=7 Hz, 1H), 4.20 (s, 1H), 4.15-4.10 (d, J=7 Hz, 2H), 3.62 (q, J=5.4 Hz, 4H), 3.47 (q, J=6.7, 4.9 Hz, 4H), 2.60-2.35 (m, 21H), 2.20-2.10 (m, 1H), 2.05 (s, 1H), 0.93 (s, 9H).

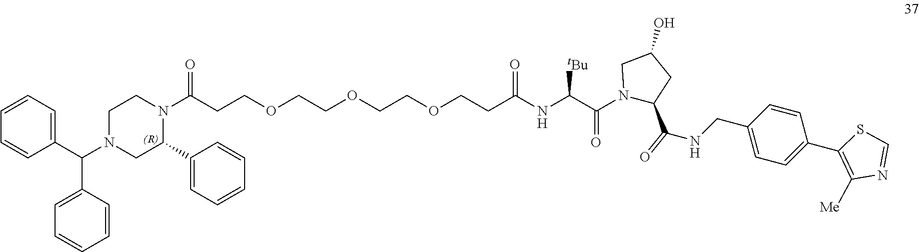

### (2S,4R)-1-((S)-16-((R)-4-benzhydryl-2-phenylpiperazin-1-yl)-2-(tert-butyl)-4,16-dioxo-7,10,13-trioxa-3-azahexadecanoyl)-4-hydroxy-N-(4-(4-methylthiazol-5-yl)benzyl) pyrrolidine-2-carboxamide (37)

General Procedure 3 then 1. Reaction scale: 15 mg (22.6 μmol, 1.0 eq.) of 12 and 8.2 mg (24.9 μmol, 1.1 eq.) of (4R)-benzhydrylpiperazine. Purified by pTLC (8% MeOH/CH₂Cl₂) to afford 37 as an off-white solid (8.3 mg, 38%). R_{f}=0.55 (8% MeOH/CH₂Cl₂, UV-active); LC-MS (ESI+) calc'd for C₅₅H₇₀N₆O₈S [M+H]⁺: 972.5, found 974.0; ¹H NMR (600 MHZ, CDCl₃) δ 8.68 (s, 1H), 7.34 (s, 13H), 7.19 (s, 6H), 7.01 (d, J=8.3 Hz, 1H), 4.73 (s, 1H), 4.47 (d, J=8.3 Hz, 2H), 4.28-4.02 (m, 3H), 3.79-3.03 (m, 19H), 2.50 (s, 8H), 2.17-2.03 (m, 4H), 0.91 (s, 9H). Note: presence of rotamers.

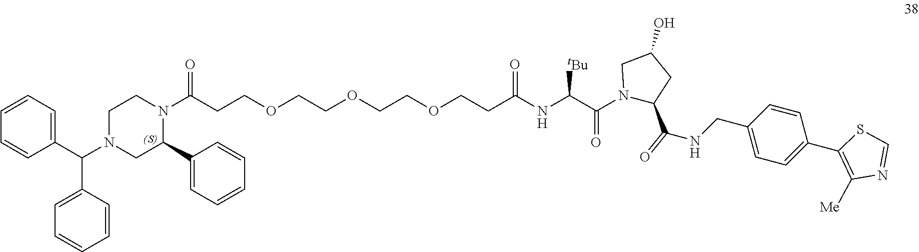

### (2S,4R)-1-((S)-16-((S)-4-benzhydryl-2-phenylpiperazin-1-yl)-2-(tert-butyl)-4,16-dioxo-7,10,13-trioxa-3-azahexadecanoyl)-4-hydroxy-N-(4-(4-methylthiazol-5-yl)benzyl) pyrrolidine-2-carboxamide (38)

General Procedure 1 and 3. Reaction scale: 21.6 mg (30.0 μmol, 1.00 equiv) of 12 and 14.9 mg (35.0 μmol, 1.17 equiv) of (4S)-phenylbenzhydrylpiperazine derivative. Purified by PTLC (10% MeOH/EtOAc) to afford 38 as a clear oil (5.5 mg, 19%). R_{f}=0.47 (10% MeOH/EtOAc, UV-active); LC-MS (ESI+) calc'd for C₅₅H₇₀N₆O₈S [M+2H]²⁺: 487.2, found 487.1; ¹H NMR (600 MHZ, CDCl₃) δ 8.67 (s, 1H), 7.41-7.27 (m, 10H), 7.23-7.13 (m, 9H), 6.34 (d, J=8.5 Hz, 1H), 5.76 (br s, 1H), 4.59 (m, 1H), 4.51-4.46 (m, 2H), 4.27-4.18 (m, 3H), 3.83-3.30 (m, 19H), 2.91 (m, 1H), 2.60-2.46 (m, 6H), 2.41 (m, 2H), 2.31-2.19 (m, 2H), 2.07 (m, 1H), 0.87-0.76 (m, 9H). Note: presence of rotamers.

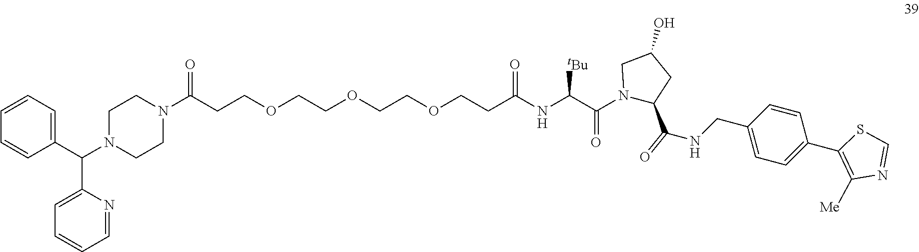 [SMILES: Cc1ncsc1-c1ccc(CNC(=O)[C@@H]2C[C@@H](O)CN2C(=O)[C@@H](NC(=O)CCOCCOCCOCCC(=O)N2CCN(C(c3ccccc3)c3ccccn3)CC2)C(C)(C)C)cc1]

### (2S,4R)-1-((2S)-2-(tert-butyl)-4,16-dioxo-16-(4-(phenyl(pyridin-2-yl)methyl) piperazin-1-yl)-7,10,13-trioxa-3-azahexadecanoyl)-4-hydroxy-N-(4-(4-methylthiazol-5-yl)benzyl) pyrrolidine-2-carboxamide (39)

General Procedure 3 then 1. Reaction scale: 20 mg (30.2 μmol, 1.0 eq.) of 12 and 8.4 mg (33.2 μmol, 1.1 eq.) of phenylpyridin-methylpiperazine. Purified by pTLC (8% MeOH/CH₂Cl₂) to afford 39 as white foam (18.2 mg, 67%). R_{f}=0.62 (8% MeOH/CH₂Cl₂, UV-active); LC-MS (ESI+) calc'd for C₄₈H₆₃N₇O₈S [M+H]⁺: 897.5, found 898.7; ¹H NMR (600 MHZ, CDCl₃) δ 8.59 (s, 1H), 8.44-8.40 (m, 1H), 7.55 (td, J=7.7, 1.8 Hz, 1H), 7.45 (dd, J=7.9, 1.0 Hz, 1H), 7.43-7.40 (m, 1H), 7.38 (dt, J=8.1, 1.5 Hz, 2H), 7.28-7.24 (m, 4H), 7.20 (dd, J=8.3, 6.9 Hz, 2H), 7.18 (s, 1H), 7.14-7.10 (m, 1H), 7.03 (ddt, J=7.3, 4.9, 1.2 Hz, 1H), 6.95 (dd, J=8.3, 1.6 Hz, 1H), 4.63 (td, J=8.0, 1.4 Hz, 1H), 4.47 (dd, J=15.0, 6.7 Hz, 1H), 4.43-4.38 (m, 2H), 4.34 (s, 1H), 4.24 (dd, J=15.0, 5.3 Hz, 1H), 4.06-3.99 (m, 2H), 3.68-3.59 (m, 4H), 3.55-3.48 (m, 12H), 3.41-3.37 (m, 2H), 2.49 (qd, J=6.8, 1.6 Hz, 2H), 2.42 (s, 3H), 2.39 (ddd, J=8.7, 6.4, 3.9 Hz, 3H), 2.28-2.23 (m, 2H), 0.85 (s, 9H). ¹³C NMR (151 MHz, CDCl₃) δ 172.20, 171.74, 171.19, 170.95, 169.49, 161.47, 150.33, 149.30, 148.43, 140.48, 138.28, 136.93, 131.66, 130.85, 129.48, 128.71, 128.23, 128.11, 127.60, 122.29, 122.25, 77.54, 77.30, 77.26, 77.05, 76.92, 76.84, 70.41, 70.32, 70.27, 70.25, 70.15, 70.10, 67.16, 67.14, 60.42, 58.44, 57.75, 56.86, 56.70, 56.00, 51.97, 51.55, 45.59, 43.18, 41.59, 36.58, 36.06, 36.03, 34.87, 33.34, 31.94, 29.71, 29.37, 29.33, 26.66, 26.42, 22.70, 21.07, 19.25, 18.17, 16.06, 14.21, 14.14.

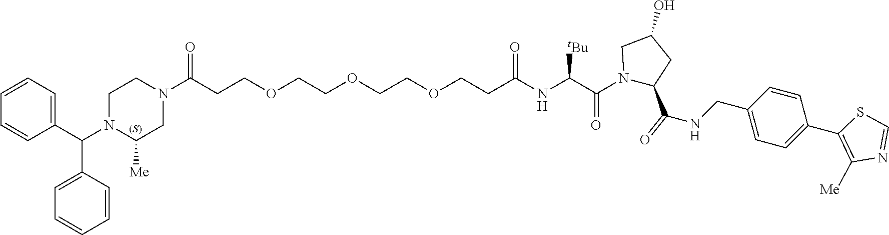

### (2S,4R)-1-((S)-16-((S)-4-benzhydryl-3-methylpiperazin-1-yl)-2-(tert-butyl)-4,16-dioxo-7,10,13-trioxa-3-azahexadecanoyl)-4-hydroxy-N-(4-(4-methylthiazol-5-yl)benzyl) pyrrolidine-2-carboxamide (40)

General Procedure 1 and 3. Reaction scale: 18.4 mg (26.0 μmol, 1.00 equiv) of 12 and 23.6 mg (64.0 μmol, 2.46 equiv) of (4S)-methyl benzhydrylpiperazine. Purified by PTLC (10% MeOH/EtOAc) to afford 40 as a clear oil (12.5 mg, 53%). R_{f}=0.39 (10% MeOH/EtOAc, UV-active); LC-MS (ESI+) calc'd for C₅₀H₆₈N₆O₈S [M+2H]²⁺: 456.2, found 456.4; ¹H NMR (600 MHz, CDCl₃) δ 8.67 (s, 1H), 7.44-7.41 (m, 2H), 7.39-7.32 (m, 7H), 7.28-7.24 (m, 5H), 7.21-7.13 (m, 3H), 4.72 (m, 1H), 4.55-4.46 (m, 3H), 4.35-4.31 (m, 1H), 4.16 (m, 1H), 4.10 (d, J=11.6 Hz, 1H), 3.76-3.54 (m, 16H), 3.46-3.37 (m, 1H), 3.00-2.91 (m, 2H), 2.63-2.39 (m, 12H), 2.16 (m, 1H), 0.94-0.91 (m, 9H).

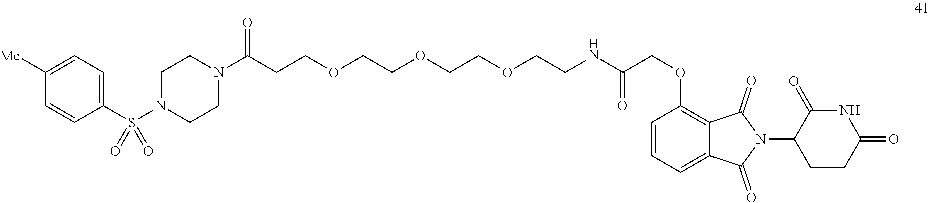 [SMILES: Cc1ccc(S(=O)(=O)N2CCN(C(=O)CCOCCOCCOCCNC(=O)COc3cccc4c3C(=O)N(C3CCC(=O)NC3=O)C4=O)CC2)cc1]

### 2-((2-(2,6-dioxopiperidin-3-yl)-1,3-dioxoisoindolin-4-yl)oxy)-N-(2-(2-(2-(3-oxo-3-(4-tosylpiperazin-1-yl) propoxy) ethoxy) ethoxy)ethyl) acetamide (41)

General Procedure 3 then 1. Reaction scale: 12 mg (22.4 μmol, 1.0 eq.) of 3 and 5.9 mg (24.7 μmol, 1.1 eq.) of tosylpiperazine. Purified by pTLC (10% MeOH/CH₂Cl₂) to afford 41 as a clear oil (4.8 mg, 28%). R_{f}=0.65 (8% MeOH/CH₂Cl₂, UV-active); LC-MS (ESI+) calc'd for C₃₅H₄₃N₅O₁₂S₂ 757.3, found [M+H]⁺ 758.2. ¹H NMR (600 MHz, CDCl₃) δ 8.95 (s, 1H), 7.74 (dd, J=8.4, 7.4 Hz, 1H), 7.63 (s, 1H), 7.62-7.60 (m, 3H), 7.55 (d, J=7.3 Hz, 1H), 7.33 (d, J=8.0 Hz, 3H), 7.19 (d, J=8.4 Hz, 1H), 4.94 (dd, J=12.4, 5.4 Hz, 1H), 3.73 (td, J=6.6, 1.4 Hz, 3H), 3.68-3.66 (m, 3H), 3.64 (dd, J=4.7, 2.7 Hz, 2H), 3.01-2.95 (m, 5H), 2.86-2.68 (m, 4H), 2.43 (s, 4H), 1.27 (s, 9H). ¹³C NMR (151 MHZ, CDCl₃) δ 171.09, 169.63, 168.16, 166.83, 166.67, 165.83, 154.47, 144.10, 137.01, 133.69, 132.33, 129.86, 127.79, 119.35, 118.06, 117.33, 77.24, 77.03, 76.82, 70.41, 70.28, 70.26, 70.24, 69.49, 67.91, 67.12, 60.42, 56.00, 53.45, 49.32, 46.16, 45.82, 45.10, 41.25, 40.90, 39.11, 38.63, 35.95, 33.71, 33.39, 31.94, 31.92, 31.47, 30.19, 29.79, 29.72, 29.54, 29.49, 29.38, 29.34, 29.25, 27.23, 25.55, 23.19, 22.70, 21.57, 21.08, 14.22, 14.14, 14.07.

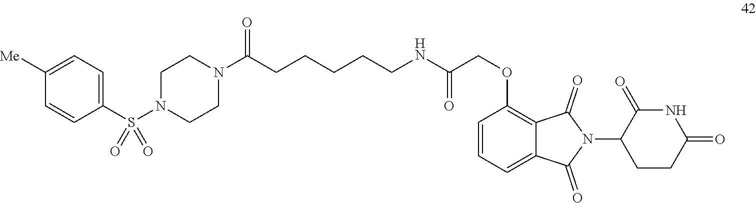 [SMILES: Cc1ccc(S(=O)(=O)N2CCN(C(=O)CCCCCNC(=O)COc3cccc4c3C(=O)N(C3CCC(=O)NC3=O)C4=O)CC2)cc1]

### 2-((2-(2,6-dioxopiperidin-3-yl)-1,3-dioxoisoindolin-4-yl)oxy)-N-(6-oxo-6-(4-tosylpiperazin-1-yl) hexyl) acetamide (42)

General Procedure 3 then 1. Reaction scale: 8 mg (18.0 μmol, 1.0 eq.) of 3 and 4.8 mg (19.8 μmol, 1.1 eq.) of tosylpiperazine. Purified by pTLC (10% MeOH/CH₂Cl₂) to afford 42 as an off-white solid (7.1 mg, 28%). R/=0.75 (8% MeOH/CH₂Cl₂, UV-active); LC-MS (ESI+) calc'd for C₃₂H₃₇N₅O₉S₂ 667.2, found [M+H]⁺ 668.3. ¹H NMR (600 MHZ, CDCl₃) δ 9.38 (s, 1H), 7.75 (d, J=1.1 Hz, 1H), 7.63-7.61 (m, 2H), 7.59 (s, 1H), 7.56 (d, J=7.4 Hz, 1H), 7.34 (s, 2H), 7.20 (d, J=8.3 Hz, 1H), 4.94 (dt, J=8.0, 4.0 Hz, 1H), 4.79-4.55 (m, 4H), 3.74-3.65 (m, 5H), 3.53 (t, J=5.2 Hz, 3H), 3.41-3.32 (m, 5H), 2.97 (dd, J=12.1, 5.3 Hz, 5H), 2.87 (d, J=3.1 Hz, 1H), 2.79-2.68 (m, 3H), 2.44 (s, 4H). ¹³C NMR (151 MHz, CDCl₃) δ 171.94, 171.21, 168.30, 166.82, 166.54, 166.29, 154.79, 144.17, 137.11, 133.55, 132.23, 129.90, 127.78, 120.27, 118.47, 117.67, 77.24, 77.03, 76.82, 68.66, 56.00, 49.31, 46.19, 45.87, 45.05, 40.93, 39.13, 33.71, 32.95, 31.95, 31.39, 30.18, 29.72, 29.38, 29.34, 28.84, 27.23, 26.65, 25.12, 23.19, 22.84, 22.75, 22.71, 21.58, 14.21, 14.14.

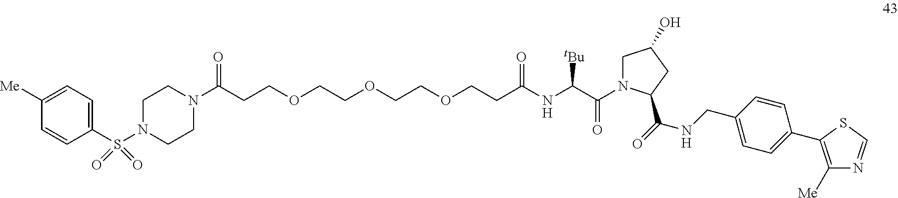 [SMILES: Cc1ccc(S(=O)(=O)N2CCN(C(=O)CCOCCOCCOCCC(=O)N[C@H](C(=O)N3C[C@H](O)C[C@H]3C(=O)NCc3ccc(-c4scnc4C)cc3)C(C)(C)C)CC2)cc1]

### (2S,4R)-1-((S)-2-(tert-butyl)-4,16-dioxo-16-(4-tosylpiperazin-1-yl)-7,10,13-trioxa-3-azahexadecanoyl)-4-hydroxy-N-(4-(4-methylthiazol-5-yl)benzyl) pyrrolidine-2-carboxamide (43)

General Procedure 3 then 2. Reaction scale: 6.0 mg (9.05 μmol) of 12 and 2.39 mg (9.96 μmol) of tosylpiperazine. Purified by pTLC (10% MeOH/DCM) to afford 43 as a white solid (3.3 mg, 41%). R_{f}=0.65 (8% MeOH/CH₂Cl₂, UV-active); LC-MS (ESI+) calc'd for C₄₃H₆₀N₆O₁₀S₂ 884.4, found [M+HCOO]⁻ 931.2. ¹H NMR (600 MHZ, CDCl₃) δ 8.68 (s, 1H), 7.64-7.60 (m, 2H), 7.46-7.41 (m, 1H), 7.36-7.30 (m, 6H), 6.95 (d, J=8.3 Hz, 1H), 4.73 (t, J=8.1 Hz, 1H), 4.62-4.56 (m, 1H), 4.51-4.45 (m, 2H), 4.35-4.29 (m, 1H), 3.77-3.64 (m, 7H), 3.65-3.51 (m, 13H), 3.06-2.88 (m, 4H), 2.54-2.50 (m, 6H), 2.15-2.11 (m, 1H), 0.94 (s, 12H). ¹³C NMR (151 MHZ, CDCl₃) δ 172.15, 171.80, 170.90, 169.80, 150.31, 148.47, 144.15, 138.25, 132.46, 132.28, 131.65, 130.89, 129.88, 129.49, 128.82, 128.11, 127.79, 77.24, 77.03, 76.82, 70.39, 70.23, 70.17, 70.13, 70.09, 68.17, 67.20, 67.06, 58.42, 57.78, 56.77, 56.00, 46.13, 45.81, 45.04, 43.21, 40.82, 38.74, 37.11, 36.54, 35.95, 34.76, 33.71, 33.45, 33.41, 32.77, 31.95, 30.37, 30.18, 30.05, 29.72, 29.50, 29.38, 29.34, 28.94, 27.23, 26.72, 26.43, 23.75, 23.19, 23.00, 22.71, 21.58, 16.08, 14.20, 14.14, 14.07, 10.97, 7.49.

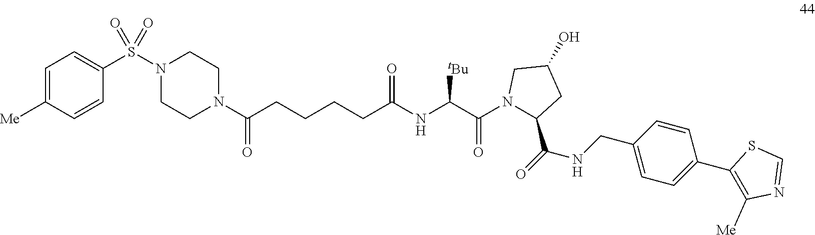 [SMILES: Cc1ccc(S(=O)(=O)N2CCN(C(=O)CCCCC(=O)N[C@H](C(=O)N3C[C@H](O)C[C@H]3C(=O)NCc3ccc(-c4scnc4C)cc3)C(C)(C)C)CC2)cc1]

### (2S,4R)-1-((S)-3,3-dimethyl-2-(6-oxo-6-(4-tosylpiperazin-1-yl) hexanamido) butanoyl)-4-hydroxy-N-(4-(4-methylthiazol-5-yl)benzyl) pyrrolidine-2-carboxamide (44)

General Procedure 3 then 1. Reaction scale: 10 mg (17.9 μmol, 1.0 eq.) of 3 and 4.7 mg (19.7 μmol, 1.1 eq.) of tosylpiperazine. Purified by pTLC (10% MeOH/CH₂Cl₂) to afford 41 as a white solid (4.0 mg, 29%). R_{f}=0.65 (8% MeOH/CH₂Cl₂, UV-active); LC-MS (ESI+) calc'd for C₃₉H₅₂N₆O₇S₂ 780.3, found [M+H]⁺ 781.8. ¹H NMR (600 MHZ, CDCl₃) δ 8.69 (s, 1H), 7.61 (d, J=8.4 Hz, 2H), 7.37-7.31 (m, 7H), 6.25 (d, J=8.5 Hz, 1H), 4.76-4.71 (m, 2H), 4.59 (dd, J=14.9, 6.7 Hz, 3H), 4.52-4.47 (m, 4H), 4.40-4.26 (m, 5H), 4.15-4.08 (m, 3H), 3.35 (s, 13H), 2.97 (dd, J=7.0, 3.9 Hz, 1H), 0.92 (s, 9H). Note: presence of rotamers.

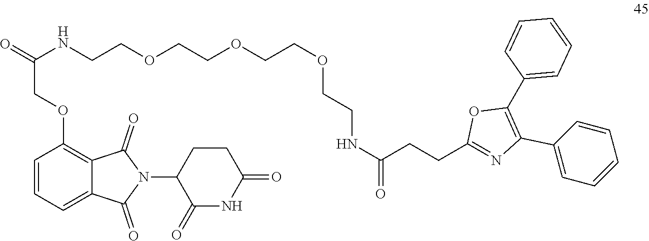 [SMILES: O=C(CCc1nc(-c2ccccc2)c(-c2ccccc2)o1)NCCOCCOCCOCCNC(=O)COc1cccc2c1C(=O)N(C1CCC(=O)NC1=O)C2=O]

### N-(1-((2-(2,6-dioxopiperidin-3-yl)-1,3-dioxoisoindolin-4-yl)oxy)-2-oxo-6,9,12-trioxa-3-azatetradecan-14-yl)-3-(4,5-diphenyloxazol-2-yl) propenamide (45)

General Procedure 1 and 3. Reaction scale: 13.4 mg (22.0 μmol, 1.00 equiv) of 4 and 7.6 mg (26.0 μmol, 1.20 equiv) of oxaprozin. Purified by PTLC (10% MeOH/EtOAc) to afford 45 as a clear oil (9.7 mg, 57%). R_{f}=0.31 (10% MeOH/EtOAc, UV-active); LC-MS (ESI+) calc'd for C₄₁H₄₄N₅O₁₁ [M+H]⁺: 782.3, found 782.1; ¹H NMR (600 MHz, CDCl₃) δ 9.27 (br s, 1H), 7.71-7.68 (m, 2H), 7.61-7.59 (m, 2H), 7.55-7.51 (m, 3H), 7.36-7.29 (m, 6H), 7.14 (d, J=7.8 Hz, 1H), 6.93 (m, 1H), 4.92 (dd, J=12.4, 5.5 Hz, 1H), 4.61 (s, 2H), 3.66-3.50 (m, 16H), 3.19 (m, 2H), 2.85-2.81 (m, 1H), 2.78-2.65 (m, 4H), 2.11-2.07 (m, 1H).

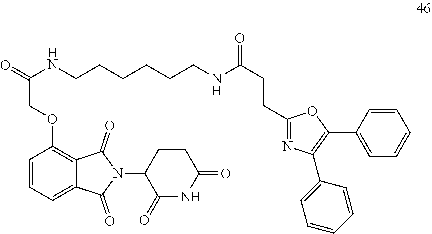 [SMILES: O=C(CCc1nc(-c2ccccc2)c(-c2ccccc2)o1)NCCCCCCNC(=O)COc1cccc2c1C(=O)N(C1CCC(=O)NC1=O)C2=O]

### N-(6-(2-((2-(2,6-dioxopiperidin-3-yl)-1,3-dioxoisoindolin-4-yl)oxy) acetamido) hexyl)-3-(4,5-diphenyloxazol-2-yl) propenamide (46)

General Procedure 1 and 3. Reaction scale: 8.7 mg (16.0 μmol, 1.00 equiv) of 7 and 7.0 mg (24.0 μmol, 1.50 equiv) of oxaprozin. Purified by PTLC (10% MeOH/CH₂Cl₂) to afford 46 as a white foam (5.7 mg, 50%). LC-MS (ESI−) calc'd for C₃₉H₃₈N₅O₈ [M−H]⁻: 704.3, found 704.0; ¹H NMR (600 MHZ, CDCl₃) δ 9.34 (br s, 1H), 7.71 (m, 1H), 7.60-7.58 (m, 2H), 7.55-7.52 (m, 3H), 7.46 (m, 1H), 7.36-7.29 (m, 5H), 7.17 (d, J=8.3 Hz, 1H), 6.36 (m, 1H), 4.95 (m, 1H), 4.62 (m, 2H), 3.37 (m, 1H), 3.29 (m, 2H), 3.21-3.16 (m, 3H), 2.85 (m, 1H), 2.78-2.70 (m, 4H), 2.13-2.09 (m, 1H), 1.55-1.45 (m, 4H), 1.37-1.29 (m, 4H).

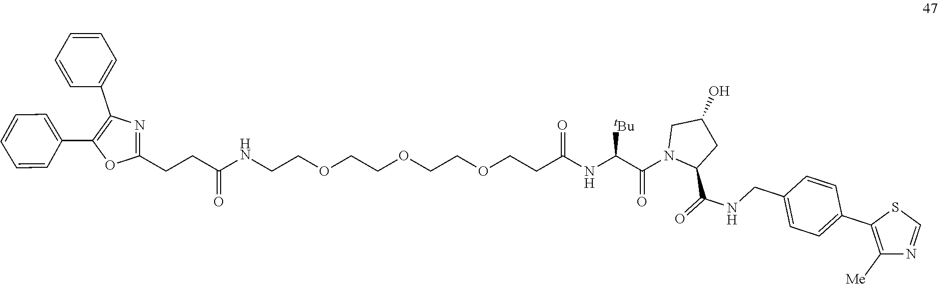 [SMILES: Cc1ncsc1-c1ccc(CNC(=O)[C@@H]2C[C@@H](O)CN2C(=O)[C@@H](NC(=O)CCOCCOCCOCCNC(=O)CCc2nc(-c3ccccc3)c(-c3ccccc3)o2)C(C)(C)C)cc1]

### (2S,4R)-1-((S)-2-(tert-butyl)-19-(4,5-diphenyloxazol-2-yl)-4,17-dioxo-7,10,13-trioxa-3,16-diazanonadecanoyl)-4-hydroxy-N-(4-(4-methylthiazol-5-yl)benzyl) pyrrolidine-2-carboxamide (47)

General Procedure 3 then 1. Reaction scale: 8 mg (12.6 μmol, 1.0 eq.) of 14 and 4.1 mg (13.9 μmol, 1.1 eq.) of oxaprozin. Purified by pTLC (10% MeOH/CH₂Cl₂) to afford 47 as white powder (6.2 mg, 54%). R_{f}=0.58 (10% MeOH/CH₂Cl₂, UV-active); LC-MS (ESI−) calc'd for C₄₉H₆₀N₆O₉S 908.4, found [M+HCOO]⁻ 954.2. ¹H NMR (400 MHZ, CDCl₃) δ 8.67 (s, 1H), 7.55 (ddt, J=14.2, 6.2, 1.9 Hz, 5H), 7.37-7.30 (m, 10H), 7.15 (s, 1H), 7.09 (d, J=8.4 Hz, 1H), 4.67 (t, J=8.2 Hz, 1H), 4.56-4.48 (m, 2H), 4.42 (s, 1H), 4.32 (dd, J=15.0, 5.4 Hz, 1H), 4.13-4.07 (m, 1H), 3.74 (q, J=5.2 Hz, 1H), 3.68-3.64 (m, 1H), 3.62-3.53 (m, 11H), 3.43 (tt, J=8.8, 4.7 Hz, 2H), 3.18 (t, J=7.6 Hz, 2H), 2.77 (t, J=7.7 Hz, 2H), 2.46 (t, J=5.6 Hz, 2H), 2.36 (ddd, J=13.1, 8.5, 4.3 Hz, 2H), 2.06 (d, J=14.3 Hz, 3H), 0.94 (s, 9H). ¹³C NMR (151 MHz, CDCl₃) δ 172.16, 171.66, 171.62, 171.11, 162.85, 150.35, 148.44, 145.47, 138.26, 134.85, 133.32, 132.40, 130.85, 129.55, 129.47, 128.87, 128.73, 128.70, 128.66, 128.63, 128.50, 128.43, 128.22, 128.17, 128.12, 128.06, 127.90, 126.51, 126.33, 77.24, 77.03, 76.82, 70.51, 70.40, 70.30, 70.08, 69.92, 68.86, 67.23, 58.55, 57.82, 57.72, 56.87, 56.00, 45.33, 43.28, 43.13, 39.78, 39.46, 36.54, 36.40, 35.82, 34.91, 33.71, 32.74, 31.95, 30.18, 30.06, 29.72, 29.68, 29.63, 29.38, 29.34, 27.69, 27.23, 26.72, 26.43, 26.38, 23.95, 23.20, 22.72, 21.51, 16.05, 14.21, 14.14.

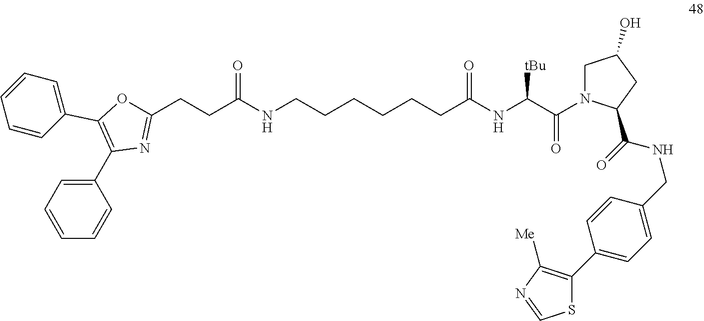 [SMILES: Cc1ncsc1-c1ccc(CNC(=O)[C@@H]2C[C@@H](O)CN2C(=O)[C@@H](NC(=O)CCCCCCNC(=O)CCc2nc(-c3ccccc3)c(-c3ccccc3)o2)C(C)(C)C)cc1]

### (2S,4R)-1-((S)-2-(7-(3-(4,5-diphenyloxazol-2-yl) propanamido) heptanamido)-3,3-dimethylbutanoyl)-4-hydroxy-N-(4-(4-methylthiazol-5-yl)benzyl) pyrrolidine-2-carboxamide (48)

General Procedure 1 and 3. Reaction scale: 15.2 mg (23.0 μmol, 1.00 equiv) of 18 and 9.9 mg (34.0 μmol, 1.20 equiv) of oxaprozin. Purified by PTLC (10% MeOH/EtOAc) to afford 48 as a clear oil (14.0 mg, 73%). R_{f}=0.23 (10% MeOH/EtOAc, UV-active); LC-MS (ESI+) calc'd for C₄₇H₅₇N₆O₆S [M+H]⁺: 833.4, found 833.4; ¹H NMR (600 MHz, CDCl₃) δ 8.65 (s, 1H), 7.58-7.49 (m, 5H), 7.36-7.30 (m, 10H), 6.49 (m, 1H), 6.28 (d, J=8.7 Hz, 1H), 4.67 (t, J=8.1 Hz, 1H), 4.55-4.50 (m, 2H), 4.45 (m, 1H), 4.31 (dd, J=15.0, 5.4 Hz, 1H), 4.05 (d, J=11.4 Hz, 1H), 3.58 (dd, J=11.3, 3.6 Hz, 1H), 3.22-3.12 (m, 4H), 2.71 (t, J=7.4 Hz, 2H), 2.49 (s, 3H), 2.38-2.34 (m, 1H), 2.21-2.16 (m, 1H), 2.13-2.07 (m, 2H), 1.60-1.42 (m, 4H), 1.29-1.20 (m, 5H), 0.93 (s, 9H).

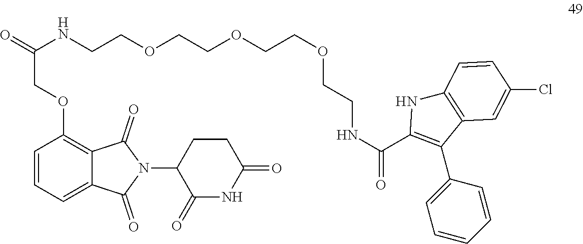 [SMILES: O=C(COc1cccc2c1C(=O)N(C1CCC(=O)NC1=O)C2=O)NCCOCCOCCOCCNC(=O)c1[nH]c2ccc(Cl)cc2c1-c1ccccc1]

### 5-chloro-N-(1-((2-(2,6-dioxopiperidin-3-yl)-1,3-dioxoisoindolin-4-yl)oxy)-2-oxo-6,9,12-trioxa-3-azatetradecan-14-yl)-3-phenyl-1H-indole-2-carboxamide (49)

General Procedure 1 and 3. Reaction scale: 9.5 mg (18.0 μmol, 1.00 equiv) of 4 and 6.0 mg (22.0 μmol, 1.20 equiv) of SM2. Purified by PTLC (10% MeOH/EtOAc) to afford 49 as a yellow foam (9.2 mg, 75%). R_{f}=0.55 (10% MeOH/EtOAc, UV-active); LC-MS (ESI−) calc'd for C₃₆H₃₃N₅O₇Cl [M−H]⁻: 682.2, found 682.0; ¹H NMR (600 MHZ, CDCl₃) δ 10.22 (s, 1H), 9.76 (br s, 1H), 7.71 (m, 1H), 7.54-7.45 (m, 7H), 7.40-7.37 (m, 2H), 7.22-7.17 (m, 2H), 6.02 (m, 1H), 5.01-4.98 (m, 1H), 4.62 (m, 2H), 3.41-3.17 (m, 4H), 2.90-2.73 (m, 3H), 2.13 (m, 1H), 1.52 (m, 2H), 1.43-1.30 (m, 4H), 1.22-1.18 (m, 2H).

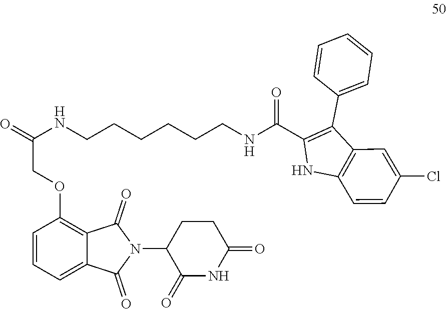 [SMILES: O=C(COc1cccc2c1C(=O)N(C1CCC(=O)NC1=O)C2=O)NCCCCCCNC(=O)c1[nH]c2ccc(Cl)cc2c1-c1ccccc1]

### 5-chloro-N-(6-(2-((2-(2,6-dioxopiperidin-3-yl)-1,3-dioxoisoindolin-4-yl)oxy) acetamido) hexyl)-3-phenyl-1H-indole-2-carboxamide (50)

General Procedure 1 and 3. Reaction scale: 9.5 mg (18.0 μmol, 1.00 equiv) of 7 and 6.0 mg (22.0 μmol, 1.20 equiv) of SM2. Purified by PTLC (10% MeOH/EtOAc) to afford 50 as a yellow foam (9.2 mg, 75%). R_{f}=0.55 (10% MeOH/EtOAc, UV-active); LC-MS (ESI−) calc'd for C₃₆H₃₃N₅O₇Cl [M−H]⁻: 682.2, found 682.0; ¹H NMR (600 MHZ, CDCl₃) δ 10.22 (s, 1H), 9.76 (br s, 1H), 7.71 (m, 1H), 7.54-7.45 (m, 7H), 7.40-7.37 (m, 2H), 7.22-7.17 (m, 2H), 6.02 (m, 1H), 5.01-4.98 (m, 1H), 4.62 (m, 2H), 3.41-3.17 (m, 4H), 2.90-2.73 (m, 3H), 2.13 (m, 1H), 1.52 (m, 2H), 1.43-1.30 (m, 4H), 1.22-1.18 (m, 2H).

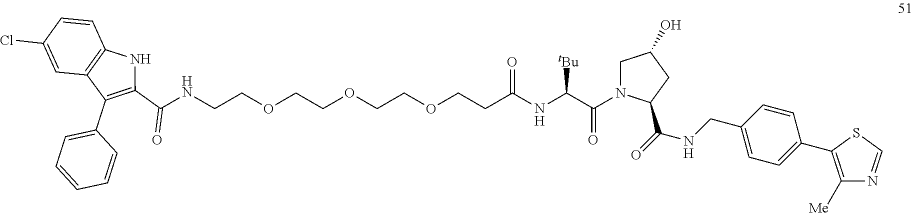 [SMILES: Cc1ncsc1-c1ccc(CNC(=O)[C@@H]2C[C@@H](O)CN2C(=O)[C@@H](NC(=O)CCOCCOCCOCCNC(=O)c2[nH]c3ccc(Cl)cc3c2-c2ccccc2)C(C)(C)C)cc1]

### 5-chloro-N-((S)-14-((2S,4R)-4-hydroxy-2-((4-(4-methylthiazol-5-yl)benzyl) carbamoyl) pyrrolidine-1-carbonyl)-15,15-dimethyl-12-oxo-3,6,9-trioxa-13-azahexadecyl)-3-phenyl-1H-indole-2-carboxamide (51)

General Procedure 3 then 1. Reaction scale: 8 mg (12.6 μmol, 1.0 eq.) of 14 and 4.9 mg (13.9 μmol, 1.1 eq.) of chlorophenylindole. Purified by pTLC (10% MeOH/CH₂Cl₂) to afford 51 as yellow powder (5.7 mg, 51%). R_{f}=0.57 (10% MeOH/CH₂Cl₂, UV-active); LC-MS (ESI−) calc'd for C₄₆H₅₅ClN₆O₈S 886.4, found [M−H]⁻ 885.5. ¹H NMR (400 MHZ, CDCl₃) δ 10.22 (s, 1H), 8.67 (s, 1H), 7.55-7.47 (m, 4H), 7.46-7.37 (m, 3H), 7.33 (s, 4H), 7.21 (dd, J=8.7, 2.0 Hz, 1H), 7.11 (d, J=8.6 Hz, 1H), 6.37 (s, 1H), 4.81 (t, J=8.2 Hz, 1H), 4.60-4.54 (m, 2H), 4.50 (s, 1H), 4.37 (dd, J=15.0, 5.3 Hz, 1H), 4.17 (d, J=11.4 Hz, 1H), 3.65-3.56 (m, 5H), 3.47 (d, J=6.5 Hz, 10H), 2.48 (s, 3H), 2.38 (dd, J=14.4, 8.3 Hz, 2H), 2.17 (s, 3H), 0.97 (s, 9H).

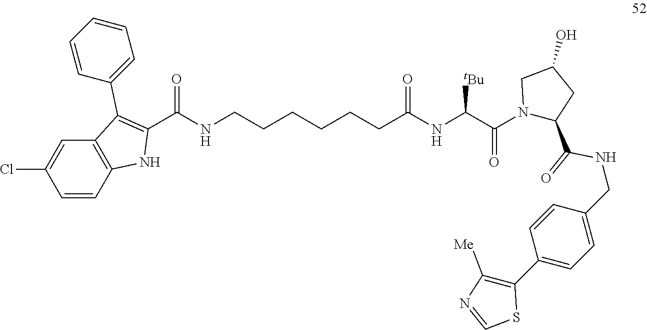 [SMILES: Cc1ncsc1-c1ccc(CNC(=O)[C@@H]2C[C@@H](O)CN2C(=O)[C@@H](NC(=O)CCCCCCNC(=O)c2[nH]c3ccc(Cl)cc3c2-c2ccccc2)C(C)(C)C)cc1]

### 5-chloro-N-(7-(((S)-1-((2S,4R)-4-hydroxy-2-((4-(4-methylthiazol-5-yl)benzyl) carbamoyl) pyrrolidin-1-yl)-3,3-dimethyl-1-oxobutan-2-yl)amino)-7-oxoheptyl)-3-phenyl-1H-indole-2-carboxamide (52)

General Procedure 1 and 3. Reaction scale: 18.3 mg (28.0 μmol, 1.00 equiv) of 18 and 11.6 mg (43.0 μmol, 1.54 equiv) of SM2. Purified by PTLC (10% MeOH/EtOAc) to afford 52 as a yellow solid (17.9 mg, 79%). R_{f}=0.38 (10% MeOH/EtOAc, UV-active); LC-MS (ESI+) calc'd for C₄₄H₅₂N₆O₅SCl [M+H]⁺: 811.3, found 811.1; ¹H NMR (600 MHZ, CDCl₃) δ 10.40 (m, 1H), 8.65 (s, 1H), 7.49-7.28 (m, 12H), 7.16 (m, 1H), 6.57 (m, 1H), 5.91 (m, 1H), 4.74 (m, 1H), 4.59 (d, J=9.0 Hz, 1H), 4.51-4.47 (m, 2H), 4.35 (dd, J=15.0, 5.6 Hz, 1H), 4.09 (d, J=11.3 Hz, 1H), 3.62 (m, 1H), 3.16 (m, 2H), 2.46 (s, 3H), 2.38 (m, 1H), 2.19-2.06 (m, 3H), 1.53-1.46 (m, 2H), 1.27-1.22 (m, 3H), 1.17 (m, 2H), 1.06 (m, 2H), 0.96 (s, 9H).

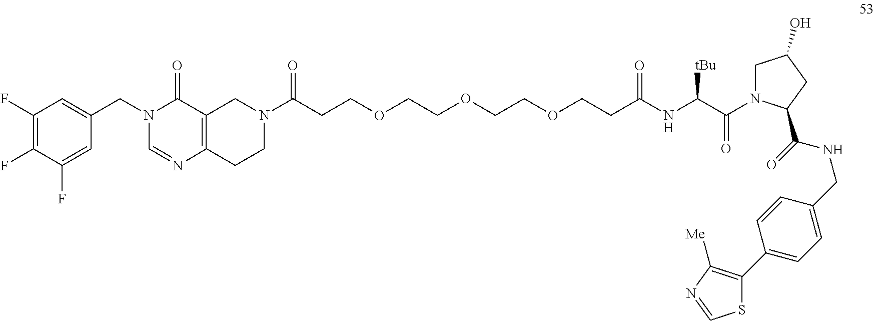 [SMILES: Cc1ncsc1-c1ccc(CNC(=O)[C@@H]2C[C@@H](O)CN2C(=O)[C@@H](NC(=O)CCOCCOCCOCCC(=O)N2CCc3ncn(Cc4cc(F)c(F)c(F)c4)c(=O)c3C2)C(C)(C)C)cc1]

### (2S,4R)-1-((S)-2-(tert-butyl)-4,16-dioxo-16-(4-oxo-3-(3,4,5-trifluorobenzyl)-3,5,7,8-tetrahydropyrido[4,3-d]pyrimidin-6 (4H)-yl)-7,10,13-trioxa-3-azahexadecanoyl)-4-hydroxy-N-(4-(4-methylthiazol-5-yl)benzyl) pyrrolidine-2-carboxamide (53)

General Procedure 1 and 3. Reaction scale: 14.2 mg (20.0 μmol, 1.00 equiv) of 12 and 15.2 mg (38.0 μmol, 1.90 equiv) of SM2. Purified by PTLC (100% EtOAc) then plate was dried and run again (10% MeOH/CH₂Cl₂) to afford 53 as a white foam (4.3 mg, 23%). ¹H NMR (400 MHZ, CDCl₃) δ 8.86 (s, 1H), 8.09-8.05 (m, 1H), 7.53-7.45 (m, 1H), 7.36 (m, 4H), 7.07-6.96 (m, 3H), 5.03-4.95 (m, 2H), 4.74 (m, 1H), 4.62-4.42 (m, 5H), 4.33 (dd, J=15.0, 5.2 Hz, 1H), 4.12 (m, 1H), 3.85-3.76 (m, 3H), 3.74-3.68 (m, 3H), 3.63-3.55 (m, 10H), 2.77 (m, 1H), 2.72-2.69 (m, 3H), 2.54-2.51 (m, 4H), 2.49-2.44 (m, 2H), 2.19-2.10 (m, 1H), 0.93 (s, 9H).

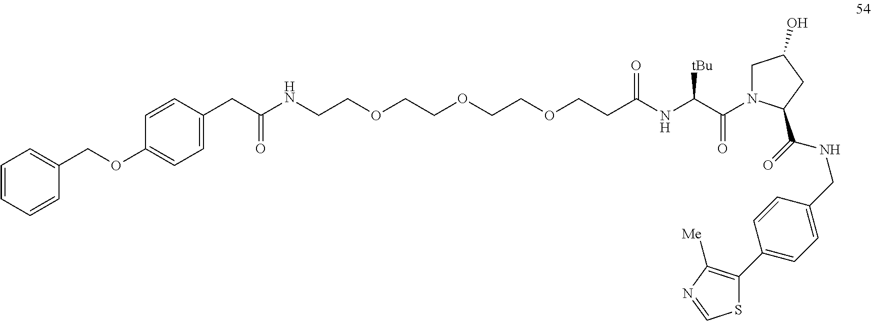 [SMILES: Cc1ncsc1-c1ccc(CNC(=O)[C@@H]2C[C@@H](O)CN2C(=O)[C@@H](NC(=O)CCOCCOCCOCCNC(=O)Cc2ccc(OCc3ccccc3)cc2)C(C)(C)C)cc1]

### (2S,4R)-1-((S)-1-(4-(benzyloxy)phenyl)-17-(tert-butyl)-2,15-dioxo-6,9,12-trioxa-3,16-diazaoctadecan-18-oyl)-4-hydroxy-N-(4-(4-methylthiazol-5-yl)benzyl) pyrrolidine-2-carboxamide (54)

General Procedure 1 and 3. Reaction scale: 8.6 mg (12.0 μmol, 1.00 equiv) of 14 and 7.5 mg (31.0 μmol, 2.58 equiv) of SM2. Purified by PTLC (10% MeOH/EtOAc) to afford 54 as a white foam (8.5 mg, 84%). 1H NMR (400 MHZ, CDCl₃) δ 8.69 (s, 1H), 7.43-7.30 (m, 11H), 7.17 (d, J=8.58 Hz, 2H), 6.92 (d, J=8.58 Hz, 2H), 5.04 (s, 2H), 4.70 (m, 1H), 4.58-4.48 (m, 3H), 4.33 (dd, J=14.8, 5.2 Hz, 1H), 4.11 (d, J=11.3 Hz, 1H), 3.74-3.35 (m, 19H), 2.50-2.44 (m, 6H), 1.42 (d, J=5.7 Hz, 1H), 0.93 (s, 9H).

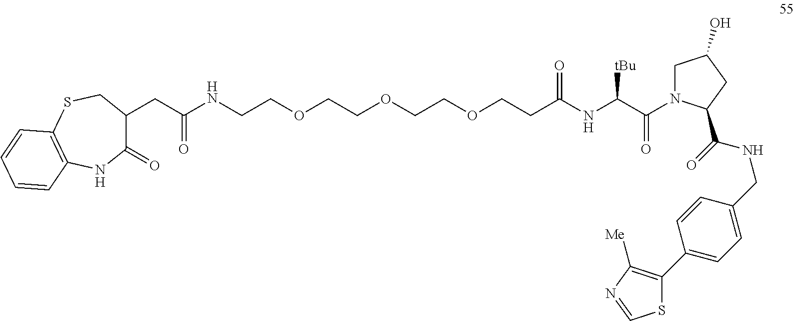 [SMILES: Cc1ncsc1-c1ccc(CNC(=O)[C@@H]2C[C@@H](O)CN2C(=O)[C@@H](NC(=O)CCOCCOCCOCCNC(=O)CC2CSc3ccccc3NC2=O)C(C)(C)C)cc1]

### (2S,4R)-1-((S)-19-(tert-butyl)-4,17-dioxo-1-(4-oxo-3,5,6,7-tetrahydro-4H-cyclopenta[4,5]thieno[2,3-d]pyrimidin-2-yl)-8,11,14-trioxa-2-thia-5,18-diazaicosan-20-oyl)-4-hydroxy-N-(4-(4-methylthiazol-5-yl)benzyl) pyrrolidine-2-carboxamide (55)

General Procedure 1 and 3. Reaction scale: 10.4 mg (14.0 μmol, 1.00 equiv) of 14 and 4.2 mg (18.0 μmol, 1.29 equiv) of SM2. Purified by PTLC (10% MeOH/CH₂Cl₂) to afford 55 as a yellow oil (9.0 mg, 77%). LC-MS (ESI+) calc'd for C₄₂H₅₇N₆O₉S₂ [M+H]⁺: 853.4, found 853.7.

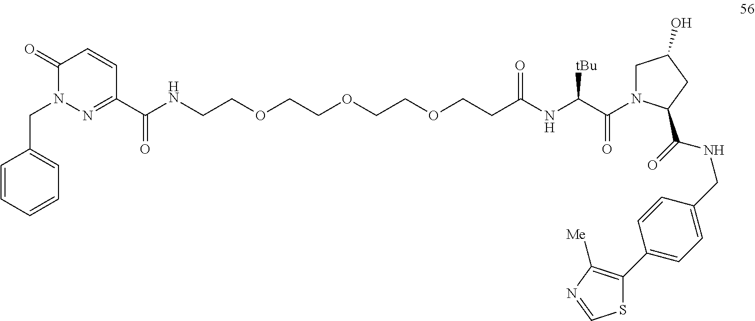 [SMILES: Cc1ncsc1-c1ccc(CNC(=O)[C@@H]2C[C@@H](O)CN2C(=O)[C@@H](NC(=O)CCOCCOCCOCCNC(=O)c2ccc(=O)n(Cc3ccccc3)n2)C(C)(C)C)cc1]

### 1-benzyl-N-((S)-14-((2S,4R)-4-hydroxy-2-((4-(4-methylthiazol-5-yl)benzyl) carbamoyl) pyrrolidine-1-carbonyl)-15,15-dimethyl-12-oxo-3,6,9-trioxa-13-azahexadecyl)-6-oxo-1,6-dihydropyridazine-3-carboxamide (56)

General Procedure 1 and 3. Reaction scale: 10.3 mg (14.0 μmol, 1.00 equiv) of 14 and 5.0 mg (21.0 μmol, 1.50 equiv) of SM2. Purified by PTLC (10% MeOH/CH₂Cl₂) to afford 56 as a white solid (8.4 mg, 71%). ¹H NMR (400 MHZ, CDCl₃) δ 8.67 (s, 1H), 7.89 (d, J=9.6 Hz, 1H), 7.52 (m, 1H), 7.41 (m, 2H), 7.36-7.30 (m, 7H), 6.97 (d, J=9.6 Hz, 1H), 6.81 (d, J=8.4 Hz, 1H), 5.35 (s, 2H), 4.68 (m, 1H), 4.54-4.49 (m, 3H), 4.35 (dd, J=14.9, 5.6 Hz, 1H), 4.15-4.09 (m, 2H), 3.67-3.57 (m, 16H), 2.52-2.50 (m, 6H), 2.42 (m, 1H), 0.94 (s, 9H).

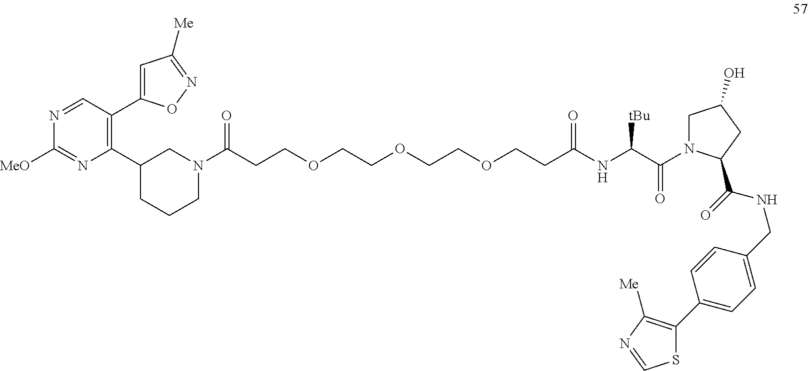 [SMILES: COc1ncc(-c2cc(C)no2)c(C2CCCN(C(=O)CCOCCOCCOCCC(=O)N[C@H](C(=O)N3C[C@H](O)C[C@H]3C(=O)NCc3ccc(-c4scnc4C)cc3)C(C)(C)C)C2)n1]

### (2S,4R)-1-((2S)-2-(tert-butyl)-16-(3-(2-methoxy-5-(3-methylisoxazol-5-yl)pyrimidin-4-yl) piperidin-1-yl)-4,16-dioxo-7,10,13-trioxa-3-azahexadecanoyl)-4-hydroxy-N-(4-(4-methylthiazol-5-yl)benzyl) pyrrolidine-2-carboxamide (57)

General Procedure 1 and 3. Reaction scale: 7.0 mg (10.0 μmol, 1.00 equiv) of 12 and 6.0 mg (15.0 μmol, 1.50 equiv) of SM2. Purified by PTLC (10% MeOH/CH₂Cl₂) to afford 57 as a white foam (6.8 mg, 74%). R_{f}=0.68 (10% MeOH/CH₂Cl₂, UV-active); LC-MS (ESI−) calc'd for C₄₇H₆₃N₈O₁₂S [M+HCO₂]⁻: 963.4, found 963.0; ¹H NMR (600 MHZ, CDCl₃) δ 8.67 (s, 1H), 8.66 (d, J=2.7 Hz), 7.54-7.44 (m, 1H), 7.36-7.32 (m, 4H), 7.06-7.00 (s, 1H), 6.43 (s, 1H), 4.73-4.69 (m, 1H), 4.59-4.54 (m, 1H), 4.51-4.45 (m, 2H), 4.36-4.29 (m, 1H), 4.10 (br d, J=11.5 Hz, 1H), 4.07-4.05 (m, 3H), 4.00-3.90 (m, 1H), 3.80-3.66 (m, 5H), 3.64-3.55 (m, 11H), 3.11-3.05 (m, 1H), 2.67-2.63 (m, 1H), 2.62-2.57 (m, 1H), 2.51 (s, 3H), 2.49-2.44 (m, 3H), 2.38-2.36 (m, 3H), 2.16-2.10 (m, 1H), 1.97-1.76 (m, 5H), 0.93 (s, 9H).

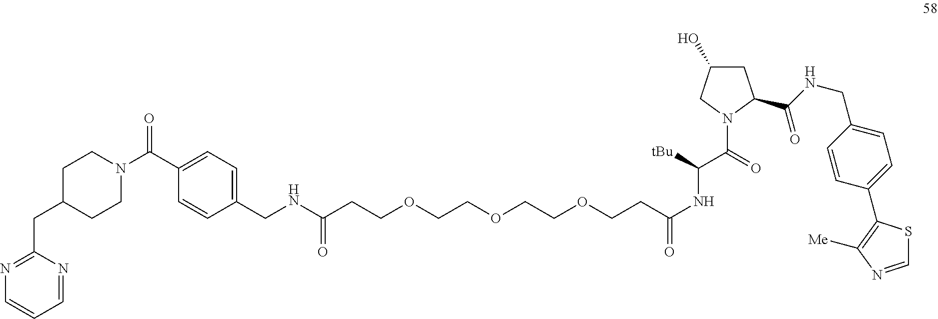 [SMILES: Cc1ncsc1-c1ccc(CNC(=O)[C@@H]2C[C@@H](O)CN2C(=O)[C@@H](NC(=O)CCOCCOCCOCCC(=O)NCc2ccc(C(=O)N3CCC(Cc4ncccn4)CC3)cc2)C(C)(C)C)cc1]

### (2S,4R)-1-((S)-17-(tert-butyl)-3,15-dioxo-1-(4-(4-(pyrimidin-2-ylmethyl) piperidine-1-carbonyl)phenyl)-6,9,12-trioxa-2,16-diazaoctadecan-18-oyl)-4-hydroxy-N-(4-(4-methylthiazol-5-yl)benzyl) pyrrolidine-2-carboxamide (58)

General Procedure 1 and 3. Reaction scale: 8.0 mg (11.0 μmol, 1.00 equiv) of 12 and 9.2 mg (22.0 μmol, 2.00 equiv) of SM2. Purified by PTLC (100% EtOAc) then plate was dried and run again (10% MeOH/CH₂Cl₂) to afford 58 as a yellow oil (1.3 mg, 13%). LC-MS (ESI+) calc'd for C₅₀H₆₇N₈O₉S [M+H]⁺: 956.0, found 956.4; ¹H NMR (600 MHz, CDCl₃) δ 8.66 (m, 3H), 7.38-7.27 (m, 9H), 7.14 (t, J=4.9 Hz, 1H), 6.98 (d, J=8.4 Hz, 1H), 4.63 (m, 1H), 4.54-4.45 (m, 4H), 4.34 (td, J=15.7, 5.4 Hz, 2H), 3.98 (d, J=11.4 Hz, 1H), 3.79-3.52 (m, 14H), 3.10 (q, J=7.4 Hz, 1H), 2.99-2.87 (m, 3H), 2.76 (m, 1H), 2.54-2.44 (m, 5H), 2.40-2.32 (m, 2H), 2.24 (m, 1H), 1.48-1.39 (m, 8H), 0.94 (s, 9H).

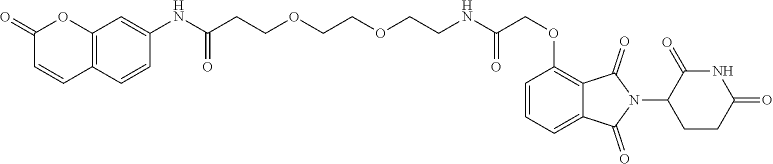 [SMILES: O=C(COc1cccc2c1C(=O)N(C1CCC(=O)NC1=O)C2=O)NCCOCCOCCC(=O)Nc1ccc2ccc(=O)oc2c1]

### 3-(2-(2-(2-((2-(2,6-dioxopiperidin-3-yl)-1,3-dioxoisoindolin-4-yl)oxy) acetamido) ethoxy) ethoxy)-N-(2-oxo-2H-chromen-7-yl) propenamide (59)

General Procedure 2. Reaction scale: 8.0 μmol of 2 and 1.4 mg (8.8 μmol) of coumarin. Purified by pTLC (EtOAc, then 10% MeOH/CH₂Cl₂) to afford 59 as a glassy solid (4.3 mg, 85%). Molecular formula: C₃₁H₃₀N₄O₁₁ ¹H NMR (400 MHZ, CDCl₃) δ_{H} 2.14-2.25 (1H, m, CL1, diastereotopic proton), 2.65 (2H, t, J 5.6, C(O)CH₂CH₂O), 2.71-2.94 (2H, m, CL1 diastereotopic proton, other proton), 3.51-3.66 (8H, m, O(CH₂)₂O(CH₂)₂NH), 3.85 (2H, t, J 5.6, C(O)CH₂CH₂O), 4.56 (2H, s, C(O)CH₂O), 4.96 (1H, dd, J 5.5, 12.4, CL1 stereocenter proton), 6.36 (1H, d, J 9.5, coumarin double bond nearest carbonyl), 7.13 (1H, d, J 8.7, ortho-linker), 7.20 (1H d, J 8.9, coumarin proton), 7.41 (1H, dd, J 2.4, 8.9, coumarin proton nearest linker), 7.51 (1H, d, J 7.4, CL1, para-linker), 7.56 (1H, br t, J 5.6, linker NH), 7.64 (1H, J 9.5, coumarin double bond farthest from carbonyl), 7.72 (1H dd, J 8.7, 7.4, CL1, meta-linker), 8.06 (1H, d, J 2.4, coumarin proton no neighbours), 8.86 (1H, br. s, NH).

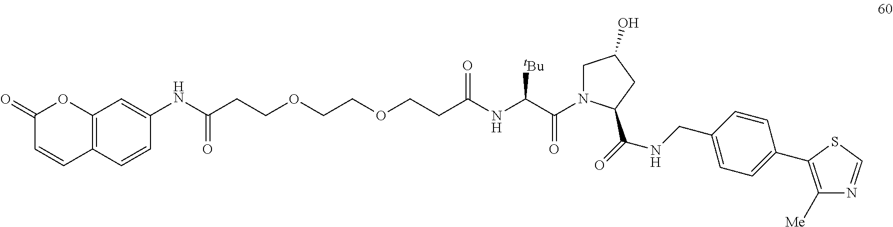 [SMILES: Cc1ncsc1-c1ccc(CNC(=O)[C@@H]2C[C@@H](O)CN2C(=O)[C@@H](NC(=O)CCOCCOCCC(=O)Nc2ccc3ccc(=O)oc3c2)C(C)(C)C)cc1]

### (2S,4R)-1-((S)-3,3-dimethyl-2-(3-(2-(3-oxo-3-((2-oxo-2H-chromen-7-yl)amino) propoxy) ethoxy) propanamido) butanoyl)-4-hydroxy-N-(4-(4-methylthiazol-5-yl)benzyl) pyrrolidine-2-carboxamide (60)

General Procedure 3 then 2. Reaction scale: 11.1 μmol of 2 and 2.0 mg (12.2 μmol) of coumarin. Purified by pTLC (EtOAc, then 10% MeOH/CH₂Cl₂) to afford 60 as a translucent glass (5.1 mg, 60%). Molecular formula: C₃₉H₄₇N₅O₉S. ¹H NMR (400 MHZ, CDCl₃) δ 9.27 (s, 1H), 8.68 (s, 1H), 7.92 (d, J=2.2 Hz, 1H), 7.75-7.51 (m, 2H), 7.44 (d, J=8.7 Hz, 1H), 7.37 (q, J=8.3 Hz, 4H), 7.21-7.09 (m, 2H), 6.33 (d, J=9.6 Hz, 1H), 4.79 (t, J=8.3 Hz, 1H), 4.71-4.55 (m, 3H), 4.32 (dd, J=15.1, 5.2 Hz, 1H), 4.20 (d, J=11.3 Hz, 1H), 3.84 (td, J=9.9, 3.2 Hz, 1H), 3.79-3.44 (m, 7H), 2.78 (ddd, J=14.2, 9.9, 4.2 Hz, 1H), 2.52 (s, 3H), 2.49-2.30 (m, 2H), 2.30-2.13 (m, 3H), 1.02 (s, 9H).

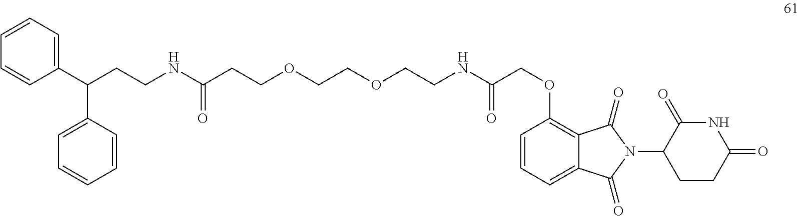 [SMILES: O=C(CCOCCOCCNC(=O)COc1cccc2c1C(=O)N(C1CCC(=O)NC1=O)C2=O)NCCC(c1ccccc1)c1ccccc1]

### 3-(2-(2-(2-((2-(2,6-dioxopiperidin-3-yl)-1,3-dioxoisoindolin-4-yl)oxy) acetamido) ethoxy) ethoxy)-N-(3,3-diphenylpropyl) propenamide (61)

General Procedure 3 then 2. Molecular formula: C₃₇H₄₀N₄O₉ Yield 61:4.3 mg (78%). ¹H NMR (400 MHZ, CDCl₃) δ 8.68 (s, 1H), 7.42-7.30 (m, 4H), 7.30-7.19 (m, 11H), 7.19-7.13 (m, 2H), 6.98 (d, J=8.5 Hz, 1H), 6.51 (t, J=5.8 Hz, 1H), 4.62 (t, J=8.1 Hz, 1H), 4.56 (dd, J=15.0, 6.7 Hz, 1H), 4.50 (d, J=8.6 Hz, 2H), 4.31 (dd, J=15.0, 5.2 Hz, 1H), 4.04 (d, J=11.3 Hz, 1H), 3.94 (t, J=7.8 Hz, 1H), 3.75-3.45 (m, 10H), 3.16 (dt, J=8.1, 6.1 Hz, 2H), 2.51 (s, 3H), 2.48-2.27 (m, 4H), 2.23 (td, J=8.0, 6.3 Hz, 2H), 2.15-2.03 (m, 1H), 0.94 (s, 9H).

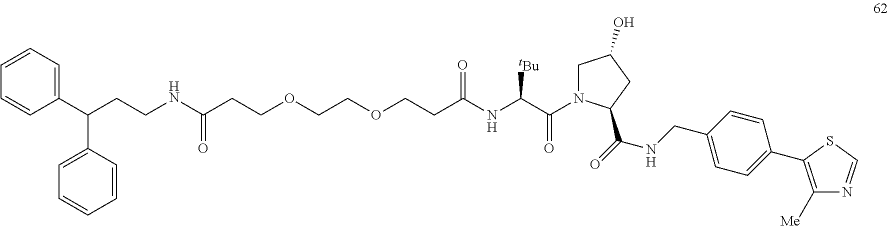 [SMILES: Cc1ncsc1-c1ccc(CNC(=O)[C@@H]2C[C@@H](O)CN2C(=O)[C@@H](NC(=O)CCOCCOCCC(=O)NCCC(c2ccccc2)c2ccccc2)C(C)(C)C)cc1]

### (2S,4R)-1-((S)-2-(tert-butyl)-4,13-dioxo-17,17-diphenyl-7,10-dioxa-3,14-diazaheptadecanoyl)-4-hydroxy-N-(4-(4-methylthiazol-5-yl)benzyl) pyrrolidine-2-carboxamide (62)

General Procedure 3 then 2. Molecular formula: C₄₅H₅₇N₅O₇S. Yield 62:7.7 mg (80%).

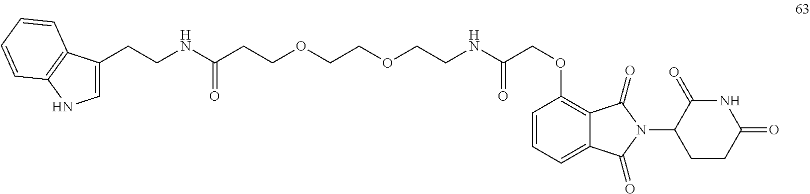 [SMILES: O=C(CCOCCOCCNC(=O)COc1cccc2c1C(=O)N(C1CCC(=O)NC1=O)C2=O)NCCc1c[nH]c2ccccc12]

### N-(2-(1H-indol-3-yl)ethyl)-3-(2-(2-(2-((2-(2,6-dioxopiperidin-3-yl)-1,3-dioxoisoindolin-4-yl)oxy) acetamido) ethoxy) ethoxy) propenamide (63)

General Procedure 3 then 2. Reaction scale: 17.6 μmol of 2 and 3.2 mg (20 μmol) of indole amine. Purified by pTLC (EtOAc, then 10% MeOH/CH₂Cl₂) to afford 63 as a yellow translucent glass (2.8 mg, 25%). Molecular formula: C₃₂H₃₅N₅O₉ ¹H NMR (400 MHz, CDCl₃) δ_{H} 2.04-2.12 (1H, m, CL1, diastereotopic proton), 2.41 (2H, t, J 5.9, tryptamine NHCH₂CH₂), 2.57-2.84 (3H, m, CL1 diastereotopic proton, imide-CH₂), 2.95 (2H, t, J6.5, C(O)CH₂CH₂O), 3.46-3.64 (10H, m, O(CH₂)₂O(CH₂)₂NH, tryptamine CH₂NH), 3.70 (2H, t, J5.8, C(O)CH₂CH₂O), 4.58 (2H, d, J2.2, C(O)CH₂O), 4.90 (1H, dd, J 5.4, 12.2, CL1 stereocenter proton), 6.36 (1H, t, J 5.5, tryptamine amide NHC(O)), 7.04-7.18 (3H, m, indole), 7.13 (1H, d, J 8.7, ortho-linker), 7.32 (1H, d, J 8.1, indole), 7.52 (1H, d, J 7.4, CL1, para-linker), 7.56 (1H, d, J 7.7, indole), 7.62 (1H, br t, J 5.6, linker NH), 7.72 (1H dd, J 8.7, 7.4, CL1, meta-linker), 8.41 (1H, br s, imide NH), 8.82 (1H, br s, indole NH).

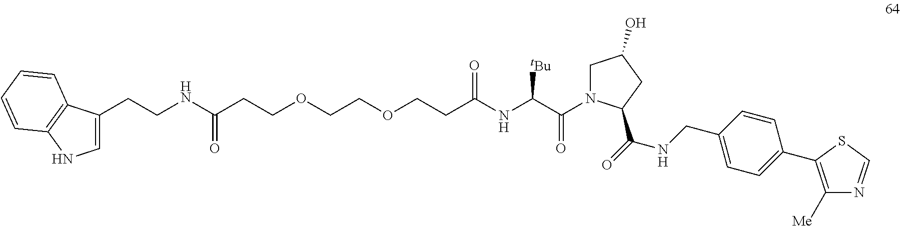 [SMILES: Cc1ncsc1-c1ccc(CNC(=O)[C@@H]2C[C@@H](O)CN2C(=O)[C@@H](NC(=O)CCOCCOCCC(=O)NCCc2c[nH]c3ccccc23)C(C)(C)C)cc1]

### (2S,4R)-1-((S)-2-(tert-butyl)-16-(1H-indol-3-yl)-4,13-dioxo-7,10-dioxa-3,14-diazahexadecanoyl)-4-hydroxy-N-(4-(4-methylthiazol-5-yl)benzyl) pyrrolidine-2-carboxamide (64)

General Procedure 3 then 2. Molecular formula: C₄₀H₅₂N₆O₇S. Yield 64:4.3 mg (45%).

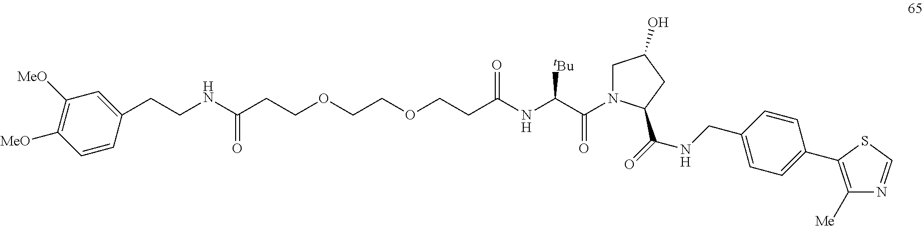 [SMILES: COc1ccc(CCNC(=O)CCOCCOCCC(=O)N[C@H](C(=O)N2C[C@H](O)C[C@H]2C(=O)NCc2ccc(-c3scnc3C)cc2)C(C)(C)C)cc1OC]

### (2S,4R)-1-((S)-2-(tert-butyl)-16-(3,4-dimethoxyphenyl)-4,13-dioxo-7,10-dioxa-3,14-diazahexadecanoyl)-4-hydroxy-N-(4-(4-methylthiazol-5-yl)benzyl) pyrrolidine-2-carboxamide (65)

General Procedure 3 then 2. Molecular formula: C₄₀H₅₅N₅O₉S. Yield 65:7.9 mg (78%). ¹H NMR (400 MHZ, CDCl₃) δ 8.68 (s, 1H), 7.36 (s, 3H), 6.98 (d, J=8.4 Hz, 1H), 6.85-6.65 (m, 4H), 6.53 (d, J=5.9 Hz, 1H), 4.60 (dt, J=23.2, 7.4 Hz, 2H), 4.49 (d, J=8.5 Hz, 2H), 4.33 (dd, J=15.0, 5.1 Hz, 1H), 4.08 (d, J=11.4 Hz, 1H), 3.86 (s, 3H), 3.84 (s, 3H), 3.76-3.38 (m, 12H), 2.74 (q, J=7.4 Hz, 2H), 2.54-2.30 (m, 9H), 2.19-2.09 (m, 1H), 0.94 (s, 9H).

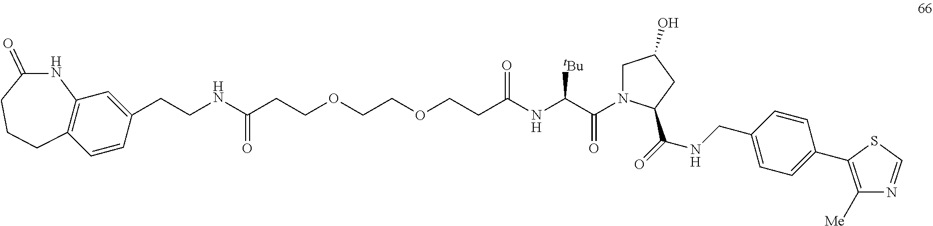 [SMILES: Cc1ncsc1-c1ccc(CNC(=O)[C@@H]2C[C@@H](O)CN2C(=O)[C@@H](NC(=O)CCOCCOCCC(=O)NCCc2ccc3c(c2)NC(=O)CCC3)C(C)(C)C)cc1]

### (2S,4R)-1-((S)-2-(tert-butyl)-4,13-dioxo-16-(2-oxo-2,3,4,5-tetrahydro-1H-benzo[b]azepin-8-yl)-7,10-dioxa-3,14-diazahexadecanoyl)-4-hydroxy-N-(4-(4-methylthiazol-5-yl)benzyl) pyrrolidine-2-carboxamide (66)

General Procedure 3 then 2. Molecular formula: C₄₂H₅₆N₆O₈S. Yield 66:5.0 mg (50%).

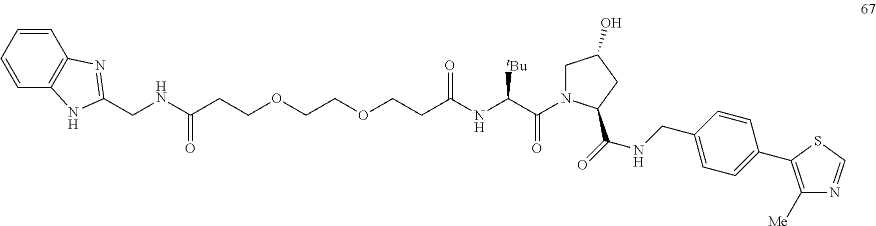 [SMILES: Cc1ncsc1-c1ccc(CNC(=O)[C@@H]2C[C@@H](O)CN2C(=O)[C@@H](NC(=O)CCOCCOCCC(=O)NCc2nc3ccccc3[nH]2)C(C)(C)C)cc1]

### (2S,4R)-1-((S)-1-(1H-benzo[d]imidazol-2-yl)-14-(tert-butyl)-3,12-dioxo-6,9-dioxa-2,13-diazapentadecan-15-oyl)-4-hydroxy-N-(4-(4-methylthiazol-5-yl)benzyl) pyrrolidine-2-carboxamide (67)

General Procedure 3 then 2. Molecular formula: C₃₈H₄₉N₇O₇S. Yield 67:4.3 mg (45%).

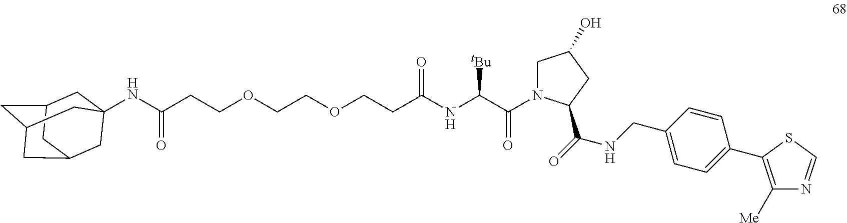 [SMILES: Cc1ncsc1-c1ccc(CNC(=O)[C@@H]2C[C@@H](O)CN2C(=O)[C@@H](NC(=O)CCOCCOCCC(=O)NC23CC4CC(CC(C4)C2)C3)C(C)(C)C)cc1]

### (2S,4R)-1-((S)-2-(3-(2-(3-(((3s,5s,7s)-adamantan-1-yl)amino)-3-oxopropoxy) ethoxy) propanamido)-3,3-dimethylbutanoyl)-4-hydroxy-N-(4-(4-methylthiazol-5-yl)benzyl) pyrrolidine-2-carboxamide (68)

General Procedure 3 then 2. Molecular formula: C₄₀H₅₇N₅O₇S. Yield 68:4.2 mg (43%).

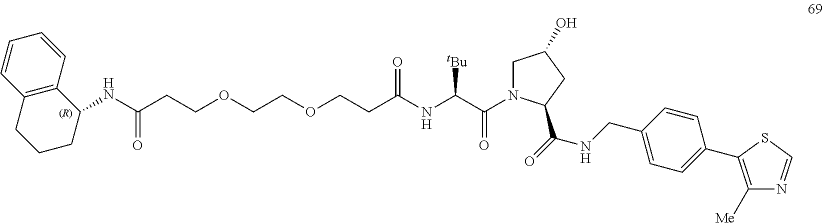

### (2S,4R)-1-((S)-3,3-dimethyl-2-(3-(2-(3-oxo-3-(((R)-1,2,3,4-tetrahydronaphthalen-1-yl)amino) propoxy) ethoxy) propanamido) butanoyl)-4-hydroxy-N-(4-(4-methylthiazol-5-yl)benzyl) pyrrolidine-2-carboxamide (69)

General Procedure 3 then 2. Molecular formula: C₄₀H₅₃N₅O₇S. Yield 69:5.2 mg (41%).

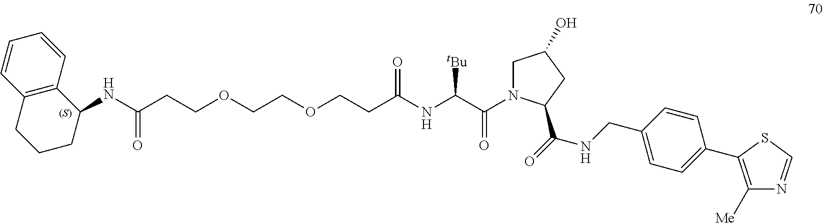

### (2S,4R)-1-((S)-3,3-dimethyl-2-(3-(2-(3-oxo-3-(((S)-1,2,3,4-tetrahydronaphthalen-1-yl)amino) propoxy) ethoxy) propanamido) butanoyl)-4-hydroxy-N-(4-(4-methylthiazol-5-yl)benzyl) pyrrolidine-2-carboxamide (70)

General Procedure 3 then 2. Molecular formula: C₄₀H₅₃N₅O₇S. Yield 70:8.2 mg (96%).

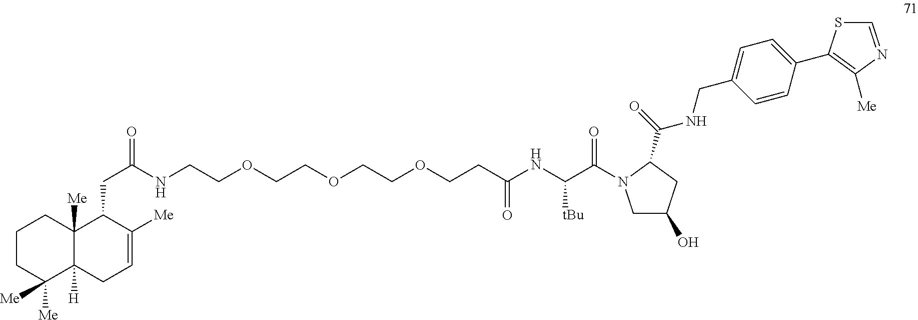

### (2S,4R)-1-((S)-16-(tert-butyl)-1,14-dioxo-1-((1S,4S)-4,7,7-trimethyl-3-oxo-2-oxabicyclo[2.2.1]heptan-1-yl)-5,8,11-trioxa-2,15-diazaheptadecan-17-oyl)-4-hydroxy-N-(4-(4-methylthiazol-5-yl)benzyl) pyrrolidine-2-carboxamide (71)

General Procedure 1 and 3. Reaction scale: 17.0 mg (23.0 μmol, 1.00 equiv) of 14 and 11.8 mg (47.0 μmol, 2.04 equiv) of (+)-sclereolide. Purified by PTLC (50% EtOAc/hexanes) to afford 71 as a yellow oil (16.8 mg, 84%). ¹H NMR (400 MHZ, CDCl₃) δ 8.67 (s, 1H), 7.40-7.32 (m, 5H), 6.97 (d, J=8.4 Hz, 1H), 6.33 (m, 1H), 5.31 (m, 1H), 4.70 (t, J=8.1 Hz, 1H), 4.58-4.48 (m, 3H), 4.33 (dd, J=15.1, 5.3 Hz, 1H), 3.76-3.56 (m, 13H), 3.53-3.49 (m, 2H), 3.45-3.38 (m, 2H), 2.50-2.44 (m, 7H), 2.35-2.29 (m, 1H), 2.17-2.06 (m, 3H), 1.65 (m, 3H), 1.42-1.36 (m, 4H), 1.24-1.07 (m, 4H), 0.93 (s, 9H), 0.92 (s, 3H), 0.87 (s, 3H), 0.84 (s, 3H).

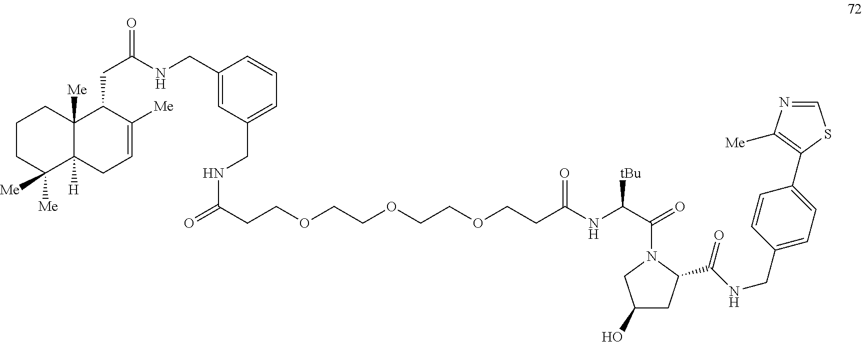

### (2S,4R)-1-((S)-17-(tert-butyl)-3,15-dioxo-1-(3-((2-((1R,4aS,8aS)-2,5,5,8a-tetramethyl-1,4,4a,5,6,7,8,8a-octahydronaphthalen-1-yl) acetamido)methyl)phenyl)-6,9,12-trioxa-2,16-diazaoctadecan-18-oyl)-4-hydroxy-N-(4-(4-methylthiazol-5-yl)benzyl) pyrrolidine-2-carboxamide (72)

Reaction scale: 12.3 mg (17.0 μmol, 1.00 equiv) of 12 and 10.8 mg (23.0 μmol, 1.35 equiv) of (+)-sclereolide phenyl derivative. Purified by PTLC (100% EtOAc) to afford 72 as a yellow oil (3.8 mg, 22%).

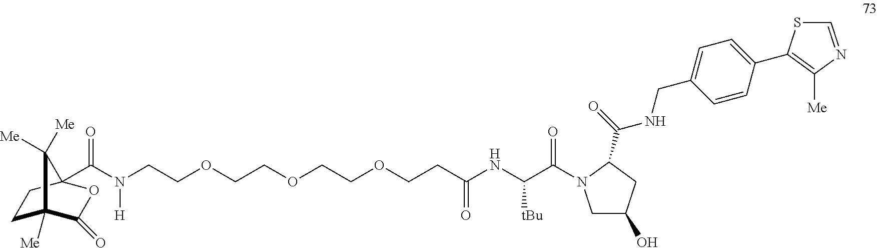

### (2S,4R)-1-((S)-16-(tert-butyl)-1,14-dioxo-1-((1S,4S)-4,7,7-trimethyl-3-oxo-2-oxabicyclo[2.2.1]heptan-1-yl)-5,8,11-trioxa-2,15-diazaheptadecan-17-oyl)-4-hydroxy-N-(4-(4-methylthiazol-5-yl)benzyl) pyrrolidine-2-carboxamide (73)

General Procedure 1 and 3. Reaction scale: 14.4 mg (20.0 μmol, 1.00 equiv) of 14 and 7.0 mg (35.0 μmol, 1.75 equiv) of (S)-camphanic acid. Purified by PTLC (50% EtOAc/hexanes) to afford 73 as a yellow oil (13.1 mg, 80%). 1H NMR (400 MHZ, CDCl₃) δ 8.67 (s, 1H), 7.45 (t, J=5.9 Hz, 1H), 7.37-7.32 (m, 4H), 7.02-6.96 (m, 2H), 4.72 (t, J=8.1 Hz, 1H), 4.55 (dd, J=15.0, 6.6 Hz, 1H), 4.51-4.45 (m, 2H), 4.33 (dd, J=15.1, 5.3 Hz, 1H), 4.11 (m, 1H), 3.71 (m, 2H), 3.63-3.44 (m, 14H), 2.52-2.47 (m, 7H), 2.16-2.11 (m, 1H), 1.95-1.84 (m, 2H), 1.68-1.62 (m, 1H), 1.08 (s, 3H), 1.08 (s, 3H), 0.94 (s, 9H), 0.88 (s, 3H).

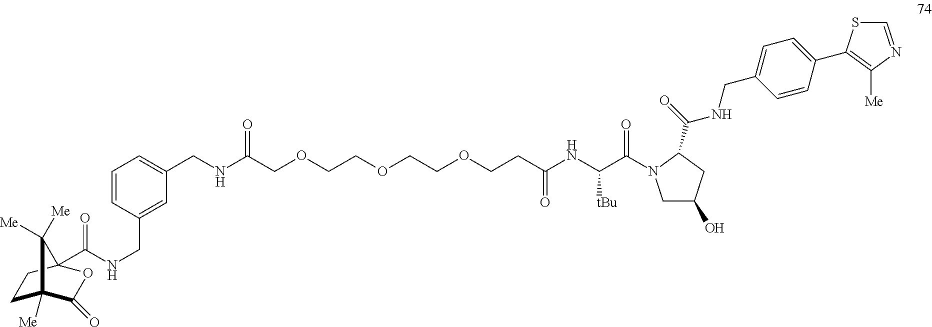

### (2S,4R)-1-((S)-18-(tert-butyl)-3,16-dioxo-1-(3-(((1S,4S)-4,7,7-trimethyl-3-oxo-2-oxabicyclo[2.2.1]heptane-1-carboxamido)methyl)phenyl)-7,10,13-trioxa-2,17-diazanonadecan-19-oyl)-4-hydroxy-N-(4-(4-methylthiazol-5-yl)benzyl) pyrrolidine-2-carboxamide (74)

General Procedure 1 and 3. Reaction scale: 15.4 mg (21.0 μmol, 1.00 equiv) of 12 and 12.5 mg (30.0 μmol, 1.43 equiv) of(S)-camphanic acid phenyl derivative. Purified by PTLC (100% EtOAc) to afford 74 as a yellow oil (13.9 mg, 68%). ¹H NMR (400 MHz, CDCl₃) δ 8.66 (s, 1H), 7.36-7.28 (m, 6H), 7.19-7.13 (m, 3H), 7.08-6.98 (m, 2H), 6.91 (d, J=8.6 Hz, 1H), 4.61 (t, J=8.1 Hz, 1H), 4.56-4.38 (m, 8H), 4.32 (dd, J=15.1, 5.3 Hz, 1H), 4.06 (d, J=11.5 Hz, 1H), 3.74 (m, 2H), 3.68-3.50 (m, 12H), 2.51-2.49 (m, 6H), 2.44-2.38 (m, 2H), 2.12-2.07 (m, 2H), 1.97-1.87 (m, 3H), 1.70-1.63 (m, 1H), 1.11 (s, 3H), 1.09 (s, 3H), 0.93 (s, 9H), 0.89 (s, 3H).

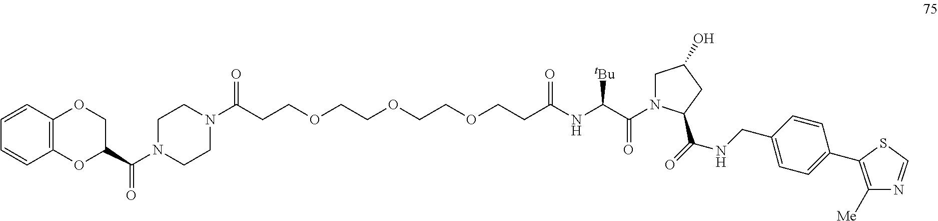

### (2S,4R)-1-((S)-2-(tert-butyl)-16-(4-((R)-2,3-dihydrobenzo[b][1,4]dioxine-2-carbonyl) piperazin-1-yl)-4,16-dioxo-7,10,13-trioxa-3-azahexadecanoyl)-4-hydroxy-N-(4-(4-methylthiazol-5-yl)benzyl) pyrrolidine-2-carboxamide (75)

General Procedure 3 then 2. Reaction scale: 6.0 mg (9.1 μmol, 1.0 eq.) of 12 and 2.4 mg (10.0 μmol, 1.1 eq.) of(S)-benzodioxanpiperazine methanone. Purified by pTLC (10% MeOH/CH₂Cl₂) to afford 75 as a white solid (3.3 mg, 41%). R/=0.49 (8% MeOH/CH₂Cl₂, UV-active); LC-MS (ESI+) calc'd for C₄₅H₆₀N₆O₁₁S 892.4, found [M+H]⁺ 893.9. ¹H NMR (400 MHZ, CDCl₃) δ 8.86 (br. s, 1H), 7.83-7.23 (m, 4H), 7.50 (br. t, 2H), 7.30 (s, 1H), 7.25 (br. t, 2H), 5.04 (t, 1H), 4.72 (t, 2H), 4.63 (d, J=3.1 Hz, 2H), 4.50 (s, 1H), 4.60-4.30 (m, 3H), 4.20 (s, 1H), 4.15-4.10 (d, J 7 Hz, 2H), 3.78 (ddd, J=9.1, 7.1, 2.4 Hz, 2H), 3.62 (q, J=5.4 Hz, 4H), 3.66-3.51 (m, 8H), 3.47 (q, J=6.7, 4.9 Hz, 4H), 2.60-2.35 (m, 9H), 2.20-2.10 (m, 1H), 2.05 (s, 1H), 0.93 (s, 9H).

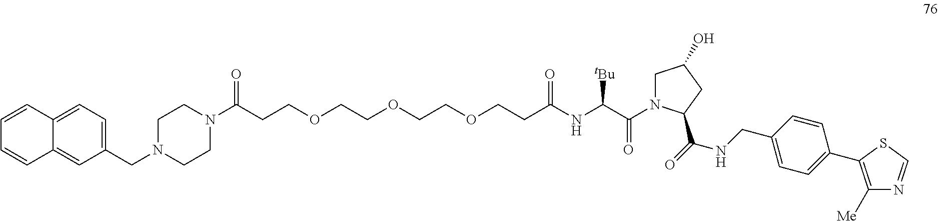 [SMILES: Cc1ncsc1-c1ccc(CNC(=O)[C@@H]2C[C@@H](O)CN2C(=O)[C@@H](NC(=O)CCOCCOCCOCCC(=O)N2CCN(Cc3ccc4ccccc4c3)CC2)C(C)(C)C)cc1]

### (2S,4R)-1-((S)-2-(tert-butyl)-16-(4-(naphthalen-2-ylmethyl) piperazin-1-yl)-4,16-dioxo-7,10,13-trioxa-3-azahexadecanoyl)-4-hydroxy-N-(4-(4-methylthiazol-5-yl)benzyl) pyrrolidine-2-carboxamide (76)

General Procedure 3 then 2. Reaction scale: 6.0 mg (9.1 μmol, 1.0 eq.) of 12 and 2.25 mg (10.0 μmol, 1.1 eq.) of naphthalene piperazine. Purified by pTLC (10% MeOH/CH₂Cl₂) to afford 76 as a slightly yellow solid (3.1 mg, 40%). R_{f}=0.55 (8% MeOH/CH₂Cl₂, UV-active); LC-MS (ESI+) calc'd for C₄₇H₆₂N₆O₈S 870.4, found [M+NH₄]⁺ 888.8. ¹H NMR (400 MHZ, CDCl₃) δ 8.86 (br. s, 1H), 7.80-7.35 (m, 7H), 7.50 (br. t, 2H), 7.30 (s, 1H), 7.25 (br. t, 2H), 5.04 (t, 1H), 4.72 (t, 2H), 4.63 (d, J=3.1 Hz, 2H), 4.50 (s, 1H), 4.60-4.30 (m, 3H), 4.20 (s, 1H), 4.15-4.10 (d, J 7 Hz, 2H), 3.92 (s, 2H), 3.78 (ddd, J=9.1, 7.1, 2.4 Hz, 2H), 3.62 (q, J=5.4 Hz, 4H), 3.66-3.51 (m, 16H), 3.47 (q, J=6.7, 4.9 Hz, 4H), 2.20-2.10 (m, 1H), 2.05 (s, 1H), 0.93 (s, 9H).

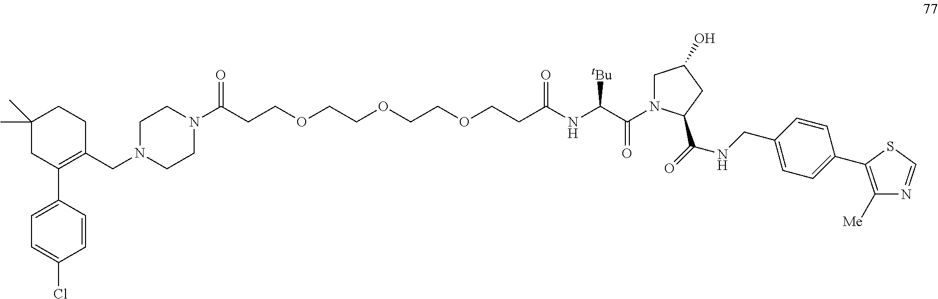 [SMILES: Cc1ncsc1-c1ccc(CNC(=O)[C@@H]2C[C@@H](O)CN2C(=O)[C@@H](NC(=O)CCOCCOCCOCCC(=O)N2CCN(CC3=C(c4ccc(Cl)cc4)CC(C)(C)CC3)CC2)C(C)(C)C)cc1]

### (2S,4R)-1-((S)-2-(tert-butyl)-16-(4-((4′-chloro-5,5-dimethyl-3,4,5,6-tetrahydro-[1,1′-biphenyl]-2-yl)methyl) piperazin-1-yl)-4,16-dioxo-7,10,13-trioxa-3-azahexadecanoyl)-4-hydroxy-N-(4-(4-methylthiazol-5-yl)benzyl) pyrrolidine-2-carboxamide (77)

General Procedure 3 then 2. Reaction scale: 6.5 mg (9.8 μmol, 1.0 eq.) of 12 and 3.4 mg (10.8 μmol, 1.1 eq.) of chlorophenyldimethylcyclohexenpiperazine. Purified by pTLC (10% MeOH/CH₂Cl₂) to afford 77 as a white solid (3.1 mg, 33%). R_{f}=0.61 (8% MeOH/CH₂Cl₂, UV-active); LC-MS (ESI−) calc'd for C₅₁H₇₁ClN₆O₈S 962.5, found [M+HCOO]⁻: 1007.3. ¹H NMR (400 MHZ, CDCl₃) δ 8.86 (br. s, 1H), 7.80-7.35 (m, 4H), 7.50 (br. t, 2H), 7.30 (s, 1H), 7.25 (br. t, 2H), 5.04 (t, 1H), 4.72 (t, 2H), 4.63 (d, J=3.1 Hz, 2H), 4.50 (s, 1H), 4.60-4.30 (m, 3H), 4.20 (s, 1H), 4.15-4.10 (d, J 7 Hz, 2H), 3.78 (ddd, J=9.1, 7.1, 2.4 Hz, 2H), 3.62 (q, J=5.4 Hz, 4H), 3.66-3.51 (m, 8H), 3.47 (q, J=6.7, 4.9 Hz, 4H), 3.10 (s, 2H), 2.60-2.35 (m, 8H), 2.20-2.10 (m, 5H), 2.05 (s, 1H), 1.53 (m 1H), 0.93 (s, 9H), 0.88 (m, 6H).

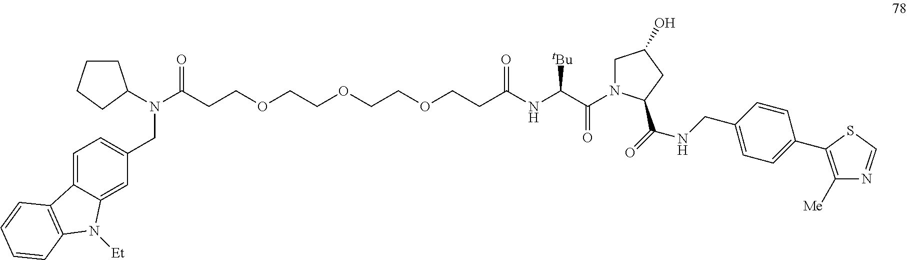 [SMILES: CCn1c2ccccc2c2ccc(CN(C(=O)CCOCCOCCOCCC(=O)N[C@H](C(=O)N3C[C@H](O)C[C@H]3C(=O)NCc3ccc(-c4scnc4C)cc3)C(C)(C)C)C3CCCC3)cc21]

### (2S,4R)-1-((S)-17-(tert-butyl)-2-cyclopentyl-1-(9-ethyl-9H-carbazol-2-yl)-3,15-dioxo-6,9,12-trioxa-2,16-diazaoctadecan-18-oyl)-4-hydroxy-N-(4-(4-methylthiazol-5-yl)benzyl) pyrrolidine-2-carboxamide (78)

General Procedure 3 then 2. Reaction scale: 6.0 mg (9.05 μmol) of 12 and 5.29 mg (18.11 μmol) of carbazolecyclopentanamine. Purified by pTLC (8% MeOH/DCM) to afford 78 as a white solid (5.3 mg, 63%). R_{f}=0.53 (8% MeOH/CH₂Cl₂, UV-active); LC-MS (ESI+) calc'd for C₅₂H₆₈N₆O₈S 936.5, found [M+H]⁺ 938.2. ¹H NMR (400 MHZ, CDCl₃) δ 8.86 (br. s, 1H), 7.80-7.35 (m, 8H), 7.50 (br. t, 2H), 7.30 (s, 1H), 7.25 (br. t, 2H), 5.04 (t, 1H), 4.72 (t, 2H), 4.63 (d, J=3.1 Hz, 2H), 4.50 (s, 1H), 4.60-4.30 (m, 5H), 4.20 (s, 1H), 4.15-4.10 (d, J=7 Hz, 2H), 3.78 (ddd, J=9.1, 7.1, 2.4 Hz, 2H), 3.66-3.51 (m, 11H), 2.60-2.35 (m, 6H), 2.20-2.10 (m, 1H), 2.05 (s, 1H), 1.60-1.87 (8H), 1.26 (t, 2H), 0.93 (s, 9H).

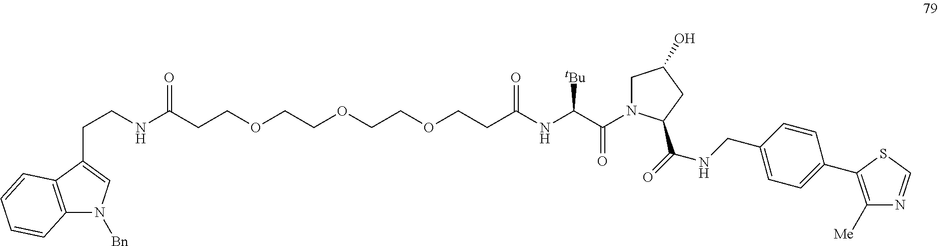 [SMILES: Cc1ncsc1-c1ccc(CNC(=O)[C@@H]2C[C@@H](O)CN2C(=O)[C@@H](NC(=O)CCOCCOCCOCCC(=O)NCCc2cn(Cc3ccccc3)c3ccccc23)C(C)(C)C)cc1]

### (2S,4R)-1-((S)-19-(1-benzyl-1H-indol-3-yl)-2-(tert-butyl)-4,16-dioxo-7,10,13-trioxa-3,17-diazanonadecanoyl)-4-hydroxy-N-(4-(4-methylthiazol-5-yl)benzyl) pyrrolidine-2-carboxamide (79)

General Procedure 2. Reaction scale: 6.0 mg (9.05 μmol) of 12 and 4.79 mg (18.11 μmol) of benzyl indole amine. Purified by pTLC (8% MeOH/DCM) to afford 79 as a slightly orange solid (mg, %). Molecular formula: C₅₀H₆₄N₆O₈S. MS calc'd 908.5, found [M+H]⁺ 909.7. ¹H NMR (400 MHZ, CDCl₃) δ 8.86 (br. s, 1H), 7.80-7.35 (m, 9H), 7.50 (br. t, 2H), 7.30 (s, 1H), 7.25 (br. t, 2H), 5.04 (t, 1H), 4.72 (t, 2H), 4.63 (d, J=3.1 Hz, 2H), 4.50 (s, 1H), 4.60-4.30 (m, 3H), 4.20 (s, 1H), 4.15-4.10 (d, J=7 Hz, 2H), 3.78 (ddd, J=9.1, 7.1, 2.4 Hz, 2H), 3.66-3.51 (m, 12H), 2.60-2.35 (m, 8H), 2.20-2.10 (m, 1H), 2.13 (s, 3H), 2.05 (s, 1H), 0.93 (s, 9H).

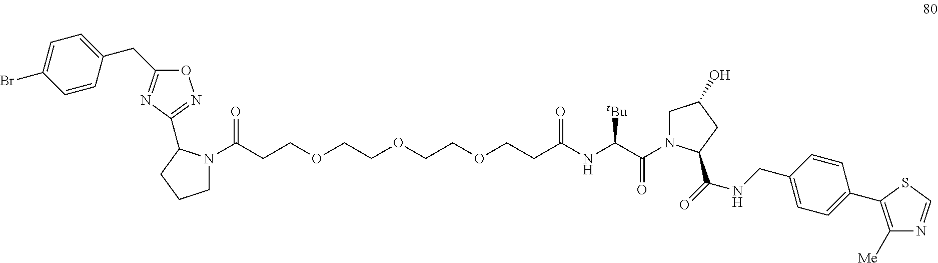 [SMILES: Cc1ncsc1-c1ccc(CNC(=O)[C@@H]2C[C@@H](O)CN2C(=O)[C@@H](NC(=O)CCOCCOCCOCCC(=O)N2CCCC2c2noc(Cc3ccc(Br)cc3)n2)C(C)(C)C)cc1]

### (2S,4R)-1-((2S)-16-(2-(5-(4-bromobenzyl)-1,2,4-oxadiazol-3-yl) pyrrolidin-1-yl)-2-(tert-butyl)-4,16-dioxo-7,10,13-trioxa-3-azahexadecanoyl)-4-hydroxy-N-(4-(4-methylthiazol-5-yl)benzyl) pyrrolidine-2-carboxamide (80)

General Procedure 3 then 2. Reaction scale: 6.0 mg (9.05 μmol) of 12 and 5.58 mg (18.11 μmol) of bromobenzyloxadiazolepyrrolidine. Purified by pTLC (8% MeOH/DCM) to afford 80 as a slightly yellow solid (mg, %). Molecular formula: C₄₅H₅₈BrN₇O₉S. MS calc'd 951.3, found [M+H]⁺ 952.4. ¹H NMR (400 MHZ, CDCl₃) δ 8.86 (br. s, 1H), 7.80-7.35 (m, 4H), 7.50 (br. t, 2H), 7.30 (s, 1H), 7.25 (br. t, 1H), 4.72 (t, 2H), 4.63 (d, J=3.1 Hz, 2H), 4.50 (s, 1H), 4.60-4.30 (m, 4H), 4.20 (s, 1H), 4.15-4.10 (d, J=7 Hz, 2H), 3.78 (ddd, J=9.1, 7.1, 2.4 Hz, 2H), 3.66-3.51 (m, 12H), 2.60-2.35 (m, 10H), 2.20-2.10 (m, 1H), 2.05 (s, 1H), 0.93 (s, 9H).

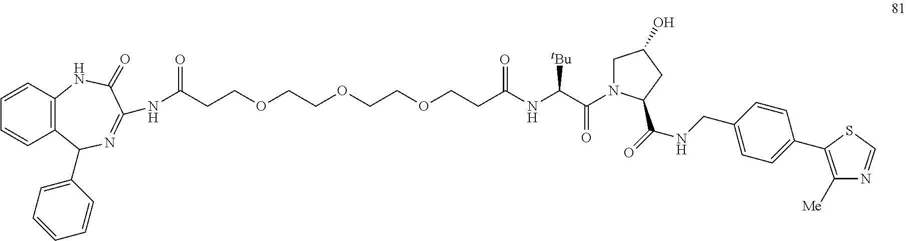 [SMILES: Cc1ncsc1-c1ccc(CNC(=O)[C@@H]2C[C@@H](O)CN2C(=O)[C@@H](NC(=O)CCOCCOCCOCCC(=O)NC2=NC(c3ccccc3)c3ccccc3NC2=O)C(C)(C)C)cc1]

### (2S,4R)-1-((2S)-2-(tert-butyl)-4,16-dioxo-16-((2-oxo-5-phenyl-2,5-dihydro-1H-benzo[e][1,4]diazepin-3-yl)amino)-7,10,13-trioxa-3-azahexadecanoyl)-4-hydroxy-N-(4-(4-methylthiazol-5-yl)benzyl) pyrrolidine-2-carboxamide (81)

General Procedure 3 then 1. Reaction scale: 8 mg (12.1 μmol, 1.0 eq.) of 12 and 3.3 mg (13.3 μmol, 1.1 eq.) of benzodiazepam. Purified by pTLC (10% MeOH/CH₂Cl₂) to afford 81 as a white solid (7 mg, 68%). R/=0.57 (10% MeOH/CH₂Cl₂, UV-active); LC-MS (ESI+) calc'd for C₄₇H₅₇N₇O₉S 895.4, found [M+H]⁺ 896.2. ¹H NMR (400 MHZ, CDCl₃) δ 9.96 (s, 1H), 8.67 (d, J=6.2 Hz, 2H), 8.42 (d, J=7.8 Hz, 1H), 7.58-7.49 (m, 5H), 7.37 (s, 6H), 7.13 (t, J=8.6 Hz, 4H), 5.56-5.46 (m, 2H), 4.71-4.62 (m, 3H), 4.56-4.49 (m, 3H), 4.36-4.29 (m, 1H), 4.22 (d, J=11.5 Hz, 2H), 4.12 (q, J=7.2 Hz, 4H), 3.86 (q, J=4.4 Hz, 2H), 3.67 (d, J=5.0 Hz, 7H), 2.50 (s, 5H), 0.92 (s, 9H). Note: presence of rotamers.

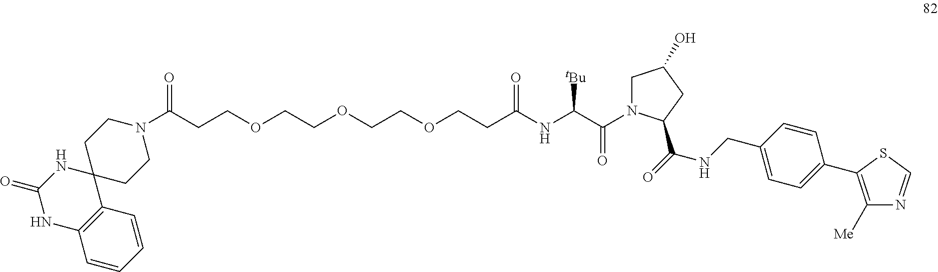 [SMILES: Cc1ncsc1-c1ccc(CNC(=O)[C@@H]2C[C@@H](O)CN2C(=O)[C@@H](NC(=O)CCOCCOCCOCCC(=O)N2CCC3(CC2)NC(=O)Nc2ccccc23)C(C)(C)C)cc1]

### (2S,4R)-1-((S)-2-(tert-butyl)-4,16-dioxo-16-(2′-oxo-2′,3′-dihydro-1′H-spiro[piperidine-4,4′-quinazolin]-1-yl)-7,10,13-trioxa-3-azahexadecanoyl)-4-hydroxy-N-(4-(4-methylthiazol-5-yl)benzyl) pyrrolidine-2-carboxamide (82)

General Procedure 3 then 1. Reaction scale: 8 mg (12.1 μmol, 1.0 eq.) of 12 and 2.88 mg (13.3 μmol, 1.1 eq.) of spiroquinazoline. Purified by pTLC (10% MeOH/CH₂Cl₂) to afford 82 as a yellow solid (5 mg, 48%). R_{f}=0.40 (10% MeOH/CH₂Cl₂, UV-active); LC-MS (ESI+) calc'd for C₄₄H₅₉N₇O₉S 861.4, found [M+H]⁺ 862.4. ¹H NMR (400 MHZ, CDCl₃) δ 8.65 (d, J=16.1 Hz, 1H), 7.55 (s, 1H), 7.41-7.30 (m, 5H), 7.22-7.08 (m, 2H), 6.99 (d, J=5.5 Hz, 1H), 6.74 (d, J=7.8 Hz, 1H), 4.71 (t, J=8.6 Hz, 2H), 4.59-4.47 (m, 3H), 4.38-4.24 (m, 1H), 4.18-4.05 (m, 2H), 3.92-3.73 (m, 5H), 3.67-3.44 (m, 12H), 2.76 (dt, J=13.4, 6.3 Hz, 1H), 2.58 (dd, J=15.2, 5.5 Hz, 1H), 2.44 (s, 3H), 2.26 (dd, J=14.0, 5.5 Hz, 2H), 1.94 (q, J=14.0 Hz, 6H), 0.88 (s, 9H). Note: presence of rotamers.

TABLE 3
Non-limiting Examples of FragTAC Probes and Intermediates

| Compound | | | |
|---|---|---|---|
| Example | Compound | | |
| Number | Code | Structure | Chemical Name |
| 1 | IF-012 | 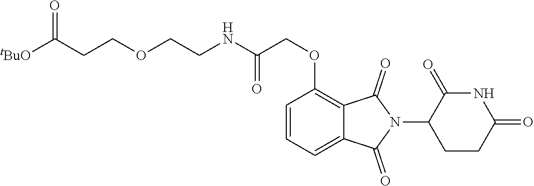 [SMILES: CC(C)(C)OC(=O)CCOCCNC(=O)COc1cccc2c1C(=O)N(C1CCC(=O)NC1=O)C2=O] | Tert-butyl 3-(2-(2-((2-(2,6- dioxopiperidin-3-yl)-1,3- dioxoisoindolin- 4yl)oxy)acetamido)ethoxy) propanoate |
| 2 | LPC002 | 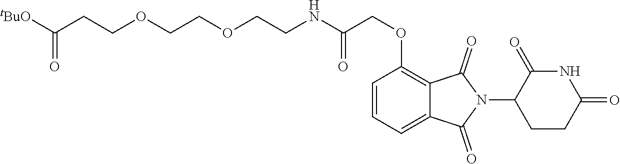 [SMILES: CC(C)(C)OC(=O)CCOCCOCCNC(=O)COc1cccc2c1C(=O)N(C1CCC(=O)NC1=O)C2=O] | Tert-butyl 3-(2-(2-(2-((2-(2,6- dioxopiperidin-3-yl)-1,3- dioxoisoindolin-4- yl)oxy)acetamido)ethoxy)ethoxy) propanoate |
| 3 | IF-001 | 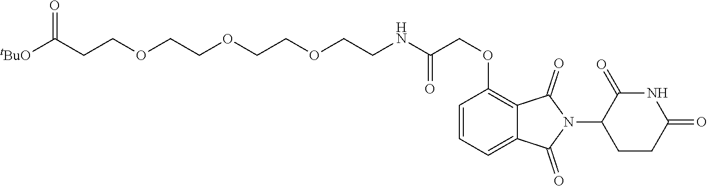 [SMILES: CC(C)(C)OC(=O)CCOCCOCCOCCNC(=O)COc1cccc2c1C(=O)N(C1CCC(=O)NC1=O)C2=O] | Tert-butyl 1-((2-(2,6- dioxopiperidin-3-yl)-1,3- dioxoisoindolin-4-yl)oxy)-2- oxo-6,9,12-trioxa-3- azapentadecan-15-oate |
| 4 | IF-092 | 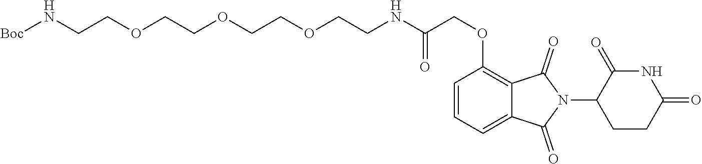 [SMILES: CC(C)(C)OC(=O)NCCOCCOCCOCCNC(=O)COc1cccc2c1C(=O)N(C1CCC(=O)NC1=O)C2=O] | Tert-butyl (1-((2-(2,6- dioxopiperidin-3-yl)-1,3- dioxoisoindolin-4-yl)oxy)-2- oxo-6,9, 12-trioxa-3- azatetradecan-14-yl)carbamate |
| 5 | IF-015 | 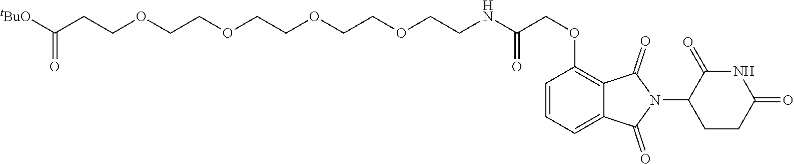 [SMILES: CC(C)(C)OC(=O)CCOCCOCCOCCOCCNC(=O)COc1cccc2c1C(=O)N(C1CCC(=O)NC1=O)C2=O] | Tert-butyl 1-((2-(2,6- dioxopiperidin-3-yl)-1,3- dioxoisoindolin-4-yl)oxy)-2- oxo-6,9,12,15-tetraoxa-3- azaoctadecan-18-oate |
| 6 | IF-025 | 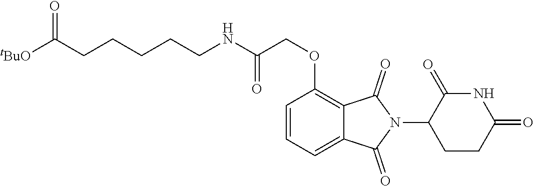 [SMILES: CC(C)(C)OC(=O)CCCCCNC(=O)COc1cccc2c1C(=O)N(C1CCC(=O)NC1=O)C2=O] | Tert-butyl 6-(2-((2-(2,6- dioxopiperidin-3-yl)-1,3- dioxoisoindolin-4-yl)oxy) acetamido)hexanoate |
| 7 | CMC-6- 194 | 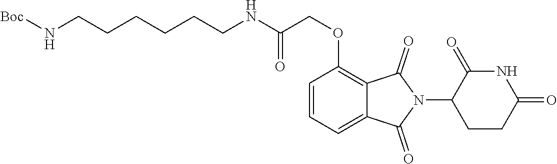 [SMILES: CC(C)(C)OC(=O)NCCCCCCNC(=O)COc1cccc2c1C(=O)N(C1CCC(=O)NC1=O)C2=O] | Tert-butyl (6-(2-((2-(2,6- dioxopiperidin-3-yl)-1,3- dioxoisoindolin-4-yl)oxy) acetamido)hexyl)carbamate |
| 8 | IF-081 | 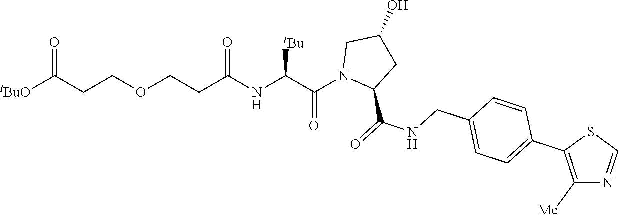 [SMILES: Cc1ncsc1-c1ccc(CNC(=O)[C@@H]2C[C@@H](O)CN2C(=O)[C@@H](NC(=O)CCOCCC(=O)OC(C)(C)C)C(C)(C)C)cc1] | Tert-butyl 3-(3-(((S)-1-((2S,4R)- 4-hydroxy-2-((4-(4- methylthiazol-5- yl)benzyl)carbamoyl)pyrrolidin- 1-yl)-3,3-dimethyl-1-oxobutan- 2-yl)amino)-3- oxopropoxy)propanoate |
| 9 | LPC115 | 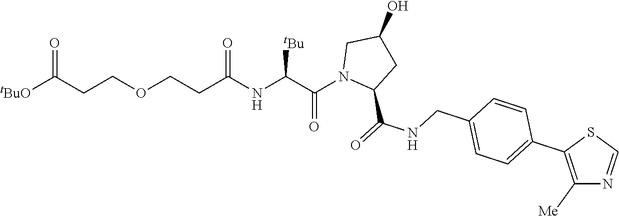 [SMILES: Cc1ncsc1-c1ccc(CNC(=O)[C@@H]2C[C@H](O)CN2C(=O)[C@@H](NC(=O)CCOCCC(=O)OC(C)(C)C)C(C)(C)C)cc1] | Tert-butyl 3-(3-(((S)-1-((2S,4S)- 4-hydroxy-2-((4-(4- methylthiazol-5- yl)benzyl)carbamoyl)pyrrolidin- 1-yl)-3,3-dimethyl-1-oxobutan- 2-yl)amino)-3- oxopropoxy)propanoate |
| 10 | LPC013 | 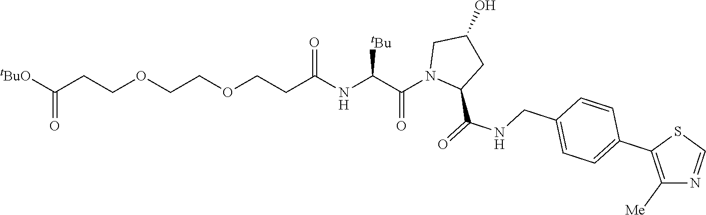 [SMILES: Cc1ncsc1-c1ccc(CNC(=O)[C@@H]2C[C@@H](O)CN2C(=O)[C@@H](NC(=O)CCOCCOCCC(=O)OC(C)(C)C)C(C)(C)C)cc1] | Tert-butyl 3-(2-(3-(((S)-1- ((2S,4R)-4-hydroxy-2-((4-(4- methylthiazol-5- yl)benzyl)carbamoyl)pyrrolidin- 1-yl)-3,3-dimethyl-1-oxobutan- 2-yl)amino)-3- oxopropoxy)ethoxy)propanoate |
| 11 | LPC114 | 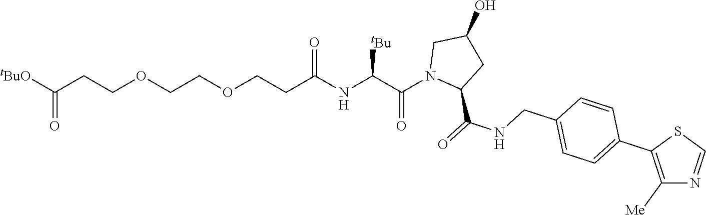 [SMILES: Cc1ncsc1-c1ccc(CNC(=O)[C@@H]2C[C@H](O)CN2C(=O)[C@@H](NC(=O)CCOCCOCCC(=O)OC(C)(C)C)C(C)(C)C)cc1] | Tert-butyl 3-(2-(3-(((S)-1- ((2S,4S)-4-hydroxy-2-((4-(4- methylthiazol-5-yl) benzyl)carbamoyl)pyrrolidin-1- yl)-3,3-dimethyl-1-oxobutan-2- yl)amino)-3- oxopropoxy)ethoxy)propanoate |
| 12 | IF-061 | 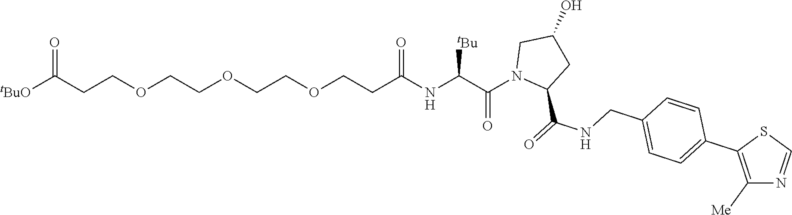 [SMILES: Cc1ncsc1-c1ccc(CNC(=O)[C@@H]2C[C@@H](O)CN2C(=O)[C@@H](NC(=O)CCOCCOCCOCCC(=O)OC(C)(C)C)C(C)(C)C)cc1] | Tert-butyl (S)-15-((2S,4R)-4- hydroxy-2-((4-(4-methylthiazol- 5-yl) benzyl) carbamoyl) pyrrolidine-1-carbonyl)-16,16- dimethyl-13-oxo-4,7,10-trioxa- 14-azaheptadecanoate |
| 13 | IF-068 | 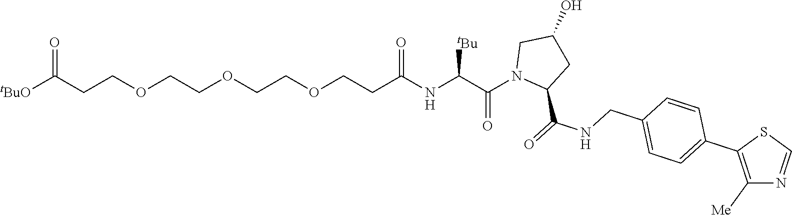 [SMILES: Cc1ncsc1-c1ccc(CNC(=O)[C@@H]2C[C@@H](O)CN2C(=O)[C@@H](NC(=O)CCOCCOCCOCCC(=O)OC(C)(C)C)C(C)(C)C)cc1] | Tert-butyl (S)-15-((2S,4S)-4- hydroxy-2-((4-(4-methylthiazol- 5-yl)benzyl)carbamoyl) pyrrolidine-1-carbonyl)-16,16- dimethyl-13-oxo-4,7,10-trioxa- 14-azaheptadecanoate |
| 14 | IF-069 | 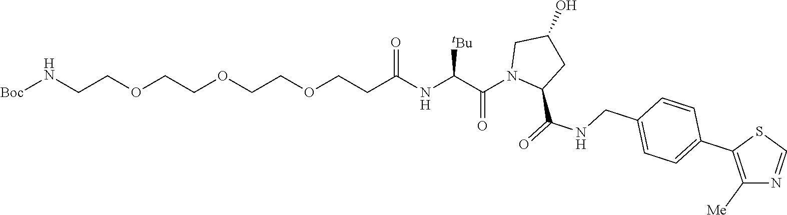 [SMILES: Cc1ncsc1-c1ccc(CNC(=O)[C@@H]2C[C@@H](O)CN2C(=O)[C@@H](NC(=O)CCOCCOCCOCCNC(=O)OC(C)(C)C)C(C)(C)C)cc1] | Tert-butyl ((S)-14-((2S,4R)-4- hydroxy-2-((4-(4-methylthiazol- 5- yl)benzyl)carbamoyl)pyrrolidine- 1-carbonyl)-15,15-dimethyl-12- oxo-3,6,9-trioxa-13- azahexadecyl)carbamate |
| 15 | IF-052 | 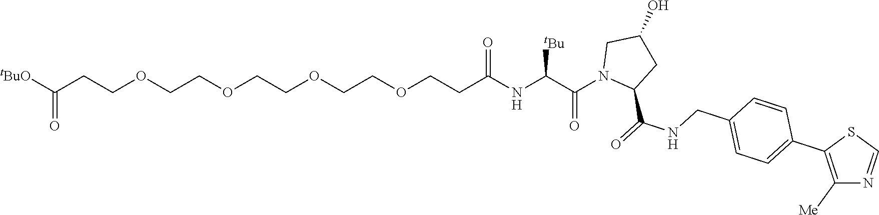 [SMILES: Cc1ncsc1-c1ccc(CNC(=O)[C@@H]2C[C@@H](O)CN2C(=O)[C@@H](NC(=O)CCOCCOCCOCCOCCC(=O)OC(C)(C)C)C(C)(C)C)cc1] | Tert-butyl (S)-18-((2S,4R)-4- hydroxy-2-((4-(4-methylthiazol- 5-yl)benzyl)carbamoyl) pyrrolidine-1-carbonyl)-19,19- dimethyl-16-oxo-4,7,10,13- tetraoxa-17-azaicosanoate |
| 16 | IF-036 | 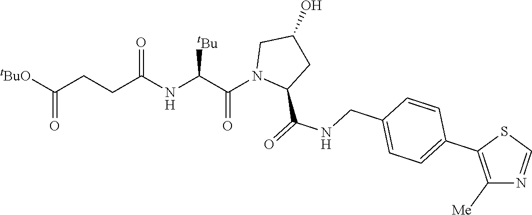 [SMILES: Cc1ncsc1-c1ccc(CNC(=O)[C@@H]2C[C@@H](O)CN2C(=O)[C@@H](NC(=O)CCC(=O)OC(C)(C)C)C(C)(C)C)cc1] | Tert-butyl 4-(((S)-1-((2S,4R)-4- hydroxy-2-((4-(4-methylthiazol- 5-yl)benzyl) carbamoyl)pyrrolidin-1-yl)-3,3- dimethyl-1-oxobutan-2- yl)amino)-4-oxobutanoate |
| 17 | IF-084 | 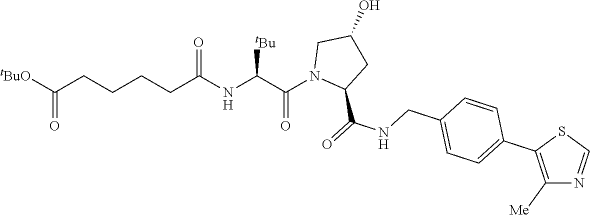 [SMILES: Cc1ncsc1-c1ccc(CNC(=O)[C@@H]2C[C@@H](O)CN2C(=O)[C@@H](NC(=O)CCCCC(=O)OC(C)(C)C)C(C)(C)C)cc1] | Tert-butyl 6-(((S)-1-((2S,4R)-4- hydroxy-2-((4-(4-methylthiazol- 5-yl)benzyl) carbamoyl)pyrrolidin-1-yl)-3,3- dimethyl-1-oxobutan-2- yl)amino)-6-oxohexanoate |
| 18 | CMC-6- 108 | 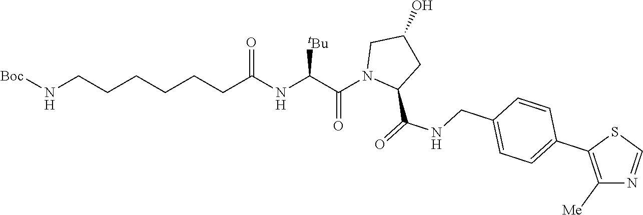 [SMILES: Cc1ncsc1-c1ccc(CNC(=O)[C@@H]2C[C@@H](O)CN2C(=O)[C@@H](NC(=O)CCCCCCNC(=O)OC(C)(C)C)C(C)(C)C)cc1] | Tert-butyl (7-(((S)-1-((2S,4R)-4- hydroxy-2-((4-(4-methylthiazol- 5-yl) benzyl) carbamoyl)pyrrolidin-1-yl)-3,3- dimethyl-1-oxobutan-2- yl)amino)-7-oxoheptyl) carbamate |
| 19 | IF-042 | 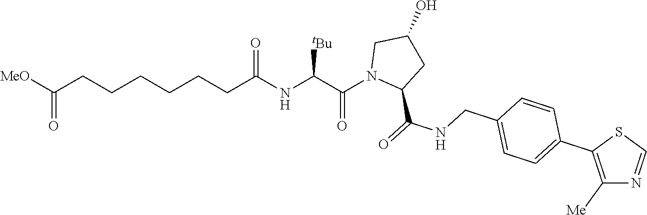 [SMILES: COC(=O)CCCCCCC(=O)N[C@H](C(=O)N1C[C@H](O)C[C@H]1C(=O)NCc1ccc(-c2scnc2C)cc1)C(C)(C)C] | Methyl 8-(((S)-1-((2S,4R)-4- hydroxy-2-((4-(4-methylthiazol- 5-yl)benzyl)carbamoyl) pyrrolidin-1-yl)-3,3-dimethyl-1- oxobutan-2-yl)amino)-8- oxooctanoate |
| 20 | IF-045 | 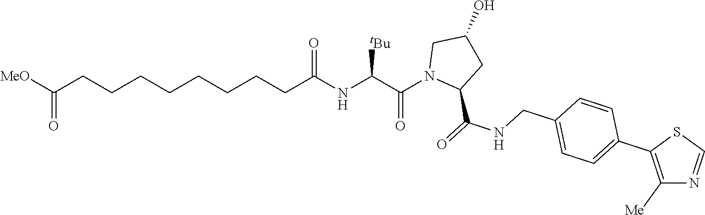 [SMILES: COC(=O)CCCCCCCCC(=O)N[C@H](C(=O)N1C[C@H](O)C[C@H]1C(=O)NCc1ccc(-c2scnc2C)cc1)C(C)(C)C] | Methyl 10-(((S)-1-((2S,4R)-4- hydroxy-2-((4-(4-methylthiazol- 5-yl) benzyl) carbamoyl)pyrrolidin-1-yl)-3,3- dimethyl-1-oxobutan-2- yl)amino)-10-oxodecanoate |
| 21 | IF-014 | 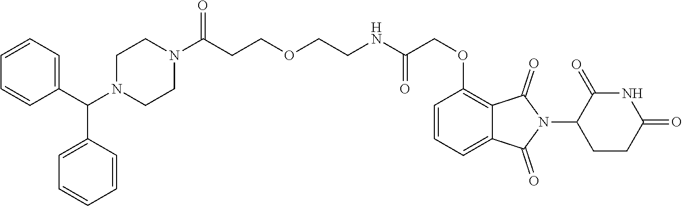 [SMILES: O=C(COc1cccc2c1C(=O)N(C1CCC(=O)NC1=O)C2=O)NCCOCCC(=O)N1CCN(C(c2ccccc2)c2ccccc2)CC1] | N-(2-(3-(4-benzhydrylpiperazin- 1-yl)-3-oxopropoxy)ethyl)-2- ((2-(2,6-dioxopiperidin-3-yl)- 1,3-dioxoisoindolin-4- yl)oxy)acetamide |
| 22 | LPC011 | 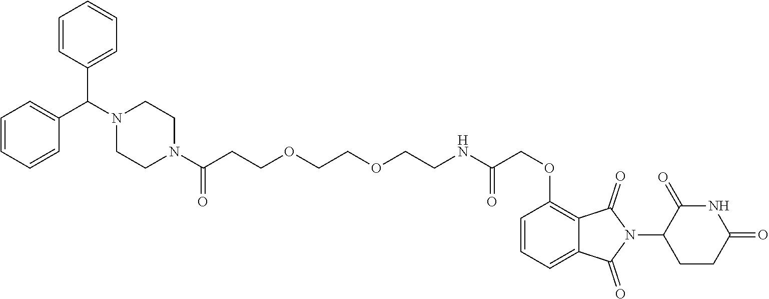 [SMILES: O=C(COc1cccc2c1C(=O)N(C1CCC(=O)NC1=O)C2=O)NCCOCCOCCC(=O)N1CCN(C(c2ccccc2)c2ccccc2)CC1] | N-(2-(2-(3-(4- benzhydrylpiperazin-1-yl)-3- oxopropoxy)ethoxy)ethyl)-2- ((2-(2,6-dioxopiperidin-3-yl)- 1,3-dioxoisoindolin-4- yl)oxy)acetamide |
| 23 | IF-003 | 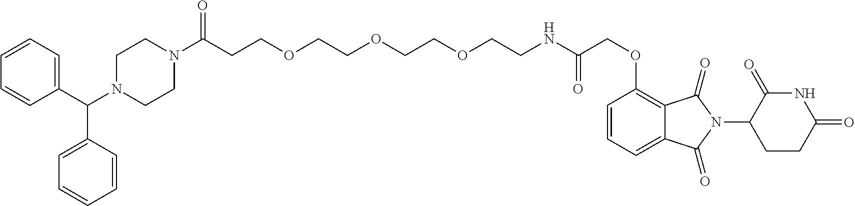 [SMILES: O=C(COc1cccc2c1C(=O)N(C1CCC(=O)NC1=O)C2=O)NCCOCCOCCOCCC(=O)N1CCN(C(c2ccccc2)c2ccccc2)CC1] | N-(2-(2-(2-(3-(4- benzhydrylpiperazin-1-yl)-3- oxopropoxy)ethoxy)ethoxy) ethyl)-2-((2-(2,6- dioxopiperidin-3- yl)-1,3-dioxoisoindolin-4- yl)oxy)acetamide |
| 24 | IF-017 | 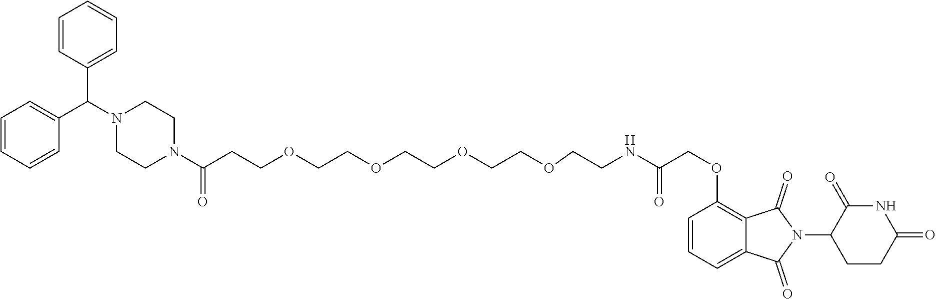 [SMILES: O=C(COc1cccc2c1C(=O)N(C1CCC(=O)NC1=O)C2=O)NCCOCCOCCOCCOCCC(=O)N1CCN(C(c2ccccc2)c2ccccc2)CC1] | N-(15-(4-benzhydrylpiperazin- 1-yl)-15-oxo-3,6,9,12- tetraoxapentadecyl)-2-((2-(2,6- dioxopiperidin-3-yl)-1,3- dioxoisoindolin-4- yl)oxy)acetamide |
| 25 | IF-027 | 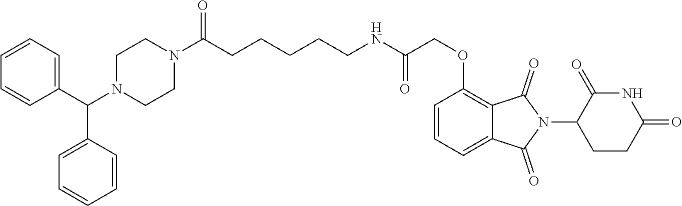 [SMILES: O=C(COc1cccc2c1C(=O)N(C1CCC(=O)NC1=O)C2=O)NCCCCCC(=O)N1CCN(C(c2ccccc2)c2ccccc2)CC1] | N-(6-(4-benzhydrylpiperazin-1- yl)-6-oxohexyl)-2-((2-(2,6- dioxopiperidin-3-yl)-1,3- dioxoisoindolin-4- yl)oxy)acetamide |
| 26 | IF-049 | 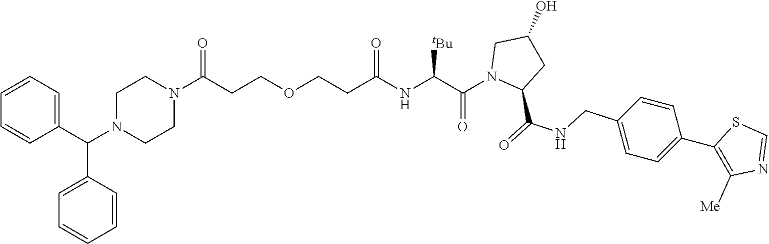 [SMILES: Cc1ncsc1-c1ccc(CNC(=O)[C@@H]2C[C@@H](O)CN2C(=O)[C@@H](NC(=O)CCOCCC(=O)N2CCN(C(c3ccccc3)c3ccccc3)CC2)C(C)(C)C)cc1] | (2S,4R)-1-((S)-2-(3-(3-(4- benzhydrylpiperazin-1-yl)-3- oxopropoxy)propanamido)-3,3- dimethylbutanoyl)-4-hydroxy- N-(4-(4-methylthiazol-5-yl) benzyl)pyrrolidine-2- carboxamide |
| 27 | LPC118 | 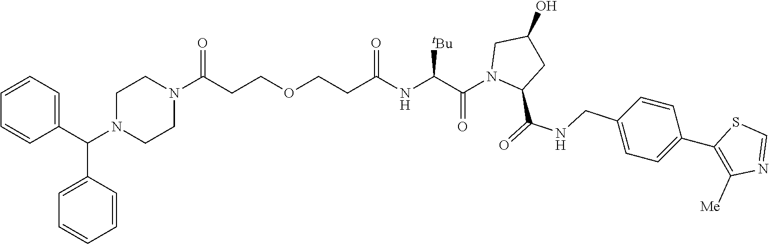 [SMILES: Cc1ncsc1-c1ccc(CNC(=O)[C@@H]2C[C@H](O)CN2C(=O)[C@@H](NC(=O)CCOCCC(=O)N2CCN(C(c3ccccc3)c3ccccc3)CC2)C(C)(C)C)cc1] | (2S,4S)-1-((S)-2-(3-(3-(4- benzhydrylpiperazin-1-yl)-3- oxopropoxy) propanamido)-3,3- dimethylbutanoyl)-4-hydroxy- N-(4-(4-methylthiazol-5-yl) benzyl)pyrrolidine-2- carboxamide |
| 28 | LPC020 | 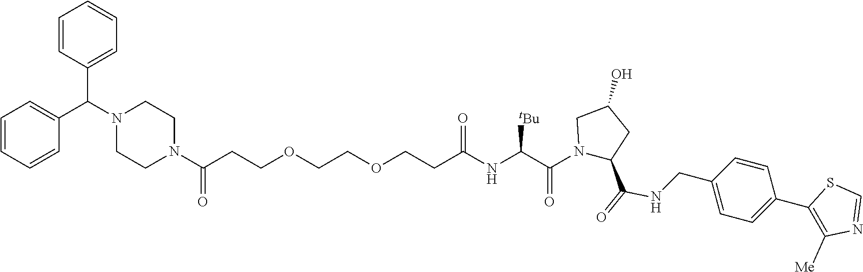 [SMILES: Cc1ncsc1-c1ccc(CNC(=O)[C@@H]2C[C@@H](O)CN2C(=O)[C@@H](NC(=O)CCOCCOCCC(=O)N2CCN(C(c3ccccc3)c3ccccc3)CC2)C(C)(C)C)cc1] | (2S,4R)-1-((S)-2-(3-(2-(3-(4- benzhydrylpiperazin-1-yl)-3- oxopropoxy) ethoxy)propanamido)-3,3- dimethylbutanoyl)-4-hydroxy- N-(4-(4-methylthiazol-5- yl)benzyl)pyrrolidine-2- carboxamide |
| 29 | LPC117 | 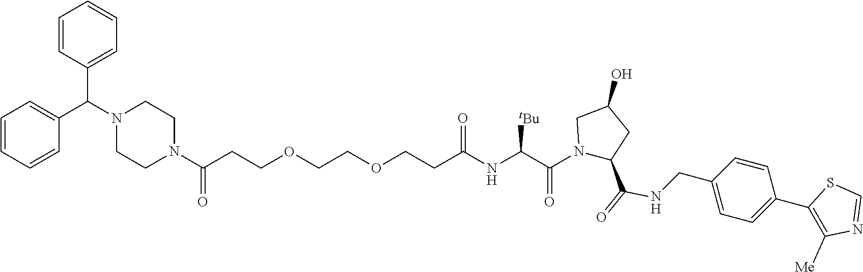 [SMILES: Cc1ncsc1-c1ccc(CNC(=O)[C@@H]2C[C@H](O)CN2C(=O)[C@@H](NC(=O)CCOCCOCCC(=O)N2CCN(C(c3ccccc3)c3ccccc3)CC2)C(C)(C)C)cc1] | (2S,4S)-1-((S)-2-(3-(2-(3-(4- benzhydrylpiperazin-1-yl)-3- oxopropoxy) ethoxy)propanamido)-3,3- dimethylbutanoyl)-4-hydroxy- N-(4-(4-methylthiazol-5- yl)benzyl)pyrrolidine-2- carboxamide |
| 30 | IF-051 | 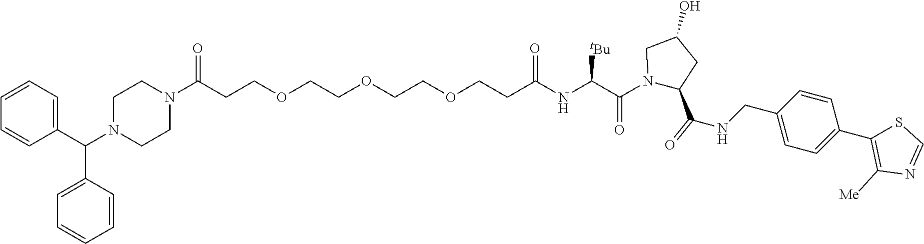 [SMILES: Cc1ncsc1-c1ccc(CNC(=O)[C@@H]2C[C@@H](O)CN2C(=O)[C@@H](NC(=O)CCOCCOCCOCCC(=O)N2CCN(C(c3ccccc3)c3ccccc3)CC2)C(C)(C)C)cc1] | (2S,4R)-1-((S)-16-(4- benzhydrylpiperazin-1-yl)-2- (tert-butyl)-4,16-dioxo-7,10,13- trioxa-3-azahexadecanoyl)-4- hydroxy-N-(4-(4-methylthiazol- 5-yl)benzyl)pyrrolidine-2- carboxamide |
| 31 | LPC116 | 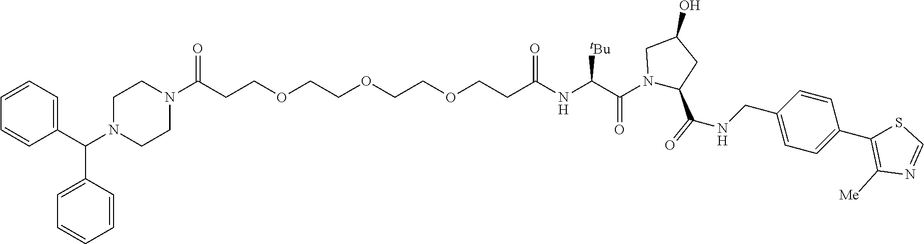 [SMILES: Cc1ncsc1-c1ccc(CNC(=O)[C@@H]2C[C@H](O)CN2C(=O)[C@@H](NC(=O)CCOCCOCCOCCC(=O)N2CCN(C(c3ccccc3)c3ccccc3)CC2)C(C)(C)C)cc1] | (2S,4S)-1-((S)-16-(4- benzhydrylpiperazin-1-yl)-2- (tert-butyl)-4,16-dioxo-7,10,13- trioxa-3-azahexadecanoyl)-4- hydroxy-N-(4-(4-methylthiazol- 5-yl)benzyl)pyrrolidine-2- carboxamide |
| 32 | IF-053 | 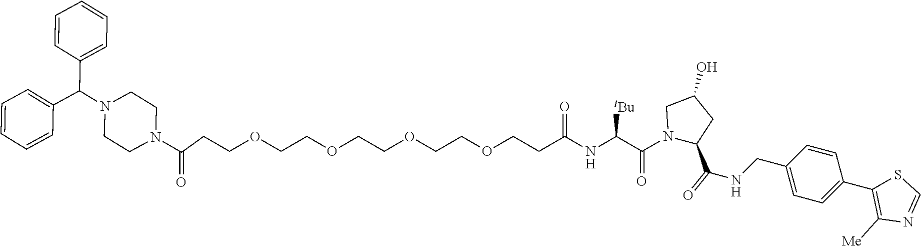 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(C(c3ccccc3)c3ccccc3)CC2)C(C)(C)C)cc1] | (2S,4R)-1-((S)-19-(4- benzhydrylpiperazin-1-yl)-2- (tert-butyl)-4,19-dioxo- 7,10,13,16-tetraoxa-3- azanonadecanoyl)-4-hydroxy-N- (4-(4-methylthiazol-5-yl) benzyl)pyrrolidine-2- carboxamide |
| 33 | IF-038 | 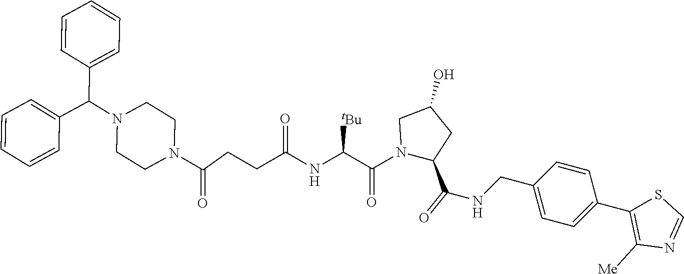 [SMILES: Cc1ncsc1-c1ccc(CNC(=O)[C@@H]2C[C@@H](O)CN2C(=O)[C@@H](NC(=O)CCC(=O)N2CCN(C(c3ccccc3)c3ccccc3)CC2)C(C)(C)C)cc1] | (2S,4R)-1-((S)-2-(4-(4- benzhydrylpiperazin-1-yl)-4- oxobutanamido)-3,3- dimethylbutanoyl)-4-hydroxy- N-(4-(4-methylthiazol-5-yl) benzyl)pyrrolidine-2- carboxamide |
| 34 | IF-041 | 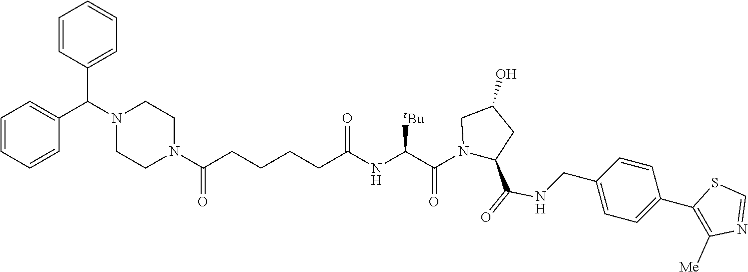 [SMILES: Cc1ncsc1-c1ccc(CNC(=O)[C@@H]2C[C@@H](O)CN2C(=O)[C@@H](NC(=O)CCCCC(=O)N2CCN(C(c3ccccc3)c3ccccc3)CC2)C(C)(C)C)cc1] | (2S,4R)-1-((S)-2-(6-(4- benzhydrylpiperazin-1-yl)-6- oxohexanamido)-3,3- dimethylbutanoyl)-4-hydroxy- N-(4-(4-methylthiazol-5-yl) benzyl)pyrrolidine-2- carboxamide |
| 35 | IF-044 | 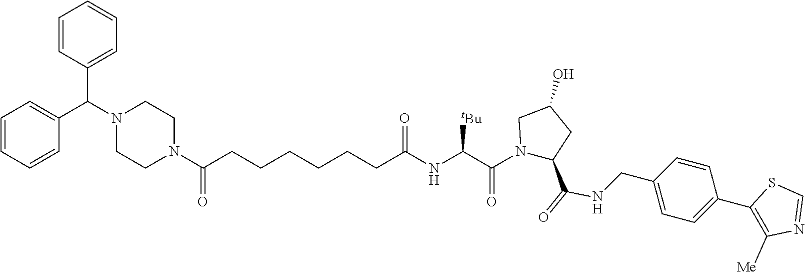 [SMILES: Cc1ncsc1-c1ccc(CNC(=O)[C@@H]2C[C@@H](O)CN2C(=O)[C@@H](NC(=O)CCCCCCC(=O)N2CCN(C(c3ccccc3)c3ccccc3)CC2)C(C)(C)C)cc1] | (2S,4R)-1-((S)-2-(8-(4- benzhydrylpiperazin-1-yl)-8- oxooctanamido)-3,3- dimethylbutanoyl)-4-hydroxy- N-(4-(4-methylthiazol-5-yl) benzyl)pyrrolidine-2- carboxamide |
| 36 | IF-047 | 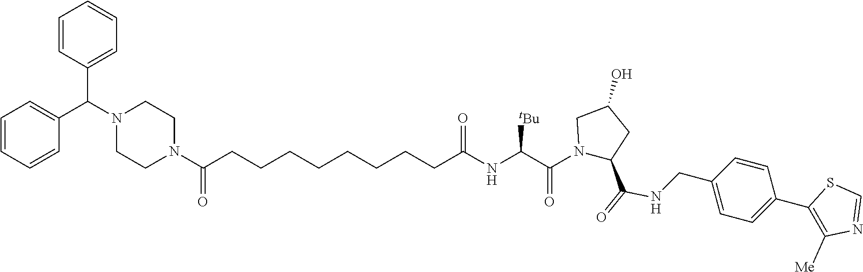 [SMILES: Cc1ncsc1-c1ccc(CNC(=O)[C@@H]2C[C@@H](O)CN2C(=O)[C@@H](NC(=O)CCCCCCCCC(=O)N2CCN(C(c3ccccc3)c3ccccc3)CC2)C(C)(C)C)cc1] | (2S,4R)-1-((S)-2-(10-(4- benzhydrylpiperazin-1-yl)-10- oxodecanamido)-3,3- dimethylbutanoyl)-4-hydroxy- N-(4-(4-methylthiazol-5-yl) benzyl)pyrrolidine-2- carboxamide |
| 37 | IF-085 | 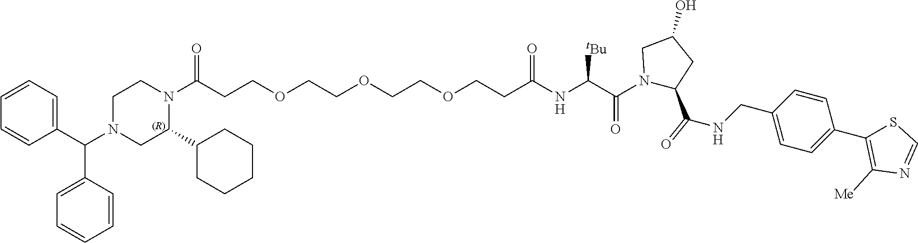 | (2S,4R)-1-((S)-16-((R)-4- benzhydryl-2-phenylpiperazin- 1-yl)-2-(tert-butyl)-4,16-dioxo- 7,10,13-trioxa-3- azahexadecanoyl)-4-hydroxy-N- (4-(4-methylthiazol-5-yl) benzyl)pyrrolidine-2- carboxamide |
| 38 | CMC-6- 123 | 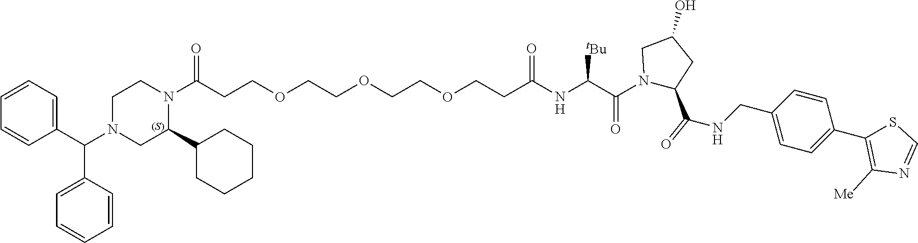 | (2S,4R)-1-((S)-16-((S)-4- benzhydryl-2-phenylpiperazin- 1-yl)-2-(tert-butyl)-4,16-dioxo- 7,10,13-trioxa-3- azahexadecanoyl)-4-hydroxy-N- (4-(4-methylthiazol-5-yl) benzyl)pyrrolidine-2- carboxamide |
| 39 | IF-088 | 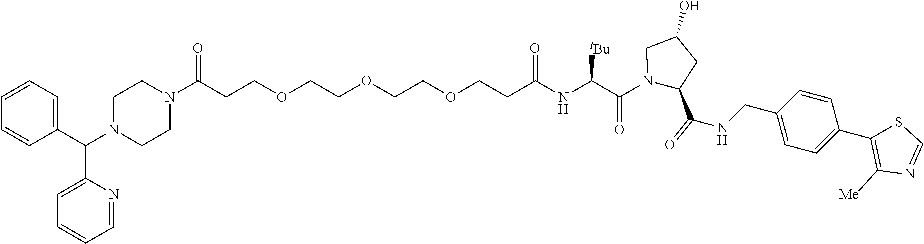 [SMILES: Cc1ncsc1-c1ccc(CNC(=O)[C@@H]2C[C@@H](O)CN2C(=O)[C@@H](NC(=O)CCOCCOCCOCCC(=O)N2CCN(C(c3ccccc3)c3ccccn3)CC2)C(C)(C)C)cc1] | (2S,4R)-1-((2S)-2-(tert-butyl)- 4,16-dioxo-16-(4- (phenyl(pyridin-2-yl)methyl) piperazin-1-yl)-7,10,13-trioxa-3- azahexadecanoyl)-4-hydroxy-N- (4-(4-methylthiazol-5- yl)benzyl)pyrrolidine-2- carboxamide |
| 40 | CMC-6- 125 | 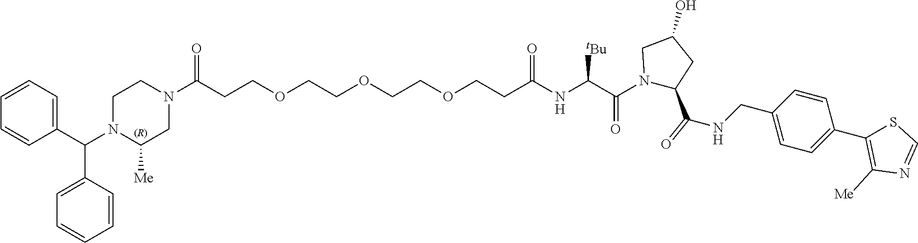 | (2S,4R)-1-((S)-16-((S)-4- benzhydryl-3-methylpiperazin- 1-yl)-2-(tert-butyl)-4,16-dioxo- 7,10,13-trioxa-3- azahexadecanoyl)-4-hydroxy-N- (4-(4-methylthiazol-5-yl) benzyl)pyrrolidine-2- carboxamide |
| 41 | IF-082 | 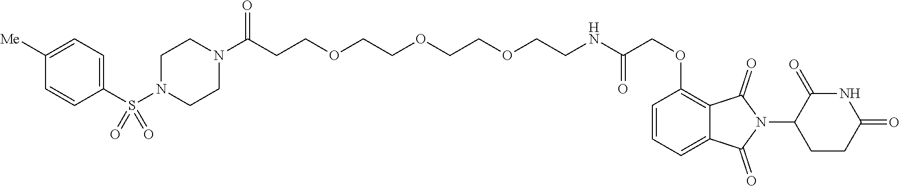 [SMILES: Cc1ccc(S(=O)(=O)N2CCN(C(=O)CCOCCOCCOCCNC(=O)COc3cccc4c3C(=O)N(C3CCC(=O)NC3=O)C4=O)CC2)cc1] | 2-((2-(2,6-dioxopiperidin-3-yl)- 1,3-dioxoisoindolin-4-yl)oxy)- N-(2-(2-(2-(3-oxo-3-(4- tosylpiperazin-1-yl) propoxy) ethoxy)ethoxy)ethyl)acetamide |
| 42 | IF-079 | 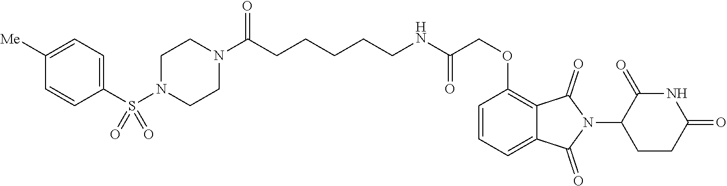 [SMILES: Cc1ccc(S(=O)(=O)N2CCN(C(=O)CCCCCNC(=O)COc3cccc4c3C(=O)N(C3CCC(=O)NC3=O)C4=O)CC2)cc1] | 2-((2-(2,6-dioxopiperidin-3-yl)- 1,3-dioxoisoindolin-4-yl)oxy)- N-(6-oxo-6-(4-tosylpiperazin-1- yl)hexyl)acetamide |
| 43 | IF-058 | 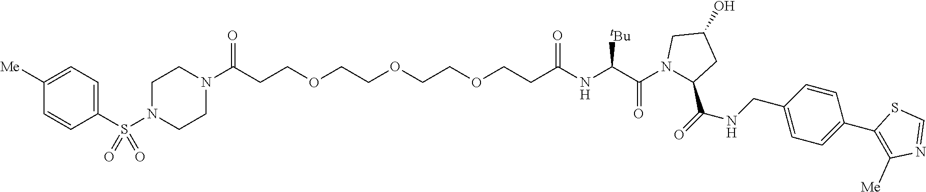 [SMILES: Cc1ccc(S(=O)(=O)N2CCN(C(=O)CCOCCOCCOCCC(=O)N[C@H](C(=O)N3C[C@H](O)C[C@H]3C(=O)NCc3ccc(-c4scnc4C)cc3)C(C)(C)C)CC2)cc1] | (2S,4R)-1-((S)-2-(tert-butyl)- 4,16-dioxo-16-(4-tosylpiperazin- 1-yl)-7,10,13-trioxa-3- azahexadecanoyl)-4-hydroxy-N- (4-(4-methylthiazol-5- yl)benzyl)pyrrolidine-2- carboxamide |
| 44 | IF-080 | 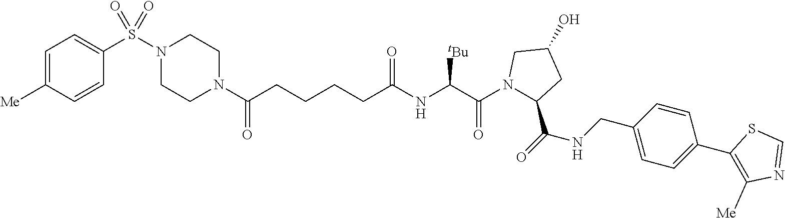 [SMILES: Cc1ccc(S(=O)(=O)N2CCN(C(=O)CCCCC(=O)N[C@H](C(=O)N3C[C@H](O)C[C@H]3C(=O)NCc3ccc(-c4scnc4C)cc3)C(C)(C)C)CC2)cc1] | (2S,4R)-1-((S)-3,3-dimethyl-2- (6-oxo-6-(4-tosylpiperazin-1- yl)hexanamido)butanoyl)-4- hydroxy-N-(4-(4-methylthiazol- 5-yl)benzyl)pyrrolidine-2- carboxamide |
| 45 | CMC-6- 109 | 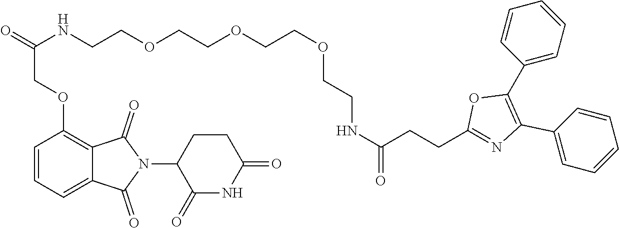 [SMILES: O=C(CCc1nc(-c2ccccc2)c(-c2ccccc2)o1)NCCOCCOCCOCCNC(=O)COc1cccc2c1C(=O)N(C1CCC(=O)NC1=O)C2=O] | N-(1-((2-(2,6-dioxopiperidin-3- yl)-1,3-dioxoisoindolin-4- yl)oxy)-2-oxo-6,9,12-trioxa-3- azatetradecan-14-yl)-3-(4,5- diphenyloxazol-2- yl)propanamide |
| 46 | CMC-6- 100 | 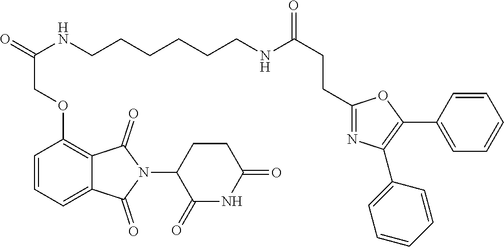 [SMILES: O=C(CCc1nc(-c2ccccc2)c(-c2ccccc2)o1)NCCCCCCNC(=O)COc1cccc2c1C(=O)N(C1CCC(=O)NC1=O)C2=O] | N-(6-(2-((2-(2,6-dioxopiperidin- 3-yl)-1,3-dioxoisoindolin-4-yl) oxy)acetamido)hexyl)-3-(4,5- diphenyloxazol-2-yl) propanamide |
| 47 | IF-072 | 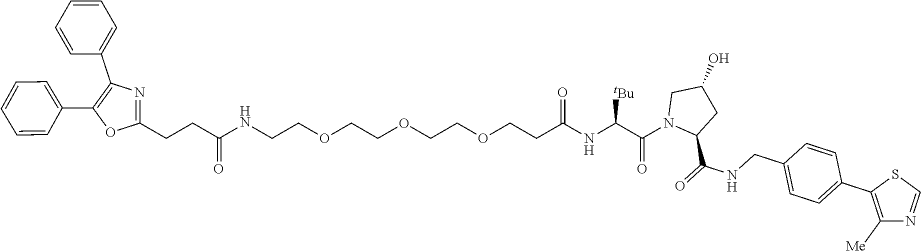 [SMILES: Cc1ncsc1-c1ccc(CNC(=O)[C@@H]2C[C@@H](O)CN2C(=O)[C@@H](NC(=O)CCOCCOCCOCCNC(=O)CCc2nc(-c3ccccc3)c(-c3ccccc3)o2)C(C)(C)C)cc1] | (2S,4R)-1-((S)-2-(tert-butyl)-19- (4,5-diphenyloxazol-2-yl)-4,17- dioxo-7,10,13-trioxa-3,16- diazanonadecanoyl)-4-hydroxy- N-(4-(4-methylthiazol-5-yl) benzyl)pyrrolidine-2- carboxamide |
| 48 | CMC-6- 111 | 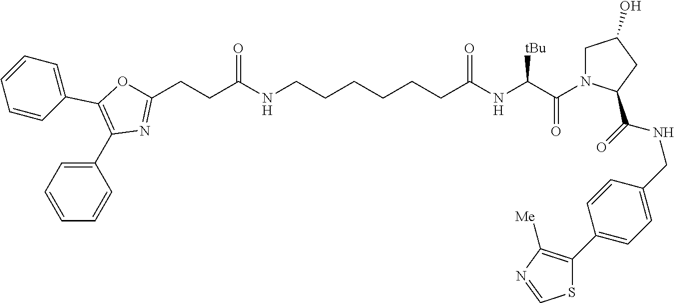 [SMILES: Cc1ncsc1-c1ccc(CNC(=O)[C@@H]2C[C@@H](O)CN2C(=O)[C@@H](NC(=O)CCCCCCNC(=O)CCc2nc(-c3ccccc3)c(-c3ccccc3)o2)C(C)(C)C)cc1] | (2S,4R)-1-((S)-2-(7-(3-(4,5- diphenyloxazol-2- yl)propanamido)heptanamido)- 3,3-dimethylbutanoyl)-4- hydroxy-N-(4-(4-methylthiazol- 5-yl)benzyl)pyrrolidine-2- carboxamide |
| 49 | CMC-6- 110 | 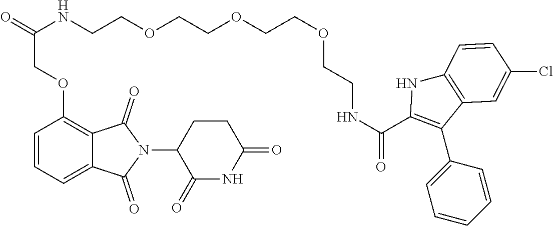 [SMILES: O=C(COc1cccc2c1C(=O)N(C1CCC(=O)NC1=O)C2=O)NCCOCCOCCOCCNC(=O)c1[nH]c2ccc(Cl)cc2c1-c1ccccc1] | 5-chloro-N-(1-((2-(2,6- dioxopiperidin-3-yl)-1,3- dioxoisoindolin-4-yl)oxy)-2- oxo-6,9,12-trioxa-3- azatetradecan-14-yl)-3-phenyl- 1H-indole-2-carboxamide |
| 50 | CMC-6- 102 | 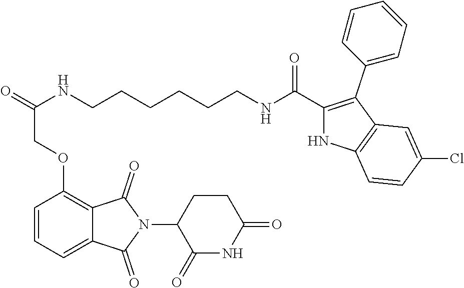 [SMILES: O=C(COc1cccc2c1C(=O)N(C1CCC(=O)NC1=O)C2=O)NCCCCCCNC(=O)c1[nH]c2ccc(Cl)cc2c1-c1ccccc1] | 5-chloro-N-(6-(2-((2-(2,6- dioxopiperidin-3-yl)-1,3- dioxoisoindolin-4-yl)oxy) acetamido)hexyl)-3-phenyl-1H- indole-2-carboxamide |
| 51 | IF-073 | 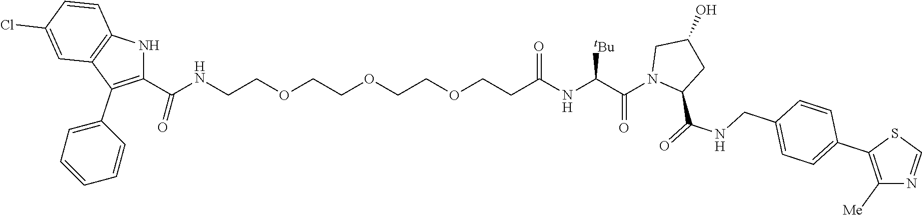 [SMILES: Cc1ncsc1-c1ccc(CNC(=O)[C@@H]2C[C@@H](O)CN2C(=O)[C@@H](NC(=O)CCOCCOCCOCCNC(=O)c2[nH]c3ccc(Cl)cc3c2-c2ccccc2)C(C)(C)C)cc1] | 5-chloro-N-((S)-14-((2S,4R)-4- hydroxy-2-((4-(4-methylthiazol- 5-yl)benzyl) carbamoyl)pyrrolidine-1- carbonyl)-15,15-dimethyl-12- oxo-3,6,9-trioxa-13- azahexadecyl)-3-phenyl-1H- indole-2-carboxamide |
| 52 | CMC-6- 112 | 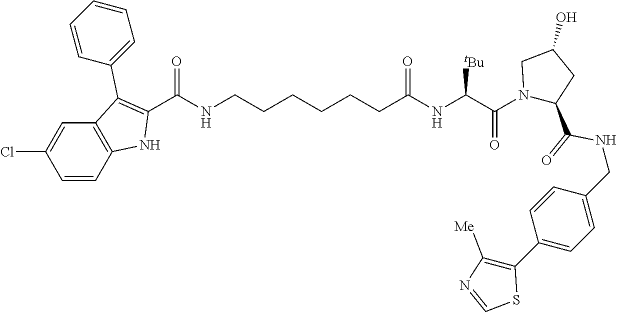 [SMILES: Cc1ncsc1-c1ccc(CNC(=O)[C@@H]2C[C@@H](O)CN2C(=O)[C@@H](NC(=O)CCCCCCNC(=O)c2[nH]c3ccc(Cl)cc3c2-c2ccccc2)C(C)(C)C)cc1] | 5-chloro-N-(7-(((S)-1-((2S,4R)- 4-hydroxy-2-((4-(4- methylthiazol-5-yl) benzyl) carbamoyl)pyrrolidin-1-yl)-3,3- dimethyl-1-oxobutan-2- yl)amino)-7-oxoheptyl)-3- phenyl-1H-indole-2- carboxamide |
| 53 | CMC-6- 27 | 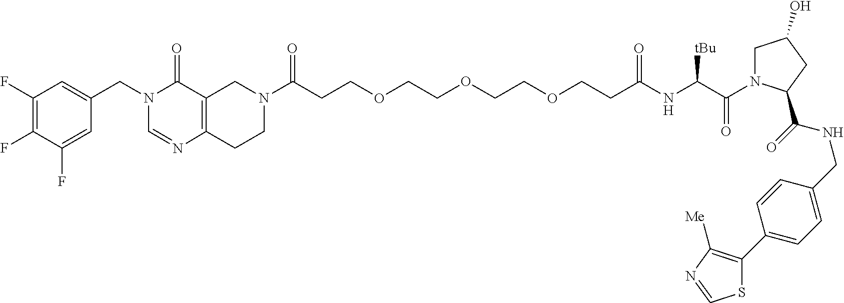 [SMILES: Cc1ncsc1-c1ccc(CNC(=O)[C@@H]2C[C@@H](O)CN2C(=O)[C@@H](NC(=O)CCOCCOCCOCCC(=O)N2CCc3ncn(Cc4cc(F)c(F)c(F)c4)c(=O)c3C2)C(C)(C)C)cc1] | (2S,4R)-1-((S)-2-(tert-butyl)- 4,16-dioxo-16-(4-oxo-3-(3,4,5- trifluorobenzyl)-3,5,7,8- tetrahydropyrido[4,3- d]pyrimidin-6(4H)-yl)-7,10,13- trioxa-3-azahexadecanoyl)-4- hydroxy-N-(4-(4-methylthiazol- 5-yl)benzyl)pyrrolidine-2- carboxamide |
| 54 | CMC-6- 35 | 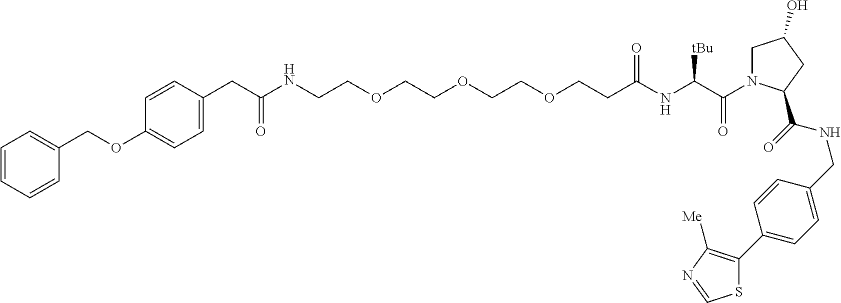 [SMILES: Cc1ncsc1-c1ccc(CNC(=O)[C@@H]2C[C@@H](O)CN2C(=O)[C@@H](NC(=O)CCOCCOCCOCCNC(=O)Cc2ccc(OCc3ccccc3)cc2)C(C)(C)C)cc1] | (2S,4R)-1-((S)-1-(4- (benzyloxy)phenyl)-17-(tert- butyl)-2,15-dioxo-6,9,12-trioxa- 3,16-diazaoctadecan-18-oyl)-4- hydroxy-N-(4-(4-methylthiazol- 5-yl)benzyl)pyrrolidine-2- carboxamide |
| 55 | CMC-6- 40 | 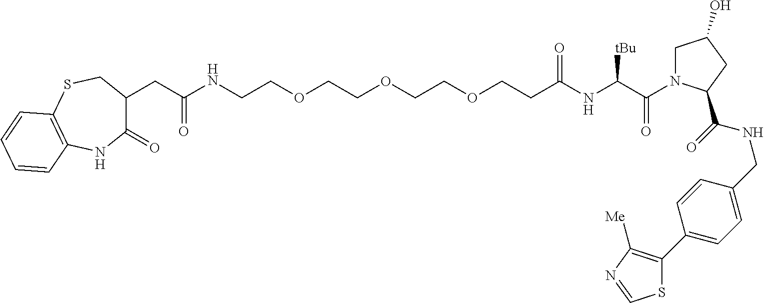 [SMILES: Cc1ncsc1-c1ccc(CNC(=O)[C@@H]2C[C@@H](O)CN2C(=O)[C@@H](NC(=O)CCOCCOCCOCCNC(=O)CC2CSc3ccccc3NC2=O)C(C)(C)C)cc1] | (2S,4R)-1-((17S)-17-(tert-butyl)- 2,15-dioxo-1-(4-oxo-2,3,4,5- tetrahydrobenzo[b][1,4] thiazepin- 3-yl)-6,9,12-trioxa-3,16- diazaoctadecan-18-oyl)-4- hydroxy-N-(4-(4-methylthiazol- 5-yl)benzyl)pyrrolidine-2- carboxamide |
| 56 | CMC-6- 46 | 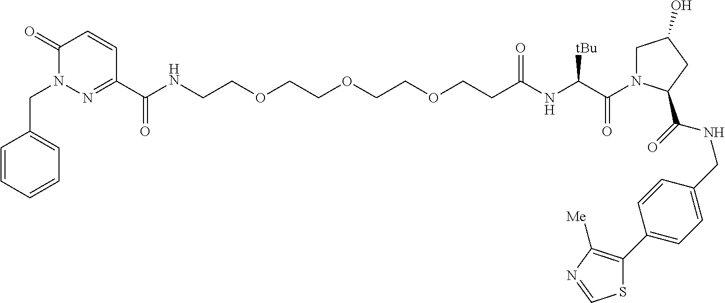 [SMILES: Cc1ncsc1-c1ccc(CNC(=O)[C@@H]2C[C@@H](O)CN2C(=O)[C@@H](NC(=O)CCOCCOCCOCCNC(=O)c2ccc(=O)n(Cc3ccccc3)n2)C(C)(C)C)cc1] | 1-benzyl-N-((S)-14-((2S,4R)-4- hydroxy-2-((4-(4-methylthiazol- 5-yl)benzyl) carbamoyl)pyrrolidine-1- carbonyl)-15,15-dimethyl-12- oxo-3,6,9-trioxa-13- azahexadecyl)-6-oxo-1,6- dihydropyridazine-3- carboxamide |
| 57 | CMC-6- 73 | 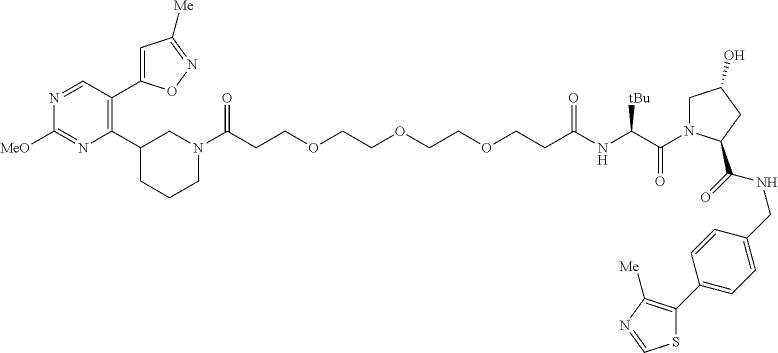 [SMILES: COc1ncc(-c2cc(C)no2)c(C2CCCN(C(=O)CCOCCOCCOCCC(=O)N[C@H](C(=O)N3C[C@H](O)C[C@H]3C(=O)NCc3ccc(-c4scnc4C)cc3)C(C)(C)C)C2)n1] | (2S,4R)-1-((2S)-2-(tert-butyl)- 16-(3-(2-methoxy-5-(3- methylisoxazol-5-yl) pyrimidin- 4-yl)piperidin-1-yl)-4,16-dioxo- 7,10,13-trioxa-3- azahexadecanoyl)-4-hydroxy-N- (4-(4-methylthiazol-5-yl) benzyl)pyrrolidine-2- carboxamide |
| 58 | CMC-6- 74 | 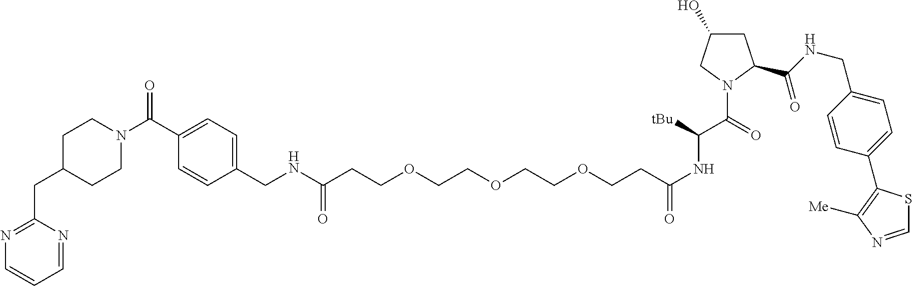 [SMILES: Cc1ncsc1-c1ccc(CNC(=O)[C@@H]2C[C@@H](O)CN2C(=O)[C@@H](NC(=O)CCOCCOCCOCCC(=O)NCc2ccc(C(=O)N3CCC(Cc4ncccn4)CC3)cc2)C(C)(C)C)cc1] | (2S,4R)-1-((S)-17-(tert-butyl)- 3,15-dioxo-1-(4-(4-(pyrimidin- 2-ylmethyl)piperidine-1- carbonyl)phenyl)-6,9,12-trioxa- 2,16-diazaoctadecan-18-oyl)-4- hydroxy-N-(4-(4-methylthiazol- 5-yl)benzyl)pyrrolidine-2- carboxamide |
| 59 | LPC004 | 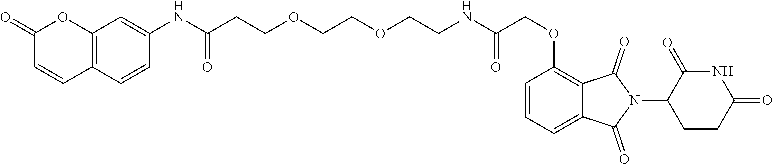 [SMILES: O=C(COc1cccc2c1C(=O)N(C1CCC(=O)NC1=O)C2=O)NCCOCCOCCC(=O)Nc1ccc2ccc(=O)oc2c1] | 3-(2-(2-(2-((2-(2,6- dioxopiperidin-3-yl)-1,3- dioxoisoindolin-4- yl)oxy)acetamido) ethoxy)ethoxy)-N-(2-oxo-2H- chromen-7-yl)propanamide |
| 60 | LPC019 | 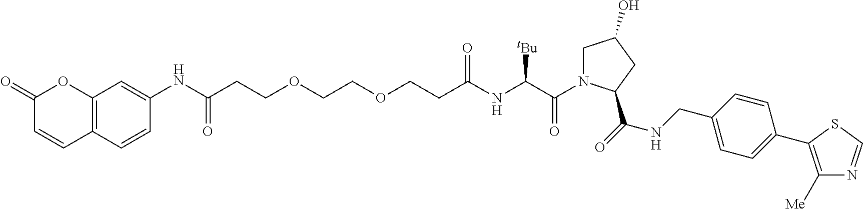 [SMILES: Cc1ncsc1-c1ccc(CNC(=O)[C@@H]2C[C@@H](O)CN2C(=O)[C@@H](NC(=O)CCOCCOCCC(=O)Nc2ccc3ccc(=O)oc3c2)C(C)(C)C)cc1] | (2S,4R)-1-((S)-3,3-dimethyl-2- (3-(2-(3-ox0-3-((2-oxo-2H- chromen-7-yl)amino) propoxy)ethoxy)propanamido) butanoyl)-4-hydroxy-N-(4-(4- methylthiazol-5- yl)benzyl)pyrrolidine-2- carboxamide |
| 61 | LPC006 | 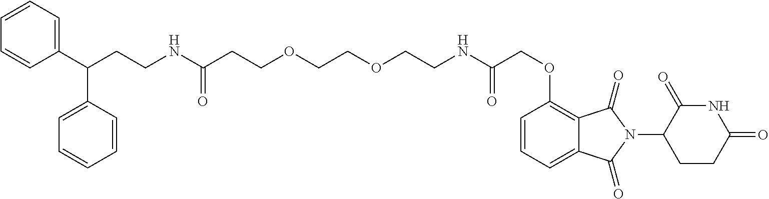 [SMILES: O=C(CCOCCOCCNC(=O)COc1cccc2c1C(=O)N(C1CCC(=O)NC1=O)C2=O)NCCC(c1ccccc1)c1ccccc1] | 3-(2-(2-(2-((2-(2,6- dioxopiperidin-3-yl)-1,3- dioxoisoindolin-4- yl)oxy)acetamido) ethoxy)ethoxy)-N-(3,3- diphenylpropyl)propanamide |
| 62 | LPC022 | 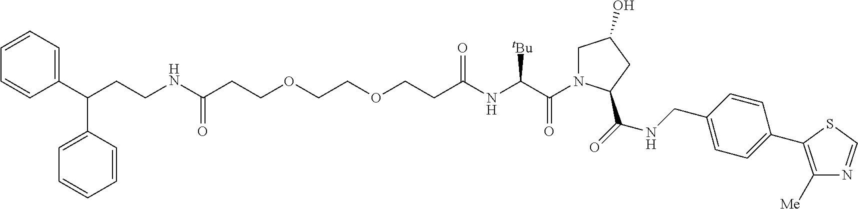 [SMILES: Cc1ncsc1-c1ccc(CNC(=O)[C@@H]2C[C@@H](O)CN2C(=O)[C@@H](NC(=O)CCOCCOCCC(=O)NCCC(c2ccccc2)c2ccccc2)C(C)(C)C)cc1] | (2S,4R)-1-((S)-2-(tert-butyl)- 4,13-dioxo-17,17-diphenyl-7,10- dioxa-3,14-diazaheptadecanoyl)- 4-hydroxy-N-(4-(4- methylthiazol-5- yl)benzyl)pyrrolidine-2- carboxamide |
| 63 | LPC015 | 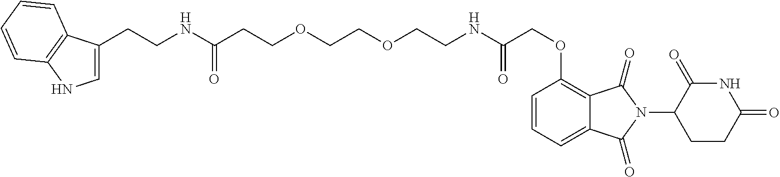 [SMILES: O=C(CCOCCOCCNC(=O)COc1cccc2c1C(=O)N(C1CCC(=O)NC1=O)C2=O)NCCc1c[nH]c2ccccc12] | N-(2-(1H-indol-3-yl)ethyl)-3-(2- (2-(2-((2-(2,6-dioxopiperidin-3- yl)-1,3-dioxoisoindolin-4- yl)oxy)acetamido) ethoxy)ethoxy)propanamide |
| 64 | LPC024 | 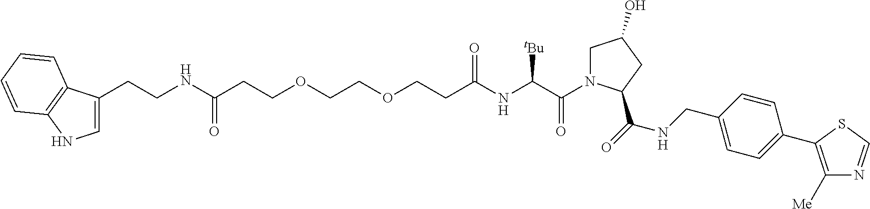 [SMILES: Cc1ncsc1-c1ccc(CNC(=O)[C@@H]2C[C@@H](O)CN2C(=O)[C@@H](NC(=O)CCOCCOCCC(=O)NCCc2c[nH]c3ccccc23)C(C)(C)C)cc1] | (2S,4R)-1-((S)-2-(tert-butyl)-16- (1H-indol-3-yl)-4,13-dioxo- 7,10-dioxa-3,14- diazahexadecanoyl)-4-hydroxy- N-(4-(4-methylthiazol-5- yl)benzyl)pyrrolidine-2- carboxamide |
| 65 | LPC033 | 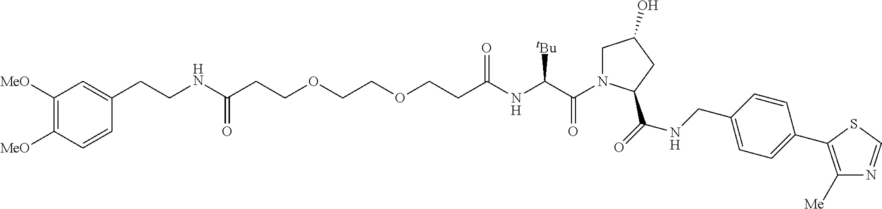 [SMILES: COc1ccc(CCNC(=O)CCOCCOCCC(=O)N[C@H](C(=O)N2C[C@H](O)C[C@H]2C(=O)NCc2ccc(-c3scnc3C)cc2)C(C)(C)C)cc1OC] | (2S,4R)-1-((S)-2-(tert-butyl)-16- (3,4-dimethoxyphenyl)-4,13- dioxo-7,10-dioxa-3,14- diazahexadecanoyl)-4-hydroxy- N-(4-(4-methylthiazol-5- yl)benzyl)pyrrolidine-2- carboxamide |
| 66 | LPC034 | 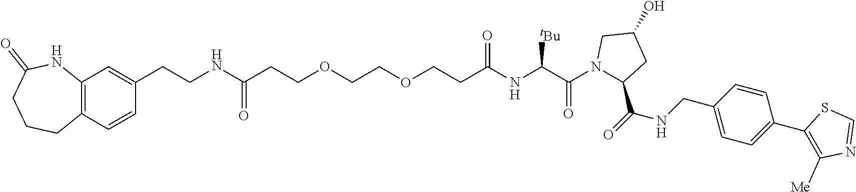 [SMILES: Cc1ncsc1-c1ccc(CNC(=O)[C@@H]2C[C@@H](O)CN2C(=O)[C@@H](NC(=O)CCOCCOCCC(=O)NCCc2ccc3c(c2)NC(=O)CCC3)C(C)(C)C)cc1] | (2S,4R)-1-((S)-2-(tert-butyl)- 4,13-dioxo-16-(2-oxo-2,3,4,5- tetrahydro-1H-benzo[b]azepin- 8-yl)-7,10-dioxa-3,14- diazahexadecanoyl)-4-hydroxy- N-(4-(4-methylthiazol-5- yl)benzyl)pyrrolidine-2- carboxamide |
| 67 | LPC036 | 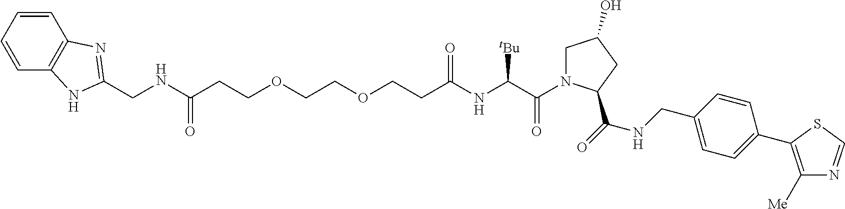 [SMILES: Cc1ncsc1-c1ccc(CNC(=O)[C@@H]2C[C@@H](O)CN2C(=O)[C@@H](NC(=O)CCOCCOCCC(=O)NCc2nc3ccccc3[nH]2)C(C)(C)C)cc1] | (2S,4R)-1-((S)-1-(1H- benzo[d]imidazol-2-yl)-14-(tert- butyl)-3,12-dioxo-6,9-dioxa- 2,13-diazapentadecan-15-oyl)-4- hydroxy-N-(4-(4-methylthiazol- 5-yl)benzyl)pyrrolidine-2- carboxamide |
| 68 | LPC042 | 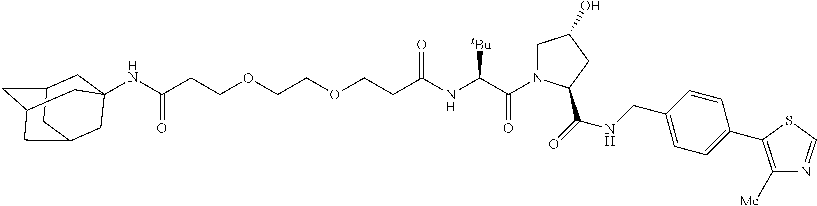 [SMILES: Cc1ncsc1-c1ccc(CNC(=O)[C@@H]2C[C@@H](O)CN2C(=O)[C@@H](NC(=O)CCOCCOCCC(=O)NC23CC4CC(CC(C4)C2)C3)C(C)(C)C)cc1] | (2S,4R)-1-((S)-2-(3-(2-(3- (((3s,5s,7s)-adamantan-1- yl)amino)-3-oxopropoxy) ethoxy)propanamido)-3,3- dimethylbutanoyl)-4-hydroxy- N-(4-(4-methylthiazol-5- yl)benzyl)pyrrolidine-2- carboxamide |
| 69 | LPC039 | 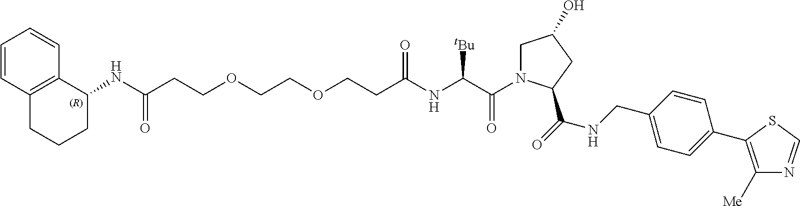 | (2S,4R)-1-((S)-3,3-dimethyl-2- (3-(2-(3-oxo-3-(((R)-1,2,3,4- tetrahydronaphthalen-1- yl)amino)propoxy)ethoxy) propanamido) butanoyl)-4-hydroxy- N-(4-(4-methylthiazol-5- yl)benzyl)pyrrolidine-2- carboxamide |
| 70 | LPC049 | 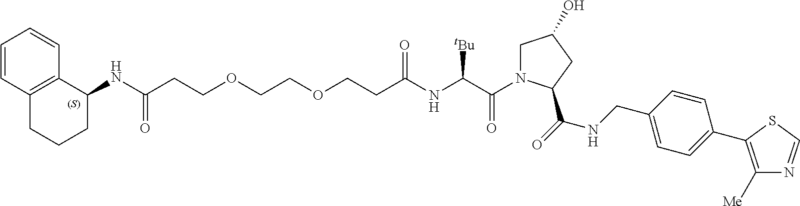 | (2S,4R)-1-((S)-3,3-dimethyl-2- (3-(2-(3-oxo-3-(((S)-1,2,3,4- tetrahydronaphthalen-1- yl)amino)propoxy)ethoxy) propanamido) butanoyl)-4-hydroxy-N- (4-(4-methylthiazol-5- yl)benzyl)pyrrolidine-2- carboxamide |
| 71 | CMC-6- 175 | 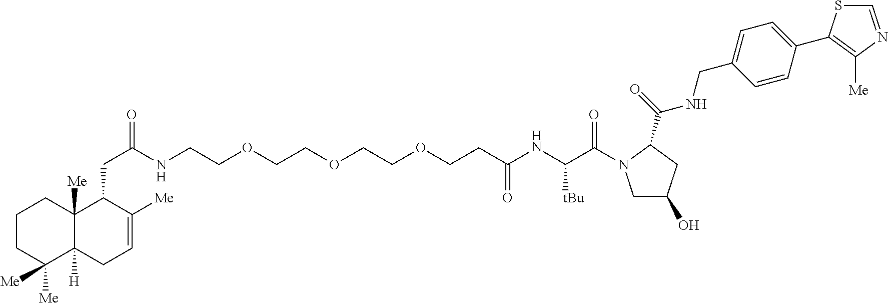 | (2S,4R)-1-((S)-16-(tert-butyl)- 1,14-dioxo-1-((1S,4S)-4,7,7- trimethyl-3-oxo-2- oxabicyclo[2.2.1]heptan-1-yl)- 5,8,11-trioxa-2,15- diazaheptadecan-17-oyl)-4- hydroxy-N-(4-(4-methylthiazol- 5-yl)benzyl)pyrrolidine-2- carboxamide |
| 72 | CMC-6- 182 | 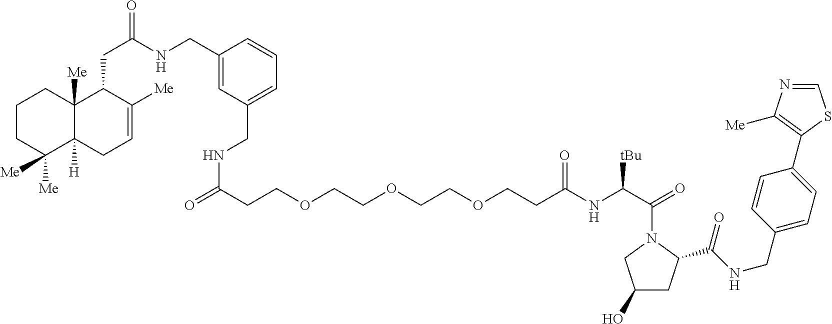 | (2S,4R)-1-((S)-17-(tert-butyl)- 3,15-dioxo-1-(3-((2- ((1R,4aS,8aS)-2,5,5,8a- tetramethyl-1,4,4a,5,6,7,8,8a- octahydronaphthalen-1- yl)acetamido)methyl)phenyl)- 6,9,12-trioxa-2,16- diazaoctadecan-18-oyl)-4- hydroxy-N-(4-(4-methylthiazol- 5-yl)benzyl)pyrrolidine-2- carboxamide |
| 73 | CMC-6- 177 | 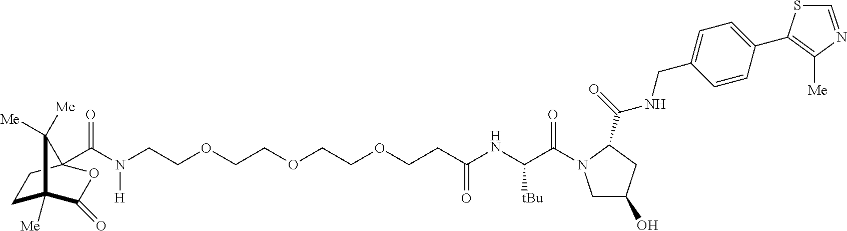 | (2S,4R)-1-((S)-16-(tert-butyl)- 1,14-dioxo-1-((1S,4S)-4,7,7- trimethyl-3-oxo-2- oxabicyclo[2.2.1]heptan-1-yl)- 5,8,11-trioxa-2,15- diazaheptadecan-17-oyl)-4- hydroxy-N-(4-(4-methylthiazol- 5-yl)benzyl)pyrrolidine-2- carboxamide |
| 74 | CMC-6- 181 | 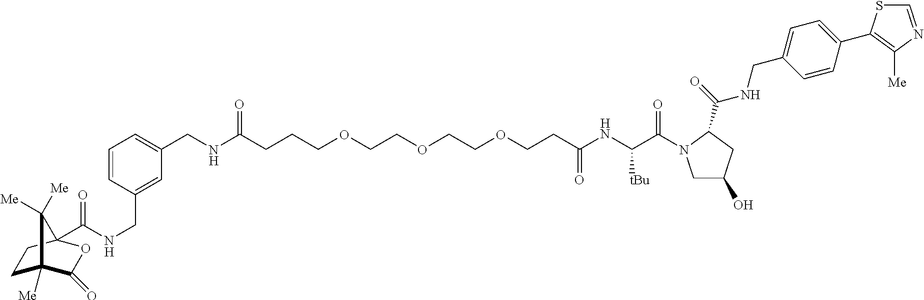 | (2S,4R)-1-((S)-18-(tert-butyl)- 3,16-dioxo-1-(3-(((1S,4S)-4,7,7- trimethyl-3-oxo-2- oxabicyclo[2.2.1]heptane-1- carboxamido)methyl)phenyl)- 7,10,13-trioxa-2,17- diazanonadecan-19-oyl)-4- hydroxy-N-(4-(4-methylthiazol- 5-yl)benzyl)pyrrolidine-2- carboxamide |
| 75 | IF-057 | 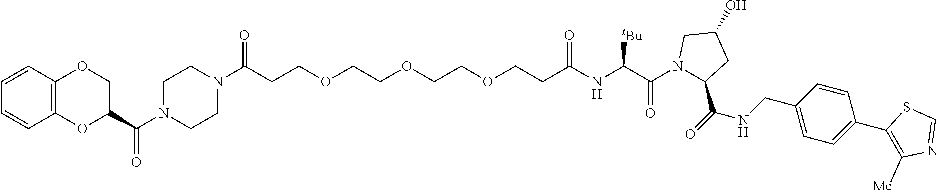 | (2S,4R)-1-((S)-2-(tert-butyl)-16- (4-((R)-2,3- dihydrobenzo[b][1,4]dioxine-2- carbonyl)piperazin-1-yl)-4,16- dioxo-7,10,13-trioxa-3- azahexadecanoyl)-4-hydroxy-N- (4-(4-methylthiazol-5- yl)benzyl)pyrrolidine-2- carboxamide |
| 76 | IF-059 | 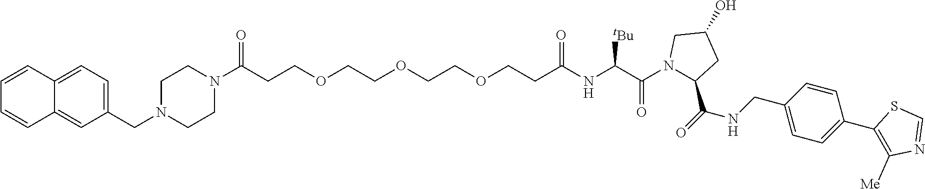 [SMILES: Cc1ncsc1-c1ccc(CNC(=O)[C@@H]2C[C@@H](O)CN2C(=O)[C@@H](NC(=O)CCOCCOCCOCCC(=O)N2CCN(Cc3ccc4ccccc4c3)CC2)C(C)(C)C)cc1] | (2S,4R)-1-((S)-2-(tert-butyl)-16- (4-(naphthalen-2- ylmethyl)piperazin-1-yl)-4,16- dioxo-7,10,13-trioxa-3- azahexadecanoyl)-4-hydroxy-N- (4-(4-methylthiazol-5- yl)benzyl)pyrrolidine-2- carboxamide |
| 77 | IF-060 | 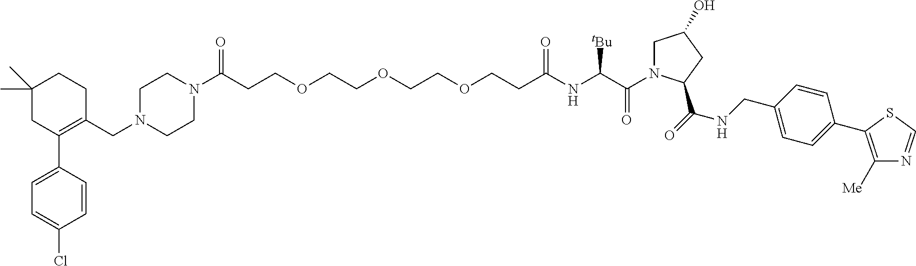 [SMILES: Cc1ncsc1-c1ccc(CNC(=O)[C@@H]2C[C@@H](O)CN2C(=O)[C@@H](NC(=O)CCOCCOCCOCCC(=O)N2CCN(CC3=C(c4ccc(Cl)cc4)CC(C)(C)CC3)CC2)C(C)(C)C)cc1] | (2S,4R)-1-((S)-2-(tert-butyl)-16- (4-((4′-chloro-5,5-dimethyl- 3,4,5,6-tetrahydro-[1,1′- biphenyl]-2- yl)methyl)piperazin-1-yl)-4,16- dioxo-7,10,13-trioxa-3- azahexadecanoyl)-4-hydroxy-N- (4-(4-methylthiazol-5- yl)benzyl)pyrrolidine-2- carboxamide |
| 78 | IF-062 | 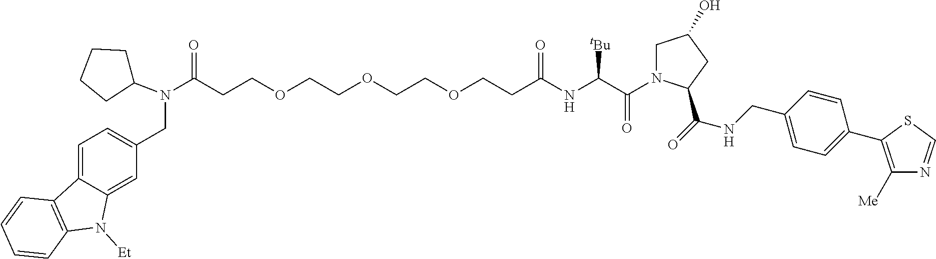 [SMILES: CCn1c2ccccc2c2ccc(CN(C(=O)CCOCCOCCOCCC(=O)N[C@H](C(=O)N3C[C@H](O)C[C@H]3C(=O)NCc3ccc(-c4scnc4C)cc3)C(C)(C)C)C3CCCC3)cc21] | (2S,4R)-1-((S)-17-(tert-butyl)-2- cyclopentyl-1-(9-ethyl-9H- carbazol-2-yl)-3,15-dioxo- 6,9,12-trioxa-2,16- diazaoctadecan-18-oyl)-4- hydroxy-N-(4-(4-methylthiazol- 5-yl)benzyl)pyrrolidine-2- carboxamide |
| 79 | IF-063 | 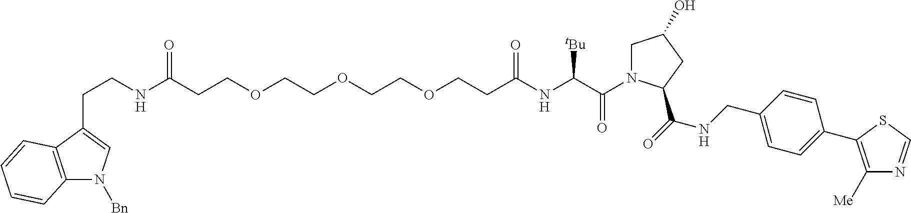 [SMILES: Cc1ncsc1-c1ccc(CNC(=O)[C@@H]2C[C@@H](O)CN2C(=O)[C@@H](NC(=O)CCOCCOCCOCCC(=O)NCCc2cn(Cc3ccccc3)c3ccccc23)C(C)(C)C)cc1] | (2S,4R)-1-((S)-19-(1-benzyl-1H- indol-3-yl)-2-(tert-butyl)-4,16- dioxo-7,10,13-trioxa-3,17- diazanonadecanoyl)-4-hydroxy- N-(4-(4-methylthiazol-5- yl)benzyl)pyrrolidine-2- carboxamide |
| 80 | IF-064 | 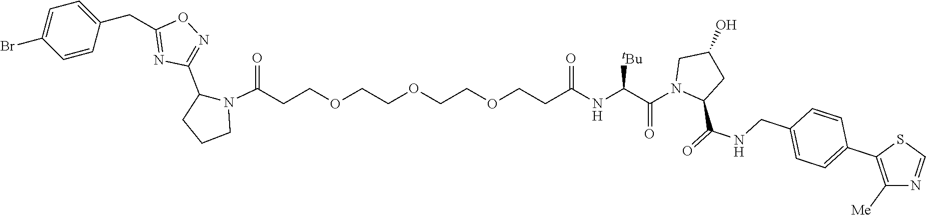 [SMILES: Cc1ncsc1-c1ccc(CNC(=O)[C@@H]2C[C@@H](O)CN2C(=O)[C@@H](NC(=O)CCOCCOCCOCCC(=O)N2CCCC2c2noc(Cc3ccc(Br)cc3)n2)C(C)(C)C)cc1] | (2S,4R)-1-((2S)-16-(2-(5-(4- bromobenzyl)-1,2,4-oxadiazol- 3-yl)pyrrolidin-1-yl)-2-(tert- butyl)-4,16-dioxo-7,10,13- trioxa-3-azahexadecanoyl)-4- hydroxy-N-(4-(4-methylthiazol- 5-yl)benzyl)pyrrolidine-2- carboxamide |
| 81 | IF-065 | 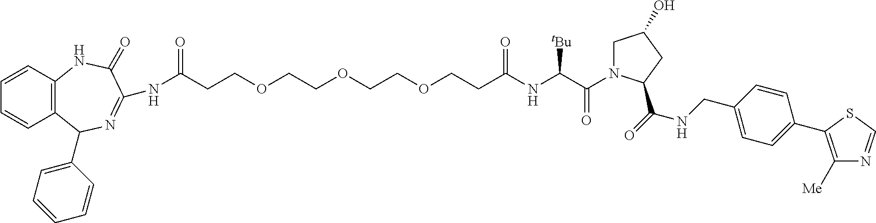 [SMILES: Cc1ncsc1-c1ccc(CNC(=O)[C@@H]2C[C@@H](O)CN2C(=O)[C@@H](NC(=O)CCOCCOCCOCCC(=O)NC2=NC(c3ccccc3)c3ccccc3NC2=O)C(C)(C)C)cc1] | (2S,4R)-1-((2S)-2-(tert-butyl)- 4,16-dioxo-16-((2-oxo-5- phenyl-2,5-dihydro-1H- benzo[e][1,4]diazepin-3- yl)amino)-7,10,13-trioxa-3- azahexadecanoyl)-4-hydroxy-N- (4-(4-methylthiazol-5-yl)benzyl) pyrrolidine-2-carboxamide |
| 82 | IF-066 | 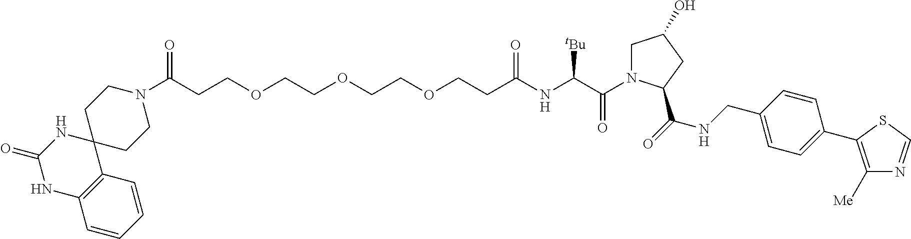 [SMILES: Cc1ncsc1-c1ccc(CNC(=O)[C@@H]2C[C@@H](O)CN2C(=O)[C@@H](NC(=O)CCOCCOCCOCCC(=O)N2CCC3(CC2)NC(=O)Nc2ccccc23)C(C)(C)C)cc1] | (2S,4R)-1-((S)-2-(tert-butyl)- 4,16-dioxo-16-(2′-oxo-2′,3′- dihydro-1′H-spiro[piperidine- 4,4′-quinazolin]-1-yl)-7,10,13- trioxa-3-azahexadecanoyl)-4- hydroxy-N-(4-(4-methylthiazol- 5-yl)benzyl)pyrrolidine-2- carboxamide |

The present invention is directed to designed bifunctional small molecules that modulate protein modifications via proximity-induced effects, such as ubiquitination-inducing small molecules. These represent a transformative therapeutic strategy. The strategy formulates FragTAC heterobifunctional compositions which integrate powerful chemical proteomic platforms with robust chemical biology tools to expedite the discovery of proteins amendable to these approaches as well as their corresponding bifunctional chemical probes. The techniques and chemical tools of the present invention provide a broad utility of powerful targeted protein degradation (TPD) methods. The new new E3 ligase systems and corresponding ligands that are capable of supporting TPD enable modification and treatment of such maladies as autoimmune disease and neoplastic disease both benign and malignant, and especially malignant such as cancer.

### REFERENCES

[1] C. G. Parker, A. Galmozzi, Y. Wang, B. E. Correia, K. Sasaki, C. M. Joslyn, A. S. Kim, C. L. Cavallaro, R. M. Lawrence, S. R. Johnson, I. Narvaiza, E. Saez, B. F. Cravatt, Cell 2017, 168, 527-541 e529.
[2] A. Galmozzi, B. P. Kok, A. S. Kim, J. R. Montenegro-Burke, J. Y. Lee, R. Spreafico, S. Mosure, V. Albert, R. Cintron-Colon, C. Godio, W. R. Webb, B. Conti, L. A. Solt, D. Kojetin, C. G. Parker, J. J. Peluso, J. K. Pru, G. Siuzdak, B. F. Cravatt, E. Saez, Nature 2019, 576, 138-+.
[3] Y. Wang, M. Dix, J. Remsberg, M. Kalocsay, S. Gygi, G. Vite, R. M. Lawrence, C. Parker, B. Cravatt, ChemRxiv 2019.
[4] B. K. Erickson, C. M. Rose, C. R. Braun, A. R. Erickson, J. Knott, G. C. McAlister, M. Wuhr, J. A. Paulo, R. A. Everley, S. P. Gygi, Mol Cell 2017, 65, 361-370.
[5] M. E. Welsch, S. A. Snyder, B. R. Stockwell, Curr Opin Chem Biol 2010, 14, 347-361.
[6] aT. W. Corson, N. Aberle, C. M. Crews, ACS Chem Biol 2008, 3, 677-692; bC. J. Gerry, S. L. Schreiber, Nat Chem Biol 2020, 16, 369-378.
[7] aG. E. Winter, D. L. Buckley, J. Paulk, J. M. Roberts, A. Souza, S. Dhe-Paganon, J. E. Bradner, Science 2015, 348, 1376-1381; bJ. S. Schneekloth, Jr., F. N. Fonseca, M. Koldobskiy, A. Mandal, R. Deshaies, K. Sakamoto, C. M. Crews, J Am Chem Soc 2004, 126, 3748-3754; cK. M. Sakamoto, K. B. Kim, A. Kumagai, F. Mercurio, C. M. Crews, R. J. Deshaies, Proc Natl Acad Sci USA 2001, 98, 8554-8559.
[8] aH. T. Huang, D. Dobrovolsky, J. Paulk, G. Yang, E. L. Weisberg, Z. M. Doctor, D. L. Buckley, J. H. Cho, E. Ko, J. Jang, K. Shi, H. G. Choi, J. D. Griffin, Y. Li, S. P. Treon, E. S. Fischer, J. E. Bradner, L. Tan, N. S. Gray, Cell Chem Biol 2018, 25, 88-99 e86; bD. P. Bondeson, B. E. Smith, G. M. Burslem, A. D. Buhimschi, J. Hines, S. Jaime-Figueroa, J. Wang, B. D. Hamman, A. Ishchenko, C. M. Crews, Cell Chem Biol 2018, 25, 78-87 e75.
[9] aR. Verma, D. Mohl, R. J. Deshaies, Mol Cell 2020, 77, 446-460; bP. P. Chamberlain, L. G. Hamann, Nat Chem Biol 2019, 15, 937-944; cA. C. Lai, C. M. Crews, Nat Rey Drug Discov 2017, 16, 101-114.
[10] T. Ito, H. Handa, Rinsho Ketsueki 2017, 58, 2067-2073.
[11] D. L. Buckley, K. Raina, N. Darricarrere, J. Hines, J. L. Gustafson, I. E. Smith, A. H. Miah, J. D. Harling, C. M. Crews, ACS Chem Biol 2015, 10, 1831-1837.
[12] J. N. Spradlin, X. R. Hu, C. C. Ward, S. M. Brittain, M. D. Jones, L. S. Ou, M. To, A. Proudfoot, E. Ornelas, M. Woldegiorgis, J. A. Olzmann, D. E. Bussiere, J. R. Thomas, J. A. Tallarico, J. M. McKenna, M. Schirle, T. J. Maimone, D. K. Nomura, Nature Chemical Biology 2019, 15, 747-+.
[13] X. Y. Zhang, V. M. Crowley, T. G. Wucherpfennig, M. M. Dix, B. F. Cravatt, Nature Chemical Biology 2019, 15, 737-+.
[14] R. J. Deshaies, C. A. Joazeiro, Annu Rev Biochem 2009, 78, 399-434.
[15] P. M. Cromm, C. M. Crews, Cell Chem Biol 2017, 24, 1181-1190.
[16] M. A. Erb, T. G. Scott, B. E. Li, H. Xie, J. Paulk, H. S. Seo, A. Souza, J. M. Roberts, S. Dastjerdi, D. L. Buckley, N. E. Sanjana, O. Shalem, B. Nabet, R. Zeid, N. K. Offei-Addo, S. Dhe-Paganon, F. Zhang, S. H. Orkin, G. E. Winter, J. E. Bradner, Nature 2017, 543, 270-274.

### SUMMARY STATEMENTS

The inventions, examples, biological assays and results described and claimed herein have may attributes and embodiments include, but not limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The term and/or means both as well as one or the other as in A and/or B means A alone, B alone and A and B together.

1. A heterobifunctional FragTAC composition comprising a compound of Formula I,
PBF-L-RBF (Formula I)
wherein PBF is a protein binding fragment of a hetero-organic molecule which is capable of binding an endogenous protein, L is an organic linker and RBF is a recruiter binding fragment of a hetero-organic molecule which is capable of binding a ligase of the polyubiquitin system.

2. A FragTAC composition according to claim 1, wherein the protein binding fragment is selected from a group consisting of

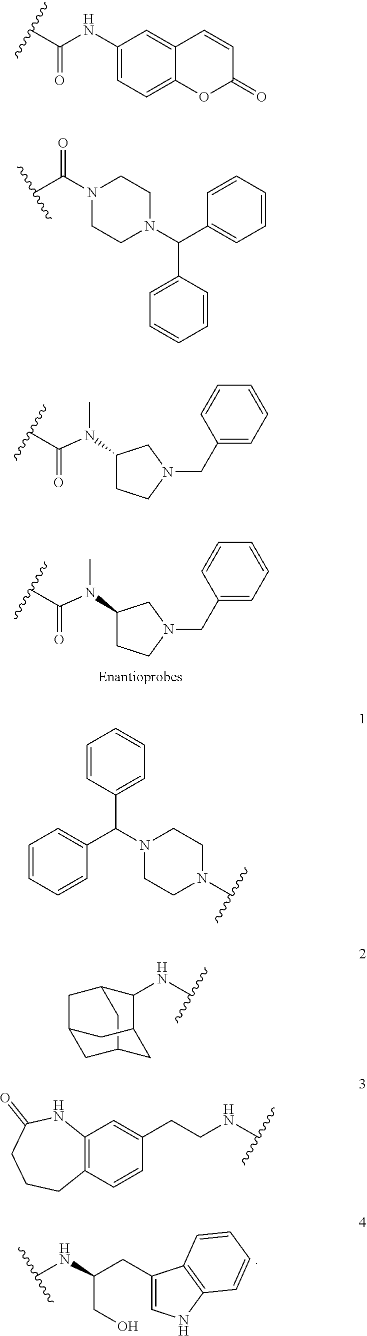

3. A FragTAC composition according to claim 1, where in the recruiter binding fragment is a fragment of thalidomide, a thalidomide derivative or a VHL ligand.

4. A FragTAC composition according to claim 1, wherein Formula I is a cereblon ligase binding molecule or a VHL ligase binding molecule of the Cereblon FragTAC structure or the VHL FragTAC structure wherein n is an integer of 1 to 10:

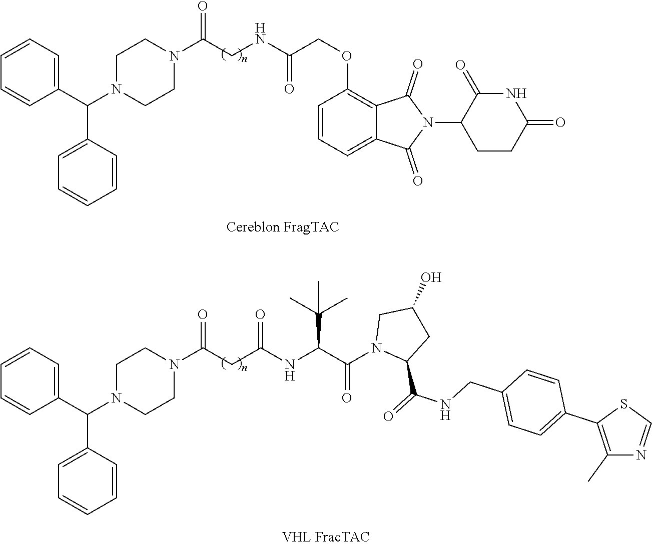

5. A composition according to claim 1, wherein the linker is a dimer, trimer, tetramer, or oligomer of an multiether, multi-PEG, multi-amide, or alkyl moiety.

6. A method for cleaving an endogenous protein comprising contacting the protein with a heterobifunctional composition of claim 1 and an E3 ligase.

7. A method according to claim 6, comprising conducting the contacting step in an aqueous medium.

8. A method according to claim 7, wherein the aqueous medium is cytoplasm.

9. A method according to claim 8, wherein the contacting is conducted in a viable cell.

10. A method according to claim 9, wherein the viable cell is a cell culture.

11. A method according to claim 9, wherein the viable cell is within a living organism.
